(19) United States
(12) Patent Application Publication
(10) Pub. No.: US 2017/0044104 A1
(43) Pub. Date: Feb. 16, 2017

(54) NEW PESTICIDAL COMPOUNDS AND USES

(71) Applicant: BAYER CROPSCIENCE AG, Monheim (DE)

(72) Inventors: Hans-Georg SCHWARZ, Dorsten (DE); Anne DECOR, Langenfeld (DE); Ulrich GOERGENS, Ratingen (DE); Kerstin ILG, Köln (DE); Martin FUESSLEIN, Düsseldorf (DE); Daniela PORTZ, Vettweiß (DE); Claudia WELZ, Duesseldorf (DE); Peter LUEMMEN, Idstein (DE); Kirsten BOERNGEN, Köln (DE); Adeline KOEHLER, Langenfeld (DE); Daniel KULKE, Leverkusen (DE)

(73) Assignee: Bayer Animal Health GmbH, Leverkusen (DE)

(21) Appl. No.: 15/039,138

(22) PCT Filed: Nov. 24, 2014
(86) PCT No.: PCT/EP14/75354

§ 371 Date: May 25, 2016

(30) Foreign Application Priority Data
Nov. 26, 2013 (EP) 13194361.5

(51) Int. Cl. C07D 213/64; C07D 213/40; C07D 213/61; C07D 213/75; C07D 401/12; C07D 307/52; A01N 43/08; C07D 231/12; A01N 43/56; C07D 277/28; A01N 43/78; C07D 241/24; A01N 43/60; C07D 213/71; C07D 213/85; C07D 239/42; A01N 43/54; C07D 239/30; C07D 239/26; C07D 405/12; C07D 213/73; A01N 43/40

(52) U.S. Cl. CPC C07D 213/64; A01N 43/40; C07D 213/40; C07D 213/61; C07D 213/75; C07D 401/12; C07D 307/52; A01N 43/08; C07D 231/12; A01N 43/56; C07D 277/28; A01N 43/78; C07D 241/24; A01N 43/60; C07D 213/71; C07D 213/85; C07D 239/42; A01N 43/54; C07D 239/30; C07D 239/26; C07D 405/12; C07D 213/73

(57) ABSTRACT

The invention relates to the use of known and novel phenalkyl carboxamide derivatives of the formula (I)

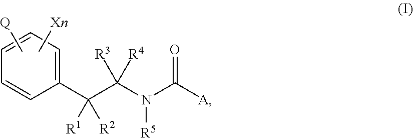

wherein the structural elements have the meaning as indicated in the description, for the control of nematodes and/or other helminths, particularly in agriculture and in the animal health field, formulations containing such compounds, particularly agrochemical formulations, and methods for the control of nematodes and helminths. The invention further relates to novel phenalkyl carboxamide derivatives, processes for their preparation, formulations containing such compounds, methods for the control of nematodes and helminths and their use as pest controlling agents, particularly their use as nematicides and their use as anthelmintics against endoparasites in animals and humans.

The present invention relates to the use of known and novel phenalkyl carboxamide derivatives for the control of nematodes and/or other helminths, particularly in agriculture and in the animal health field, formulations containing such compounds, particularly agrochemical formulations, and methods for the control of nematodes and helminths.

The present invention further relates to certain phenalkyl carboxamide derivatives as to processes for their preparation, to formulations comprising those compounds and their use in agriculture and veterinary fields and fields relying on pest management. The compounds are active for controlling plant damaging pests; they are particularly active for the control of nematodes. Furthermore, the compounds act as anthelmintic agents against endoparasites in animals and humans.

Nematodes cause a substantial loss in agricultural product including food and industrial crops and are combated with chemical compounds having nematicidal activity. These compounds should have high activity, broad spectrum activity against different strains of nematodes and should not be toxic to non-target organisms.

The occurrence of resistances against all commercial anthelmintics seems to be a growing problem in the area of veterinary medicine. Therefore, endoparasiticides with new molecular modes of actions are urgently desired. The new active ingredients should perform with excellent efficacy against a broad spectrum of helminths and nematodes without any adverse toxic effects to the treated vertebratic organism. Endoparasiticides are pharmaceuticals for combat or suppression of endoparasites in animals or humans.

The use of certain N-2-(pyridyl)ethyl-carboxamide derivatives for controlling nematodes is described in WO2007/108483 A1 and EP 2 132 987 A1.

The use of certain carboxamides as parasiticides is described in EP 1 997 800 A1, WO2012/118139 A1 WO2013/0676230 A1, WO2014/004064 A1, WO2014/034750 A1 and WO2014/034751 A1.

Furthermore, certain carboxamides are described as pesticides in WO2013/064518 A1, WO2013/064519 A1, WO2013/064520 A1, WO2013/064521 A1 or as nematicides in WO2013/064460 A1, WO2013/064461 A1 and WO2013/120940 A2.

Modern anthelmintic/nematicidal agents have to meet many demands, for example in relation to the level, duration and breadth of their action and possible use. Questions of toxicity and of combinability with other active ingredients or formulation auxiliaries play a role, as does the question of the expense that the synthesis of an active ingredient requires. In addition, resistances can occur. For all these reasons, the search for novel anthelmintic/nematicidal agents cannot be considered to be complete, and there is a constant need for novel compounds having properties which, compared to the known compounds, are improved at least in relation to individual aspects.

It was an object of the present invention to provide compounds which widen the spectrum of the pesticides in various respects. In particular, it was an object of the present invention to provide compounds which can be used as nematicides with a satisfactory or improved nematicidal activity, particularly at relatively low application rates, with a high selectivity and high compatibility in crop-plant cultures. Another particular object of the present invention was to provide compounds which can be used as endoparasiticides with a satisfactory or improved anthelmintic activity against a broad spectrum of helminths and nematodes, particularly at relatively low dosages, without any adverse toxic effects to the treated vertebratic organism.

The present invention relates to the use of a compound of formula (I)

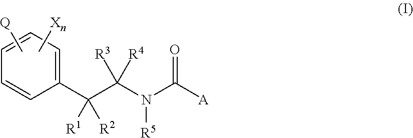

wherein (embodiment 1-1)
n is 0, 1, 2, 3 or 4, limited by the number of available positions in the ring to which a substituent X can be connected,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic or partially saturated or saturated, 5- or 6-membered heterocyclic ring containing one to four heteroatoms chosen from N, S, and O bearing the substituent Ym with
m is 0, 1, 2, 3 or 4, limited by the number of available positions in Q to which a substituent Y can be connected, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), —NH(C₁-C₈-alkylcarbonyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
R¹, R², R³ and R⁴ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R¹ and R² together with the carbon atom to which they are bonded form a 4-, 5- or 6-membered carbocycle and R³ and R⁴ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxy carbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R³ and R⁴ together with the carbon atom to which they are bonded form a 3-, 4-, 5- or 6-membered carbocycle and R¹ and R² are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R² and R⁴ together with the carbon atoms to which they are bonded form a 5- or 6-membered non-aromatic carbocycle optionally substituted by substituents selected from the group consisting of one to four C₁-C₈-alkyl groups and one to four halogen atoms, and R¹ and R³ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R¹ and R³ together with the carbon atoms to which they are bonded form a 5- or 6-membered non-aromatic carbocycle optionally substituted by substituents selected from the group consisting of one to four C₁-C₈-alkyl groups and one to four halogen atoms, and R² and R⁴ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl,
R⁵ is selected from the group consisting of hydrogen, cyano, —CHO, —OH, C₁-C₆-alkyl, C₁-C₆-halogenoalkyl having 1 to 5 halogen atoms, C₁-C₆-alkoxy, C₁-C₆-halogenoalkoxy having 1 to 5 halogen atoms, C₃-C₇-cycloalkyl, C₃-C₇-halogenocycloalkyl having 1 to 5 halogen atoms, C₃-C₇-cycloalkyl-C₁-C₆-alkyl, —CONH(C₁-C₆-alkyl), C₂-C₆-alkenyl, C₂-C₆-alkynyl, C₁-C₆-alkoxy-C₁-C₆-alkyl, C₃-C₇-cycloalkyl-C₁-C₆-alkyl, cyano-C₁-C₆-alkyl, amino-C₁-C₆-alkyl, C₁-C₆-alkylamino-C₁-C₆-alkyl, di-(C₁-C₆-alkyl)amino-C₁-C₆-alkyl, C₁-C₆-alkylcarbonyl, C₁-C₆-halogenoalkylcarbonyl having 1 to 5 halogen atoms, C₁-C₆-alkoxycarbonyl, C₁-C₆-benzyloxycarbonyl, C₁-C₆-alkoxy-C₁-C₆-alkylcarbonyl, —S—C₁-C₆-alkyl, —S—C₁-C₆-halogenoalkyl having 1 to 5 halogen atoms, —S(O)₂—C₁-C₆-alkyl, and —S(O)₂—C₁-C₆-halogenoalkyl having 1 to 5 halogen atoms,
A represents a phenyl group of the formula (A1)

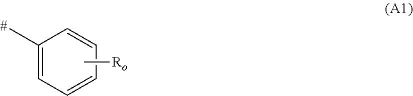

wherein
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1)

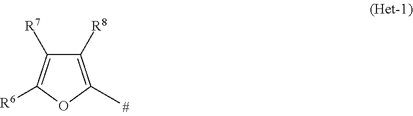

in which
R⁶ and R⁷ may be the same or different and are selected from the group consisting of hydrogen, halogen, amino, nitro, C₁-C₄-alkyl and C₁-C₄-halogenoalkyl having 1 to 5 halogen atoms, and
R⁸ is selected from the group consisting of hydrogen, halogen, nitro, C₁-C₄-alkyl and C₁-C₄-halogenoalkyl having 1 to 5 halogen atoms, or
A represents a heterocycle of the formula (Het-2)

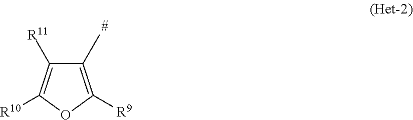

in which
R⁹ is selected from the group consisting of hydrogen, halogen, C₁-C₄-alkyl and C₁-C₄-halogenoalkyl having 1 to 5 halogen atoms, and
R¹⁰ and R¹¹ may be the same or different and are selected from the group consisting of hydrogen, halogen, amino, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-3)

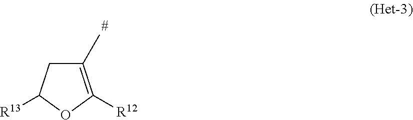

in which
R¹² is selected from the group consisting of halogen, C₁-C₄-alkyl and C₁-C₄-halogenoalkyl having 1 to 5 halogen atoms, and
R¹³ is selected from the group consisting of hydrogen, C₁-C₄-alkyl and C₁-C₄-halogenoalkyl having 1 to 5 halogen atoms, or
A represents a heterocycle of the formula (Het-4)

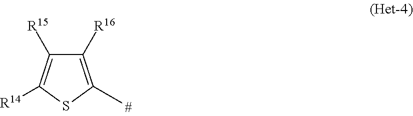

in which
R¹⁴ and R¹⁵ may be the same or different and are selected from the group consisting of hydrogen, halogen, C₁-C₄-alkyl, C₁-C₄-halogenoalkyl having 1 to 5 halogen atoms, —S—C₁-C₄-alkyl, —S(O)₂—C₁-C₄-alkyl, phenyl (optionally substituted by halogen or C₁-C₄-alkyl) and pyridyl (optionally substituted by halogen or C₁-C₄-alkyl), and
R¹⁶ is selected from the group consisting of hydrogen, halogen, cyano, C₁-C₄-alkyl, C₁-C₄-halogenoalkyl having 1 to 5 halogen atoms and C₁-C₄-halogenoalkoxy having 1 to 5 halogen atoms, or
A represents a heterocycle of the formula (Het-5)

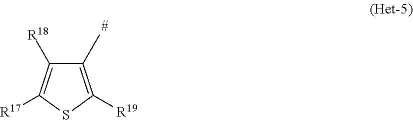

in which
R¹⁷ and R¹⁸ may be the same or different and are selected from the group consisting of hydrogen, halogen, C₁-C₄-alkyl, C₁-C₄-alkyloxy and C₁-C₄-halogenoalkyl having 1 to 5 halogen atoms, and
R¹⁹ is selected from the group consisting of hydrogen, halogen, C₁-C₄-alkyl and C₁-C₄-halogenoalkyl having 1 to 5 atoms, or
A represents a heterocycle of the formula (Het-6)

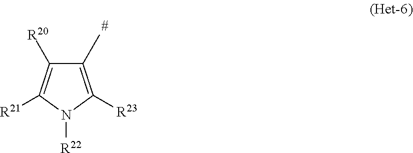

in which
R²⁰ is selected from the group consisting of hydrogen, halogen, cyano, C₁-C₄-alkyl and C₁-C₄-halogenoalkyl having 1 to 5 halogen atoms, and
R²¹ and R²³ may be the same or different and are selected from the group consisting of hydrogen, halogen, C₁-C₄-alkyl and C₁-C₄-halogenoalky having 1 to 5 halogen atoms, and
R²² is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, hydroxyl-C₁-C₄-alkyl, —S(O)₂—C₁-C₄-alkyl, —S(O)₂—N(C₁-C₄-alkyl)₂, C₁-C₆-alkylcarbonyl, —S(O)₂-phenyl (optionally substituted by halogen or C₁-C₄-alkyl) and benzoyl (optionally substituted by halogen or C₁-C₄-alkyl), or
A represents a heterocycle of the formula (Het-7)

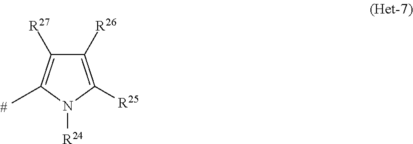

in which
R²⁴ is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, hydroxy-C₁-C₄-alkyl, —S(O)₂—C₁-C₄-alkyl, —S(O)₂—N(C₁-C₄-alkyl)₂, C₁-C₆-alkylcarbonyl, —S(O)₂-phenyl (optionally substituted by halogen or C₁-C₄-alkyl) and benzoyl (optionally substituted by halogen or a C₁-C₄-alkyl), and
R²⁵, R²⁶ and R²⁷ may be the same or different and are selected from the group consisting of hydrogen, halogen, cyano, C₁-C₄-alkyl, C₁-C₄-halogenalkyl having 1 to 5 halogen atoms and C₁-C₄-alkylcarbonyl, or
A represents a heterocycle of the formula (Het-8)

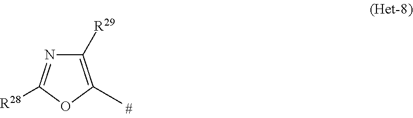

in which
R²⁸ is selected from the group consisting of hydrogen and C₁-C₄-alkyl, and
R²⁹ is selected from the group consisting of halogen, C₁-C₄-alkyl and C₁-C₄-halogenoalkyl having 1 to 5 halogen atoms, or
A represents a heterocycle of the formula (Het-9)

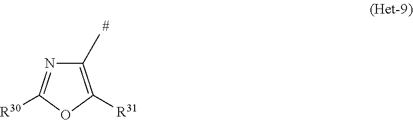

in which
R³⁰ is selected from the group consisting of hydrogen and C₁-C₄-alkyl, and
R³³ is selected from the group consisting of halogen, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-10)

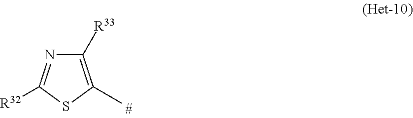

in which
R³² is selected from the group consisting of hydrogen, halogen, amino, cyano, C₁-C₄-alkylamino, di-(C₁-C₄-alkyl)amino, C₁-C₄-alkyl, C₁-C₄-halogenoalkyl having 1 to 5 halogen atoms and phenyl (optionally substituted by halogen or C₁-C₄-alkyl), and
R³³ is selected from the group consisting of halogen, C₁-C₄-alkyl and C₁-C₄-halogenoalkyl having 1 to 5 halogen atoms, C₁-C₅-halogenoalkoxy having 1 to 9 halogen atoms, amino, substituted or unsubstituted C₁-C₅-alkylamino or substituted or unsubstituted di-(C₁-C₅-alkyl)-amino, or
A represents a heterocycle of the formula (Het-11)

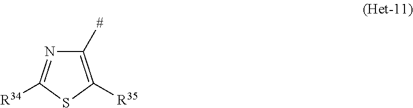

in which
R³⁴ is selected from the group consisting of hydrogen, halogen, amino, cyano, C₁-C₄-alkylamino, di-(C₁-C₄-alkyl)amino, C₁-C₄-alkyl and C₁-C₄-halogenoalkyl having 1 to 5 halogen atoms, and
R³⁵ is selected from the group consisting of halogen, C₁-C₄-alkyl and C₁-C₄-halogenoalkyl having 1 to 5 halogen atoms, or
A represents a heterocycle of the formula (Het-12)

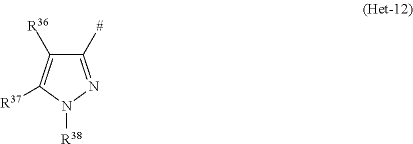

in which
R³⁶ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, —S(O)—C₁-C₄-alkyl, —S(O)₂—C₁-C₄-alkyl, —S—C₁-C₄-halogenoalkyl having 1 to 5 halogen atoms, aminocarbonyl and aminocarbonyl-C₁-C₄-alkyl, and
R³⁷ is selected from the group consisting of hydrogen, halogen, cyano, nitro, C₁-C₄-alkyl, C₁-C₄-alkoxy, —S—C₁-C₄-alkyl, —S(O)—C₁-C₄-alkyl, and —S(O)₂—C₁-C₄-alkyl, and
R³⁸ is selected from the group consisting of hydrogen, phenyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-13)

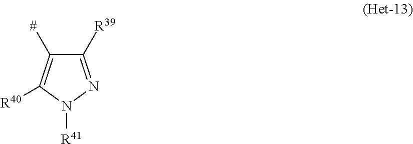

in which
R³⁹ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, S(O)—C₁-C₄-alkyl, —S(O)₂—C₁-C₄-alkyl, —S—C₁-C₄-halogenoalkyl having 1 to 5 atoms, aminocarbonyl and aminocarbonyl-C₁-C₄-alkyl, and
R⁴⁰ is selected from the group consisting of hydrogen, halogen, cyano, C₁-C₄-alkyl, C₁-C₄-alkoxy, C₁-C₄-halogenoalkoxy having 1 to 5 halogen atoms, —S—C₁-C₄-alkylS(O)—C₁-C₄-alkyl, and —S(O)₂—C₁-C₄-alkyl, and
R⁴¹ is selected from the group consisting of hydrogen, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl, C₁-C₄-halogenoalkoxy-C₁-C₄-alkyl having 1 to 5 halogen atoms and phenyl (optionally substituted by halogen, C₁-C₄-alkyl, C₁-C₄-alkoxy-C₁-C₄-alkyl or nitro), or
A represents a heterocycle of the formula (Het-14)

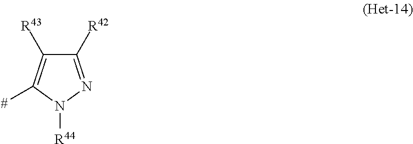

in which
R⁴² is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, S(O)—C₁-C₄-alkyl, —S(O)₂—C₁-C₄-alkyl, —S—C₁-C₄-halogenoalkyl having 1 to 5 halogen atoms, aminocarbonyl and aminocarbonyl-C₁-C₄-alkyl, and
R⁴³ is selected from the group consisting of hydrogen, halogen, cyano, C₁-C₄-alkyl, C₁-C₄-alkoxy, —S—C₁-C₄-alkyl, S(O)—C₁-C₄-alkyl, —S(O)₂—C₁-C₄-alkyl, and C₁-C₄-halogenoalkyl having 1 to 5 halogen atoms, and
R⁴⁴ is selected from the group consisting of hydrogen, phenyl, benzyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-15)

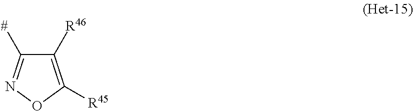

in which
R⁴⁵ and R⁴⁶ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-16)

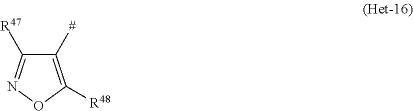

in which
R⁴⁷ and R⁴⁸ may be the same or different and are selected from the group consisting of hydrogen, halogen, C₁-C₄-alkyl, C₁-C₄-halogenoalkyl having 1 to 5 halogen atoms, phenyl (optionally substituted by halogen or a C₁-C₄-alkyl), and heterocyclyl like pyridyl, pyrimidinyl and thiadiazolyl (each optionally substituted by halogen or C₁-C₄-alkyl), or
A represents a heterocycle of the formula (Het-17)

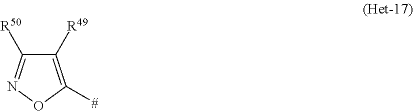

in which
R⁴⁹ and R⁵⁰ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-18)

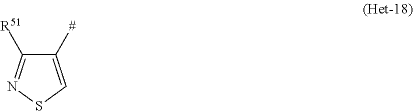

in which
R⁵¹ is selected from the group consisting of halogen, C₁-C₄-alkyl and C₁-C₄-halogenoalkyl having 1 to 5 halogen atoms, or
A represents a heterocycle of the formula (Het-19)

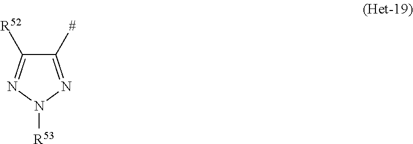

in which

R⁵² is selected from the group consisting of halogen, C₁-C₄-alkyl and C₁-C₄-halogenoalkyl having 1 to 5 halogen atoms, and
R⁵³ is selected from the group consisting of hydrogen, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-20)

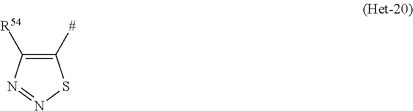

in which
R⁵⁴ is selected from the group consisting of halogen, C₁-C₄-alkyl and C₁-C₄-halogenoalkyl having 1 to 5 halogen atoms, or
A represents a heterocycle of the formula (Het-21)

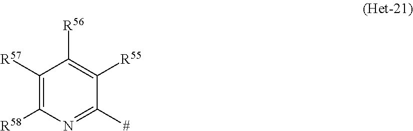

in which
R⁵⁵ is selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, S(O)—C₁-C₄-alkyl, —S(O)₂—C₁-C₄-alkyl, —S—C₁-C₄-halogenoalkyl having 1 to 5 halogen atoms and C₁-C₄-halogenoalkoxy having 1 to 5 halogen atoms, and
R⁵⁶, R⁵⁷ and R⁵⁸, which may be the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-22)

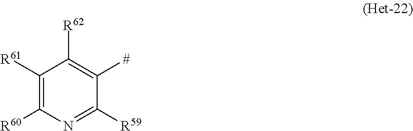

in which
R⁵⁹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and S-phenyl (optionally substituted by halogen or C₁-C₄-alkyl), and
R⁶⁰, R⁶¹ and R⁶², which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, N-morpholine optionally substituted by halogen or C₁-C₄-alkyl, and thienyl (optionally substituted by halogen or a C₁-C₄-alkyl), or
A represents a heterocycle of the formula (Het-23)

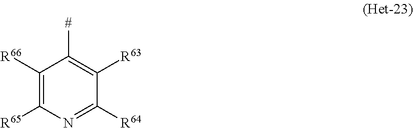

in which
R⁶³, R⁶⁴, R⁶⁵ and R⁶⁶, which may be the same or different, are selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, —S—C₁-C₄-halogenoalkyl having 1 to 5 halogen atoms, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-24)

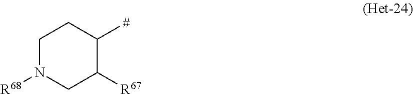

in which
R⁶⁷ is selected from the group consisting of hydrogen, halogen, C₁-C₄-alkyl and C₁-C₄-halogenoalkyl having 1 to 5 halogen atoms, and
R⁶⁸ is selected from the group consisting of hydrogen, C₁-C₄-alkyl, C₁-C₄-halogenoalkyl having 1 to 5 halogen atoms, C₁-C₆-alkoxycarbonyl, benzyl (optionally substituted by 1 to 3 halogen atoms), benzyloxycarbonyl (optionally substituted by 1 to 3 halogen atoms), and heterocyclyl like pyridyl and pyrimidinyl (each optionally substituted by halogen, C₁-C₄-alkyl, C₁-C₄-halogenoalkyl having 1 to 5 halogen atoms), or
A represents a heterocycle of the formula (Het-25)

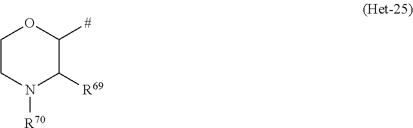

in which
R⁶⁹ is selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, —S—C₁-C₄-halogenoalkyl having 1 to 5 halogen atoms and C₁-C₄-halogenoalkoxy having 1 to 5 halogen atoms, and
R⁷⁰ is selected from the group consisting of hydrogen, C₁-C₄-alkyl, C₁-C₄-halogenoalkyl having 1 to 5 halogen atoms, and benzyl, or
A represents a heterocycle of the formula (Het-26)

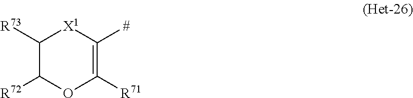

in which
X¹ is selected from the group consisting of sulphur, —SO—, —SO₂— and CH₂—, and
R⁷¹ is selected from the group consisting of C₁-C₄-alkyl and C₁-C₄-halogenoalkyl having 1 to 5 halogen atoms, and
R⁷² and R⁷³ may be the same or different and are selected from the group consisting of hydrogen and C₁-C₄-alkyl, or
A represents a heterocycle of the formula (Het-27)

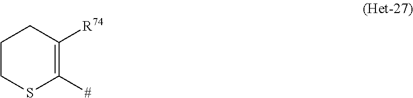

in which
R⁷⁴ is selected from the group consisting of C₁-C₄-alkyl and C₁-C₄-halogenoalkyl having 1 to 5 halogen atoms, or
A represents a heterocycle of the formula (Het-28)

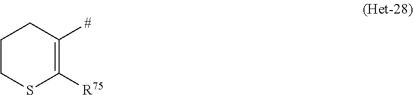

in which
R⁷⁵ is selected from the group consisting of C₁-C₄-alkyl and C₁-C₄-halogenoalkyl having 1 to 5 halogen atoms, or
A represents a heterocycle of the formula (Het-29)

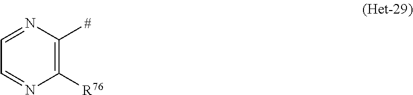

in which
R⁷⁶ is selected from the group consisting of hydrogen, halogen, C₁-C₄-alkyl and C₁-C₄-halogenoalkyl having 1 to 5 halogen atoms,
   for controlling nematodes and/or other helminths.

In formulae (Het-1) to (Het-29) # depicts the bond which connects A to the C(O)NR⁵-moiety in the compounds of formula (I). In general, in the present application # depicts the connecting bond of the structural element, unless otherwise indicated.

Any of the compounds according to the invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

The invention also relates to salts, N-oxides, metal complexes and metalloid complexes of compounds of formula (I) and the uses thereof.

Compounds of the present invention can also exist in one or more geometric isomer forms depending on the number of double bonds in the compound, especially all syn/anti (or cis/trans) isomers and to all possible syn/anti (or cis/trans) mixtures. The invention thus relates equally to all geometric isomers and to all possible mixtures, in all proportions. The geometric isomers can be separated according to general methods, which are known per se by the man ordinary skilled in the art.

Compounds of formula (I) may be found in its tautomeric form resulting from the shift of the proton of a hydroxy, sulfanyl or amino group. Such tautomeric forms of such compounds are also part of the present invention. More generally speaking, all tautomeric forms of compounds of formula (I), as well as the tautomeric forms of the compounds which can optionally be used as intermediates in the preparation processes and which will be defined in the description of these processes, are also part of the present invention.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than 10 one double bond such as 1,3- and 1,4-cyclohexadienyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, C₁CH₂, CF₃CH₂ and CF₃CCl₂. The terms “haloalkoxy”, “haloalkenyl”, “haloalkynyl”, and the like, are defined analogously to the term “haloalkyl”. Examples of “haloalkoxy” include CF₃O, CCl₃CH₂O, HCF₂CH₂CH₂O and CF₃CH₂O. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂.

The chemical abbreviation C(O) as used herein represents a carbonyl moiety. For example, C(O)CH₃ represents an acetyl group. The chemical abbreviations CO₂ and C(O)O as used herein represent an ester moiety. For example, CO₂Me and C(O)OMe represent a methyl ester. CHO represents an aldehyde moiety.

“OCN” means —O—C≡N, and “SCN” means —S—C≡N.

The total number of carbon atoms in a substituent group is indicated by the “Ci-Cj” prefix where i and j are numbers from 1 to 14. C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, e.g. n=0, 1, 2, 3 or 4. When a group contains a substituent which can be hydrogen, for example R² or R³, then when this substituent is taken as hydrogen, it is recognized that this is equivalent to said group being unsubstituted.

Unless otherwise indicated, a “ring” or “ring system” as a component of formula (I) is carbocyclic or heterocyclic. The term “ring system” denotes two or more fused rings. The term “heterocyclic ring” denotes a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. The term “heterocyclic ring system” denotes a ring system in which at least one ring of the ring system is a heterocyclic ring. Unless otherwise indicated, heterocyclic rings and ring systems can be attached through any available carbon or nitrogen by replacement of a hydrogen on said carbon or nitrogen.

As used herein, the following definitions shall apply unless otherwise indicated. The term “optionally substituted” is used interchangeably with the phrase “substituted or unsubstituted” or with the term “(un)substituted”. The expression “optionally substituted with 1 to 4 substituents” means that no substituent is present (i.e. unsubstituted) or that 1, 2, 3 or 4 substituents are present (limited by the number of available bonding positions). Unless otherwise indicated, an optionally substituted group may have a substituent at each substitutable position of the group, and each substitution is independent of the other.

In an individual embodiment (embodiment 1-2), the structural elements in the compound of formula (I) are defined as follows:
n is as defined in embodiment 1-1,
each X is as defined in embodiment 1-1,
Q represents an aromatic or partially saturated or saturated, 5- or 6-membered heterocyclic ring containing one to four heteroatoms chosen from N, S, and O bearing the substituent Ym with
m is 0, 1, 2, 3 or 4, limited by the number of available positions in Q to which a substituent Y can be connected, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), —NH(C₁-C₈-alkylcarbonyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
R¹, R², R³ and R⁴ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl,
   with the proviso that R¹ is fluorine and/or R² is fluorine,
R⁵ is as defined in embodiment 1-1, and
A is as defined in embodiment 1-1 with the proviso that for Het-21, R⁵⁵ is not CF₃.

In another individual aspect of embodiment 1-1, R¹ is fluorine. In another individual aspect of embodiment 1-1, R² is fluorine. In another individual aspect of embodiment 1-1, R¹ is fluorine and R² is fluorine. In another individual aspect of embodiment 1-1, the combination R¹/R² is fluorine/methyl. In another individual aspect of embodiment 1-1, the combination R¹/R² is fluorine/hydrogen.

In another individual aspect of embodiment 1-2, R¹ is fluorine. In another individual aspect of embodiment 1-2, R² is fluorine. In another individual aspect of embodiment 1-2, R¹ is fluorine and R² is fluorine. In another individual aspect of embodiment 1-2, the combination R¹/R² is fluorine/methyl. In another individual aspect of embodiment 1-2, the combination R¹/R² is fluorine/hydrogen.

In embodiments 1-1 and 1-2 as well as in each individual aspect of said embodiments, Q preferably represents an optionally mono- or polysubstituted heteroaromatic ring from the group consisting of Q-1 to Q-64 (embodiments 1-1a and 1-2a):

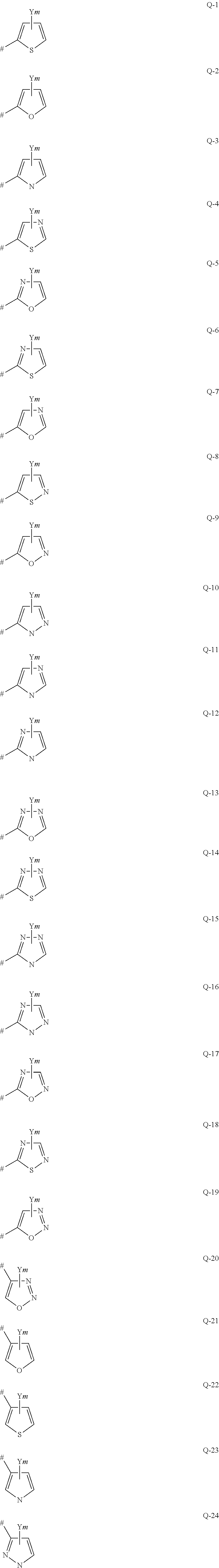

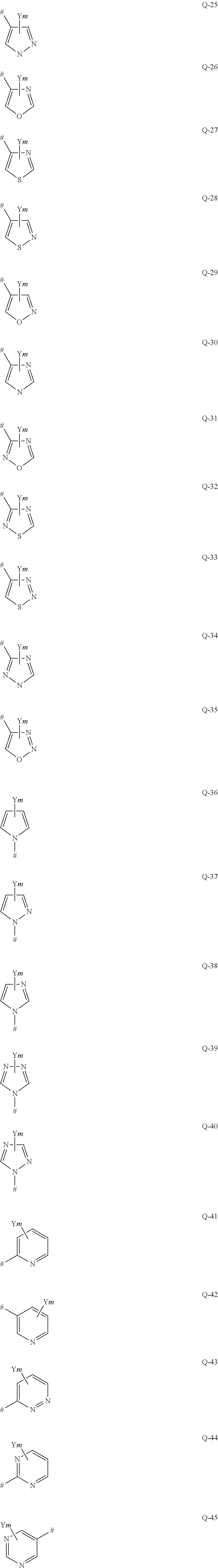

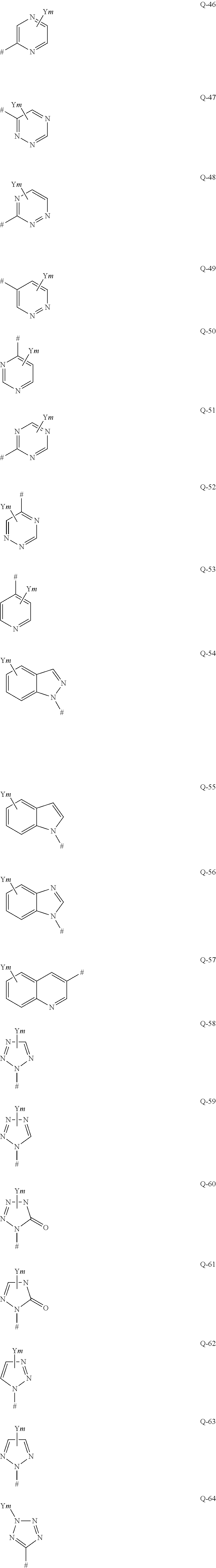

with
m is 0, 1 or 2, limited by the number of available positions in Q to which a substituent Y can be connected, and
each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), —NH(C₁-C₄-alkylcarbonyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino.

Preferred substituents or ranges of the structural elements mentioned in the compounds of formula (I) are explained below (embodiment 2-1).
n is 1 or 2, limited by the number of available positions in the ring to which a substituent X can be connected,
each X is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxy carbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
Q represents an optionally mono- or polysubstituted heteroaromatic ring from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-17, Q-18, Q-19, Q-20, Q-21, Q-22, Q-23, Q-24, Q-25, Q-26, Q-27, Q-28, Q-29, Q-30, Q-31, Q-32, Q-33, Q-34, Q-35, Q-36, Q-37, Q-38, Q-39, Q-40, Q-41, Q-42, Q-43, Q-44, Q-45, Q-46, Q-47, Q-48, Q-49, Q-50, Q-51, Q-52, Q-53, Q-54, Q-55, Q-56, Q-57, Q-58, Q-59, Q-60, Q-61, Q-62, Q-63 and Q-64
   with
m is 0, 1 or 2, limited by the number of available positions in Q to which a substituent Y can be connected, and
each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), —NH(C₁-C₄-alkylcarbonyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
   R¹, R², R³ and R⁴ are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R¹ and R² together with the carbon atom to which they are bonded form a 4- or 5-membered carbocycle and R³ and R⁴ are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R³ and R⁴ together with the carbon atom to which they are bonded form a 3-, 4- or 5-membered carbocycle and R¹ and R² are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R² and R⁴ together with the carbon atoms to which they are bonded form a 5-membered non-aromatic carbocycle optionally substituted by substituents selected from the group consisting of one to four C₁-C₄-alkyl groups and one to four halogen atoms, and R¹ and R³ are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl, or
   R¹ and R³ together with the carbon atoms to which they are bonded form a 5-membered non-aromatic carbocycle optionally substituted by substituents selected from the group consisting of one to four C₁-C₄-alkyl groups and one to four halogen atoms, and R² and R⁴ are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁—C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl,
R⁵ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₂-C₄-alkenyl, C₂-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
A represents a phenyl group of formula (A1)

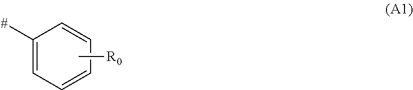

wherein
o is 0, 1 or 2, and
each R is independently selected from the group consisting of halogen, nitro, —OH, CHO, OCHO, NHCHO cyano, C₁-C₄-alkyl, C₁-C₄-halogenoalkyl having 1 to 5 halogen atoms, C₂-C₄-alkenyl, C₂-C₄-alkynyl, C₃-C₆-cycloalkyl, —S—C₁-C₄-alkyl, —S—C₁-C₄-halogenoalkyl having 1 to 5 halogen atoms, C₁-C₄-alkoxy, C₁-C₄-halogenoalkoxy having 1 to 5 halogen atoms, C₁-C₄-alkoxy-C₂-C₄-alkenyl, C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylsulfonamide, —NH(C₁-C₄-alkyl), N(C₁-C₄-alkyl)₂, phenyl (optionally substituted by C₁-C₄-alkoxy) and phenoxy, or two R bonded to adjacent carbon atoms together represent —O(CH₂)ₚO—, wherein p represents 1 or 2, or
A represents a heterocycle of the formula (Het-1)

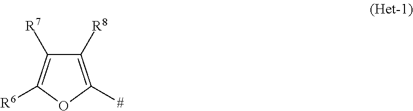

in which
R⁶ and R⁷ may be the same or different and are selected from the group consisting of hydrogen, halogen, nitro, C₁-C₄-alkyl and C₁-C₄-halogenoalkyl having 1 to 5 halogen atoms, and
R⁸ is selected from the group consisting of hydrogen, halogen, C₁-C₄-alkyl and C₁-C₄-halogenoalkyl having 1 to 5 halogen atoms, or
A represents a heterocycle of the formula (Het-2)

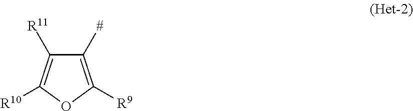

in which
R⁹ is selected from the group consisting of hydrogen, halogen, C₁-C₄-alkyl and C₁-C₄-halogenoalkyl having 1 to 5 halogen atoms, and
R¹⁰ and R¹¹ may be the same or different and are selected from the group consisting of hydrogen, halogen, C₁-C₄-alkyl and C₁-C₄-halogenoalkyl having 1 to 5 halogen atoms, phenyl optionally substituted by halogen or C₁-C₄-alkyl), or
A represents a heterocycle of the formula (Het-4)

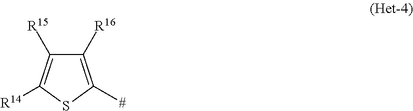

in which
R¹⁴ and R¹⁵ may be the same or different and are selected from the group consisting of hydrogen, halogen, C₁-C₄-alkyl, C₁-C₄-halogenoalkyl having 1 to 5 halogen atoms, —S—C₁-C₄-alkyl, —S(O)₂—C₁-C₄-alkyl, phenyl (optionally substituted by halogen or C₁-C₄-alkyl) and pyridyl (optionally substituted by halogen or C₁-C₄-alkyl), and
R¹⁶ is selected from the group consisting of hydrogen, halogen, cyano, C₁-C₄-alkyl, C₁-C₄-halogenoalkyl having 1 to 5 halogen atoms and C₁-C₄-halogenoalkoxy having 1 to 5 halogen atoms, or
A represents a heterocycle of the formula (Het-5)

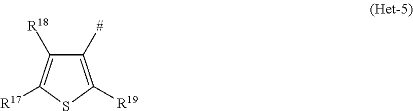

in which
R¹⁷ and R¹⁸ may be the same or different and are selected from the group consisting of hydrogen, halogen, C₁-C₄-alkyl, C₁-C₄-alkyloxy and C₁-C₄-halogenoalkyl having 1 to 5 halogen atoms, and
R¹⁹ is selected from the group consisting of hydrogen, halogen, C₁-C₄-alkyl and C₁-C₄-halogenoalkyl having 1 to 5 atoms, or
A represents a heterocycle of the formula (Het-6)

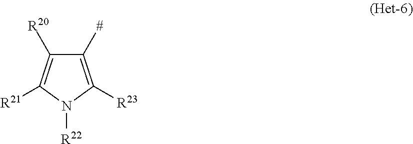

in which
R²⁰ is selected from the group consisting of hydrogen, halogen, cyano, C₁-C₄-alkyl and C₁-C₄-halogenoalkyl having 1 to 5 halogen atoms, and
R²¹ and R²³ may be the same or different and are selected from the group consisting of hydrogen, halogen, C₁-C₄-alkyl and C₁-C₄-halogenoalky having 1 to 5 halogen atoms, and
R²² is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, or
A represents a heterocycle of the formula (Het-7)

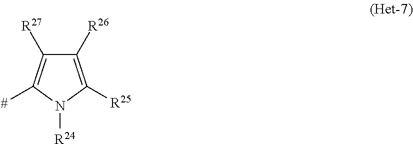

in which
R²⁴ is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, C₁-C₆-alkylcarbonyl, or benzoyl (optionally substituted by halogen or a C₁-C₄-alkyl), and
R²⁵, R²⁶ and R²⁷ may be the same or different and are selected from the group consisting of hydrogen, halogen, cyano, C₁-C₄-alkyl, C₁-C₄-halogenalkyl having 1 to 5 halogen atoms and C₁-C₄-alkylcarbonyl, or
A represents a heterocycle of the formula (Het-9)

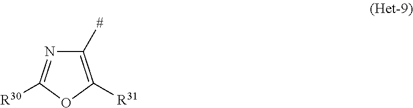

in which
R³⁰ is selected from the group consisting of hydrogen and C₁-C₄-alkyl, and
R³¹ is selected from the group consisting of halogen, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-10)

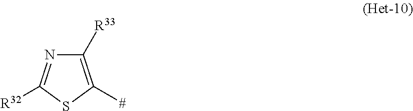

in which
R³² is selected from the group consisting of hydrogen, halogen, amino, cyano, C₁-C₄-alkylamino, di-(C₁-C₄-alkyl)amino, C₁-C₄-alkyl, C₁-C₄-halogenoalkyl having 1 to 5 halogen atoms and phenyl (optionally substituted by halogen or C₁-C₄-alkyl), and
R³³ is selected from the group consisting of halogen, C₁-C₄-alkyl and C₁-C₄-halogenoalkyl having 1 to 5 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, or
A represents a heterocycle of the formula (Het-11)

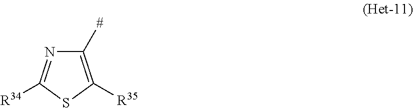

in which
R³⁴ is selected from the group consisting of hydrogen, halogen, C₁-C₄-alkylamino, di-(C₁-C₄-alkyl)amino, C₁-C₄-alkyl and C₁-C₄-halogenoalkyl having 1 to 5 halogen atoms, and
R³⁵ is selected from the group consisting of halogen, C₁-C₄-alkyl and C₁-C₄-halogenoalkyl having 1 to 5 halogen atoms, or
A represents a heterocycle of the formula (Het-12)

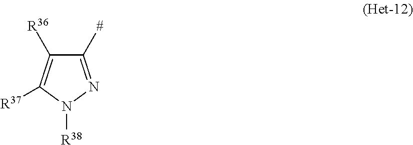

in which
R³⁶ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, —S(O)—C₁-C₄-alkyl, —S(O)₂—C₁-C₄-alkyl and —S—C₁-C₄-halogenoalkyl having 1 to 5 halogen atoms, and
R³⁷ is selected from the group consisting of hydrogen, halogen, cyano, nitro, C₁-C₄-alkyl, C₁-C₄-alkoxy and —S—C₁-C₄-alkyl, —S(O)—C₁-C₄-alkyl, —S(O)₂—C₁-C₄-alkyl, and
R³⁸ is selected from the group consisting of phenyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-13)

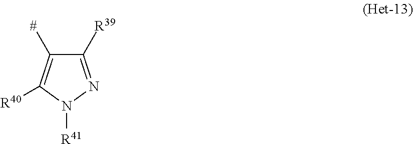

in which
R³⁹ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, —S(O)—C₁-C₄-alkyl, —S(O)₂—C₁-C₄-alkyl, —S—C₁-C₄-halogenoalkyl having 1 to 5 atoms, aminocarbonyl and aminocarbonyl-C₁-C₄-alkyl, and
R⁴⁰ is selected from the group consisting of hydrogen, halogen, cyano, C₁-C₄-alkyl, C₁-C₄-alkoxy, C₁-C₄-halogenoalkoxy having 1 to 5 halogen atoms, —S—C₁-C₄-alkyl, —S(O)—C₁-C₄-alkyl, and —S(O)₂—C₁-C₄-alkyl, and
R⁴¹ is selected from the group consisting of hydrogen, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl, C₁-C₄-halogenoalkoxy-C₁-C₄-alkyl having 1 to 5 halogen atoms and phenyl optionally substituted by halogen, C₁-C₄-alkyl, C₁-C₄-alkoxy-C₁-C₄-alkyl or nitro, or
A represents a heterocycle of the formula (Het-14)

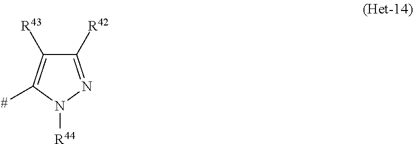

in which
R⁴² is selected from the group consisting of hydrogen, halogen, cyano, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, —S(O)—C₁-C₄-alkyl, and —S(O)₂—C₁-C₄-alkyl, —S—C₁-C₄-halogenoalkyl having 1 to 5 halogen atoms, aminocarbonyl and aminocarbonyl-C₁-C₄-alkyl, and
R⁴³ is selected from the group consisting of hydrogen, halogen, cyano, C₁-C₄-alkyl, C₁-C₄-alkoxy, —S—C₁-C₄-alkyl, —S(O)—C₁-C₄-alkyl, and —S(O)₂—C₁-C₄-alkyl, and C₁-C₄-halogenoalkyl having 1 to 5 halogen atoms, and
R⁴⁴ is selected from the group consisting of phenyl, benzyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-15)

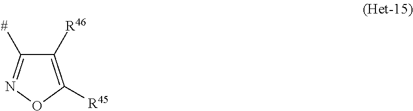

in which
R⁴⁵ and R⁴⁶ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-16)

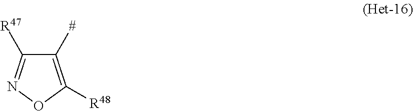

in which
R⁴⁷ and R⁴⁸ may be the same or different and are selected from the group consisting of hydrogen, halogen, C₁-C₄-alkyl, C₁-C₄-halogenoalkyl having 1 to 5 halogen atoms, phenyl (optionally substituted by halogen or a C₁-C₄-alkyl), or heterocyclyl like pyridyl, pyrimidinyl and thiadiazolyl (optionally substituted by halogen or C₁-C₄-alkyl), or
A represents a heterocycle of the formula (Het-17)

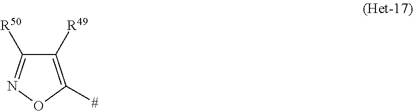

in which
R⁴⁹ and R⁵⁰ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-19)

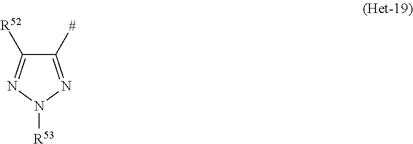

in which
R⁵² is selected from the group consisting of halogen, C₁-C₄-alkyl and C₁-C₄-halogenoalkyl having 1 to 5 halogen atoms, and
R⁵³ is selected from the group consisting of C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-20)

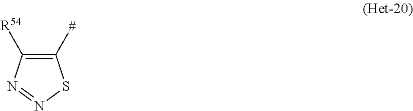

in which
R⁵⁴ is selected from the group consisting of halogen, C₁-C₄-alkyl and C₁-C₄-halogenoalkyl having 1 to 5 halogen atoms, or
A represents a heterocycle of the formula (Het-21)

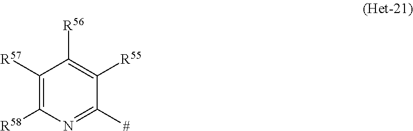

in which
R⁵⁵ is selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, —S(O)—C₁-C₄-alkyl, —S(O)₂—C₁-C₄-alkyl, —S—C₁-C₄-halogenoalkyl having 1 to 5 halogen atoms and C₁-C₄-halogenoalkoxy having 1 to 5 halogen atoms, and
R⁵⁶, R⁵⁷ and R⁵⁸, which may be the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, —S(O)—C₁-C₄-alkyl, —S(O)₂—C₁-C₄-alkyl, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-22)

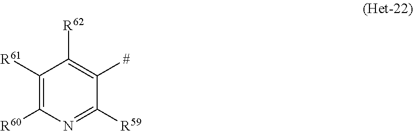

in which
R⁵⁹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and S-phenyl (optionally substituted by halogen or C₁-C₄-alkyl), and
R⁶⁰, R⁶¹ and R⁶², which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, N-morpholine (optionally substituted by halogen or C₁-C₄-alkyl) and thienyl (optionally substituted by halogen or a C₁-C₄-alkyl), or
A represents a heterocycle of the formula (Het-23)

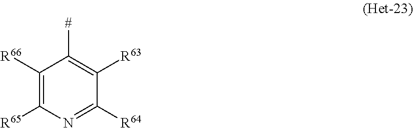

in which
R⁶³, R⁶⁴, R⁶⁵ and R⁶⁶, which may be the same or different, are selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, —S—C₁-C₄-halogenoalkyl having 1 to 5 halogen atoms, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-24)

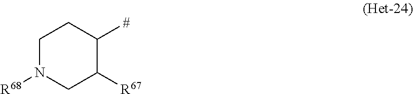

in which
R⁶⁷ is selected from the group consisting of hydrogen, halogen, C₁-C₄-alkyl and C₁-C₄-halogenoalkyl having 1 to 5 halogen atoms, and
R⁶⁸ is selected from the group consisting of C₁-C₄-alkyl, C₁-C₄-halogenoalkyl having 1 to 5 halogen atoms, C₁-C₆-alkoxycarbonyl, benzyl (optionally substituted by 1 to 3 halogen atoms), benzyloxycarbonyl (optionally substituted by 1 to 3 halogen atoms) and heterocyclyl like pyrimidinyl, (optionally substituted by halogen, C₁-C₄-alkyl, C₁-C₄-halogenoalkyl having 1 to 5 halogen atoms), or
A represents a heterocycle of the formula (Het-25)

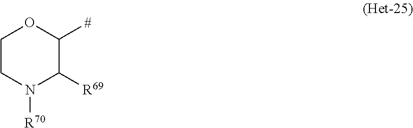

in which
R⁶⁹ is selected from the group consisting of hydrogen, C₁-C₄-alkyl and C₁-C₄-halogenoalkyl having 1 to 5 halogen atoms, and
R⁷⁰ is selected from the group consisting of hydrogen, C₁-C₄-alkyl, C₁-C₄-halogenoalkyl having 1 to 5 halogen atoms and benzyl, or
A represents a heterocycle of the formula (Het-26)

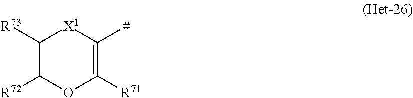

in which
X¹ is selected from the group consisting of sulphur, —SO—, or —SO₂—, and
R⁷¹ is selected from the group consisting of C₁-C₄-alkyl and C₁-C₄-halogenoalkyl having 1 to 5 halogen atoms, and
R⁷² and R⁷³ may be the same or different and are selected from the group consisting of hydrogen and C₁-C₄-alkyl, or
A represents a heterocycle of the formula (Het-29)

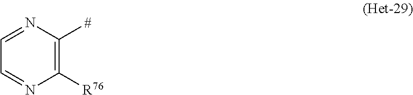

in which
R⁷⁶ is selected from the group consisting of hydrogen, halogen, C₁-C₄-alkyl and C₁-C₄-halogenoalkyl having 1 to 5 halogen atoms.

In another individual embodiment (embodiment 2-2), the structural elements in the compound of formula (I) are preferably defined as follows:
n is as defined in embodiment 2-1,
each X is as defined in embodiment 2-1,
Q represents an optionally mono- or polysubstituted heteroaromatic ring from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-17, Q-18, Q-19, Q-20, Q-21, Q-22, Q-23, Q-24, Q-25, Q-26, Q-27, Q-28, Q-29, Q-30, Q-31, Q-32, Q-33, Q-34, Q-35, Q-36, Q-37, Q-38, Q-39, Q-40, Q-41, Q-42, Q-43, Q-44, Q-45, Q-46, Q-47, Q-48, Q-49, Q-50, Q-51, Q-52, Q-53, Q-54, Q-55, Q-56, Q-57, Q-58, Q-59, Q-60, Q-61, Q-62, Q-63 and Q-64
   with
m is 0, 1 or 2, limited by the number of available positions in Q to which a substituent Y can be connected, and
each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl) (C₁-C₄-alkyl), —NH(C₁-C₄-alkylcarbonyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
R¹, R², R³ and R⁴ are the same or different and are selected from the group consisting of hydrogen, halogen, cyano, hydroxy, amino, —CHO, —COOH, —CONH₂, C₁-C₄-alkyl, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkyl having 1 to 5 halogen atoms, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₃-alkyl, C₃-C₆-halogenocycloalkyl-C₁-C₃-alkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, —OC(O)—C₁-C₄-alkyl, —OC(O)—C₁-C₄-halogenoalkyl having 1 to 5 halogen atoms, —NHC(O)—C₁-C₄-alkyl, —NHC(O)—C₁-C₄-halogenoalkyl having 1 to 5 halogen atoms, —OCONH(C₁-C₄-alkyl), —OCON(C₁-C₄-alkyl)₂, —OCONH(OC₁-C₄-alkyl), OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, benzyl, benzyloxy, —S-benzyl, —S(O)-benzyl, —S(O)₂-benzyl, benzylamino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl,
   with the proviso that R¹ is fluorine and/or R² is fluorine,
R⁵ is as defined in embodiment 2-1, and
A is as defined in embodiment 2-1 with the proviso that for Het-21, R⁵⁵ is not CF₃.

In another individual aspect of embodiment 2-1, R¹ is fluorine. In another individual aspect of embodiment 2-1, R² is fluorine. In another individual aspect of embodiment 2-1, R¹ is fluorine and R² is fluorine. In another individual aspect of embodiment 2-1, the combination R¹/R² is fluorine/methyl. In another individual aspect of embodiment 2-1, the combination R¹/R² is fluorine/hydrogen.

In another individual aspect of embodiment 2-2, R¹ is fluorine. In another individual aspect of embodiment 2-2, R² is fluorine. In another individual aspect of embodiment 2-2, R¹ is fluorine and R² is fluorine. In another individual aspect of embodiment 2-2, the combination R¹/R² is fluorine/methyl. In another individual aspect of embodiment 2-2, the combination R¹/R² is fluorine/hydrogen.

More preferred substituents or ranges of the structural elements mentioned in the compounds of formula (I) are explained below (embodiment 3-1).
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents an optionally mono- or polysubstituted heteroaromatic ring from the group consisting of Q-4, Q-11, Q-21, Q-22, Q-25, Q-36, Q-37, Q-38, Q-40, Q-41, Q-42, Q-53, Q-58, Q-62, Q-63 and Q-64,
   with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine, bromine, iodine, cyano, —OCH₃, —OCH₂CH₃, —OCH(CH₃)₂, —OCH₂CF₃, S(O)₂—CH₃, NHC(O)CH₃,
R¹ and R² are the same or different and are selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, and phenyl,
R³ and R⁴ are the same or different and are selected from the group consisting of hydrogen, —COOH, C₁-C₄-alkyl, C₁-C₄-halogenoalkyl C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl, or
R¹ and R² together with the carbon atom to which they are bonded form a 4 or 5-membered carbocycle, and R³ and R⁴ are the same or different and are selected from the group consisting of hydrogen, —COOH, C₁-C₄-alkyl, C₁-C₄-halogenoalkyl C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl,
   preferably R¹ and R² together with the carbon atom to which they are bonded form a cyclobutyl or a cyclopentyl, or
R³ and R⁴ together with the carbon atom to which they are bonded form a 3-, 4- or 5-membered carbocycle, and R¹ and R² are the same or different and are selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, 2,6-dichlorophenyl-carbonylamino, 2-chlorophenyl-carbonylamino and phenyl,
   preferably R³ and R⁴ together with the carbon atom to which they are bonded form a cyclopropyl or a cyclobutyl, or
R² and R⁴ together with the carbon atoms to which they are bonded form a 5-membered non-aromatic carbocycle optionally substituted by substituents selected from the group consisting of one to four C₁-C₃-alkyl groups and one to two halogen atoms, and R¹ is selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, 2,6-dichlorophenyl-carbonylamino, 2-chlorophenyl-carbonylamino and phenyl, and R³ is selected from the group consisting of hydrogen, —COOH, C₁-C₄-alkyl, C₁-C₄-halogenoalkyl C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl,
   preferably R² and R⁴ together with the carbon atoms to which they are bonded form a cyclopentyl, or
R¹ and R³ together with the carbon atoms to which they are bonded form a 5-membered non-aromatic carbocycle optionally substituted by substituents selected from the group consisting of one to four C₁-C₃-alkyl groups and one to two halogen atoms, and R² is selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, 2,6-dichlorophenyl-carbonylamino, 2-chlorophenyl-carbonylamino and phenyl, and R⁴ is selected from the group consisting of hydrogen, —COOH, C₁-C₄-alkyl, C₁-C₄-halogenoalkyl-C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl,
   preferably R¹ and R³ together with the carbon atoms to which they are bonded form a cyclopentyl,
R⁵ is selected from the group consisting of hydrogen, C₁-C₄-alkyl, C₃-C₆-cycloalkyl, C₁-C₄-alkoxy, C₁-C₄-alkoxy-C₁-C₄-alkyl, C₁-C₄-alkylcarbonyl, C₁-C₄-alkoxycarbonyl,
A represents a phenyl group of formula (A1)

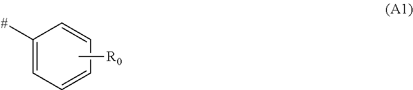

wherein
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1)

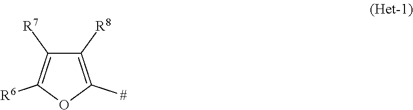

in which
R⁶ and R⁷ may be the same or different and are selected from the group consisting of hydrogen, halogen, nitro, C₁-C₄-alkyl and C₁-C₄-halogenoalkyl having 1 to 5 halogen atoms, and
R⁸ is selected from the group consisting of hydrogen, halogen, C₁-C₄-alkyl and C₁-C₄-halogenoalkyl having 1 to 5 halogen atoms, or
A represents a heterocycle of the formula (Het-2)

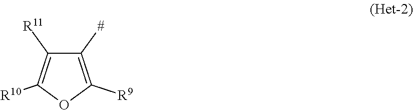

in which
R⁹ is selected from the group consisting of hydrogen, halogen, C₁-C₄-alkyl and C₁-C₄-halogenoalkyl having 1 to 5 halogen atoms, and
R¹⁰ and R¹¹ may be the same or different and are selected from the group consisting of hydrogen, halogen, C₁-C₄-alkyl and C₁-C₄-halogenoalkyl having 1 to 5 halogen atoms, phenyl optionally substituted by halogen or C₁-C₄-alkyl), or
A represents a heterocycle of the formula (Het-4)

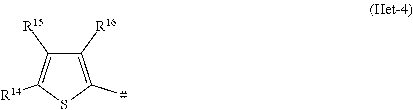

in which
R¹⁴ and R¹⁵ may be the same or different and are selected from the group consisting of hydrogen, halogen, C₁-C₄-alkyl, C₁-C₄-halogenoalkyl having 1 to 5 halogen atoms, —S—C₁-C₄-alkyl, —S(O)₂—C₁-C₄-alkyl, phenyl (optionally substituted by halogen or C₁-C₄-alkyl) and pyridyl (optionally substituted by halogen or C₁-C₄-alkyl), and
R¹⁶ is selected from the group consisting of hydrogen, halogen, cyano, C₁-C₄-alkyl, C₁-C₄-halogenoalkyl having 1 to 5 halogen atoms and C₁-C₄-halogenoalkoxy having 1 to 5 halogen atoms, or
A represents a heterocycle of the formula (Het-5)

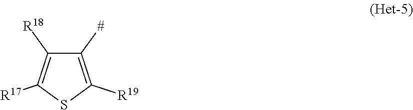

in which
R¹⁷ and R¹⁸ may be the same or different and are selected from the group consisting of hydrogen, halogen, C₁-C₄-alkyl, C₁-C₄-alkyloxy and C₁-C₄-halogenoalkyl having 1 to 5 halogen atoms, and
R¹⁹ is selected from the group consisting of hydrogen, halogen, C₁-C₄-alkyl and C₁-C₄-halogenoalkyl having 1 to 5 atoms, or
A represents a heterocycle of the formula (Het-6)

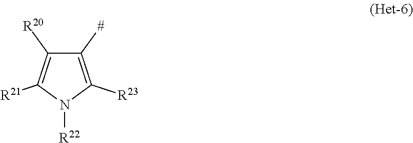

in which
R²⁰ is selected from the group consisting of hydrogen, halogen, cyano, C₁-C₄-alkyl and C₁-C₄-halogenoalkyl having 1 to 5 halogen atoms, and
R²¹ and R²³ may be the same or different and are selected from the group consisting of hydrogen, halogen, C₁-C₄-alkyl and C₁-C₄-halogenoalky having 1 to 5 halogen atoms, and
R²² is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, or
A represents a heterocycle of the formula (Het-10)

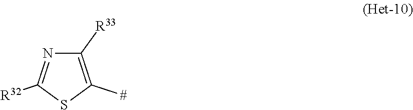

in which
R³² is selected from the group consisting of hydrogen, halogen, amino, cyano, C₁-C₄-alkylamino, di-(C₁-C₄-alkyl)amino, C₁-C₄-alkyl, C₁-C₄-halogenoalkyl having 1 to 5 halogen atoms and phenyl (optionally substituted by halogen or C₁-C₄-alkyl), and
R³³ is selected from the group consisting of halogen, C₁-C₄-alkyl and C₁-C₄-halogenoalkyl having 1 to 5 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, or
A represents a heterocycle of the formula (Het-21)

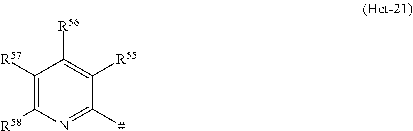

in which
R⁵⁵ is selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, —S(O)—C₁-C₄-alkyl, —S(O)₂—C₁-C₄-alkyl, —S—C₁-C₄-halogenoalkyl having 1 to 5 halogen atoms and C₁-C₄-halogenoalkoxy having 1 to 5 halogen atoms, and
R⁵⁶, R⁵⁷ and R⁵⁸, which may be the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-22)

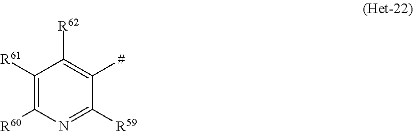

in which
R⁵⁹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and S-phenyl (optionally substituted by halogen or C₁-C₄-alkyl), and
R⁶⁰, R⁶¹ and R⁶², which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, N-morpholine (optionally substituted by halogen or C₁-C₄-alkyl) and thienyl (optionally substituted by halogen or a C₁-C₄-alkyl), or
A represents a heterocycle of the formula (Het-29)

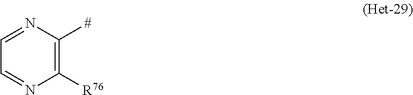

in which
R⁷⁶ is selected from the group consisting of hydrogen, halogen, C₁-C₄-alkyl and C₁-C₄-halogenoalkyl having 1 to 5 halogen atoms.

In another individual embodiment (embodiment 3-2), the structural elements in the compound of formula (I) are more preferably defined as follows:
n is 1 or 2,
each X is as defined in embodiment 3-1,
Q represents an optionally mono- or polysubstituted heteroaromatic ring from the group consisting of Q-4, Q-11, Q-21, Q-22, Q-25, Q-36, Q-37, Q-38, Q-40, Q-41, Q-42, Q-45, Q-53, Q-58, Q-62, Q-63 and Q-64,
   with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine, bromine, iodine, cyano, —OCH₃, —OCH₂CH₃, —OCH(CH₃)₂, —OCH₂CF₃, S(O)₂—CH₃, NHC(O)CH₃, NHCH₃ and N(CH₃)₂,
R¹ and R² are the same or different and are selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, and phenyl,
   with the proviso that R¹ is fluorine and/or R² is fluorine,
R³ and R⁴ are the same or different and are selected from the group consisting of hydrogen, —COOH, C₁-C₄-alkyl, C₁-C₄-halogenoalkyl C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl,
R⁵ is as defined in embodiment 3-1, and
A is as defined in embodiment 3-1 with the proviso that for Het-21, R⁵⁵ is not CF₃.

In another individual aspect of embodiment 3-1, R¹ is fluorine. In another individual aspect of embodiment 3-1, R² is fluorine. In another individual aspect of embodiment 3-1, R¹ is fluorine and R² is fluorine. In another individual aspect of embodiment 3-1, the combination R¹/R² is fluorine/methyl. In another individual aspect of embodiment 3-1, the combination R¹/R² is fluorine/hydrogen.

In another individual aspect of embodiment 3-2, R¹ is fluorine. In another individual aspect of embodiment 3-2, R² is fluorine. In another individual aspect of embodiment 3-2, R¹ is fluorine and R² is fluorine. In another individual aspect of embodiment 3-2, the combination R¹/R² is fluorine/methyl. In another individual aspect of embodiment 3-2, the combination R¹/R² is fluorine/hydrogen.

Especially preferred substituents or ranges of the structural elements mentioned in the compounds of formula (I) are explained below (embodiment 4-1).
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q is selected from:

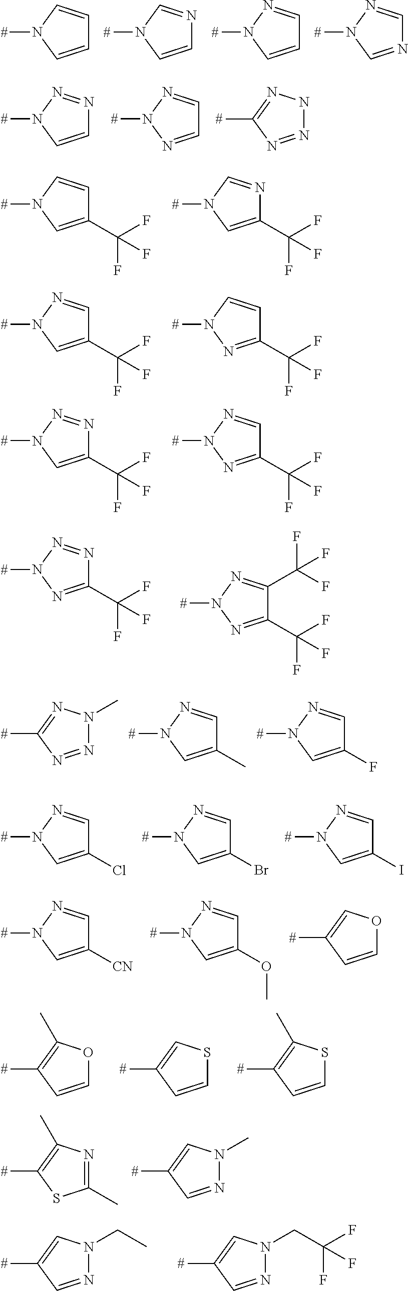

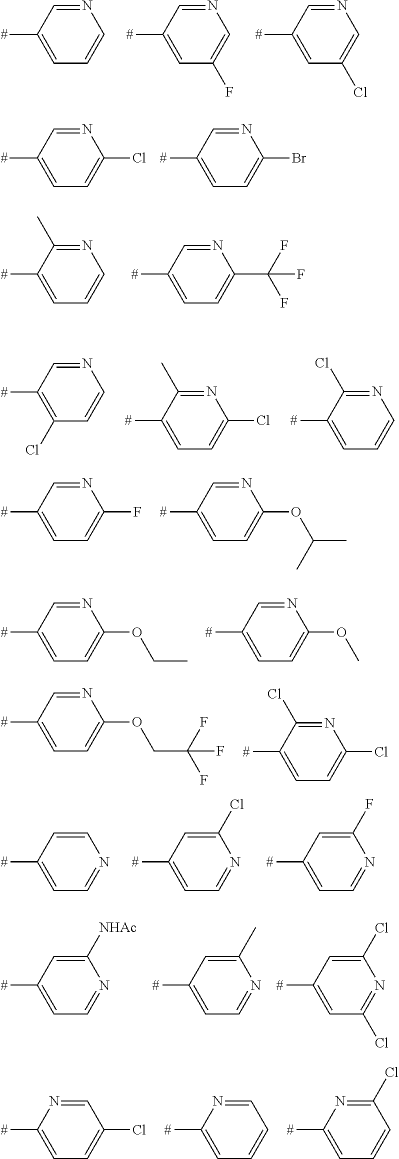

R¹ and R² are the same or different and are selected from the group consisting of hydrogen, methyl, ethyl, methoxy, ethoxy or fluorine, particularly wherein both R¹ and R² are not hydrogen,
R³ and R⁴ are the same or different and are selected from the group consisting of hydrogen, methyl or ethyl,
R⁵ is hydrogen,
A is selected from:

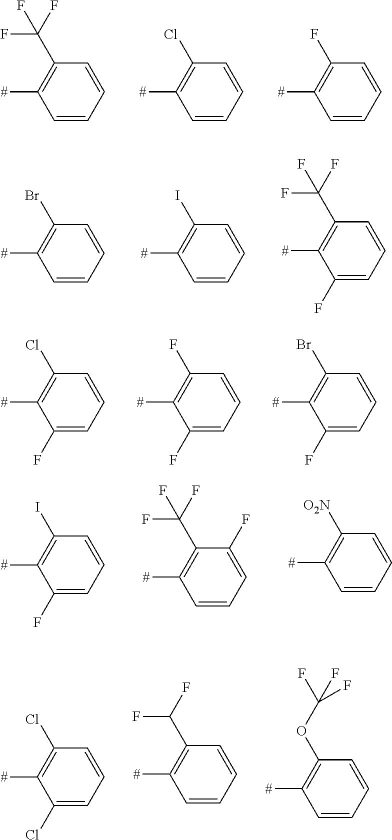

or
A is selected from:

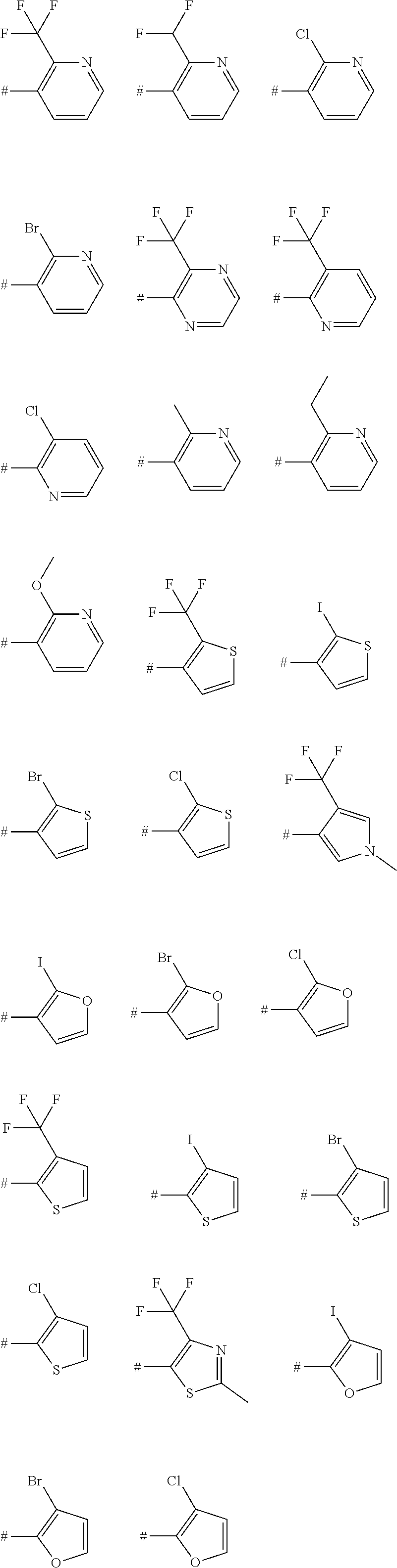

In another individual embodiment (embodiment 4-2), the structural elements in the compound of formula (I) are especially preferably defined as follows:
n is 1 or 2,
each X is as defined in embodiment 4-1,
Q is selected from:

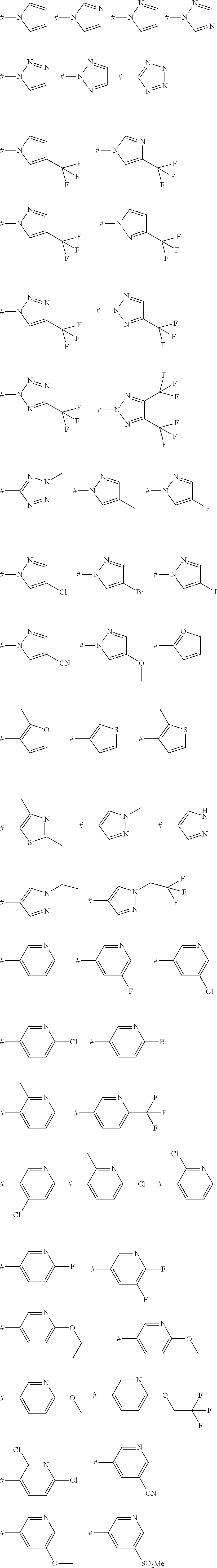

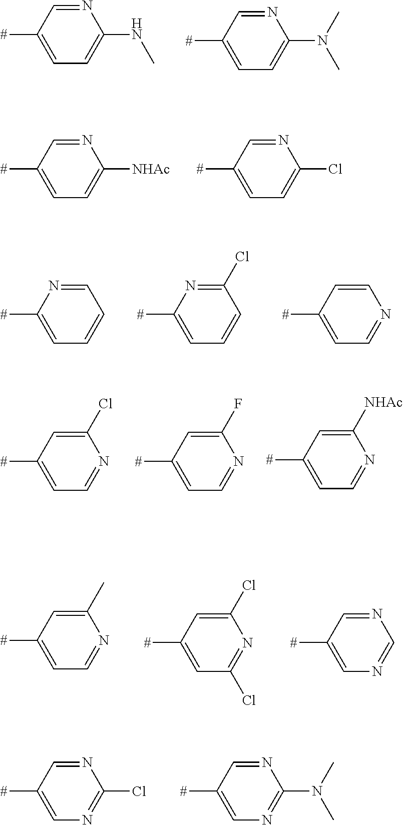

R¹ and R² are the same or different and are selected from the group consisting of hydrogen, methyl, ethyl, methoxy, ethoxy or fluorine,
   with the proviso that R¹ is fluorine and/or R² is fluorine,
R³ and R⁴ are the same or different and are selected from the group consisting of hydrogen, methyl or ethyl,
R⁵ is as defined in embodiment 4-1, and
A is selected from:

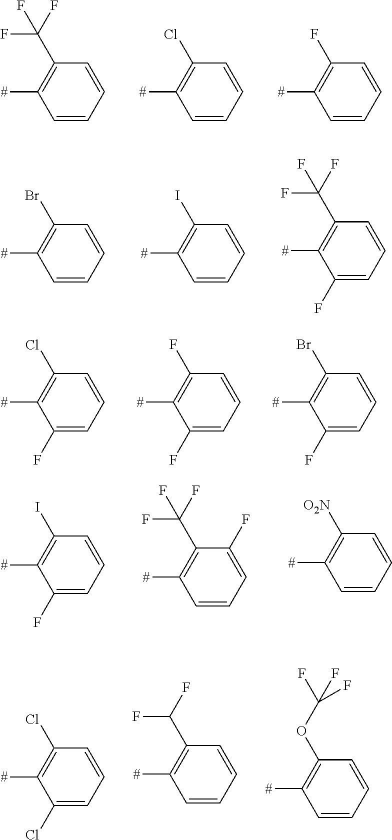

or
A is selected from:

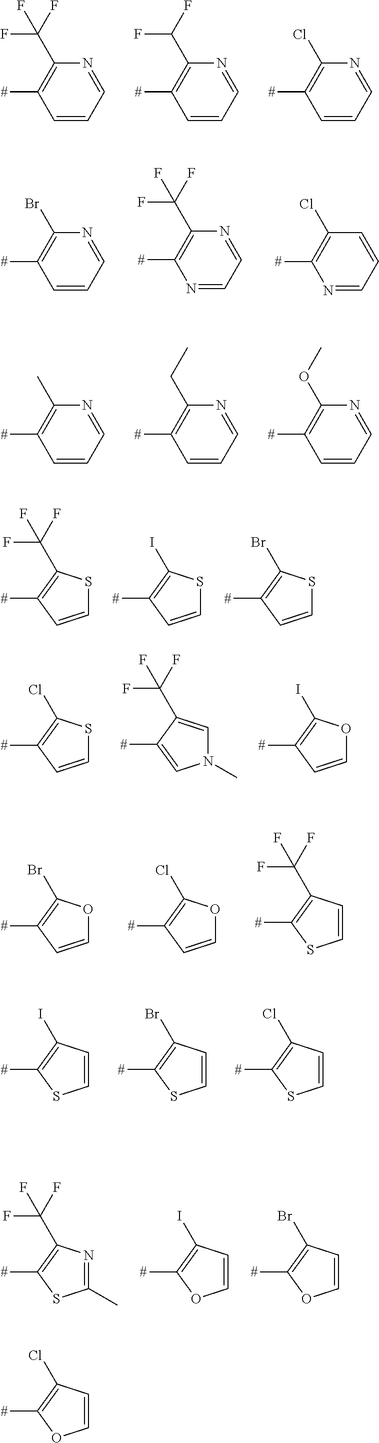

In another individual aspect of embodiment 4-1, R¹ is fluorine. In another individual aspect of embodiment 4-1, R² is fluorine. In another individual aspect of embodiment 4-1, R¹ is fluorine and R² is fluorine. In another individual aspect of embodiment 4-1, the combination R¹/R² is fluorine/methyl. In another individual aspect of embodiment 4-1, the combination R¹/R² is fluorine/hydrogen.

In another individual aspect of embodiment 4-2, R¹ is fluorine. In another individual aspect of embodiment 4-2, R² is fluorine. In another individual aspect of embodiment 4-2, R¹ is fluorine and R² is fluorine. In another individual aspect of embodiment 4-2, the combination R¹/R² is fluorine/methyl. In another individual aspect of embodiment 4-2, the combination R¹/R² is fluorine/hydrogen.

In a first very specific aspect (embodiment 5-1) of the especially preferred substituents or ranges of the structural elements mentioned in the compounds of formula (I),
n is 1,
X is selected from the group consisting of hydrogen or chlorine,
Q is selected from:

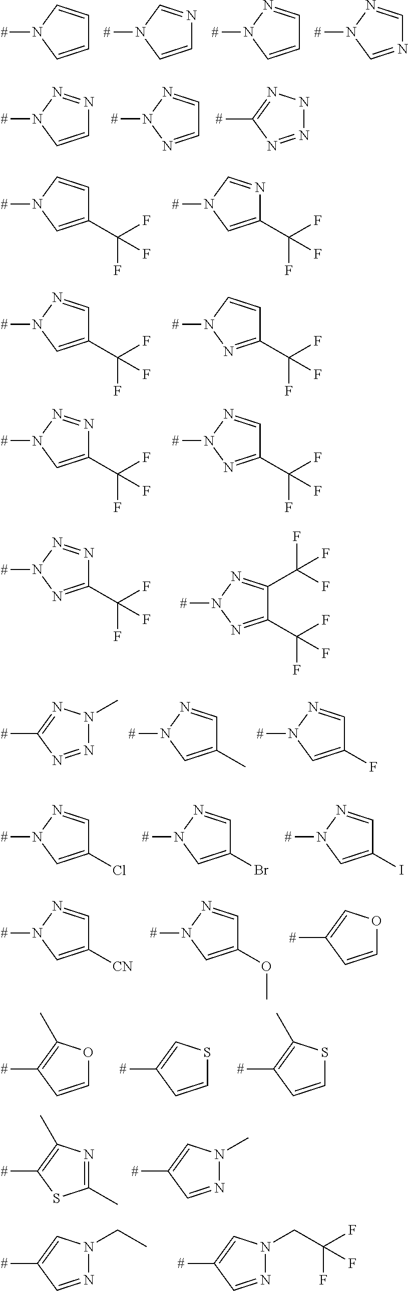

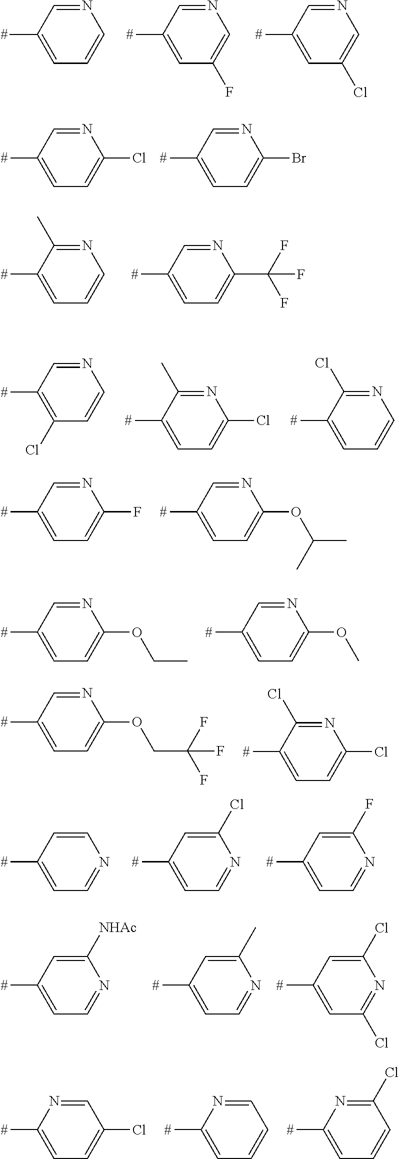

R¹, R², R³, R⁴ and R⁵ are hydrogen, or
R¹ and R² are fluorine and R³, R⁴ and R⁵ are hydrogen,
A is

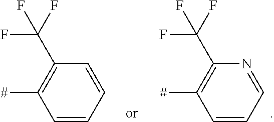

In a second very specific aspect (embodiment 5-2) of the especially preferred substituents or ranges of the structural elements mentioned in the compounds of formula (I),
n is 1 or 2,
each X is selected from the group consisting of hydrogen, chlorine, cyano, fluorine, methyl, trifluoromethyl and methoxy,
   preferably if n is 1 then X is selected from the group consisting of hydrogen, chlorine, fluorine, methyl, trifluormethyl and methoxy; if n is 2 then X is fluorine,
Q is selected from:

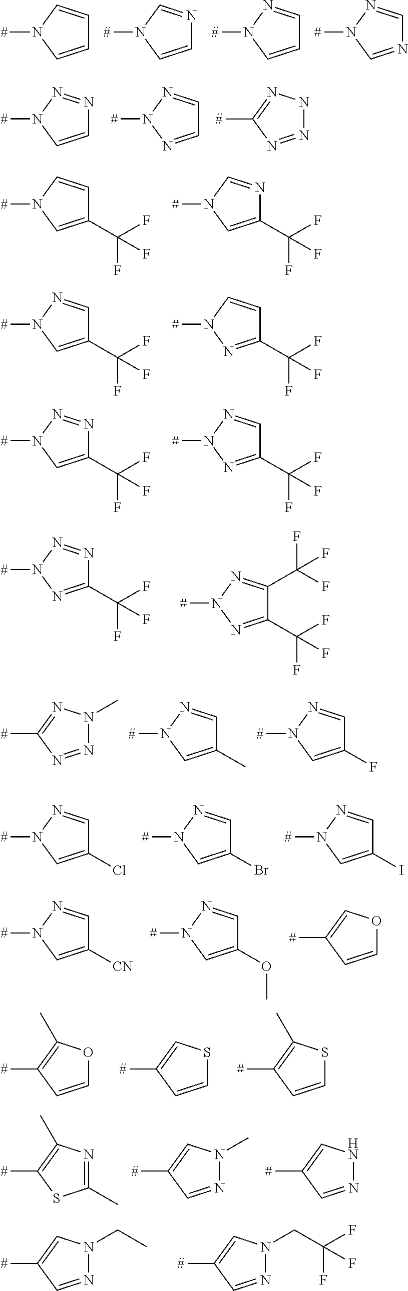

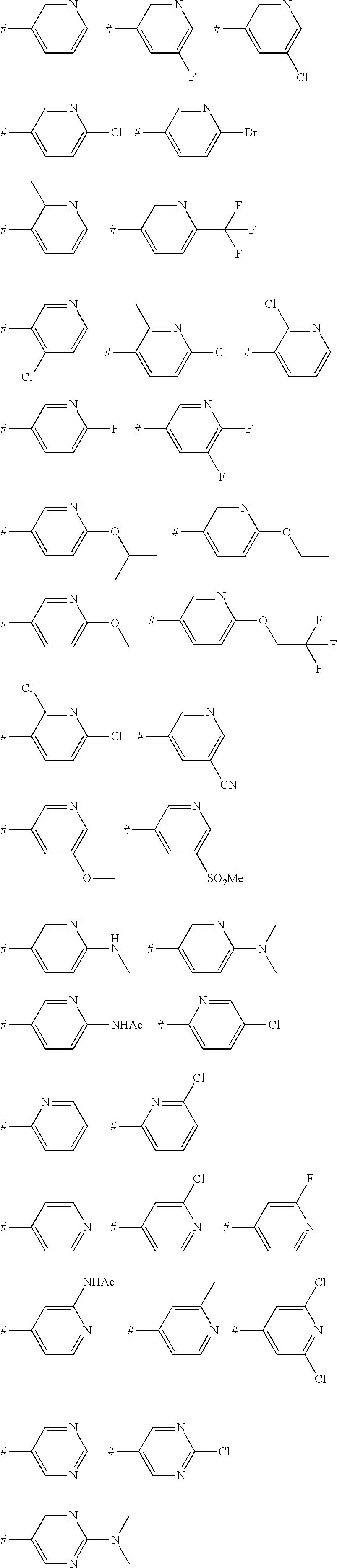

R¹ and R² are the same or different and are selected from the group consisting of hydrogen, methyl or fluorine,
   with the proviso that R¹ is fluorine and/or R² is fluorine,
R³, R⁴ and R⁵ are hydrogen,
A is

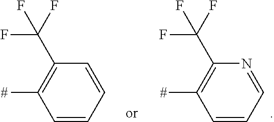

In a third very specific aspect (embodiment 5-3) of the especially preferred substituents or ranges of the structural elements mentioned in the compounds of formula (I),
n is 1,
X is selected from the group consisting of hydrogen or chlorine,
Q is selected from:

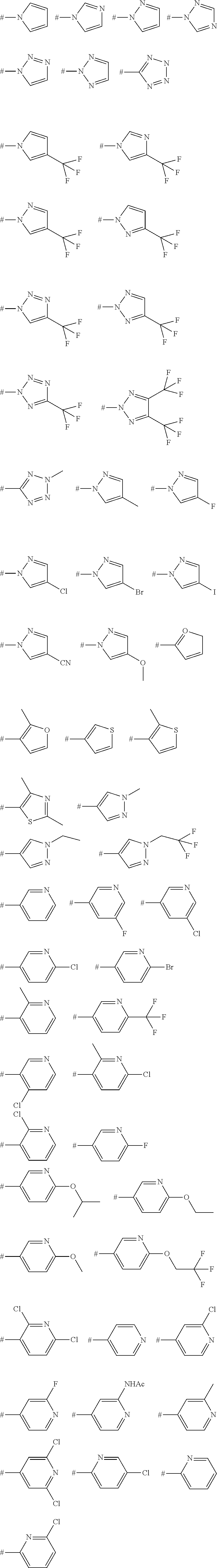

R¹ and R² are both fluorine, or
R¹ and R² are one fluorine and one hydrogen, or
R¹ and R² are one fluorine and one methyl,
R³, R⁴ and R⁵ are hydrogen,
A is

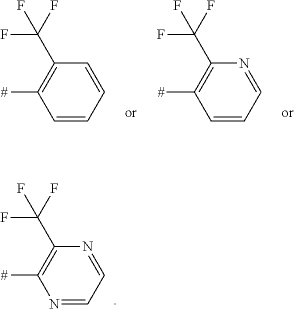

In a fourth very specific aspect (embodiment 5-4) of the especially preferred substituents or ranges of the structural elements mentioned in the compounds of formula (I),
n is 1 or 2,
each X is selected from the group consisting of hydrogen, chlorine, cyano, fluorine, methyl, trifluoromethyl and methoxy,
   preferably if n is 1 then X is selected from the group consisting of hydrogen, chlorine, fluorine, methyl, trifluormethyl and methoxy; if n is 2 then X is fluorine,
Q is selected from:

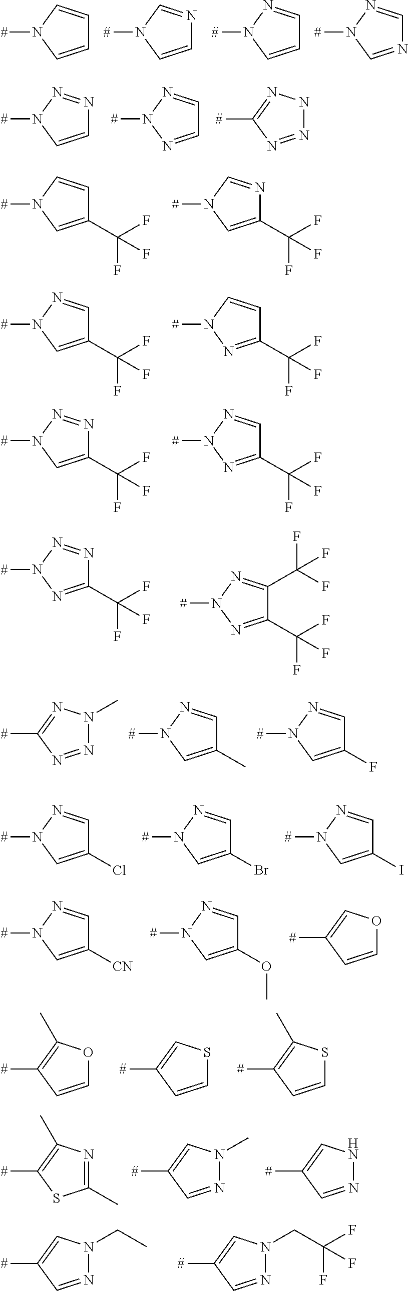

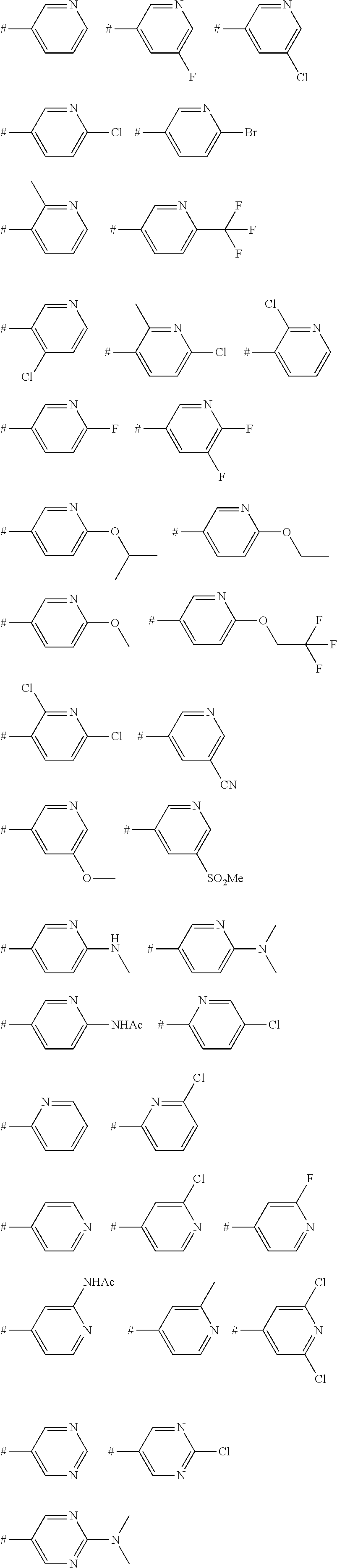

R¹ and R² are the same or different and are selected from the group consisting of hydrogen, methyl or fluorine,
   with the proviso that R¹ is fluorine and/or R² is fluorine,
R³, R⁴ and R⁵ are hydrogen,
A is selected from

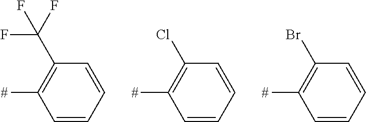

### or A is

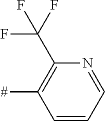

### or A is

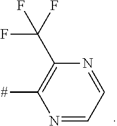

In another individual aspect of embodiment 5-1, R¹ is fluorine and R² is fluorine.

In another individual aspect of embodiment 5-2, R¹ is fluorine. In another individual aspect of embodiment 5-2, R² is fluorine. In another individual aspect of embodiment 5-2, R¹ is fluorine and R² is fluorine. In another individual aspect of embodiment 5-2, the combination R¹/R² is fluorine/methyl. In another individual aspect of embodiment 5-2, the combination R¹/R² is fluorine/hydrogen.

In another individual aspect of embodiment 5-3, R¹ is fluorine and R² is fluorine. In another individual aspect of embodiment 5-3, the combination R¹/R² is fluorine/methyl. In another individual aspect of embodiment 5-3, the combination R¹/R² is fluorine/hydrogen.

In another individual aspect of embodiment 5-4, R¹ is fluorine. In another individual aspect of embodiment 5-4, R² is fluorine. In another individual aspect of embodiment 5-4, R¹ is fluorine and R² is fluorine. In another individual aspect of embodiment 5-4, the combination R¹/R² is fluorine/methyl. In another individual aspect of embodiment 5-4, the combination R¹/R² is fluorine/hydrogen.

The definitions of radicals, and explanations, that are given above in general or in ranges of preference may be combined arbitrarily with one another, thus including combinations between the respective ranges and ranges of preference. Combinations of preferred features thus provide sub-classes of compounds according to the invention. The definitions and explanations apply to the end products and also to the precursors and intermediates accordingly.

Preferred in accordance with the invention are the compounds of the formula (I) in which there is a combination of the definitions given above as being preferred (preferably), wherein each embodiment described above as being preferred constitutes an individual combination.

More preferred in accordance with the invention are the compounds of the formula (I) in which there is a combination of the definitions given above as being more preferred (more preferably), wherein each embodiment described above as being more preferred constitutes an individual combination.

Especially preferred in accordance with the invention are the compounds of the formula (I) in which there is a combination of the definitions given above as being especially preferred (especially preferably), wherein each embodiment described above as being especially preferred constitutes an individual combination.

A very specific aspect in accordance with the invention are the compounds of the formula (I) in which there is a combination of the definitions given above as being a first very specific aspect (embodiment 5-1) of the especially preferred substituents or ranges of the structural elements.

Another very specific aspect in accordance with the invention are the compounds of the formula (I) in which there is a combination of the definitions given above as being a second very specific aspect (embodiment 5-2) of the especially preferred substituents or ranges of the structural elements.

Another very specific aspect in accordance with the invention are the compounds of the formula (I) in which there is a combination of the definitions given above as being a third very specific aspect (embodiment 5-3) of the especially preferred substituents or ranges of the structural elements.

Another very specific aspect in accordance with the invention are the compounds of the formula (I) in which there is a combination of the definitions given above as being a fourth very specific aspect (embodiment 5-4) of the especially preferred substituents or ranges of the structural elements.

Saturated or unsaturated hydrocarbon radicals such as alkyl, alkanediyl or alkenyl may in each case, both alone and in conjunction with heteroatoms, as in alkoxy, for example, be where possible either straight-chain or branched.

Any substituted radicals may, unless indicated otherwise, be substituted one or more times, and the substituents in the case of multiple substitutions may be alike or different.

In the definitions of radicals that are stated as being preferred, halogen (halo) is fluoro, chloro, bromo and iodo, very preferably fluoro, chloro and bromo, and especially preferably fluoro and chloro.

Further specific embodiments of the invention are described hereafter.

In further preferred embodiments, the invention relates to the use of compounds of formula (I), in which R³ and R⁴ and R⁵ are hydrogen, i.e. compounds of the formula (I-1).

Therefore, a specific embodiment (embodiment 6-1) of the invention is the use of a compound of the formula (I-1)

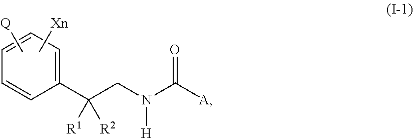

wherein R¹, R², Q, X, n and A are as defined above in embodiment 1-1, for controlling nematodes and/or other helminths.

Another specific embodiment (embodiment 6-2) of the invention is the use of a compound of the formula (I-1) wherein R¹, R², Q, X, n and A are as defined above in embodiment 1-2, for controlling nematodes and/or other helminths.

Another specific embodiment (embodiment 6-1a) of the invention is the use of a compound of the formula (I-1) wherein R¹, R², Q, X, n and A are as defined above in embodiment 1-1a, for controlling nematodes and/or other helminths.

Another specific embodiment (embodiment 6-2a) of the invention is the use of a compound of the formula (I-1) wherein R¹, R², Q, X, n and A are as defined above in embodiment 1-2a, for controlling nematodes and/or other helminths.

Another specific embodiment (embodiment 7-1) of the invention is the use of a compound of the formula (I-1) wherein R¹, R², Q, X, n and A are as defined above in embodiment 2-1, for controlling nematodes and/or other helminths.

Another specific embodiment (embodiment 7-2) of the invention is the use of a compound of the formula (I-1) wherein R¹, R², Q, X, n and A are as defined above in embodiment 2-2, for controlling nematodes and/or other helminths.

Another specific embodiment (embodiment 8-1) of the invention is the use of a compound of the formula (I-1) wherein R¹, R², Q, X, n and A are as defined above in embodiment 3-1, for controlling nematodes and/or other helminths.

Another specific embodiment (embodiment 8-2) of the invention is the use of a compound of the formula (I-1) wherein R¹, R², Q, X, n and A are as defined above in embodiment 3-2, for controlling nematodes and/or other helminths.

Another specific embodiment (embodiment 9-1) of the invention is the use of a compound of the formula (I-1) wherein R¹, R², Q, X, n and A are as defined above in embodiment 4-1, for controlling nematodes and/or other helminths.

Another specific embodiment (embodiment 9-2) of the invention is the use of a compound of the formula (I-1) wherein R¹, R², Q, X, n and A are as defined above in embodiment 4-2, for controlling nematodes and/or other helminths.

Another specific embodiment (embodiment 10-1) of the invention is the use of a compound of the formula (I-1) wherein R¹, R², Q, X, n and A are as defined above in embodiment 5-1, for controlling nematodes and/or other helminths.

Another specific embodiment (embodiment 10-2) of the invention is the use of a compound of the formula (I-1) wherein R¹, R², Q, X, n and A are as defined above in embodiment 5-2, for controlling nematodes and/or other helminths.

Another specific embodiment (embodiment 10-3) of the invention is the use of a compound of the formula (I-1) wherein R¹, R², Q, X, n and A are as defined above in embodiment 5-3, for controlling nematodes and/or other helminths.

Another specific embodiment (embodiment 10-4) of the invention is the use of a compound of the formula (I-1) wherein R¹, R², Q, X, n and A are as defined above in embodiment 5-4, for controlling nematodes and/or other helminths.

In embodiment 1-1 as well as in each individual aspect of said embodiment, Q preferably is in para-position (embodiment 1-1.1).

In embodiment 1-2 as well as in each individual aspect of said embodiment, Q preferably is in para-position (embodiment 1-2.1).

In embodiment 1-1a as well as in each individual aspect of said embodiment, Q preferably is in para-position (embodiment 1-1a.1).

In embodiment 1-2a as well as in each individual aspect of said embodiment, Q preferably is in para-position (embodiment 1-2a.1).

In embodiment 2-1 as well as in each individual aspect of said embodiment, Q preferably is in para-position (embodiment 2-1.1).

In embodiment 2-2 as well as in each individual aspect of said embodiment, Q preferably is in para-position (embodiment 2-2.1).

In embodiment 3-1 as well as in each individual aspect of said embodiment, Q preferably is in para-position (embodiment 3-1.1).

In embodiment 3-2 as well as in each individual aspect of said embodiment, Q preferably is in para-position (embodiment 3-2.1).

In embodiment 4-1 as well as in each individual aspect of said embodiment, Q preferably is in para-position (embodiment 4-1.1).

In embodiment 4-2 as well as in each individual aspect of said embodiment, Q preferably is in para-position (embodiment 4-2.1).

In embodiment 5-1 as well as in each individual aspect of said embodiment, Q preferably is in para-position (embodiment 5-1.1).

In embodiment 5-2 as well as in each individual aspect of said embodiment, Q preferably is in para-position (embodiment 5-2.1).

In embodiment 5-3 as well as in each individual aspect of said embodiment, Q preferably is in para-position (embodiment 5-3.1).

In embodiment 5-4 as well as in each individual aspect of said embodiment, Q preferably is in para-position (embodiment 5-4.1).

In embodiment 6-1 as well as in each individual aspect of said embodiment, Q preferably is in para-position (embodiment 6-1.1).

In embodiment 6-2 as well as in each individual aspect of said embodiment, Q preferably is in para-position (embodiment 6-2.1).

In embodiment 6-1a as well as in each individual aspect of said embodiment, Q preferably is in para-position (embodiment 6-1a.1).

In embodiment 6-2a as well as in each individual aspect of said embodiment, Q preferably is in para-position (embodiment 6-2a.1).

In embodiment 7-1 as well as in each individual aspect of said embodiment, Q preferably is in para-position (embodiment 7-1.1).

In embodiment 7-2 as well as in each individual aspect of said embodiment, Q preferably is in para-position (embodiment 7-2.1).

In embodiment 8-1 as well as in each individual aspect of said embodiment, Q preferably is in para-position (embodiment 8-1.1).

In embodiment 8-2 as well as in each individual aspect of said embodiment, Q preferably is in para-position (embodiment 8-2.1).

In embodiment 9-1 as well as in each individual aspect of said embodiment, Q preferably is in para-position (embodiment 9-1.1).

In embodiment 9-2 as well as in each individual aspect of said embodiment, Q preferably is in para-position (embodiment 9-2.1).

In embodiment 10-1 as well as in each individual aspect of said embodiment, Q preferably is in para-position (embodiment 10-1.1).

In embodiment 10-2 as well as in each individual aspect of said embodiment, Q preferably is in para-position (embodiment 10-2.1).

In embodiment 10-3 as well as in each individual aspect of said embodiment, Q preferably is in para-position (embodiment 10-3.1).

In embodiment 10-4 as well as in each individual aspect of said embodiment, Q preferably is in para-position (embodiment 10-4.1).

The present invention is also directed to novel compounds of formula (Ia)

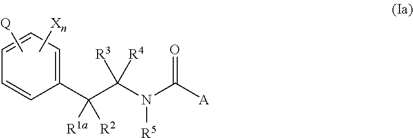

wherein
n, X, Q, R², R³, R⁴, R⁵ and A are as defined above in embodiment 1-1 and
R^{1a} is fluorine (embodiment Ia-1-1), preferably as defined above in embodiment 1-1.1 and R^{1a} is fluorine (embodiment Ia-1-1.1).

Another embodiment (embodiment Ia-1-2) is a compound of formula (Ia) wherein n, X, Q, R², R³, R⁴, R⁵ and A are as defined above in embodiment 1-2 and R^{1a} is fluorine, preferably as defined above in embodiment 1-2.1 and R^{1a} is fluorine (embodiment Ia-1-2.1).

Another embodiment (embodiment Ia-1-1a) is a compound of formula (Ia) wherein n, X, Q, R², R³, R⁴, R⁵ and A are as defined above in embodiment 1-1a and R^{1a} is fluorine, preferably as defined above in embodiment 1-1a.1 and lea is fluorine (embodiment Ia-1-1a.1).

Another embodiment (embodiment Ia-1-2a) is a compound of formula (Ia) wherein n, X, Q, R², R³, R⁴, R⁵ and A are as defined above in embodiment 1-2a and R^{1a} is fluorine, preferably as defined above in embodiment 1-2a.1 and R^{1a} is fluorine (embodiment Ia-1-2a.1).

Another embodiment (embodiment Ia-2-1) is a compound of formula (Ia) wherein n, X, Q, R², R³, R⁴, R⁵ and A are as defined above in embodiment 2-1 and R^{1a} is fluorine, preferably as defined above in embodiment 2-1.1 and R^{1a} is fluorine (embodiment Ia-2-1.1).

Another embodiment (embodiment Ia-2-2) is a compound of formula (Ia) wherein n, X, Q, R², R³, R⁴, R⁵ and A are as defined above in embodiment 2-2 and R^{1a} is fluorine, preferably as defined above in embodiment 2-2.1 and R^{1a} is fluorine (embodiment Ia-2-2.1).

Another embodiment (embodiment Ia-3-1) is a compound of formula (Ia) wherein n, X, Q, R², R³, R⁴, R⁵ and A are as defined above in embodiment 3-1 and R^{1a} is fluorine, preferably as defined above in embodiment 3-1.1 and R^{1a} is fluorine (embodiment Ia-3-1.1).

Another embodiment (embodiment Ia-3-2) is a compound of formula (Ia) wherein n, X, Q, R², R³, R⁴, R⁵ and A are as defined above in embodiment 3-2 and R^{1a} is fluorine, preferably as defined above in embodiment 3-2.1 and R^{1a} is fluorine (embodiment Ia-3-2.1).

Another embodiment (embodiment Ia-4-1) is a compound of formula (Ia) wherein n, X, Q, R², R³, R⁴, R⁵ and A are as defined above in embodiment 4-1 and R^{1a} is fluorine, preferably as defined above in embodiment 4-1.1 and R^{1a} is fluorine (embodiment Ia-4-1.1).

Another embodiment (embodiment Ia-4-2) is a compound of formula (Ia) wherein n, X, Q, R², R³, R⁴, R⁵ and A are as defined above in embodiment 4-2 and R^{1a} is fluorine, preferably as defined above in embodiment 4-2.1 and R^{1a} is fluorine (embodiment Ia-4-2.1).

Another embodiment (embodiment Ia-5-1) is a compound of formula (Ia) wherein n, X, Q, R², R³, R⁴, R⁵ and A are as defined above in embodiment 5-1 and R^{1a} is fluorine, preferably as defined above in embodiment 5-1.1 and R^{1a} is fluorine (embodiment Ia-5-1.1).

Another embodiment (embodiment Ia-5-2) is a compound of formula (Ia) wherein n, X, Q, R², R³, R⁴, R⁵ and A are as defined above in embodiment 5-2 and R^{1a} is fluorine, preferably as defined above in embodiment 5-2.1 and R^{1a} is fluorine (embodiment Ia-5-2.1).

Another embodiment (embodiment Ia-5-3) is a compound of formula (Ia) wherein n, X, Q, R², R³, R⁴, R⁵ and A are as defined above in embodiment 5-3 and R^{1a} is fluorine, preferably as defined above in embodiment 5-3.1 and R^{1a} is fluorine (embodiment Ia-5-3.1).

Another embodiment (embodiment Ia-5-4) is a compound of formula (Ia) wherein n, X, Q, R², R³, R⁴, R⁵ and A are as defined above in embodiment 5-4 and R^{1a} is fluorine, preferably as defined above in embodiment 5-4.1 and R^{1a} is fluorine (embodiment Ia-5-4.1).

Another embodiment (embodiment Ia-6-1) is a compound of formula (Ia) wherein n, X, Q, R², R³, R⁴, R⁵ and A are as defined above in embodiment 6-1 and R^{1a} is fluorine, preferably as defined above in embodiment 6-1.1 and R^{1a} is fluorine (embodiment Ia-6-1.1).

Another embodiment (embodiment Ia-6-2) is a compound of formula (Ia) wherein n, X, Q, R², R³, R⁴, R⁵ and A are as defined above in embodiment 6-2 and R^{1a} is fluorine, preferably as defined above in embodiment 6-2.1 and R^{1a} is fluorine (embodiment Ia-6-2.1).

Another embodiment (embodiment Ia-6-la) is a compound of formula (Ia) wherein n, X, Q, R², R³, R⁴, R⁵ and A are as defined above in embodiment 6-la and R^{1a} is fluorine, preferably as defined above in embodiment 6-1a.1 and R^{1a} is fluorine (embodiment Ia-6-1a.1).

Another embodiment (embodiment Ia-6-2a) is a compound of formula (Ia) wherein n, X, Q, R², R³, R⁴, R⁵ and A are as defined above in embodiment 6-2a and R^{1a} is fluorine, preferably as defined above in embodiment 6-2a.1 and R^{1a} is fluorine (embodiment Ia-6-2a.1).

Another embodiment (embodiment Ia-7-1) is a compound of formula (Ia) wherein n, X, Q, R², R³, R⁴, R⁵ and A are as defined above in embodiment 7-1 and R^{1a} is fluorine, preferably as defined above in embodiment 7-1.1 and R^{1a} is fluorine (embodiment Ia-7-1.1).

Another embodiment (embodiment Ia-7-2) is a compound of formula (Ia) wherein n, X, Q, R², R³, R⁴, R⁵ and A are as defined above in embodiment 7-2 and R^{1a} is fluorine, preferably as defined above in embodiment 7-2.1 and R^{1a} is fluorine (embodiment Ia-7-2.1).

Another embodiment (embodiment Ia-8-1) is a compound of formula (Ia) wherein n, X, Q, R², R³, R⁴, R⁵ and A are as defined above in embodiment 8-1 and R^{1a} is fluorine, preferably as defined above in embodiment 8-1.1 and R^{1a} is fluorine (embodiment Ia-8-1.1).

Another embodiment (embodiment Ia-8-2) is a compound of formula (Ia) wherein n, X, Q, R², R³, R⁴, R⁵ and A are as defined above in embodiment 8-2 and R^{1a} is fluorine, preferably as defined above in embodiment 8-2.1 and R^{1a} is fluorine (embodiment Ia-8-2.1).

Another embodiment (embodiment Ia-9-1) is a compound of formula (Ia) wherein n, X, Q, R², R³, R⁴, R⁵ and A are as defined above in embodiment 9-1 and R^{1a} is fluorine, preferably as defined above in embodiment 9-1.1 and R^{1a} is fluorine (embodiment Ia-9-1.1).

Another embodiment (embodiment Ia-9-2) is a compound of formula (Ia) wherein n, X, Q, R², R³, le, R⁵ and A are as defined above in embodiment 9-2 and R^{1a} is fluorine, preferably as defined above in embodiment 9-2.1 and R^{1a} is fluorine (embodiment Ia-9-2.1).

Another embodiment (embodiment Ia-10-1) is a compound of formula (Ia) wherein n, X, Q, R², R³, le, R⁵ and A are as defined above in embodiment 10-1 and R^{1a} is fluorine, preferably as defined above in embodiment 10-1.1 and lea is fluorine (embodiment Ia-10-1.1).

Another embodiment (embodiment Ia-10-2) is a compound of formula (Ia) wherein n, X, Q, R², R³, le, R⁵ and A are as defined above in embodiment 10-2 and R^{1a} is fluorine, preferably as defined above in embodiment 10-2.1 and R^{1a} is fluorine (embodiment Ia-10-2.1).

Another embodiment (embodiment Ia-10-3) is a compound of formula (Ia) wherein n, X, Q, R², R³, le, R⁵ and A are as defined above in embodiment 10-3 and R^{1a} is fluorine, preferably as defined above in embodiment 10-3.1 and R^{1a} is fluorine (embodiment Ia-10-3.1).

Another embodiment (embodiment Ia-10-4) is a compound of formula (Ia) wherein n, X, Q, R², R³, le, R⁵ and A are as defined above in embodiment 10-4 and R^{1a} is fluorine, preferably as defined above in embodiment 10-4.1 and R^{1a} is fluorine (embodiment Ia-10-4.1).

In especially preferred compounds of formula (Ia) the structural elements n, X, Q, R^{1a}, R² and A are as described in the embodiments above (Ia-1-1 to Ia-10-4.1) and R³ and R⁴ and R⁵ are hydrogen.

The present invention is also directed to novel compounds of formula (Ib)

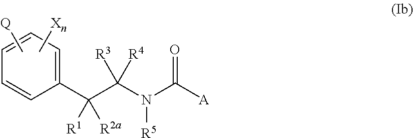

wherein
n, X, Q, R³, R⁴, R⁵ and A are as defined above in embodiment 1-1 and
R^{2a} is fluorine (embodiment Ib-1-1), preferably as defined above in embodiment 1-1.1 and R^{2a} is fluorine (embodiment Ib-1-1.1).

Another embodiment (embodiment Ib-1-2) is a compound of formula (Ib) wherein n, X, Q, R¹, R³, R⁴, R⁵ and A are as defined above in embodiment 1-2 and R^{2a} is fluorine, preferably as defined above in embodiment 1-2.1 and R^{2a} is fluorine (embodiment Ib-1-2.1).

Another embodiment (embodiment Ib-1-1a) is a compound of formula (Ib) wherein n, X, Q, R¹, R³, R⁴, R⁵ and A are as defined above in embodiment 1-1a and R^{2a} is fluorine, preferably as defined above in embodiment 1-1a.1 and R^{2a} is fluorine (embodiment Ib-1-1a.1).

Another embodiment (embodiment Ib-1-2a) is a compound of formula (Ib) wherein n, X, Q, R¹, R³, R⁴, R⁵ and A are as defined above in embodiment 1-2a and R^{2a} is fluorine, preferably as defined above in embodiment 1-2a.1 and R^{2a} is fluorine (embodiment Ib-1-2a.1).

Another embodiment (embodiment Ib-2-1) is a compound of formula (Ib) wherein n, X, Q, R¹, R³, R⁴, R⁵ and A are as defined above in embodiment 2-1 and R^{2a} is fluorine, preferably as defined above in embodiment 2-1.1 and R^{2a} is fluorine (embodiment Ib-2-1.1).

Another embodiment (embodiment Ib-2-2) is a compound of formula (Ib) wherein n, X, Q, R¹, R³, R⁴, R⁵ and A are as defined above in embodiment 2-2 and R^{2a} is fluorine, preferably as defined above in embodiment 2-2.1 and R^{2a} is fluorine (embodiment Ib-2-2.1).

Another embodiment (embodiment Ib-3-1) is a compound of formula (Ib) wherein n, X, Q, R¹, R³, R⁴, R⁵ and A are as defined above in embodiment 3-1 and R^{2a} is fluorine, preferably as defined above in embodiment 3-1.1 and R^{2a} is fluorine (embodiment Ib-3-1.1).

Another embodiment (embodiment Ib-3-2) is a compound of formula (Ib) wherein n, X, Q, R¹, R³, R⁴, R⁵ and A are as defined above in embodiment 3-2 and R^{2a} is fluorine, preferably as defined above in embodiment 3-2.1 and R^{2a} is fluorine (embodiment Ib-3-2.1).

Another embodiment (embodiment Ib-4-1) is a compound of formula (Ib) wherein n, X, Q, R¹, R³, R⁴, R⁵ and A are as defined above in embodiment 4-1 and R^{2a} is fluorine, preferably as defined above in embodiment 4-1.1 and R^{2a} is fluorine (embodiment Ib-4-1.1).

Another embodiment (embodiment Ib-4-2) is a compound of formula (Ib) wherein n, X, Q, R¹, R³, R⁴, R⁵ and A are as defined above in embodiment 4-2 and R^{2a} is fluorine, preferably as defined above in embodiment 4-2.1 and R^{2a} is fluorine (embodiment Ib-4-2.1).

Another embodiment (embodiment Ib-5-1) is a compound of formula (Ib) wherein n, X, Q, R¹, R³, R⁴, R⁵ and A are as defined above in embodiment 5-1 and R^{2a} is fluorine, preferably as defined above in embodiment 5-1.1 and R^{2a} is fluorine (embodiment Ib-5-1.1).

Another embodiment (embodiment Ib-5-2) is a compound of formula (Ib) wherein n, X, Q, R¹, R³, R⁴, R⁵ and A are as defined above in embodiment 5-2 and R^{2a} is fluorine, preferably as defined above in embodiment 5-2.1 and R^{2a} is fluorine (embodiment Ib-5-2.1).

Another embodiment (embodiment Ib-5-3) is a compound of formula (Ib) wherein n, X, Q, R¹, R³, R⁴, R⁵ and A are as defined above in embodiment 5-3 and R^{2a} is fluorine, preferably as defined above in embodiment 5-3.1 and R^{2a} is fluorine (embodiment Ib-5-3.1).

Another embodiment (embodiment Ib-5-4) is a compound of formula (Ib) wherein n, X, Q, R¹, R³, R⁴, R⁵ and A are as defined above in embodiment 5-4 and R^{2a} is fluorine, preferably as defined above in embodiment 5-4.1 and R^{2a} is fluorine (embodiment Ib-5-4.1).

Another embodiment (embodiment Ib-6-1) is a compound of formula (Ib) wherein n, X, Q, R¹, R³, R⁴, R⁵ and A are as defined above in embodiment 6-1 and R^{2a} is fluorine, preferably as defined above in embodiment 6-1.1 and R^{2a} is fluorine (embodiment Ib-6-1.1).

Another embodiment (embodiment Ib-6-2) is a compound of formula (Ib) wherein n, X, Q, R¹, R³, R⁴, R⁵ and A are as defined above in embodiment 6-2 and R^{2a} is fluorine, preferably as defined above in embodiment 6-2.1 and R^{2a} is fluorine (embodiment Ib-6-2.1).

Another embodiment (embodiment Ib-6-1a) is a compound of formula (Ib) wherein n, X, Q, R¹, R³, R⁴, R⁵ and A are as defined above in embodiment 6-la and R^{2a} is fluorine, preferably as defined above in embodiment 6-1a.1 and R^{2a} is fluorine (embodiment Ib-6-1a.1).

Another embodiment (embodiment Ib-6-2a) is a compound of formula (Ib) wherein n, X, Q, R¹, R³, R⁴, R⁵ and A are as defined above in embodiment 6-2a and R^{2a} is fluorine, preferably as defined above in embodiment 6-2a.1 and R^{2a} is fluorine (embodiment Ib-6-2a.1).

Another embodiment (embodiment Ib-7-1) is a compound of formula (Ib) wherein n, X, Q, R¹, R³, R⁴, R⁵ and A are as defined above in embodiment 7-1 and R^{2a} is fluorine, preferably as defined above in embodiment 7-1.1 and R^{2a} is fluorine (embodiment Ib-7-1.1).

Another embodiment (embodiment Ib-7-2) is a compound of formula (Ib) wherein n, X, Q, R¹, R³, R⁴, R⁵ and A are as defined above in embodiment 7-2 and R^{2a} is fluorine, preferably as defined above in embodiment 7-2.1 and R^{2a} is fluorine (embodiment Ib-7-2.1).

Another embodiment (embodiment Ib-8-1) is a compound of formula (Ib) wherein n, X, Q, R¹, R³, R⁴, R⁵ and A are as defined above in embodiment 8-1 and R^{2a} is fluorine, preferably as defined above in embodiment 8-1.1 and R^{2a} is fluorine (embodiment Ib-8-1.1).

Another embodiment (embodiment Ib-8-2) is a compound of formula (Ib) wherein n, X, Q, R¹, R³, R⁴, R⁵ and A are as defined above in embodiment 8-2 and R^{2a} is fluorine, preferably as defined above in embodiment 8-2.1 and R^{2a} is fluorine (embodiment Ib-8-2.1).

Another embodiment (embodiment Ib-9-1) is a compound of formula (Ib) wherein n, X, Q, R¹, R³, R⁴, R⁵ and A are as defined above in embodiment 9-1 and R^{2a} is fluorine, preferably as defined above in embodiment 9-1.1 and R^{2a} is fluorine (embodiment Ib-9-1.1).

Another embodiment (embodiment Ib-9-2) is a compound of formula (Ib) wherein n, X, Q, R¹, R³, R⁴, R⁵ and A are as defined above in embodiment 9-2 and R^{2a} is fluorine, preferably as defined above in embodiment 9-2.1 and R^{2a} is fluorine (embodiment Ib-9-2.1).

Another embodiment (embodiment Ib-10-1) is a compound of formula (Ib) wherein n, X, Q, R¹, R³, R⁴, R⁵ and A are as defined above in embodiment 10-1 and R^{2a} is fluorine, preferably as defined above in embodiment 10-1.1 and R^{2a} is fluorine (embodiment Ib-10-1.1).

Another embodiment (embodiment Ib-10-2) is a compound of formula (Ib) wherein n, X, Q, R¹, R³, R⁴, R⁵ and A are as defined above in embodiment 10-2 and R^{2a} is fluorine, preferably as defined above in embodiment 10-2.1 and R^{2a} is fluorine (embodiment Ib-10-2.1).

Another embodiment (embodiment Ib-10-3) is a compound of formula (Ib) wherein n, X, Q, R¹, R³, R⁴, R⁵ and A are as defined above in embodiment 10-3 and R^{2a} is fluorine, preferably as defined above in embodiment 10-3.1 and R^{2a} is fluorine (embodiment Ib-10-3.1).

Another embodiment (embodiment Ib-10-4) is a compound of formula (Ib) wherein n, X, Q, R¹, R³, R⁴, R⁵ and A are as defined above in embodiment 10-4 and R^{2a} is fluorine, preferably as defined above in embodiment 10-4.1 and R^{2a} is fluorine (embodiment Ib-10-4.1).

In especially preferred compounds of formula (Ib) the structural elements n, X, Q, R^{2a}, R¹ and A are as described in the embodiments above (Ib-1-1 to Ib-10-4.1) and R³ and R⁴ and R⁵ are hydrogen.

The present invention is also directed to novel compounds of formula (Ic)

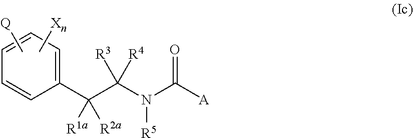

wherein
n, X, Q, R³, R⁴, R⁵ and A are as defined above in embodiment 1-1 and both R^{1a} and R^{2a} are fluorine (embodiment Ic-1-1), preferably as defined above in embodiment 1-1.1 and both R^{1a} and R^{2a} are fluorine (embodiment Ic-1-1.1).

Another embodiment (embodiment Ic-1-2) is a compound of formula (Ic) wherein n, X, Q, R³, R⁴, R⁵ and A are as defined above in embodiment 1-2 and both R^{1a} and R^{2a} are fluorine, preferably as defined above in embodiment 1-2.1 and both R^{1a} and R^{2a} are fluorine (embodiment Ic-1-2.1).

Another embodiment (embodiment Ic-1-1a) is a compound of formula (Ic) wherein n, X, Q, R³, R⁴, R⁵ and A are as defined above in embodiment 1-la and both R^{1a} and R^{2a} are fluorine, preferably as defined above in embodiment 1-1a.1 and both R^{1a} and R^{2a} are fluorine (embodiment Ic-1-1a.1).

Another embodiment (embodiment Ic-1-2a) is a compound of formula (Ic) wherein n, X, Q, R³, R⁴, R⁵ and A are as defined above in embodiment 1-2a and both R^{1a} and R^{2a} are fluorine, preferably as defined above in embodiment 1-2a.1 and both R^{1a} and R^{2a} are fluorine (embodiment Ic-1-2a.1).

Another embodiment (embodiment Ic-2-1) is a compound of formula (Ic) wherein n, X, Q, R³, R⁴, R⁵ and A are as defined above in embodiment 2-1 and both R^{1a} and R^{2a} are fluorine, preferably as defined above in embodiment 2-1.1 and both R^{1a} and R^{2a} are fluorine (embodiment Ic-2-1.1).

Another embodiment (embodiment Ic-2-2) is a compound of formula (Ic) wherein n, X, Q, R³, R⁴, R⁵ and A are as defined above in embodiment 2-2 and both R^{1a} and R^{2a} are fluorine, preferably as defined above in embodiment 2-2.1 and both R^{1a} and R^{2a} are fluorine (embodiment Ic-2-2.1).

Another embodiment (embodiment Ic-3-1) is a compound of formula (Ic) wherein n, X, Q, R³, R⁴, R⁵ and A are as defined above in embodiment 3-1 and both R^{1a} and R^{2a} are fluorine, preferably as defined above in embodiment 3-1.1 and both R^{1a} and R^{2a} are fluorine (embodiment Ic-3-1.1).

Another embodiment (embodiment Ic-3-2) is a compound of formula (Ic) wherein n, X, Q, R³, R⁴, R⁵ and A are as defined above in embodiment 3-2 and both R^{1a} and R^{2a} are fluorine, preferably as defined above in embodiment 3-2.1 and both R^{1a} and R^{2a} are fluorine (embodiment Ic-3-2.1).

Another embodiment (embodiment Ic-4-1) is a compound of formula (Ic) wherein n, X, Q, R³, R⁴, R⁵ and A are as defined above in embodiment 4-1 and both R^{1a} and R^{2a} are fluorine, preferably as defined above in embodiment 4-1.1 and both R^{1a} and R^{2a} are fluorine (embodiment Ic-4-1.1).

Another embodiment (embodiment Ic-4-2) is a compound of formula (Ic) wherein n, X, Q, R³, R⁴, R⁵ and A are as defined above in embodiment 4-2 and both R^{1a} and R^{2a} are fluorine, preferably as defined above in embodiment 4-2.1 and both R^{1a} and R^{2a} are fluorine (embodiment Ic-4-2.1).

Another embodiment (embodiment Ic-5-1) is a compound of formula (Ic) wherein n, X, Q, R³, R⁴, R⁵ and A are as defined above in embodiment 5-1 and both R^{1a} and R^{2a} are fluorine, preferably as defined above in embodiment 5-1.1 and both R^{1a} and R^{2a} are fluorine (embodiment Ic-5-1.1).

Another embodiment (embodiment Ic-5-2) is a compound of formula (Ic) wherein n, X, Q, R³, R⁴, R⁵ and A are as defined above in embodiment 5-2 and both R^{1a} and R^{2a} are fluorine, preferably as defined above in embodiment 5-2.1 and both R^{1a} and R^{2a} are fluorine (embodiment Ic-5-2.1).

Another embodiment (embodiment Ic-5-3) is a compound of formula (Ic) wherein n, X, Q, R³, R⁴, R⁵ and A are as defined above in embodiment 5-3 and both R^{1a} and R^{2a} are fluorine, preferably as defined above in embodiment 5-3.1 and both R^{1a} and R^{2a} are fluorine (embodiment Ic-5-3.1).

Another embodiment (embodiment Ic-5-4) is a compound of formula (Ic) wherein n, X, Q, R³, R⁴, R⁵ and A are as defined above in embodiment 5-4 and both R^{1a} and R^{2a} are fluorine, preferably as defined above in embodiment 5-4.1 and both R^{1a} and R^{2a} are fluorine (embodiment Ic-5-4.1).

Another embodiment (embodiment Ic-6-1) is a compound of formula (Ic) wherein n, X, Q, R³, R⁴, R⁵ and A are as defined above in embodiment 6-1 and both R^{1a} and R^{2a} are fluorine, preferably as defined above in embodiment 6-1.1 and both R^{1a} and R^{2a} are fluorine (embodiment Ic-6-1.1).

Another embodiment (embodiment Ic-6-2) is a compound of formula (Ic) wherein n, X, Q, R³, R⁴, R⁵ and A are as defined above in embodiment 6-2 and both R^{1a} and R^{2a} are fluorine, preferably as defined above in embodiment 6-2.1 and both R^{1a} and R^{2a} are fluorine (embodiment Ic-6-2.1).

Another embodiment (embodiment Ic-6-1a) is a compound of formula (Ic) wherein n, X, Q, R³, R⁴, R⁵ and A are as defined above in embodiment 6-la and both R^{1a} and R^{2a} are fluorine, preferably as defined above in embodiment 6-1a.1 and both R^{1a} and R^{2a} are fluorine (embodiment Ic-6-1a.1).

Another embodiment (embodiment Ic-6-2a) is a compound of formula (Ic) wherein n, X, Q, R³, R⁴, R⁵ and A are as defined above in embodiment 6-2a and both R^{1a} and R^{2a} are fluorine, preferably as defined above in embodiment 6-2a.1 and both R^{1a} and R^{2a} are fluorine (embodiment Ic-6-2a.1).

Another embodiment (embodiment Ic-7-1) is a compound of formula (Ic) wherein n, X, Q, R³, R⁴, R⁵ and A are as defined above in embodiment 7-1 and both R^{1a} and R^{2a} are fluorine, preferably as defined above in embodiment 7-1.1 and both R^{1a} and R^{2a} are fluorine (embodiment Ic-7-1.1).

Another embodiment (embodiment Ic-7-2) is a compound of formula (Ic) wherein n, X, Q, R³, R⁴, R⁵ and A are as defined above in embodiment 7-2 and both R^{1a} and R^{2a} are fluorine, preferably as defined above in embodiment 7-2.1 and both R^{1a} and R^{2a} are fluorine (embodiment Ic-7-2.1).

Another embodiment (embodiment Ic-8-1) is a compound of formula (Ic) wherein n, X, Q, R³, R⁴, R⁵ and A are as defined above in embodiment 8-1 and both R^{1a} and R^{2a} are fluorine, preferably as defined above in embodiment 8-1.1 and both R^{1a} and R^{2a} are fluorine (embodiment Ic-8-1.1).

Another embodiment (embodiment Ic-8-2) is a compound of formula (Ic) wherein n, X, Q, R³, R⁴, R⁵ and A are as defined above in embodiment 8-2 and both R^{1a} and R^{2a} are fluorine, preferably as defined above in embodiment 8-2.1 and both R^{1a} and R^{2a} are fluorine (embodiment Ic-8-2.1).

Another embodiment (embodiment Ic-9-1) is a compound of formula (Ic) wherein n, X, Q, R³, R⁴, R⁵ and A are as defined above in embodiment 9-1 and both R^{1a} and R^{2a} are fluorine, preferably as defined above in embodiment 9-1.1 and both R^{1a} and R^{2a} are fluorine (embodiment Ic-9-1.1).

Another embodiment (embodiment Ic-9-2) is a compound of formula (Ic) wherein n, X, Q, R³, R⁴, R⁵ and A are as defined above in embodiment 9-2 and both R^{1a} and R^{2a} are fluorine, preferably as defined above in embodiment 9-2.1 and both R^{1a} and R^{2a} are fluorine (embodiment Ic-9-2.1).

Another embodiment (embodiment Ic-10-1) is a compound of formula (Ic) wherein n, X, Q, R³, R⁴, R⁵ and A are as defined above in embodiment 10-1 and both R^{1a} and R^{2a} are fluorine, preferably as defined above in embodiment 10-1.1 and both R^{1a} and R^{2a} are fluorine (embodiment Ic-10-1.1).

Another embodiment (embodiment Ic-10-2) is a compound of formula (Ic) wherein n, X, Q, R³, R⁴, R⁵ and A are as defined above in embodiment 10-2 and both R^{1a} and R^{2a} are fluorine, preferably as defined above in embodiment 10-2.1 and both R^{1a} and R^{2a} are fluorine (embodiment Ic-10-2.1).

Another embodiment (embodiment Ic-10-3) is a compound of formula (Ic) wherein n, X, Q, R³, R⁴, R⁵ and A are as defined above in embodiment 10-3 and both R^{1a} and R^{2a} are fluorine, preferably as defined above in embodiment 10-3.1 and both R^{1a} and R^{2a} are fluorine (embodiment Ic-10-3.1).

Another embodiment (embodiment Ic-10-4) is a compound of formula (Ic) wherein n, X, Q, R³, R⁴, R⁵ and A are as defined above in embodiment 10-4 and both R^{1a} and R^{2a} are fluorine, preferably as defined above in embodiment 10-4.1 and both R^{1a} and R^{2a} are fluorine (embodiment Ic-10-4.1).

In especially preferred compounds of formula (Ic) the structural elements n, X, Q, R^{1a}, R^{2a} and A are as described in the embodiments above (Ic-1-1 to Ic-10-4.1) and R³ and R⁴ and R⁵ are hydrogen.

### Procedures and Methods

The synthesis of the compounds of the formula (I) can be performed according to or in analogy to scheme 1, 2 or 3. The required starting materials are known or accessible via generally known procedures which are described in more detail in WO 2001/011965 A1 (P1), WO 2005/058828 A1 (P2), WO2005/014545 A2 (P3), WO 2005/103004 A1 (P4), WO 2006/122952 A1 (P5), EP 2 289 880 A1 (P6), WO 2006/008191 A1 (P7), WO 2006/008192 A1 (P8), WO 2004/074280 A1 (P9), WO 2005/058833 A2 (P10), WO 2005/085238 A1 (P11), WO 2005/103006 A1 (P12), WO 2006/122955 A1 (P13), WO 2006/008194 A1 (P14), WO 2006/008193 A1 (P15), WO 2006/067103 A2 (P16) and in case of R¹═R²=fluorine WO 2013/064460 A1 (P17) or R¹=fluorine and R²=alkyl or hydrogen WO 2007141009 A1 (P18).

Benzylnitriles like (II) are commercial available or are synthesized as described in WO2013043232, followed by reduction to the Boc-protected amines (III) with sodium borohydride in the presence of nickel chloride and Boc-anhydride. The protected amines (III) can be cleaved with hydrogen chloride in methanol to the amine-hydrochlorides (IV). In principle such phenethylamines and their salts are also commercially available.

The amine-hydrochlorides or amines (IV), (VIII), (XIII) or (XV) are then coupled with the appropriate acids and a coupling reagent such as HOBT-EDC or TBTU to yield for example the amides (V), (IX) or (XIV) (wherein B³ or B⁶ represents N or CH and Rₒ is defined as described before) or the amide (I-d). Alternatively amides (V), (IX), (XIV) or (I-d) can be prepared using the appropriate acylchloride moiety and a base e.g. Hünig's one.

The compounds of the formula (I-a), (I-b) or (I-c) are then synthesized by a coupling reaction. In the case of Q=N-bonded azoles, a copper-mediated process with copper-(I)-oxide, salicylaldoxime as ligand in a solvent as acetonitrile in the presence of a base as cesium carbonate may be used. In the case of Q=carbon-bonded heterocycles, a Suzuki-type coupling with the appropriate boronic acid or ester in the presence of a palladium catalyst and a base may be used. Alternatively for the synthesis of compounds (I-d) the Q moiety can also be introduced earlier in the synthesis, following a synthetic pathway similar to the one exemplified in scheme 4.

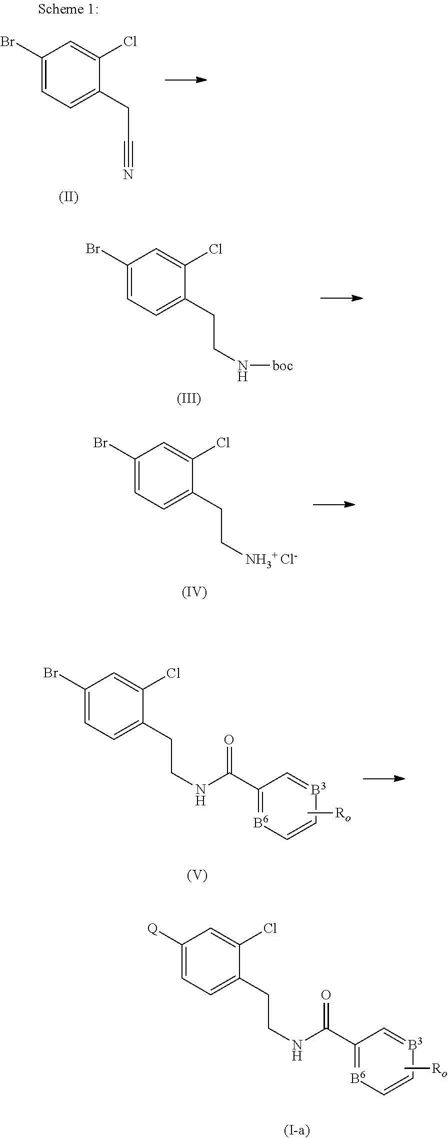

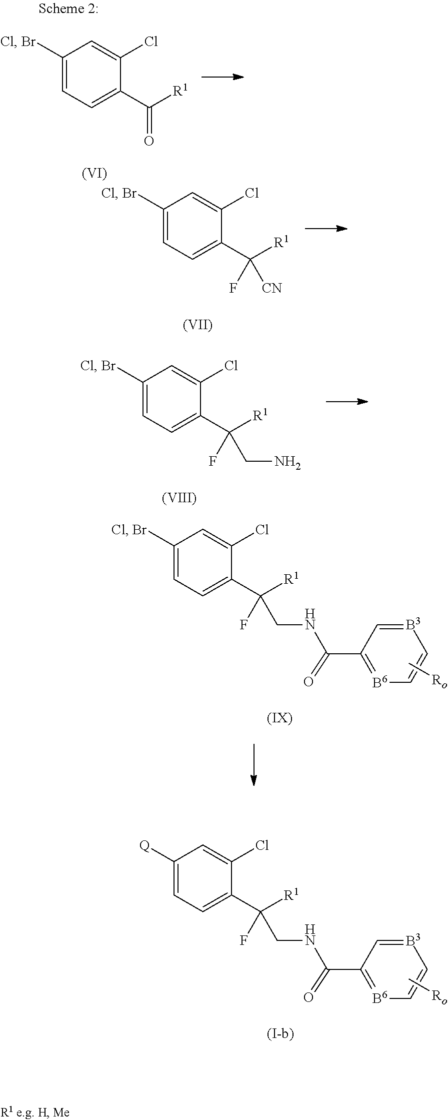

Compounds of formula (VIII) (scheme 2) can be prepared from nitrites (VII) by reduction using for example borane-THF. Nitrites of formula (VII) can be prepared from ketones or aldehydes (VI) in a solvent such as dichloromethane using in a first time zinc iodide and an addition of trimethylsilylcyanide and in a second time diethylaminosulfur trifluoride to convert the in situ formed alcohol into fluoro derivatives (VII) as it is described in P18.

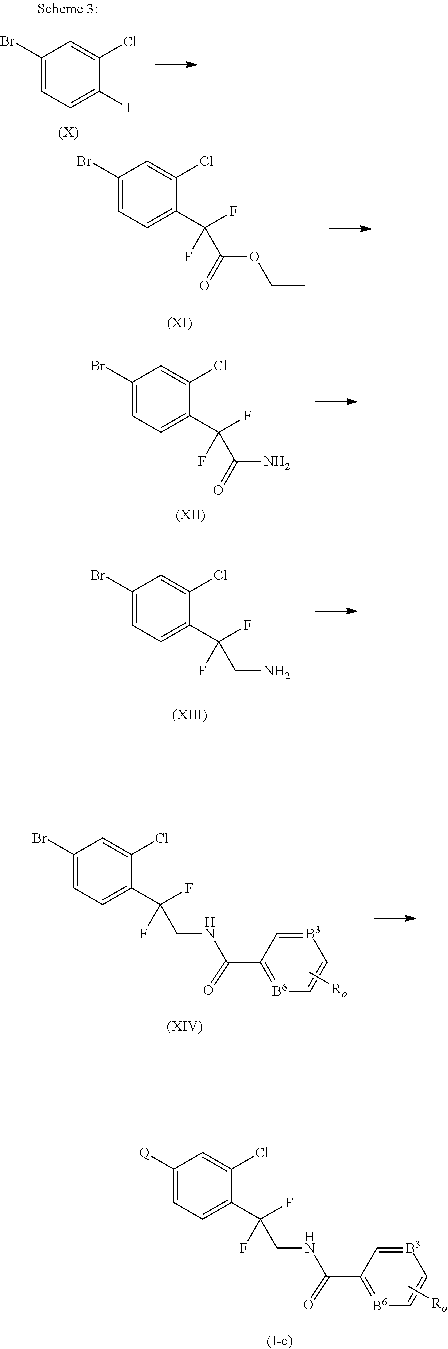

Amines of formula (XIII) (scheme 3) can be prepared from carboxamides (XII) by reduction using for example borane-THF. Carboxamides of formula (XII) are easily prepared by the reaction of ethyl esters of formula (XI) and ammonia dissolved in methanol. Ethyl esters of formula (XI) can be prepared by copper-mediated reaction of phenyl iodides e.g. formula (X) with bromdifluoroacetic acid ethylester as it is described in P17.

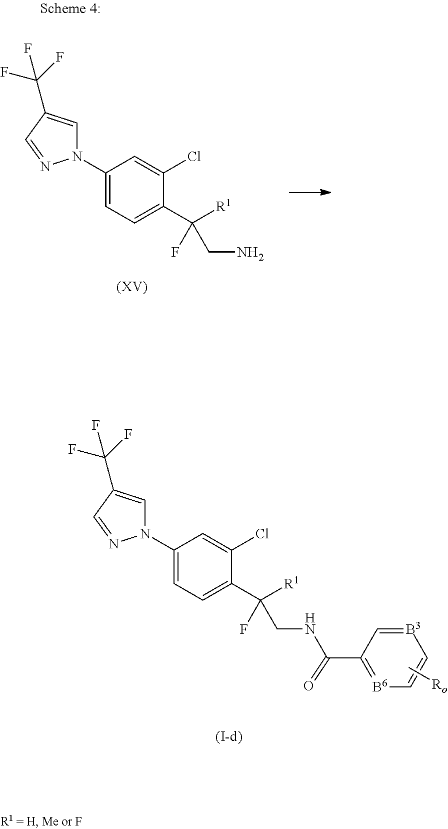

In principle, the synthesis of amines with N-bonded 5-membered heterocycles like (XV) according to scheme 4 are described in WO2014/004064 (P19).

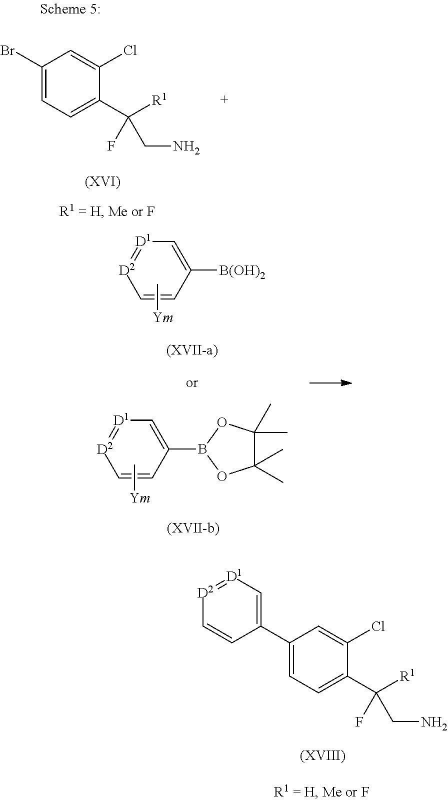

Amines with pyridyl substitution like (XVIII; D¹, D² are CH or N for 3- or 4-pyridyl, Y and m are as described as before) are synthesized via Suzuki type couplings of phenyl bromides like (XVI) with the appropriate boronic acid (XVII-a; D¹, D² are CH or N for 3- or 4-pyridyl, Y and m are as described as before) or pinacol ester (XVII-b; D¹, D² are CH or N for 3- or 4-pyridyl, Y and m are as described as before) in the presence of a palladium catalyst and a base may be used (scheme 5).

Of particular interest are intermediates of the procedures and methods described herein. These intermediates are further individual embodiments of the invention. In addition to the intermediates described above, further intermediates are described in the following.

Another embodiment of the invention is a compound of formula (INT)

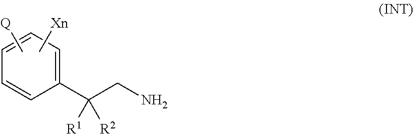

wherein R¹, R², Q, X and n are as defined above.

In an individual aspect, R¹, R², Q, X and n are as defined above in embodiment 1-1, preferably as defined above in embodiment 1-1.1.

In an individual aspect, R¹, R², Q, X and n are as defined above in embodiment 1-2, preferably as defined above in embodiment 1-2.1.

In an individual aspect, R¹, R², Q, X and n are as defined above in embodiment 1-1a, preferably as defined above in embodiment 1-1a.1.

In an individual aspect, R¹, R², Q, X and n are as defined above in embodiment 1-2a, preferably as defined above in embodiment 1-2a.1.

In an individual aspect, R¹, R², Q, X and n are as defined above in embodiment 2-1, preferably as defined above in embodiment 2-1.1.

In an individual aspect, R¹, R², Q, X and n are as defined above in embodiment 2-2 and R^{1a} is fluorine, preferably as defined above in embodiment 2-2.1.

In an individual aspect, R¹, R², Q, X and n are as defined above in embodiment 3-1, preferably as defined above in embodiment 3-1.1.

In an individual aspect, R¹, R², Q, X and n are as defined above in embodiment 3-2, preferably as defined above in embodiment 3-2.1.

In an individual aspect, R¹, R², Q, X and n are as defined above in embodiment 4-1, preferably as defined above in embodiment 4-1.1.

In an individual aspect, R¹, R², Q, X and n are as defined above in embodiment 4-2, preferably as defined above in embodiment 4-2.1.

In an individual aspect, R¹, R², Q, X and n are as defined above in embodiment 5-1, preferably as defined above in embodiment 5-1.1.

In an individual aspect, R¹, R², Q, X and n are as defined above in embodiment 5-2, preferably as defined above in embodiment 5-2.1.

In an individual aspect, R¹, R², Q, X and n are as defined above in embodiment 5-3, preferably as defined above in embodiment 5-3.1.

In an individual aspect, R¹, R², Q, X and n are as defined above in embodiment 5-4, preferably as defined above in embodiment 5-4.1.

In an individual aspect, R¹, R², Q, X and n are as defined above in embodiment 6-1, preferably as defined above in embodiment 6-1.1.

In an individual aspect, R¹, R², Q, X and n are as defined above in embodiment 6-2, preferably as defined above in embodiment 6-2.1.

In an individual aspect, R¹, R², Q, X and n are as defined above in embodiment 6-1a, preferably as defined above in embodiment 6-1a.1.

In an individual aspect, R¹, R², Q, X and n are as defined above in embodiment 6-2a, preferably as defined above in embodiment 6-2a.1.

In an individual aspect, R¹, R², Q, X and n are as defined above in embodiment 7-1, preferably as defined above in embodiment 7-1.1.

In an individual aspect, R¹, R², Q, X and n are as defined above in embodiment 7-2, preferably as defined above in embodiment 7-2.1.

In an individual aspect, R¹, R², Q, X and n are as defined above in embodiment 8-1, preferably as defined above in embodiment 8-1.1.

In an individual aspect, R¹, R², Q, X and n are as defined above in embodiment 8-2, preferably as defined above in embodiment 8-2.1.

In an individual aspect, R¹, R², Q, X and n are as defined above in embodiment 9-1, preferably as defined above in embodiment 9-1.1.

In an individual aspect, R¹, R², Q, X and n are as defined above in embodiment 9-2, preferably as defined above in embodiment 9-2.1.

In an individual aspect, R¹, R², Q, X and n are as defined above in embodiment 10-1, preferably as defined above in embodiment 10-1.1.

In an individual aspect, R¹, R², Q, X and n are as defined above in embodiment 10-2, preferably as defined above in embodiment 10-2.1.

In an individual aspect, R¹, R², Q, X and n are as defined above in embodiment 10-3, preferably as defined above in embodiment 10-3.1.

In an individual aspect, R¹, R², Q, X and n are as defined above in embodiment 10-4, preferably as defined above in embodiment 10-4.1.

An example of a preferred compound of formula (INT) is a compound which is represented by formula (INT-1)

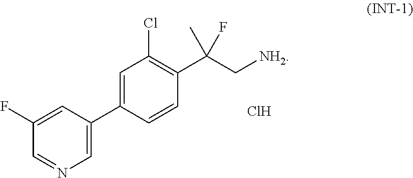 [SMILES: CC(F)(CN)c1ccc(-c2cncc(F)c2)cc1Cl ; Cl]

Another example of a preferred compound of formula (INT) is a compound which is represented by formula (INT-2)

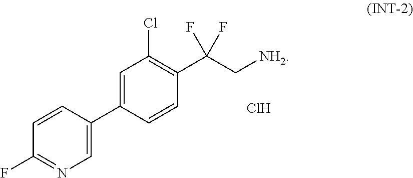 [SMILES: NCC(F)(F)c1ccc(-c2ccc(F)nc2)cc1Cl ; Cl]

The compound according to the present invention can be prepared according to the processes described above. It will nevertheless be understood that, on the basis of his general knowledge and of available publications, the skilled worker will be able to adapt this method according to the specifics of each of the compounds, which it is desired to synthesize.

Amongst others, this patent application is directed to the use of compounds of formula (I) as well as to specific compounds of formula (Ia), (Ib) or (Ic). Therefore the following explanations shall be understood to refer to all subjects of the invention equally, especially to the use of the compounds of formula (I) as well as to the compounds of formula (Ia), (Ib) or (Ic).

### Isomers

Depending on the nature of the substituents, the compounds of the formula (I), (Ia), (Ib) or (Ic) may be in the form of geometric and/or optically active isomers or corresponding isomer mixtures in different compositions. These stereoisomers are, for example, enantiomers, diastereomers, atropisomers or geometric isomers. Accordingly, the invention encompasses both pure stereoisomers and any mixture of these isomers.

### Methods and Uses

The invention also relates to methods for controlling animal pests, in which compounds of the formula (I), (Ia), (Ib) or (Ic) are allowed to act on animal pests and/or their habitat. The control of the animal pests is preferably conducted in agriculture and forestry, and in material protection. Preferably excluded herefrom are methods for the surgical or therapeutic treatment of the human or animal body and diagnostic methods carried out on the human or animal body.

The invention furthermore relates to the use of the compounds of the formula (I), (Ia), (Ib) or (Ic) as pesticides, in particular crop protection agents.

In the context of the present application, the term “pesticide” in each case also always comprises the term “crop protection agent”.

The compounds of the formula (I), (Ia), (Ib) or (Ic), having good plant tolerance, favourable homeotherm toxicity and good environmental compatibility, are suitable for protecting plants and plant organs against biotic and abiotic stressors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector. They can preferably be used as pesticides. They are active against normally sensitive and resistant species and against all or some stages of development. The abovementioned pests include:

Pests from the phylum of the Arthropoda, in particular from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;

from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, for example Blatta orientalis, Blattella asahinai, Blattella germanica, Leucophaea maderae, Panchlora spp., Parcoblatta spp., Periplaneta spp., for example Periplaneta americana, Periplaneta australasiae, Supella longipalpa;
   from the order of the Coleoptera, for example Acalymma vittatum, Acanthoscelides obtectus, Adoretus spp., Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Buis caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella, Lissorhoptrus oryzophilus, Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;
   from the order of the Diptera, for example Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta, Pegomya spp., for example Pegomya betae, Pegomya hyoscyami, Pegomya rubivora, Phlebotomus spp., Phorbia spp., Phormia spp., Piophila casei, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex;
   from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma pin, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes citri, Diaphorina citri, Diaspis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium corni), Lepidosaphes spp., for example Lepidosaphes ulmi, Lipaphis erysimi, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Nephotettix spp., for example Nephotettix cincticeps Nephotettix nigropictus, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla malt, Psylla pyri, Pteromalus spp., Pyrilla spp., Quadraspidiotus spp., for example Quadraspidiotus juglansregiae, Quadraspidiotus ostreaeformis, Quadraspidiotus perniciosus, Que sada gigas, Rastrococcus spp., Rhopalosiphum spp., for example Rhopalosiphum maidis, Rhopalosiphum oxyacanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;
   from the suborder of the Heteroptera, for example Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Monalonion atratum, Nezara spp., for example Nezara viridula, Oebalus spp., Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
   from the order of the Hymenoptera, for example Acromyrmex spp., Athalia spp., for example Athalia rosae, Atta spp., Diprion spp., for example Diprion similis, Hoplocampa spp., for example Hoplocampa cookei, Hoplocampa testudinea, Lasius spp., Monomorium pharaonis, Sirex spp., Solenopsis invicta, Tapinoma spp., Urocerus spp., Vespa spp., for example Vespa crabro, Xeris spp.;
   from the order of the Isopoda, for example Armadillidium vulgare, Oniscus asellus, Porcellio scaber;
   from the order of the Isoptera, for example Coptotermes spp., for example Coptotermes formosanus, Cornitermes cumulans, Cryptotermes spp., Incisitermes spp., Microtermes obesi, Odontotermes spp., Reticulitermes spp., for example Reticulitermes flavipes, Reticulitermes hesperus;
   from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diatraea saccharalis, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Etiella spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens Hofmannophila pseudospretella, Homoeosoma spp., Homona spp., Hyponomeuta padella, Kakivoria flavofasciata, Laphygma spp., Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Oria spp., Orthaga spp., Ostrinia spp., for example Ostrinia nubilalis, Oulema melanopus, Oulema oryzae, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.; from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;

from the order of the Phthiraptera, for example Damalinia spp., Haematopinus spp., Linognathus spp., Pediculus spp., Phylloxera vastatrix, Phthirus pubis, Trichodectes spp.;

from the order of the Psocoptera, for example Lepinotus spp., Liposcelis spp.;
from the order of the Siphonaptera, for example, Ceratophyllus spp., Ctenocephalides spp., for example Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Heliothrips spp., Hercinothrips femoralis, Rhipiphorothrips cruentatus, Scirtothrips spp., Taeniothrips cardamomi, Thrips spp., for example Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., for example Scutigerella immaculata;
pests from the phylum of the Mollusca, for example from the class of the Bivalvia, for example Dreissena spp.,
and also from the class of the Gastropoda, for example Anion spp., for example Anion ater rufus, Biomphalaria spp., Bulinus spp., Deroceras spp., for example Deroceras laeve, Galba spp., Lymnaea spp., Oncomelania spp., Pomacea spp., Succinea spp.;
animal and human parasites from the phyla of the Platylminthes and Nematoda, for example Aelurostrongylus spp., Amidostomum spp., Ancylostoma spp, Angiostrongylus spp., Anisakis spp., Anoplocephala spp., Ascaris spp., Ascaridia spp., Baylisascaris spp., Brugia spp., Bunostomum spp., Capillaria spp., Chabertia spp., Clonorchis spp., Cooperia spp., Creno soma spp., Cyathostoma spp., Dicrocoelium spp., Dictyocaulus spp., Diphyllobothrium spp., Dipylidium spp., Dirofilaria spp., Dracunculus spp., Echinococcus spp., Echinostoma spp., Enterobius spp., Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., Hy ostrongylus spp., Litomosoides spp., Loa spp., Metastrongylus spp., Metorchis spp., Mesocestoides spp., Moniezia spp., Muellerius spp., Necator spp., Nematodirus spp., Nippostrongylus spp., Oesophagostomum spp., Ollulanus spp., Onchocerca spp, Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., Strongylus spp., Syngamus spp., Taenia spp., Teladorsagia spp., Thelazia spp., Toxascaris spp., Toxocara spp., Trichinella spp., Trichobilharzia spp., Trichostrongylus spp., Trichuris spp., Uncinaria spp., Wuchereria spp.;
plant pests from the phylum of the Nematoda, i.e. phytoparasitic nematodes, in particular Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paraphelenchus spp., Paratrichodorus spp., for example Paratrichodorus minor, Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

Furthermore, it is possible to control, from the subkingdom of the Protozoa, the order of the Coccidia, for example Eimeria spp.

### Nematodes

In the present context, the term “nematodes” comprises all species of the phylum Nematoda and here in particular species acting as parasites on plants or fungi (for example species of the order Aphelenchida, Meloidogyne, Tylenchida and others) or else on humans and animals (for example species of the orders Trichinellida, Tylenchida, Rhabditina and Spirurida) and causing damage in or on these living organisms, and also other parasitic helminths.

A nematicide in crop protection, as described herein, is capable of controlling nematodes.

The term “controlling nematodes” means killing the nematodes or preventing or impeding their development or their growth or preventing or impeding their penetration into or their sucking on plant tissue.

Here, the efficacy of the compounds is determined by comparing mortalities, gall formation, cyst formation, nematode density per volume of soil, nematode density per root, number of nematode eggs per soil volume, mobility of the nematodes between a plant or plant part treated with the compound of the formula (I), (Ia), (Ib) or (Ic) or the treated soil and an untreated plant or plant part or the untreated soil (100%). Preferably, the reduction achieved is 25-50% in comparison to an untreated plant, plant part or the untreated soil, particularly preferably 51-79% and very particularly preferably the complete kill or the complete prevention of development and growth of the nematodes by a reduction of 80 to 100%. The control of nematodes as described herein also comprises the control of proliferation of the nematodes (development of cysts and/or eggs). Compounds of the formula (I), (Ia), (Ib) or (Ic) can also be used to keep the plants or animals healthy, and they can be employed curatively, preventatively or systemically for the control of nematodes.

The person skilled in the art knows methods for determining mortalities, gall formation, cyst formation, nematode density per volume of soil, nematode density per root, number of nematode eggs per volume of soil, mobility of the nematodes.

The use of a compound of the formula (I), (Ia), (Ib) or (Ic) may keep the plant healthy and also comprises a reduction of the damage caused by nematodes and an increase of the harvest yield.

In the present context, the term “nematodes” refers to plant nematodes which comprise all nematodes which damage plants. Plant nematodes comprise phytoparasitic nematodes and soil-borne nematodes. The phytoparasitic nematodes include ectoparasites such as Xiphinema spp., Longidorus spp. and Trichodorus spp.; semiparasites such as Tylenchulus spp.; migratory endoparasites such as Pratylenchus spp., Radopholus spp. and Scutellonema spp.; non-migratory parasites such as Heterodera spp., Globodera spp. and Meloidogyne spp., and also stem and leaf endoparasites such as Ditylenchus spp., Aphelenchoides spp. and Hirschmaniella spp. Particularly damaging root-parasitic soil nematodes are, for example, cyst-forming nematodes of the genera Heterodera or Globodera, and/or root gall nematodes of the genus Meloidogyne. Damaging species of these genera are, for example, Meloidogyne incognita, Heterodera glycines (soya bean cyst nematode), Globodera pallida and Globodera rostochiensis (yellow potato cyst nematode), these species being controlled effectively by the compounds described in the present text. However, the use of the compounds described in the present text is by no means restricted to these genera or species, but also extends in the same manner to other nematodes.

The plant nematodes include, for example, Aglenchus agricola, Anguina tritici, Aphelenchoides arachidis, Aphelenchoides fragaria, and the stem and leaf endoparasites Aphelenchoides spp., Belonolaimus gracilis, Belonolaimus longicaudatus, Belonolaimus nortoni, Bursaphelenchus cocophilus, Bursaphelenchus eremus, Bursaphelenchus xylophilus und Bursaphelenchus spp., Cacopaurus pestis, Criconemella curvata, Criconemella onoensis, Criconemella ornata, Criconemella rusium, Criconemella xenoplax (=Mesocriconema xenoplax) and Criconemella spp.,

Criconemoides ferniae, Criconemoides onoense, Criconemoides ornatum and Criconemoides spp., Ditylenchus destructor, Ditylenchus dipsaci, Ditylenchus myceliophagus and also the stem and leaf endoparasites Ditylenchus spp., Dolichodorus heterocephalus, Globodera pallida (=Heterodera pallida), Globodera rostochiensis (yellow potato cyst nematode), Globodera solanacearum, Globodera tabacum, Globodera virginia and the non-migratory cyst-forming parasites Globodera spp., Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp., Hemicriconemoides, Hemicycliophora arenaria, Hemicycliophora nudata, Hemicycliophora parvana, Heterodera avenae, Heterodera cruciferae, Heterodera glycines (soya bean cyst nematode), Heterodera oryzae, Heterodera schachtii, Heterodera zeae and the non-migratory cyst-forming parasites Heterodera spp., Hirschmaniella gracilis, Hirschmaniella oryzae, Hirschmaniella spinicaudata and the stem and leaf endoparasites Hirschmaniella spp., Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp., Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the non-migratory parasites Meloidogyne spp., Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp., Paratylenchus hamatus, Paratylenchus minutus, Paratylenchus projectus and Paratylenchus spp., Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp., Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp., Rotylenchulus borealis, Rotylenchulus parvus, Rotylenchulus reniformis and Rotylenchulus spp., Rotylenchus laurentinus, Rotylenchus macrodoratus, Rotylenchus robustus, Rotylenchus uniformis and Rotylenchus spp., Scutellonema brachyurum, Scutellonema bradys, Scutellonema clathricaudatum and the migratory endoparasites Scutellonema spp., Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp., Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp., Tylenchulus semipenetrans and the semiparasites Tylenchulus spp., Xiphinema americanum, Xiphinema brevicolle, Xiphinema dimorphicaudatum, Xiphinema index and the ectoparasites Xiphinema spp.

Nematodes for the control of which a compound of the formula (I), (Ia), (Ib) or (Ic) may be used include nematodes of the genus Meloidogyne such as the Southern root-knot nematode (Meloidogyne incognita), the Javanese root-knot nematode (Meloidogyne javanica), the Northern root-knot nematode (Meloidogyne hapla) and the peanut root-knot nematode (Meloidogyne arenaria); nematodes of the genus Ditylenchus such as the potato rot nematode (Ditylenchus destructor) and stem and bulb eelworm (Ditylenchus dipsaci); nematodes of the genus Pratylenchus such as the cob root-lesion nematode (Pratylenchus penetrans), the chrysanthemum root-lesion nematode (Pratylenchus fallax), the coffee root nematode (Pratylenchus coffeae), the tea root nematode (Pratylenchus loosi) and the walnut root-lesion nematode (Pratylenchus vulnus); nematodes of the genus Globodera such as the yellow potato cyst nematode (Globodera rostochiensis) and the white potato cyst nematode (Globodera pallida); nematodes of the genus Heterodera such as the soya bean cyst nematode (Heterodera glycines) and beet cyst eelworm (Heterodera schachtii); nematodes of the genus Aphelenchoides such as the rice white-tip nematode (Aphelenchoides besseyi), the chrysanthemum nematode (Aphelenchoides ritzemabosi) and the strawberry nematode (Aphelenchoides fragariae); nematodes of the genus Aphelenchus such as the fungivorous nematode (Aphelenchus avenae); nematodes of the genus Radopholus, such as the burrowing nematode (Radopholus similis); nematodes of the genus Tylenchulus such as the citrus root nematode (Tylenchulus semipenetrans); nematodes of the genus Rotylenchulus such as the reniform nematode (Rotylenchulus reniformis); tree-dwelling nematodes such as the pine wood nematode (Bursaphelenchus xylophilus) and the red ring nematode (Bursaphelenchus cocophilus) and the like.

Plants for the protection of which a compound of the formula (I), (Ia), (Ib) or (Ic) can be used include plants such as cereals (for example rice, barley, wheat, rye, oats, maize and the like), beans (soya bean, aduki bean, bean, broadbean, peas, peanuts and the like), fruit trees/fruits (apples, citrus species, pears, grapevines, peaches, Japanese apricots, cherries, walnuts, almonds, bananas, strawberries and the like), vegetable species (cabbage, tomato, spinach, broccoli, lettuce, onions, spring onion, pepper and the like), root crops (carrot, potato, sweet potato, radish, lotus root, turnip and the like), plant for industrial raw materials (cotton, hemp, paper mulberry, mitsumata, rape, beet, hops, sugar cane, sugar beet, olive, rubber, palm trees, coffee, tobacco, tea and the like), cucurbits (pumpkin, cucumber, water melon, melon and the like), meadow plants (cocksfoot, sorghum, timothy-grass, clover, alfalfa and the like), lawn grasses (mascarene grass, bentgrass and the like), spice plants etc. (lavender, rosemary, thyme, parsley, pepper, ginger and the like) and flowers (chrysanthemums, rose, orchid and the like).

The compounds of the formula (I), (Ia), (Ib) or (Ic) are particularly suitable for controlling coffee nematodes, in particular Pratylenchus brachyurus, Pratylenchus coffeae, Meloidogyne exigua, Meloidogyne incognita, Meloidogyne coffeicola, Helicotylenchus spp. and also Meloidogyne paranaensis, Rotylenchus spp., Xiphinema spp., Tylenchorhynchus spp. and Scutellonema spp.

The compounds of the formula (I), (Ia), (Ib) or (Ic) are particularly suitable for controlling potato nematodes, in particular Pratylenchus brachyurus, Pratylenchus pratensis, Pratylenchus scribneri, Pratylenchus penetrans, Pratylenchus coffeae, Ditylenchus dipsaci and of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae and Meloinema spp.

The compounds of the formula (I), (Ia), (Ib) or (Ic) are particularly suitable for controlling tomato nematodes, in particular Meloidogyne arenaria, Meloidogyne hapla, Meloidogyne javanica, Meloidogyne incognita, Pratylenchus penetrans and also Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus and Rotylenchulus reniformis.

The compounds of the formula (I), (Ia), (Ib) or (Ic) are particularly suitable for controlling cucumber plant nematodes, in particular Meloidogyne arenaria, Meloidogyne hapla, Meloidogyne javanica, Meloidogyne incognita, Rotylenchulus reniformis and Pratylenchus thornei.

The compounds of the formula (I), (Ia), (Ib) or (Ic) are particularly suitable for controlling cotton nematodes, in particular Belonolaimus longicaudatus, Meloidogyne incognita, Hoplolaimus columbus, Hoplolaimus galeatus and Rotylenchulus reniformis.

The compounds of the formula (I), (Ia), (Ib) or (Ic) are particularly suitable for controlling maize nematodes, in particular Belonolaimus longicaudatus, Paratrichodorus minor and also Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum and Subanguina radiciola.

The compounds of the formula (I), (Ia), (Ib) or (Ic) are particularly suitable for controlling soya bean nematodes, in particular Pratylenchus brachyurus, Pratylenchus pratensis, Pratylenchus penetrans, Pratylenchus scribneri, Belonolaimus longicaudatus, Heterodera glycines, Hoplolaimus columbus and also Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus and Rotylenchulus reniformis.

The compounds of the formula (I), (Ia), (Ib) or (Ic) are particularly suitable for controlling tobacco nematodes, in particular Meloidogyne incognita, Meloidogyne javanica and also Pratylenchus neglectus, Pratylenchus crenatus, Pratylenchus thornei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp. and Tetylenchus nicotianae.

The compounds of the formula (I), (Ia), (Ib) or (Ic) are particularly suitable for controlling citrus nematodes, in particular Pratylenchus coffeae and also Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similis and Radopholus citrophilus, Hemicycliophora arenaria, Hemicycliophora nudata and Tylenchulus semipenetrans.

The compounds of the formula (I), (Ia), (Ib) or (Ic) are particularly suitable for controlling banana nematodes, in particular Pratylenchus coffeae, Radopholus similis and also Pratylenchus giibbicaudatus, Pratylenchus loosi, Meloidogyne spp., Helicotylenchus multicinctus, Helicotylenchus dihystera and Rotylenchulus spp.

The compounds of the formula (I), (Ia), (Ib) or (Ic) are particularly suitable for controlling pineapple nematodes, in particular Pratylenchus zeae, Pratylenchus pratensis, Pratylenchus brachyurus, Pratylenchus goodeyi., Meloidogyne spp., Rotylenchulus reniformis and also Longidorus elongatus, Longidorus laevicapitatus, Trichodorus primitivus, Trichodorus minor, Heterodera spp., Ditylenchus myceliophagus, Hoplolaimus californicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides ferniae, Criconemoides onoense and Criconemoides ornatum.

The compounds of the formula (I), (Ia), (Ib) or (Ic) are particularly suitable for controlling grapevine nematodes, in particular Pratylenchus vulnus, Meloidogyne arenaria, Meloidogyne incognita, Meloidogyne javanica, Xiphinema americanum, Xiphinema index and also Pratylenchus pratensis, Pratylenchus scribneri, Pratylenchus neglectus, Pratylenchus brachyurus, Pratylenchus thornei and Tylenchulus semipenetrans.

The compounds of the formula (I), (Ia), (Ib) or (Ic) are particularly suitable for controlling nematodes in tree crops—pome fruit, in particular Pratylenchus penetrans and also Pratylenchus vulnus, Longidorus elongatus, Meloidogyne incognita and Meloidogyne hapla.

The compounds of the formula (I), (Ia), (Ib) or (Ic) are particularly suitable for controlling nematodes in tree crops—stone fruit, in particular Pratylenchus penetrans, Pratylenchus vulnus, Meloidogyne arenaria, Meloidogyne hapla, Meloidogyne javanica, Meloidogyne incognita, Criconemella xenoplax and of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum and Hoplolaimus galeatus.

The compounds of the formula (I), (Ia), (Ib) or (Ic) are particularly suitable for controlling nematodes in tree crops, sugar cane and rice, in particular Trichodorus spp., Criconemella spp. and also Pratylenchus spp., Paratrichodorus spp., Meloidogyne spp., Helicotylenchus spp., Tylenchorhynchus spp., Aphelenchoides spp., Heterodera spp, Xiphinema spp. and Cacopaurus pestis.

In the present context, the term “nematodes” also refers to nematodes damaging humans or animals.

Specific nematode species harmful to humans or to animals are:

Trichinellida, for example Trichuris spp., Capillaria spp., Paracapillaria spp., Eucoleus spp., Trichomosoides spp., Trichinella spp.;
from the order of the Tylenchida, for example: Micronema spp., Strongyloides spp.;
from the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;
from the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp.;

### Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.

Many known nematicides also act against other parasitic helminths and are therefore used for controlling worms—not necessarily belonging to the group Nematoda—which are parasites in humans and animals. The present invention also relates to the use of the compounds of the formula (I), (Ia), (Ib) or (Ic) as anthelmintic medicaments. The pathogenic endoparasitic helminths include Platyhelminthes (e.g. Monogenea, cestodes and trematodes), Acanthocephala and Pentastoma. The following helminths may be mentioned as being preferred:

### Monogenea: e.g.: Gyrodactylus spp., Dactylogyrus spp., Polystoma spp.;

Cestodes: from the order of the Pseudophyllidea, for example: Diphyllobothrium spp., Spirometra spp., Schistocephalus spp., Ligula spp., Bothridium spp., Diplogonoporus spp.;
from the order of the Cyclophyllida, for example: Mesocestoides spp., Anoplocephala spp., Paranoplocephala spp., Moniezia spp., Thysanosoma spp., Thy saniezia spp., Avitellina spp., Stilesia spp., Cittotaenia spp., Andyra spp., Bernella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.;

Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.;

Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.;
from the order of the Echinorhynchida, for example, Acanthocephalus spp., Echinorhynchus spp., Leptorhynchoides spp.;
Pentastoma: from the order of the Porocephalida, for example, Linguatula spp.

In the veterinary field and in animal keeping, the administration of the compounds of the formula (I), (Ia), (Ib) or (Ic) is carried out by methods generally known in the art, directly or enterally, parenterally, dermally or nasally in the form of suitable preparations. Administration can be carried out prophylactically or therapeutically.

The compounds of the formula (I), (Ia), (Ib) or (Ic) can optionally, at certain concentrations or application rates, also be used as herbicides, safeners, growth regulators or agents to improve plant properties, as microbicides or gametocides, for example as fungicides, antimycotics, bactericides, viricides (including agents against viroids) or as agents against MLO (mycoplasma-like organisms) and RLO (rickettsia-like organisms). If appropriate, they can also be used as intermediates or precursors for the synthesis of other active compounds.

### Formulations

The present invention further relates to formulations and use forms prepared therefrom as pesticides, for example drench, drip and spray liquors, comprising at least one compound of the formula (I), (Ia), (Ib) or (Ic). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropyl guar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers 173, prepared by the FAO/WHO Joint Meeting on Pesticide Specifications, 2004, ISBN: 9251048576. The formulations, in addition to one or more compounds of the formula (I), (Ia), (Ib) or (Ic), optionally comprise further agrochemically active compounds.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds of the formula (I), (Ia), (Ib) or (Ic) with auxiliaries such as, for example, extenders, solvents and/or solid carriers and/or other auxiliaries such as, for example, surfactants. The formulations are prepared either in suitable facilities or else before or during application.

The auxiliaries used may be substances suitable for imparting special properties, such as certain physical, technical and/or biological properties, to the formulation of the compounds of the formula (I), (Ia), (Ib) or (Ic), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, such as xylene, toluene or alkylnaphthalenes, chlorinated aromatic or chlorinated aliphatic hydrocarbons, such as chlorobenzene, chloroethylene or methylene chloride, aliphatic hydrocarbons, such as cyclohexane, paraffins, petroleum fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers include especially: for example ammonium salts and ground natural minerals such as kaolins, clays, talc, chalk, quartz, attapulgite, montmorillonite or diatomaceous earth, and ground synthetic materials such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Particularly suitable extenders or carriers are those which are gaseous at ambient temperature and under atmospheric pressure, for example aerosol propellant gases, such as halohydrocarbons, and also butane, propane, nitrogen and carbon dioxide.

Examples of emulsifiers and/or foam-formers, dispersants or wetting agents with ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I), (Ia), (Ib) or (Ic) and/or one of the inert carriers is insoluble in water and when the application takes place in water.

It is possible to use colorants such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc as further auxiliaries in the formulations and the use forms derived therefrom.

Additional components may be stabilizers, such as low-temperature stabilizers, preservatives, antioxidants, light stabilizers or other agents which improve chemical and/or physical stability. Foam formers or antifoams may also be present.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids may also be present as additional auxiliaries in the formulations and the use forms derived therefrom. Further possible auxiliaries are mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the compounds of the formula (I), (Ia), (Ib) or (Ic) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Suitable penetrants in the present context are all those substances which are usually used for improving the penetration of agrochemical active compounds into plants. Penetrants are defined in this context by their ability to penetrate from the (generally aqueous) application liquor and/or from the spray coating into the cuticle of the plant and thereby increase the mobility of active compounds in the cuticle. The method described in the literature (Baur et al., 1997, Pesticide Science 51, 131-152) can be used to determine this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001 and 98% by weight of the respective compound of the formula (I), (Ia), (Ib) or (Ic) or, with particular preference, between 0.01% and 95% by weight of the respective compound of the formula (I), (Ia), (Ib) or (Ic), more preferably between 0.5% and 90% by weight of the respective compound of the formula (I), (Ia), (Ib) or (Ic), based on the weight of the formulation.

The content of the respective compound of the formula (I), (Ia), (Ib) or (Ic) in the use forms prepared from the formulations (in particular pesticides) may vary within wide ranges. The concentration of the respective compound of the formula (I), (Ia), (Ib) or (Ic) in the use forms is usually between 0.00000001 and 95% by weight, preferably between 0.00001 and 1% by weight, based on the weight of the use form. The compounds are employed in a customary manner appropriate for the use forms.

### Mixtures

The compounds of the formula (I), (Ia), (Ib) or (Ic) may also be employed as a mixture with one or more suitable fungicides, bactericides, acaricides, molluscicides, nematicides, insecticides, microbiologicals, beneficial species, herbicides, fertilizers, bird repellents, phytotonics, sterilants, safeners, semiochemicals and/or plant growth regulators, in order thus, for example, to broaden the spectrum of action, to prolong the duration of action, to increase the rate of action, to prevent repulsion or prevent evolution of resistance. In addition, such active compound combinations may improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

Furthermore, the compounds of the formula (I), (Ia), (Ib) or (Ic) can be present in a mixture with other active compounds or semiochemicals such as attractants and/or bird repellants and/or plant activators and/or growth regulators and/or fertilizers. Likewise, the compounds of the formula (I), (Ia), (Ib) or (Ic) can be used to improve plant properties such as, for example, growth, yield and quality of the harvested material.

In a particular embodiment according to the invention, the compounds of the formula (I), (Ia), (Ib) or (Ic) are present in formulations or the use forms prepared from these formulations in a mixture with further compounds, preferably those as described below.

If one of the compounds mentioned below can occur in different tautomeric forms, these forms are also included even if not explicitly mentioned in each case.

### Insecticides/Acaricides/Nematicides

The active compounds identified here by their common names are known and are described, for example, in the pesticide handbook (“The Pesticide Manual” 16th Ed., British Crop Protection Council 2012) or can be found on the Internet (e.g. http://www.alanwood.net/pesticides).

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel antagonists, such as, for example, cyclodiene-organochlorines, for example chlordane and endosulfan or phenylpyrazoles (fiproles), for example ethiprole and fipronil.
(3) Sodium channel modulators/voltage-gated sodium channel blockers such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.
(4) Nicotinergic acetylcholine receptor (nAChR) agonists, such as, for example, neonicotinoids, e.g. acetamiprid, clothianidin, dinotefuran, imidacloprid, nitenpyram, thiacloprid and thiamethoxam or nicotine or sulfoxaflor.
(5) Allosteric activators of the nicotinergic acetylcholine receptor (nAChR) such as, for example, spinosyns, e.g. spinetoram and spinosad.
(6) Chloride channel activators, such as, for example, avermectins/milbemycins, for example abamectin, emamectin benzoate, lepimectin and milbemectin.
(7) Juvenile hormone imitators such as, for example, juvenile hormone analogues, e.g. hydroprene, kinoprene and methoprene or fenoxycarb or pyriproxyfen.
(8) Active compounds with unknown or nonspecific mechanisms of action such as, for example, alkyl halides, e.g. methyl bromide and other alkyl halides; or chloropicrine or sulphuryl fluoride or borax or tartar emetic.
(9) Selective antifeedants, for example pymetrozine or flonicamid.
(10) Mite growth inhibitors, for example clofentezine, hexythiazox and diflovidazin or etoxazole.
(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.
(12) Oxidative phosphorylation inhibitors, ATP disruptors such as, for example, diafenthiuron or organotin compounds, for example azocyclotin, cyhexatin and fenbutatin oxide or propargite or tetradifon;
(13) Oxidative phosphorylation decouplers acting by interrupting the H proton gradient such as, for example, chlorfenapyr, DNOC and sulfluramid.
(14) Nicotinergic acetylcholine receptor antagonists such as, for example, bensultap, cartap hydrochloride, thiocylam, and thiosultap-sodium.
(15) Chitin biosynthesis inhibitors, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.
(16) Chitin biosynthesis inhibitors, type 1, for example buprofezin.
(17) Moulting inhibitors (in particular for Diptera, i.e. dipterans) such as, for example, cyromazine.
(18) Ecdysone receptor agonists such as, for example, chromafenozide, halofenozide, methoxyfenozide and tebufenozide.
(19) Octopaminergic agonists such as, for example, amitraz.
(20) Complex-III electron transport inhibitors such as, for example, hydramethylnone or acequinocyl or fluacrypyrim.
(21) Complex-I electron transport inhibitors, for example from the group of the METI acaricides, e.g. fenazaquin, fenpyroximate, pyrimidifen, pyridaben, tebufenpyrad and tolfenpyrad or rotenone (Derris).
(22) Voltage-gated sodium channel blockers, for example indoxacarb or metaflumizone.
(23) Inhibitors of acetyl-CoA carboxylase such as, for example, tetronic and tetramic acid derivatives, e.g. spirodiclofen, spiromesifen and spirotetramat.
(24) Complex-IV electron transport inhibitors such as, for example, phosphines, e.g. aluminium phosphide, calcium phosphide, phosphine and zinc phosphide or cyanide.
(25) Complex II electron transport inhibitors, such as, for example, cyenopyrafen and cyflumetofen.
(28) Ryanodine receptor effectors, such as, for example, diamides, e.g. chlorantraniliprole, cyantraniliprole and flubendiamide,
further active compounds such as, for example, afidopyropen, azadirachtin, benclothiaz, benzoximate, bifenazate, bromopropylate, chinomethionat, cryolite,
dicofol, diflovidazin, fluensulfone, flometoquin, flufenerim, flufenoxystrobin, flufiprole, fluopyram, flupyradifurone, fufenozide, heptafluthrin, imidaclothiz, iprodione, meperfluthrin, paichongding, pyflubumide, pyrifluquinazon, pyriminostrobin, tetramethylfluthrin and iodomethane; furthermore preparations based on Bacillus firmus (I-1582, BioNeem, Votivo), and also the following compounds: 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) and 1-{2-fluoro-4-methyl-5-[(2,2,2-trifluoroethyl) sulphinyl]phenyl}-3-(trifluoromethyl)-1H-1,2,4-triazole-5-amine (known from WO2006/043635), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl-ethylcarbonate (known from WO2009/049851), 4-(but-2-yn-1-yloxy)-6-(3,5-dimethylpiperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS Reg. No. 1204776-60-2), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216), 4-{5-[3-chloro-5-(trifluoromethyl)phenyl]-5-(trifluoromethyl)-4,5-dihydro-1,2-oxazol-3-yl}-N-{2-oxo-2-[(2,2,2-trifluoroethyl)amino]ethyl}-1-naphthamide (known from WO2009/002809), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-24 {[3-bromo-1-(3-chloropyridin-2-yl)-1H-pyrazol-5-yl]carbonyl}amino)benzoyl]-2-ethylhydrazinecarboxylate (known from WO2005/085216), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO2010/069502), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 3-chloro-N-(2-cyanopropan-2-yl)-N44-(1,1,1,2,3,3,3-heptafluoropropan-2-yl)-2-methylphenyllphthalamide (known from WO2012/034472), 8-chloro-N-[(2-chloro-5-methoxyphenyl)sulphonyl]-6-(trifluoromethyl)imidazo[1,2-a]pyridine-2-carboxamide (known from WO2010/129500), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009/080250), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), (5S,8R)-1-[(6-chloropyridin-3-yl)methyl]-9-nitro-2,3,5,6,7,8-hexahydro-1H-5,8-epoxyimidazo[1,2-a]azepine (known from WO2010/069266), (2E)-1-[(6-chloropyridin-3-yl)methyl]-N′-nitro-2-pentylidenehydrazinecarboximidamide (known from WO2010/060231), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969).

### Fungicides

The active compounds specified herein by their common name are known and described, for example, in “Pesticide Manual” or on the Internet (for example: http://www.alanwood.net/pesticides).

(1) Inhibitors of ergosterol biosynthesis such as, for example, (1.1) aldimorph, (1.2) azaconazole, (1.3) bitertanol, (1.4) bromuconazole, (1.5) cyproconazole, (1.6) diclobutrazole, (1.7) difenoconazole, (1.8) diniconazole, (1.9) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamid, (1.17) fenpropidin, (1.18) fenpropimorph, (1.19) fluquinconazole, (1.20) flurprimidol, (1.21) flusilazole, (1.22) flutriafole, (1.23) furconazole, (1.24) furconazole-cis, (1.25) hexaconazole, (1.26) imazalil, (1.27) imazalil sulphate, (1.28) imibenconazole, (1.29) ipconazole, (1.30) metconazole, (1.31) myclobutanil, (1.32) naftifin, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafin, (1.49) tetraconazole, (1.50) triadimefon, (1.51) triadimenol, (1.52) tridemorph, (1.53) triflumizole, (1.54) triforine, (1.55) triticonazole, (1.56) uniconazole, (1.57) uniconazole-P, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxylate, (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide and (1.64) O-[1-(4-methoxyphenoxy)-3,3-dimethylbutan-2-yl]-1H-imidazole-1-carbothioate, (1.65) pyrisoxazole.
(2) Respiration inhibitors (respiratory chain inhibitors) such as, for example, (2.1) bixafen, (2.2) boscalid, (2.3) carboxin, (2.4) diflumetorim, (2.5) fenfuram, (2.6) fluopyram, (2.7) flutolanil, (2.8) fluxapyroxad, (2.9) furametpyr, (2.10) furmecyclox, (2.11) isopyrazam mixture of the syn-epimeric racemate 1RS,4SR,9RS and the anti-empimeric racemate 1RS,4SR,9SR, (2.12) isopyrazam (anti-epimeric racemate), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazoline-4-amine, (2.29) benzovindiflupyr, (2.30) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31) N-[(1R,4 S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3 S)-1,1,3-trimethyl-2,3-dihydro-1H-inden-4-yl]-1H-pyrazole-4-carboxamide, (2.37) 3-(difluoromethyl)-1-methyl-N-[(3 S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) isofetamid
(3) Respiration inhibitors (respiratory chain inhibitors) acting on complex III of the respiratory chain such as, for example, (3.1) ametoctradin, (3.2) amisulbrom, (3.3) azoxystrobin, (3.4) cyazofamid, (3.5) coumethoxystrobin, (3.6) coumoxystrobin, (3.5) dimoxystrobin, (3.8) enestroburin, (3.9) famoxadone, (3.10) fenamidone, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylethanamide, (3.24) (2E)-2-(methoxyimino)-N-methyl-2-(2-{[({(1E)-1-[3-(trifluoromethyl)phenyl]ethylidene}amino)oxy]methyl}phenyl)ethanamide, (3.25) (2E)-2-(methoxyimino)-N-methyl-2-{2-[(E)-({1-[3-(trifluoromethyl)phenyl]ethoxy}imino)methyl]phenyl}ethanamide, (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide, (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulphanyl)methyl]phenyl}-3-methoxyprop-2-enoate, (3.31) N-(3-ethyl-3,5,5-trimethylcyclohexyl)-3-(formylamino)-2-hydroxybenzamide, (3.32) 2-{2-[(2,5-dimethylphenoxy)methyl]phenyl}-2-methoxy-N-methylacetamide, (4) inhibitors of mitosis and cell division such as, for example, (4.1) benomyl, (4.2) carbendazim, (4.3) chlorfenazole, (4.4) diethofencarb, (4.5) ethaboxam, (4.6) fluopicolid, (4.7) fuberidazole, (4.8) pencycuron, (4.9) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl)[1,2,4]triazolo[1,5-a]pyrimidine and (4.14) 3-chloro-5-(6-chloropyridin-3-yl)-6-methyl-4-(2,4,6-trifluorophenyl)pyridazine.
(5) Compounds having multisite activity such as, for example, (5.1) Bordeaux mixture, (5.2) captafol, (5.3) captan, (5.4) chlorothalonil, (5.5) copper preparations such as copper hydroxide, (5.6) copper naphthenate, (5.7) copper oxide, (5.8) copper oxychloride, (5.9) copper sulphate, (5.10) dichlofluanid, (5.11) dithianon, (5.12) dodine, (5.13) dodine free base, (5.14) ferbam, (5.15) fluorfolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) zinc metiram, (5.27) copper-oxine, (5.28) propamidine, (5.29) propineb, (5.30) sulphur and sulphur preparations such as, for example calcium polysulphide, (5.31) thiram, (5.32) tolylfluanid, (5.33) zineb, (5.34) ziram and (5.35) anilazine.
(6) Resistance inducers such as, for example, (6.1) acibenzolar-S-methyl, (6.2) isotianil, (6.3) probenazole, (6.4) tiadinil and (6.5) laminarin.
(7) Inhibitors of amino acid and protein biosynthesis such as, for example, (7.1), (7.2) blasticidin-S, (7.3) cyprodinil, (7.4) kasugamycin, (7.5) kasugamycin hydrochloride hydrate, (7.6) mepanipyrim, (7.7) pyrimethanil, (7.8) 3-(5-fluoro-3,3,4,4-tetramethyl-3,4-dihydroisoquinolin-1-yl)quinoline and (7.9) oxytetracycline and (7.10) streptomycin.
(8) ATP production inhibitors such as, for example, (8.1) fentin acetate, (8.2) fentin chloride, (8.3) fentin hydroxide and (8.4) silthiofam.
(9) Inhibitors of cell wall synthesis such as, for example, (9.1) benthiavalicarb, (9.2) dimethomorph, (9.3) flumorph, (9.4) iprovalicarb, (9.5) mandipropamid, (9.6) polyoxins, (9.7) polyoxorim, (9.8) validamycin A, (9.9) valifenalate and (9.10) polyoxin B.
(10) Inhibitors of lipid and membrane synthesis such as, for example, (10.1) biphenyl, (10.2) chlorneb, (10.3) dicloran, (10.4) edifenphos, (10.5) etridiazole, (10.6) iodocarb, (10.7) iprobenfos, (10.8) isoprothiolane, (10.9) propamocarb, (10.10) propamocarb hydrochloride, (10.11) prothiocarb (10.12) pyrazophos, (10.13) quintozene, (10.14) tecnazene and (10.15) tolclofos-methyl.
(11) Melanin biosynthesis inhibitors, for example (11.1) carpropamid, (11.2) diclocymet, (11.3) fenoxanil, (11.4) fthalide, (11.5) pyroquilon, (11.6) tricyclazole and (11.7) 2,2,2-trifluoroethyl {3-methyl-1-[(4-methylbenzoyl)amino]butan-2-yl}carbamate.
(12) Inhibitors of nucleic acid synthesis such as, for example, (12.1) benalaxyl, (12.2) benalaxyl-M (kiralaxyl), (12.3) bupirimate, (12.4) clozylacon, (12.5) dimethirimol, (12.6) ethirimol, (12.7) furalaxyl, (12.8) hymexazole, (12.9) metalaxyl, (12.10) metalaxyl-M (mefenoxam), (12.11) ofurace, (12.12) oxadixyl, (12.13) oxolinic acid and (12.14) octhilinone.
(13) Signal transduction inhibitors such as, for example, (13.1) chlozolinate, (13.2) fenpiclonil, (13.3) fludioxonil, (13.4) iprodione, (13.5) procymidone, (13.6) quinoxyfen, (13.7) vinclozolin and (13.8) proquinazid.
(14) Decouplers such as, for example, (14.1) binapacryl, (14.2) dinocap, (14.3) ferimzone, (14.4) fluazinam and (14.5) meptyldinocap.
(15) Further compounds such as, for example, (15.1) benthiazole, (15.2) bethoxazine, (15.3) capsimycin, (15.4) carvone, (15.5) chinomethionat, (15.6) pyriofenone (chlazafenone), (15.7) cufraneb, (15.8) cyflufenamid, (15.9) cymoxanil, (15.10) cyprosulfamide, (15.11) dazomet, (15.12) debacarb, (15.13) dichlorophen, (15.14) diclomezine, (15.15) difenzoquat, (15.16) difenzoquat methylsulphate, (15.17) diphenylamine, (15.18) EcoMate, (15.19) fenpyrazamine, (15.20) flumetover, (15.21) fluorimid, (15.22) flusulfamide, (15.23) flutianil, (15.24) fosetyl-aluminium, (15.25) fosetyl-calcium, (15.26) fosetyl-sodium, (15.27) hexachlorobenzene, (15.28) irumamycin, (15.29) methasulfocarb, (15.30) methyl isothiocyanate, (15.31) metrafenone, (15.32) mildiomycin, (15.33) natamycin, (15.34) nickel dimethyldithiocarbamate, (15.35) nitrothal-isopropyl, (15.36) octhilinone, (15.37) oxamocarb, (15.38) oxyfenthiin, (15.39) pentachlorophenol and its salts, (15.40) phenothrin, (15.41) phosphoric acid and its salts, (15.42) propamocarb-fosetylate, (15.43) propanosine-sodium, (15.44) pyrimorph, (15.45) (2E)-3-(4-tert-butylphenyl)-3-(2-chloropyridin-4-yl)-1-(morpholin-4-yl)prop-2-en-1-one, (15.46) (2Z)-3-(4-tert-butylphenyl)-3-(2-chloropyridin-4-yl)-1-(morpholin-4-yl)prop-2-en-1-one, (15.47) pyrrolnitrin, (15.48) tebufloquin, (15.49) tecloftalam, (15.50) tolnifanide, (15.51) triazoxide, (15.52) trichlamide, (15.53) zarilamid, (15.54) (3 S,6 S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (15.55) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.56) 1-(4-{4-[(5 S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.57) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.58) 1-(4-methoxyphenoxy)-3,3-dimethylbutan-2-yl 1H-imidazole-1-carboxy late, (15.59) 2,3,5,6-tetrachloro-4-(methyl sulphonyl)pyridine, (15.60) 2,3-dibutyl-6-chlorothieno[2,3-d]pyrimidin-4(3H)-one, (15.61) 2,6-dimethyl-1H,5H-[1,4]dithiino[2,3-c:5,6-c′]dipyrrole-1,3,5,7(2H,6H)-tetrone, (15.62) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.63) 2-[5-methyl-3-(trifluoromethyl)-1H-pyrazol-1-yl]-1-(4-{4-[(5 S)-5-phenyl-4,5-dihydro-1,2-oxazol-3-yl]-1,3-thiazol-2-yl}piperidin-1-yl)ethanone, (15.64) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.65) 2-butoxy-6-iodo-3-propyl-4H-chromen-4-one, (15.66) 2-chloro-5-[2-chloro-1-(2,6-difluoro-4-methoxyphenyl)-4-methyl-1H-imidazol-5-yl]pyridine, (15.67) 2-phenylphenol and salts, (15.68) 3-(4,4,5-trifluoro-3,3-dimethyl-3,4-dihydroisoquinolin-1-yl)quinoline, (15.69) 3,4,5-trichloropyridine-2,6-dicarbonitrile, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulphonohydrazide, (15.74) 5-fluoro-2-[(4-fluorobenzyl)oxy]pyrimidine-4-amine, (15.75) 5-fluoro-2-[(4-methylbenzyl)oxy]pyrimidine-4-amine, (15.76) 5-methyl-6-octyl[1,2,4]triazolo[1,5-a]pyrimidine-7-amine, (15.77) ethyl (2Z)-3-amino-2-cyano-3-phenylacrylate, (15.78) N′-(4-{[3-(4-chlorobenzyl)-1,2,4-thiadiazol-5-yl]oxy}-2,5-dimethylphenyl)-N-ethyl-N-methylimidoformamide, (15.79) N-(4-chlorobenzyl)-3-[3-methoxy-4-(prop-2-yn-1-yloxy)phenyl]propanamide, (15.80) N-[(4-chlorophenyl)(cyano)methyl]-3-[3-methoxy-4-(prop-2-yn-1-yloxy)phenyl]propanamide, (15.81) N-[(5-bromo-3-chloropyridin-2-yl)methyl]-2,4-dichloronicotinamide, (15.82) N-[1-(5-bromo-3-chloropyridin-2-yl)ethyl]-2,4-dichloronicotinamide, (15.83) N-[1-(5-bromo-3-chloropyridin-2-yl)ethyl]-2-fluoro-4-iodonicotinamide, (15.84) N-{(E)-[(cyclopropylmethoxy)imino][6-(difluoromethoxy)-2,3-difluorophenyl]methyl}-2-phenylacetamide, (15.85) N-{(Z)-[(cyclopropylmethoxy)imino][6-(difluoromethoxy)-2,3-difluorophenyl]methyl}-2-phenylacetamide, (15.86) N′-{4-[(3-tert-butyl-4-cyano-1,2-thiazol-5-yl)oxy]-2-chloro-5-methylphenyl}-N-ethyl-N-methylimidoformamide, (15.87) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.88) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.89) N-methyl-2-(1-{[5-methyl-3-(trifluoromethyl)-1H-pyrazol-1-yl]acetyl}piperidin-4-yl)-N-[(1 S)-1,2,3,4-tetrahydronaphthalen-1-yl]-1,3-thiazole-4-carboxamide, (15.90) pentyl{6-[({[(1-methyl-1H-tetrazol-5-yl)(phenyl)methylene]amino}oxy)methyl]pyridin-2-yl}carbamate, (15.91) phenazine-1-carboxylic acid, (15.92) quinolin-8-ol, (15.93) quinolin-8-ol sulphate (2:1), (15.94) tert-butyl 6-[({[(1-methyl-1H-tetrazol-5-yl)(phenyl)methylene]amino}oxy)methyl]pyridin-2-yl carbamate, (15.95) 1-methyl-3-(trifluoromethyl)-N-[2′-(trifluoromethyl)biphenyl-2-yl]-1H-pyrazole-4-carboxamide, (15.96) N-(4′-chlorobiphenyl-2-yl)-3-(difluoromethyl)-1-methyl-1H-pyrazole-4-carboxamide, (15.97) N-(2′,4′-dichlorobiphenyl-2-yl)-3-(difluoromethyl)-1-methyl-1H-pyrazole-4-carboxamide, (15.98) 3-(difluoromethyl)-1-methyl-N-[4′-(trifluoromethyl)biphenyl-2-yl]-1H-pyrazole-4-carboxamide, (15.99) N-(2′,5′-difluorobiphenyl-2-yl)-1-methyl-3-(trifluoromethyl)-1H-pyrazole-4-carboxamide, (15.100) 3-(difluoromethyl)-1-methyl-N-[4′-(prop-1-yn-1-yl)biphenyl-2-yl]-1H-pyrazole-4-carboxamide, (15.101) 5-fluoro-1,3-dimethyl-N-[4′-(prop-1-yn-1-yl)biphenyl-2-yl]-1H-pyrazole-4-carboxamide, (15.102) 2-chloro-N-[4′-(prop-1-yn-1-yl)biphenyl-2-yl]nicotinamide, (15.103) 3-(difluoromethyl)-N-[4′-(3,3-dimethylbut-1-yn-1-yl)biphenyl-2-yl]-1-methyl-1H-pyrazole-4-carboxamide, (15.104) N-[4′-(3,3-dimethylbut-1-yn-1-yl)biphenyl-2-yl]-5-fluoro-1,3-dimethyl-1H-pyrazole-4-carboxamide, (15.105) 3-(difluoromethyl)-N-(4′-ethynylbiphenyl-2-yl)-1-methyl-1H-pyrazole-4-carboxamide, (15.106) N-(4′-ethynylbiphenyl-2-yl)-5-fluoro-1,3-dimethyl-1H-pyrazole-4-carboxamide, (15.107) 2-chloro-N-(4′-ethynylbiphenyl-2-yl)nicotinamide, (15.108) 2-chloro-N-[4′-(3,3-dimethylbut-1-yn-1-yl)biphenyl-2-yl]nicotinamide, (15.109) 4-(difluoromethyl)-2-methyl-N-[4′-(trifluoromethyl)biphenyl-2-yl]-1,3-thiazole-5-carboxamide, (15.110) 5-fluoro-N-[4′-(3-hydroxy-3-methylbut-1-yn-1-yl)biphenyl-2-yl]-1,3-dimethyl-1H-pyrazole-4-carboxamide, (15.111) 2-chloro-N-[4′-(3-hydroxy-3-methylbut-1-yn-1-yl)biphenyl-2-yl]nicotinamide, (15.112) 3-(difluoromethyl)-N-[4′-(3-methoxy-3-methylbut-1-yn-1-yl)biphenyl-2-yl]-1-methyl-1H-pyrazole-4-carboxamide, (15.113) 5-fluoro-N-[4′-(3-methoxy-3-methylbut-1-yn-1-yl)biphenyl-2-yl]-1,3-dimethyl-1H-pyrazole-4-carboxamide, (15.114) 2-chloro-N-[4′-(3-methoxy-3-methylbut-1-yn-1-yl)biphenyl-2-yl]nicotinamide, (15.115) (5-bromo-2-methoxy-4-methylpyridin-3-yl)(2,3,4-trimethoxy-6-methylphenyl)methanone, (15.116) N-[2-(4-{[3-(4-chlorophenyl)prop-2-yn-1-yl]oxy}-3-methoxyphenyl)ethyl]-N2-(methylsulphonyl)valinamide, (15.117) 4-oxo-4-[(2-phenylethyl)amino]butanoic acid, (15.118) but-3-yn-1-yl{6-[({[(Z)-(1-methyl-1H-tetrazol-5-yl)(phenyl)methylene]amino}oxy)methyl]pyridin-2-yl}carbamate, (15.119) 4-amino-5-fluoropyrimidin-2-ol (tautomeric form: 4-amino-5-fluoropyrimidin-2(1H)-one), (15.120) propyl 3,4,5-trihydroxybenzoate, (15.121) 1,3-dimethyl-N-(1,1,3-trimethyl-2,3-dihydro-1H-inden-4-yl)-1H-pyrazole-4-carboxamide, (15.122) 1,3-dimethyl-N-[(3R)-1,1,3-trimethyl-2,3-dihydro-1H-inden-4-yl]-1H-pyrazole-4-carboxamide, (15.123) 1,3-dimethyl-N-[(3 S)-1,1,3-trimethyl-2,3-dihydro-1H-inden-4-yl]-1H-pyrazole-4-carboxamide, (15.124) [3-(4-chloro-2-fluorophenyl)-5-(2,4-difluorophenyl)-1,2-oxazol-4-yl](pyridin-3-yl)methanol, (15.125) (S)-[3-(4-chloro-2-fluorophenyl)-5-(2,4-difluorophenyl)-1,2-oxazol-4-yl](pyridin-3-yl)methanol, (15.126) (R)-[3-(4-chloro-2-fluorophenyl)-5-(2,4-difluorophenyl)-1,2-oxazol-4-yl](pyridin-3-yl)methanol, (15.127) 2-{[3-(2-chlorophenyl)-2-(2,4-difluorophenyl)oxiran-2-yl]methyl}-2,4-dihydro-3H-1,2,4-triazole-3-thione, (15.128) 1-{[3-(2-chlorophenyl)-2-(2,4-difluorophenyl)oxiran-2-yl]methyl}-1H-1,2,4-triazol-5-yl thiocyanate, (15.129) 5-(allylsulfanyl)-1-{[3-(2-chlorophenyl)-2-(2,4-difluorophenyl)oxiran-2-yl]methyl}-1H-1,2,4-triazole, (15.130) 2-[1-(2,4-dichlorophenyl)-5-hydroxy-2,6,6-trimethylheptan-4-yl]-2,4-dihydro-3H-1,2,4-triazole-3-thione, (15.131) 2-{[rel(2R,3 S)-3-(2-chlorophenyl)-2-(2,4-difluorophenyl)oxiran-2-yl]methyl}-2,4-dihydro-3H-1,2,4-triazole-3-thione, (15.132) 2-{[rel(2R,3R)-3-(2-chlorophenyl)-2-(2,4-difluorophenyl)oxiran-2-yl]methyl}-2,4-dihydro-3H-1,2,4-triazole-3-thione, (15.133) 1-{[rel(2R,3S)-3-(2-chlorophenyl)-2-(2,4-difluorophenyl)oxiran-2-yl]methyl}-1H-1,2,4-triazol-5-yl thiocyanate, (15.134) 1-{[rel(2R,3R)-3-(2-chlorophenyl)-2-(2,4-difluorophenyl)oxiran-2-yl]methyl}-1H-1,2,4-triazol-5-yl thiocyanate, (15.135) 5-(allylsulphanyl)-1-{[rel(2R,3 S)-3-(2-chlorophenyl)-2-(2,4-difluorophenyl)oxiran-2-yl]methyl}-1H-1,2,4-triazole, (15.136) 5-(allylsulphanyl)-1-{[rel(2R,3R)-3-(2-chlorophenyl)-2-(2,4-difluorophenyl)oxiran-2-yl]methyl}-1H-1,2,4-triazole, (15.137) 2-[(2 S,4S,5 S)-1-(2,4-dichlorophenyl)-5-hydroxy-2,6,6-trimethylheptan-4-yl]-2,4-dihydro-3H-1,2,4-triazole-3-thione, (15.138) 2-[(2R,4S,5S)-1-(2,4-dichlorophenyl)-5-hydroxy-2,6,6-trimethylheptan-4-yl]-2,4-dihydro-3H-1,2,4-triazole-3-thione, (15.139) 2-[(2R,4R,5R)-1-(2,4-dichlorophenyl)-5-hydroxy-2,6,6-trimethylheptan-4-yl]-2,4-dihydro-3H-1,2,4-triazole-3-thione, (15.140) 2-[(2S,4R,5R)-1-(2,4-dichlorophenyl)-5-hydroxy-2,6,6-trimethylheptan-4-yl]-2,4-dihydro-3H-1,2,4-triazole-3-thione, (15.141) 2-[(2S,4 S,5R)-1-(2,4-dichlorophenyl)-5-hydroxy-2,6,6-trimethylheptan-4-yl]-2,4-dihydro-3H-1,2,4-triazole-3-thione, (15.142) 2-[(2R,4S,5R)-1-(2,4-dichlorophenyl)-5-hydroxy-2,6,6-trimethylheptan-4-yl]-2,4-dihydro-3H-1,2,4-triazole-3-thione, (15.143) 2-[(2R,4R,5 S)-1-(2,4-dichlorophenyl)-5-hydroxy-2,6,6-trimethylheptan-4-yl]-2,4-dihydro-3H-1,2,4-triazole-3-thione, (15.144) 2-[(2S,4R,5S)-1-(2,4-dichlorophenyl)-5-hydroxy-2,6,6-trimethylheptan-4-yl]-2,4-dihydro-3H-1,2,4-triazole-3-thione, (15.145) 2-fluoro-6-(trifluoromethyl)-N-(1,1,3-trimethyl-2,3-dihydro-1H-inden-4-yl)benzamide, (15.146) 2-(6-benzylpyridin-2-yl)quinazoline, (15.147) 2-[6-(3-fluoro-4-methoxyphenyl)-5-methylpyridin-2-yl]quinazoline, (15.148) 3-(4,4-difluoro-3,3-dimethyl-3,4-dihydroisoquinolin-1-yl)quinoline, (15.149) abscisic acid, (15.150) 3-(difluoromethyl)-N-methoxy-1-methyl-N-[1-(2,4,6-trichlorophenyl)propan-2-yl]-1H-pyrazole-4-carboxamide, (15.151) N′-[5-bromo-6-(2,3-dihydro-1H-inden-2-yloxy)-2-methylpyridin-3-yl]-N-ethyl-N-methylimidoformamide, (15.152) N′-{5-bromo-6-[1-(3,5-difluorophenyl)ethoxy]-2-methylpyridin-3-yl}-N-ethyl-N-methylimidoformamide, (15.153) N′-{5-bromo-6-[(1R)-1-(3,5-difluorophenyl)ethoxy]-2-methylpyridin-3-yl}-N-ethyl-N-methylimidoformamide, (15.154) N′-{5-bromo-6-[(1 S)-1-(3,5-difluorophenyl)ethoxy]-2-methylpyridin-3-yl}-N-ethyl-N-methylimidoformamide, (15.155) N′-{5-bromo-6-[(cis-4-isopropylcyclohexyl)oxy]-2-methylpyridin-3-yl}-N-ethyl-N-methylimidoformamide, (15.156) N′-{5-bromo-6-[(trans-4-isopropylcyclohexyl)oxy]-2-methylpyridin-3-yl}-N-ethyl-N-methylimidoformamide, (15.157) N-cyclopropyl-3-(difluoromethyl)-5-fluoro-N-(2-isopropylbenzyl)-1-methyl-1H-pyrazole-4-carboxamide, (15.158) N-cyclopropyl-N-(2-cyclopropylbenzyl)-3-(difluoromethyl)-5-fluoro-1-methyl-1H-pyrazole-4-carboxamide, (15.159) N-(2-tert-butylbenzyl)-N-cyclopropyl-3-(difluoromethyl)-5-fluoro-1-methyl-1H-pyrazole-4-carboxamide, (15.160) N-(5-chloro-2-ethylbenzyl)-N-cyclopropyl-3-(difluoromethyl)-5-fluoro-1-methyl-1H-pyrazole-4-carboxamide, (15.161) N-(5-chloro-2-isopropylbenzyl)-N-cyclopropyl-3-(difluoromethyl)-5-fluoro-1-methyl-1H-pyrazole-4-carboxamide, (15.162) N-cyclopropyl-3-(difluoromethyl)-N-(2-ethyl-5-fluorobenzyl)-5-fluoro-1-methyl-1H-pyrazole-4-carboxamide, (15.163) N-cyclopropyl-3-(difluoromethyl)-5-fluoro-N-(5-fluoro-2-isopropylbenzyl)-1-methyl-1H-pyrazole-4-carboxamide, (15.164) N-cyclopropyl-N-(2-cyclopropyl-5-fluorobenzyl)-3-(difluoromethyl)-5-fluoro-1-methyl-1H-pyrazole-4-carboxamide, (15.165) N-(2-cyclopentyl-5-fluorobenzyl)-N-cyclopropyl-3-(difluoromethyl)-5-fluoro-1-methyl-1H-pyrazole-4-carboxamide, (15.166) N-cyclopropyl-3-(difluoromethyl)-5-fluoro-N-(2-fluoro-6-isopropylbenzyl)-1-methyl-1H-pyrazole-4-carboxamide, (15.167) N-cyclopropyl-3-(difluoromethyl)-N-(2-ethyl-5-methylbenzyl)-5-fluoro-1-methyl-1H-pyrazole-4-carboxamide, (15.168) N-cyclopropyl-3-(difluoromethyl)-5-fluoro-N-(2-isopropyl-5-methylbenzyl)-1-methyl-1H-pyrazole-4-carboxamide, (15.169) N-cyclopropyl-N-(2-cyclopropyl-5-methylbenzyl)-3-(difluoromethyl)-5-fluoro-1-methyl-1H-pyrazole-4-carboxamide, (15.170) N-(2-tert-butyl-5-methylbenzyl)-N-cyclopropyl-3-(difluoromethyl)-5-fluoro-1-methyl-1H-pyrazole-4-carboxamide, (15.171) N-[5-chloro-2-(trifluoromethyl)benzyl]-N-cyclopropyl-3-(difluoromethyl)-5-fluoro-1-methyl-1H-pyrazole-4-carboxamide, (15.172) N-cyclopropyl-3-(difluoromethyl)-5-fluoro-1-methyl-N-[5-methyl-2-(trifluoromethyl)benzyl]-1H-pyrazole-4-carboxamide, (15.173) N-[2-chloro-6-(trifluoromethyl)benzyl]-N-cyclopropyl-3-(difluoromethyl)-5-fluoro-1-methyl-1H-pyrazole-4-carboxamide, (15.174) N-[3-chloro-2-fluoro-6-(trifluoromethyl)benzyl]-N-cyclopropyl-3-(difluoromethyl)-5-fluoro-1-methyl-1H-pyrazole-4-carboxamide, (15.175) N-cyclopropyl-3-(difluoromethyl)-N-(2-ethyl-4,5-dimethylbenzyl)-5-fluoro-1-methyl-1H-pyrazole-4-carboxamide, (15.176) N-cyclopropyl-3-(difluoromethyl)-5-fluoro-N-(2-isopropylbenzyl)-1-methyl-1H-pyrazol-4-carbothioamide, (15.177) 3-(difluoromethyl)-N-(7-fluoro-1,1,3-trimethyl-2,3-dihydro-1H-inden-4-yl)-1-methyl-1H-pyrazole-4-carboxamide, (15.178) 3-(difluoromethyl)-N-[(3R)-7-fluoro-1,1,3-trimethyl-2,3-dihydro-1H-inden-4-yl]-1-methyl-1H-pyrazole-4-carboxamide, (15.179) 3-(difluoromethyl)-N-[(3 S)-7-fluoro-1, 1,3-trimethyl-2,3-dihydro-1H-inden-4-yl]-1-methyl-1H-pyrazole-4-carboxamide, (15.180) N′-(2,5-dimethyl-4-phenoxyphenyl)-N-ethyl-N-methylimidoformamide, (15.181) N′-{4-[(4,5-dichloro-1,3-thiazol-2-yl)oxy]-2,5-dimethylphenyl}-N-ethyl-N-methylimidoformamide, (15.182) N-(4-chloro-2,6-difluorophenyl)-4-(2-chloro-4-fluorophenyl)-1,3-dimethyl-1H-pyrazole-5-amine. All mixing components mentioned in classes (1) to (15) can, if they are capable on the basis of their functional groups, optionally form salts with suitable bases or acids.

### Biological Pesticides as Mixing Components

The compounds of the formula (I), (Ia), (Ib) or (Ic) can be combined with biological pesticides.

Biological pesticides comprise in particular bacteria, fungi, yeasts, plant extracts and products formed by microorganisms, including proteins and secondary metabolites.

Biological pesticides comprise bacteria such as spore-forming bacteria, root-colonising bacteria and bacteria which act as biological insecticides, fungicides or nematicides.

Examples of such bacteria which are employed or can be used as biological pesticides are:

Bacillus amyloliquefaciens, strain FZB42 (DSM 231179), or Bacillus cereus, in particular B. cereus strain CNCM 1-1562 or Bacillus firmus, strain 1-1582 (Accession number CNCM 1-1582) or Bacillus pumilus, in particular strain GB34 (Accession No. ATCC 700814) and strain QST2808 (Accession No. NRRL B-30087), or Bacillus subtilis, in particular strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, in particular B. thuringiensis subspecies israelensis (serotype H-14), strain AM65-52 (Accession No. ATCC 1276), or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are employed or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KV01, Metarhizium anisopliae, in particular strain F52 (DSM3884/ATCC 90448), Metschnikowia fructi cola, in particular strain NRRL Y-30752, Paecilomyces fumosoroseus (now: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39. (Accession Number CNCM 1-952).

Examples of viruses which are employed or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples which may be mentioned are:

Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium Rhizopogon spp., Scleroderma spp., Sullins spp., Streptomyces spp.

Examples of plant extracts and products formed by microorganisms including proteins and secondary metabolites which are employed or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, in particular oilseed rape powder or mustard powder.

### Safener as Mixing Components

The compounds of the formula (I), (Ia), (Ib) or (Ic) can be combined with safeners such as, for example, benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Here, plants are to be understood to mean all plants and plant parts such as wanted and unwanted wild plants or crop plants (including naturally occurring crop plants), for example cereals (wheat, rice, triticale, barley, rye, oats), maize, soya bean, potato, sugar beet, sugar cane, tomatoes, peas and other vegetable species, cotton, tobacco, oilseed rape, and also fruit plants (with the fruits apples, pears, citrus fruits and grapevines). Crop plants can be plants which can be obtained by conventional breeding and optimization methods or by biotechnological and genetic engineering methods or combinations of these methods, including the transgenic plants and including the plant varieties which can or cannot be protected by varietal property rights. Plant parts should be understood to mean all parts and organs of the plants above and below ground, such as shoot, leaf, flower and root, examples given being leaves, needles, stalks, stems, flowers, fruit bodies, fruits and seeds, and also tubers, roots and rhizomes. Parts of plants also include harvested plants and vegetative and generative propagation material, for example seedlings, tubers, rhizomes, cuttings and seeds.

Treatment according to the invention of the plants and plant parts with the compounds of the formula (I), (Ia), (Ib) or (Ic) is carried out directly or by allowing the compounds to act on the surroundings, environment or storage space by the customary treatment methods, for example by immersion, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. The invention is used with particular preference to treat plants of the respective commercially customary cultivars or those that are in use. Plant cultivars are to be understood as meaning plants having new properties (“traits”) and which have been obtained by conventional breeding, by mutagenesis or by recombinant DNA techniques. They can be cultivars, varieties, bio- or genotypes.

### Transgenic Plant, Seed Treatment and Integration Events

The transgenic plants or plant cultivars (those obtained by genetic engineering) which are to be treated with preference in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products. Further and particularly emphasized examples of such properties are increased resistance of the plants against animal and microbial pests, such as against insects, arachnids, nematodes, mites, slugs and snails owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof), furthermore increased resistance of the plants against phytopathogenic fungi, bacteria and/or viruses owing, for example, to systemic acquired resistance (SAR), systemin, phytoalexins, elicitors and also resistance genes and correspondingly expressed proteins and toxins, and also increased tolerance of the plants to certain herbicidally active compounds, for example imidazolinones, sulphonylureas, glyphosate or phosphinothricin (for example the “PAT” gene). The genes which impart the desired traits in question may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails.

### Crop Protection Types of Treatment

The treatment of the plants and plant parts with the compounds of the formula (I), (Ia), (Ib) or (Ic) is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc. It is furthermore possible to apply the compounds of the formula (I), (Ia), (Ib) or (Ic) by the ultra-low volume method or to inject the application form or the compound of the formula (I), (Ia), (Ib) or (Ic) itself into the soil.

A preferred direct treatment of the plants is foliar application, i.e. the compounds of the formula (I), (Ia), (Ib) or (Ic) are applied to the foliage, where treatment frequency and the application rate should be adjusted according to the level of infestation with the pest in question.

In the case of systemically active compounds, the compounds of the formula (I), (Ia), (Ib) or (Ic) also access the plants via the root system. The plants are then treated by the action of the compounds of the formula (I), (Ia), (Ib) or (Ic) on the habitat of the plant. This may be done, for example, by drenching, or by mixing into the soil or the nutrient solution, i.e. the locus of the plant (e.g. soil or hydroponic systems) is impregnated with a liquid form of the compounds of the formula (I), (Ia), (Ib) or (Ic), or by soil application, i.e. the compounds of the formula (I), (Ia), (Ib) or (Ic) according to the invention are introduced in solid form (e.g. in the form of granules) into the locus of the plants. In the case of paddy rice crops, this can also be done by metering the compound of the formula (I), (Ia), (Ib) or (Ic) in a solid application form (for example as granules) into a flooded paddy field.

### Treatment of Seed

The control of animal pests by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is furthermore desirable to optimize the amount of active compound employed in such a way as to provide optimum protection for the seed and the germinating plant from attack by animal pests, but without damaging the plant itself by the active compound employed. In particular, methods for the treatment of seed should also take into consideration the intrinsic insecticidal or nematicidal properties of pest-resistant or -tolerant transgenic plants in order to achieve optimum protection of the seed and also the germinating plant with a minimum of pesticides being employed.

The present invention therefore in particular also relates to a method for the protection of seed and germinating plants, from attack by pests, by treating the seed with one of the compounds of the formula (I), (Ia), (Ib) or (Ic). The method according to the invention for protecting seed and germinating plants against attack by pests furthermore comprises a method where the seed is treated simultaneously in one operation or sequentially with a compound of the formula (I), (Ia), (Ib) or (Ic) and a mixing component. It also comprises a method where the seed is treated at different times with a compound of the formula (I), (Ia), (Ib) or (Ic) and a mixing component.

The invention likewise relates to the use of the compounds of the formula (I), (Ia), (Ib) or (Ic) for the treatment of seed for protecting the seed and the resulting plant from animal pests.

Furthermore, the invention relates to seed which has been treated with a compound of the formula (I), (Ia), (Ib) or (Ic) according to the invention so as to afford protection from animal pests. The invention also relates to seed which has been treated simultaneously with a compound of the formula (I), (Ia), (Ib) or (Ic) and a mixing component. The invention furthermore relates to seed which has been treated at different times with a compound of the formula (I), (Ia), (Ib) or (Ic) and a mixing component. In the case of seed which has been treated at different points in time with a compound of the formula (I), (Ia), (Ib) or (Ic) and a mixing component, the individual substances may be present on the seed in different layers. Here, the layers comprising a compound of the formula (I), (Ia), (Ib) or (Ic) and mixing components may optionally be separated by an intermediate layer. The invention also relates to seed where a compound of the formula (I), (Ia), (Ib) or (Ic) and a mixing component have been applied as component of a coating or as a further layer or further layers in addition to a coating.

Furthermore, the invention relates to seed which, after the treatment with a compound of the formula (I), (Ia), (Ib) or (Ic), is subjected to a film-coating process to prevent dust abrasion on the seed.

One of the advantages encountered with a systemically acting compound of the formula (I), (Ia), (Ib) or (Ic) is the fact that, by treating the seed, not only the seed itself but also the plants resulting therefrom are, after emergence, protected against animal pests. In this manner, the immediate treatment of the crop at the time of sowing or shortly thereafter can be dispensed with.

It has to be considered a further advantage that by treatment of the seed with a compound of the formula (I), (Ia), (Ib) or (Ic), germination and emergence of the treated seed may be enhanced.

It is likewise to be considered advantageous that compounds of the formula (I), (Ia), (Ib) or (Ic) can be used in particular also for transgenic seed.

Furthermore, compounds of the formula (I), (Ia), (Ib) or (Ic) can be employed in combination with compositions or compounds of signalling technology, leading to better colonization by symbionts such as, for example, rhizobia, mycorrhizae and/or endophytic bacteria or fungi, and/or to optimized nitrogen fixation.

The compounds of the formula (I), (Ia), (Ib) or (Ic) are suitable for protection of seed of any plant variety which is used in agriculture, in the greenhouse, in forests or in horticulture. In particular, this takes the form of seed of cereals (for example wheat, barley, rye, millet and oats), corn, cotton, soya beans, rice, potatoes, sunflowers, coffee, tobacco, canola, oilseed rape, beets (for example sugarbeets and fodder beets), peanuts, vegetables (for example tomatoes, cucumbers, bean, cruciferous vegetables, onions and lettuce), fruit plants, lawns and ornamental plants. The treatment of the seed of cereals (such as wheat, barley, rye and oats), maize, soya beans, cotton, canola, oilseed rape and rice is of particular importance.

As already mentioned above, the treatment of transgenic seed with a compound of the formula (I), (Ia), (Ib) or (Ic) is also of particular importance. This takes the form of seed of plants which, as a rule, comprise at least one heterologous gene which governs the expression of a polypeptide with in particular insecticidal and/or nematicidal properties. The heterologous genes in transgenic seed can originate from microorganisms such as Bacillus, Rhizobium, Pseudomonas, Serratia, Trichoderma, Clavibacter, Glomus or Gliocladium. The present invention is particularly suitable for the treatment of transgenic seed which comprises at least one heterologous gene originating from Bacillus sp. It is particularly preferably a heterologous gene derived from Bacillus thuringiensis.

In the context of the present invention, the compound of the formula (I), (Ia), (Ib) or (Ic) is applied to the seed. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming.

When treating the seed, care must generally be taken that the amount of the compound of the formula (I), (Ia), (Ib) or (Ic) applied to the seed and/or the amount of further additives is chosen in such a way that the germination of the seed is not adversely affected, or that the resulting plant is not damaged. This must be ensured particularly in the case of active compounds which can exhibit phytotoxic effects at certain application rates.

In general, the compounds of the formula (I), (Ia), (Ib) or (Ic) are applied to the seed in a suitable formulation. Suitable formulations and processes for seed treatment are known to the person skilled in the art.

The compounds of the formula (I), (Ia), (Ib) or (Ic) can be converted to the customary seed dressing formulations, such as solutions, emulsions, suspensions, powders, foams, slurries or other coating compositions for seed, and also ULV formulations.

These formulations are prepared in a known manner, by mixing the compounds of the formula (I), (Ia), (Ib) or (Ic) with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of agrochemically active compounds. Preference is given to using alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristryrylphenol polyglycol ethers, and the phosphated or sulphated derivatives thereof. Suitable anionic dispersants are in particular lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Preference is given to using silicone antifoams and magnesium stearate.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances which can be used for such purposes in agrochemical compositions. Cellulose derivatives, acrylic acid derivatives, xanthan, modified clays and finely divided silica are preferred.

Adhesives which may be present in the seed dressing formulations usable in accordance with the invention are all customary binders usable in seed dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schädlingsbekämpfungsmittel”, vol. 2, Springer Verlag, 1970, pp. 401-412).

The seed dressing formulations usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also the seed of maize, rice, oilseed rape, peas, beans, cotton, sunflowers, soya beans and beets, or else a wide variety of different vegetable seed. The seed dressing formulations usable in accordance with the invention, or the dilute use forms thereof, can also be used to dress seed of transgenic plants.

For treatment of seed with the seed dressing formulations usable in accordance with the invention, or the use forms prepared therefrom by adding water, all mixing units usable customarily for the seed dressing are useful. Specifically, the procedure in the seed dressing is to place the seed into a mixer, operated batch-wise or continously, to add the particular desired amount of seed dressing formulations, either as such or after prior dilution with water, and to mix everything until the formulation is distributed homogeneously on the seed. If appropriate, this is followed by a drying operation.

The application rate of the seed dressing formulations usable in accordance with the invention can be varied within a relatively wide range. It is guided by the particular content of the compounds of the formula (I), (Ia), (Ib) or (Ic) in the formulations and by the seed. The application rates of the respective compound of the formula (I), (Ia), (Ib) or (Ic) are generally between 0.001 and 50 g per kilogram of seed, preferably between 0.01 and 15 g per kilogram of seed.

### Animal Health

In the animal health field, i.e. in the field of veterinary medicine, the compounds of the formula (I), (Ia), (Ib) or (Ic) are active against animal parasites, in particular ectoparasites or endoparasites. The term endoparasites includes in particular helminths and protozoans, such as coccidia. Ectoparasites are typically and preferably arthropods, in particular insects and acarids.

In the field of veterinary medicine the compounds of the formula (I), (Ia), (Ib) or (Ic) are suitable, with favourable homeotherm toxicity, for controlling parasites which occur in animal breeding and animal husbandry in livestock, breeding, zoo, laboratory, experimental and domestic animals They are active against all or specific stages of development of the parasites.

Agricultural livestock include, for example, mammals, such as sheep, goats, horses, donkeys, camels, buffaloes, rabbits, reindeers, fallow deers, and in particular cattle and pigs; or poultry such as turkeys, ducks, geese, and in particular chickens; fish and crustaceans, for example in aquaculture; and also insects such as bees.

Domestic animals include, for example, mammals, such as hamsters, guinea pigs, rats, mice, chinchillas, ferrets and in particular dogs, cats, cage birds, reptiles, amphibians and aquarium fish.

According to a preferred embodiment, the compounds of the formula (I), (Ia), (Ib) or (Ic) are administered to mammals.

According to another preferred embodiment, the compounds of the formula (I), (Ia), (Ib) or (Ic) are administered to birds, namely cage birds and in particular poultry.

By using the compounds of the formula (I), (Ia), (Ib) or (Ic) to control animal parasites, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling” as used herein with regard to the animal health field, means that the compounds of the formula (I), (Ia), (Ib) or (Ic) are effective in reducing the incidence of the respective parasite in an animal infected with such parasites to innocuous levels. More specifically, “controlling”, as used herein, means that the compound of the formula (I), (Ia), (Ib) or (Ic) is effective in killing the respective parasite, inhibiting its growth, or inhibiting its proliferation.

Arthropods include:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.;
from the order of the Heteropterida, for example Cimex spp., Triatoma spp., Rhodnius spp., Panstrongylus spp.; as well as nuisance and hygiene pests from the order of the Blattarida.

Arthropods furthermore include:

from the subclass of the Acari (Acarina) and the order of the Metastigmata, for example from the family of argasidae like Argas spp., Ornithodorus spp., Otobius spp., from the family of Ixodidae like Ixodes spp., Amblyomma spp., Rhipicephalus (Boophilus) spp Dermacentor spp., Haemophysalis spp., Hyalomma spp., Rhipicephalus spp. (the original genus of multi-host ticks); from the order of mesostigmata like Dermanyssus spp., Ornithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp, Laminosioptes spp.

Parasitic Protozoa include:

Mastigophora (Flagellata) such as, for example, Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example, Giardia lamblia, G. canis;
Sarcomastigophora (Rhizopoda) such as Entamoebidae, for example, Entamoeba histolytica, Hartmanellidae, for example, Acanthamoeba sp., Harmanella sp.;
Apicomplexa (Sporozoa) such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example, Toxoplasma gondii, Hammondia heydornii, Neospora caninum, Besnoitia besnoitii; such as Sarcocystidae, for example, Sarcocystis bovicanis, S. bovihominis, S. ovicanis, S. ovifelis, S. neurona, S. spec., S. suihominis, such as Leucozoidae, for example, Leucozytozoon simondi, such as Plasmodiidae, for example, Plasmodium berghei, P. falciparum, P. malariae, P. ovate, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Pathogenic endoparasites, which are helminths, include Platyhelmintha (e.g. Monogenea, cestodes and trematodes), nematodes, Acanthocephala, and Pentastoma, including:

### Monogenea: e.g.: Gyrodactylus spp., Dactylogyrus spp., Polystoma spp.;

Cestodes: from the order of the Pseudophyllidea for example: Diphyllobothrium spp., Spirometra spp., Schistocephalus spp., Ligula spp., Bothridium spp., Diplogonoporus spp.;
from the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.;
Trematodes: from the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fascioloides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.;

### Nematodes: Trichinellida zum Beispiel: Trichuris spp., Capillaria spp., Paracapillaria spp., Eucoleus spp., Trichomosoides spp., Trichinella spp.;

from the order of the Tylenchida for example: Micronema spp., Strongyloides spp.;
from the order of the Rhabditida for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;
from the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;
Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.;
from the order of the Echinorhynchida, for example, Acanthocephalus spp., Echinorhynchus spp., Leptorhynchoides spp.;
Pentastoma: from the order of the Porocephalida, for example, Linguatula spp.

In the veterinary field and in animal keeping, administration of the compounds of the formula (I), (Ia), (Ib) or (Ic) is carried out by methods generally known in the art, such as enterally, parenterally, dermally or nasally in the form of suitable preparations. Administration can be carried out prophylactically or therapeutically.

Thus, one embodiment of the present invention refers to the use of a compound of the formula (I), (Ia), (Ib) or (Ic) as medicament.

A further aspect refers to the use of a compound of the formula (I), (Ia), (Ib) or (Ic) as an antiendoparasitic agent, in particular a helminthicidal agent or antiprotozoic agent. Compounds of the formula (I), (Ia), (Ib) or (Ic) are suitable for use as an antiendoparasitic agent, in particular a helminthicidal agent or antiprotozoic agent, for example in animal husbandry, in animal breeding, in animal housing and in the hygiene sector.

A further aspect in turn relates to the use of a compound of the formula (I), (Ia), (Ib) or (Ic) as an antiectoparasitic, in particular an arthropodicide such as an insecticide or an acaricide. A further aspect relates to the use of a compound of the formula (I), (Ia), (Ib) or (Ic) as an antiectoparasitic, in particular an arthropodicide such as an insecticide or an acaricide, for example in animal husbandry, in animal breeding, in stables or in the hygiene sector.

### Anthelmintic Mixing Components

The following anthelmintic mixing components may be mentioned by way of example:

Anthelmintically active compounds including trematicidally and cestocidally active compounds:
from the class of the macrocyclic lactones, for example: abamectin, doramectin, emamectin, eprinomectin, ivermectin, milbemycin, moxidectin, nemadectin, selamectin;
from the class of the benzimidazoles and probenzimidazoles, for example: albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, triclabendazole;
from the class of the cyclooctadepsipeptides, for example: emodepside, PF1022;
from the class of the aminoacetonitrile derivatives, for example: monepantel;
from the class of the tetrahydropyrimidines, for example: morantel, pyrantel, oxantel;
from the class of the imidazothiazoles, for example: butamisole, levamisole, tetramisole;
from the class of the salicylanilides, for example: bromoxanide, brotianide, clioxanide, closantel, niclosamide, oxyclozanide, rafoxanide, tribromsalan;
from the class of the paraherquamides, for example: derquantel, paraherquamide;
from the class of the aminophenylamidines, for example: amidantel, deacylated amidantel (dAMD), tribendimidine;
from the class of the organophosphates, for example: coumaphos, crufomate, dichlorvos, haloxone, naphthalofos, trichlorfon;
from the class of the substituted phenols, for example: bithionol, disophenol, hexachlorophene, niclofolan, meniclopholan, nitroxynil;
from the class of the piperazinones, for example: praziquantel, epsiprantel;
from various other classes, for example: amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine, resorantel.

### Vector Control

The compounds of the formula (I), (Ia), (Ib) or (Ic) can also be used in vector control. For the purpose of the present invention, a vector is an arthropod, in particular an insect or arachnid, capable of transmitting pathogens such as, for example, viruses, worms, single-cell organisms and bacteria from a reservoir (plant, animal, human, etc.) to a host. The pathogens can be transmitted either mechanically (for example trachoma by non-stinging flies) to a host, or by injection (for example malaria parasites by mosquitoes) into a host.

Examples of vectors and the diseases or pathogens they transmit are:

### 1) Mosquitoes

Anopheles: malaria, filariasis;
Culex: Japanese encephalitis, filariasis, other viral diseases, transmission of worms;
Aedes: yellow fever, dengue fever, filariasis, other viral diseases;
Simuliidae: transmission of worms, in particular Onchocerca volvulus;
   2) Lice: skin infections, epidemic typhus;
   3) Fleas: plague, endemic typhus;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), CrimeanCongo haemorrhagic fever, borreliosis;
   6) Ticks: borellioses such as Borrelia duttoni, tick-borne encephalitis, Q fever (Coxiella burnetii), babesioses (Babesia canis canis).

Examples of vectors in the sense of the present invention are insects, for example aphids, flies, leafhoppers or thrips, which are capable of transmitting plant viruses to plants. Other vectors capable of transmitting plant viruses are spider mites, lice, beetles and nematodes.

Further examples of vectors in the sence of the present invention are insects and arachnids such as mosquitoes, in particular of the genera Aedes, Anopheles, for example A. gambiae, A. arabiensis, A. funestus, A. dims (malaria) and Culex, lice, fleas, flies, mites and ticks capable of transmitting pathogens to animals and/or humans.

Vector control is also possible if the compounds of the formula (I), (Ia), (Ib) or (Ic) are resistance-breaking.

Compounds of the formula (I), (Ia), (Ib) or (Ic) are suitable for use in the prevention of diseases and/or pathogens transmitted by vectors. Thus, a further aspect of the present invention is the use of compounds of the formula (I), (Ia), (Ib) or (Ic) for vector control, for example in agriculture, in horticulture, in gardens and in leisure facilities, and also in the protection of materials and stored products.

### Protection of Industrial Materials

The compounds of the formula (I), (Ia), (Ib) or (Ic) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protecting wood is particularly preferred.

In a further embodiment, the compounds of the formula (I), (Ia), (Ib) or (Ic) are used together with at least one further insecticide and/or at least one fungicide.

In a further embodiment, the compounds of the formula (I), (Ia), (Ib) or (Ic) are present as a ready-to-use pesticide, i.e. they can be applied to the material in question without further modifications. Suitable further insecticides or fungicides are in particular those mentioned above.

Surprisingly, it has also been found that the compounds of the formula (I), (Ia), (Ib) or (Ic) can be employed for protecting objects which come into contact with saltwater or brackish water, in particular hulls, screens, nets, buildings, moorings and signalling systems, against fouling. Likewise, the compounds of the formula (I), (Ia), (Ib) or (Ic), alone or in combinations with other active compounds, can be used as antifouling agents.

### Control of Animal Pests in the Hygiene Sector

The compounds of the formula (I), (Ia), (Ib) or (Ic) are suitable for controlling animal pests in the hygiene sector. In particular, the invention can be applied in the domestic sector, in the hygiene sector and in the protection of stored products, especially for controlling insects, arachnids and mites encountered in enclosed spaces such as dwellings, factory halls, offices, vehicle cabins. For controlling animal pests, the compounds of the formula (I), (Ia), (Ib) or (Ic) are used alone or in combination with other active compounds and/or auxiliaries. They are preferably used in domestic insecticide products. The compounds of the formula (I), (Ia), (Ib) or (Ic) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

They are used, for example, in aerosols, pressure-free spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

The various aspects of the invention will now be illustrated with reference to the following production and use examples in a non-limiting manner.

### PREPARATION EXAMPLES

### ¹H-NMR Data

Method M1: ¹H-NMR-data were determined with a Bruker Avance 400 equipped with a flow cell (60 μl volume) or with a Bruker AVIII 400 equipped with 1.7 mm cryo CPTCI probe head or with a Bruker AVII 600 (600.13 MHz) equipped with a 5 mm cryo TCI probe head or with a Bruker AVIII 600 (601.6 MHz) equipped with a 5 mm cryo CPMNP probe head with tetramethylsilane as reference (0.0) and the solvents CD₃CN, CDCl₃ or D₆-DMSO.

Method M2: Alternatively ¹H-NMR-data were determined with a Bruker DMX300 (¹H-NMR: 300 MHz) using tetramethylsilane as reference standard.

NMR-data of selected examples are listed in classic format (chemical shift 6, multiplicity, number of hydrogen atoms) or as NMR-peak-lists.

### Preparation Example 1

### Synthesis of N-{2-[2-chloro-4-(5-chloropyridin-3-yl)phenyl]ethyl}-2-(trifluoromethyl)benzamide (corresponds to product expl. 1-45)

### Step 1: Synthesis of N-[2-(4-bromo-2-chlorophenyl)ethyl]-2-(trifluoromethyl)benzamide

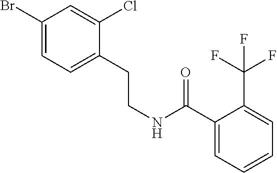 [SMILES: O=C(NCCc1ccc(Br)cc1Cl)c1ccccc1C(F)(F)F]

5.6 g (55.3 mmol) of triethylamine were added to a solution of 6 g (22.1 mmol) of 2-(4-bromo-2-chlorophenyl)ethanamine hydrochlorid in 25 mL dichloromethane at room temperature. 4.6 g (22.1 mmol) of 2-(trifluoromethyl)benzoyl chloride in 25 mL dichloromethane were slowly added to the reaction mixture at room temperature. After completion of reaction, the reaction mixture was diluted with dichloromethane and washed with water. The combined organic layers were evaporated under reduced pressure to yield 9.07 g (quantitative yield). The product has been used in step 2 without prior purification.

LCMS (M+H): 405.8, 407.8

¹H-NMR (400 MHz, d₆-DMSO, Method M1); δ 8.61 (t, 1H, NH), 7.77-7.62 (m, 3H), 7.53-7.51 (dd, 1H), 7.46 (d, 1H), 7.35 (d, 1H), 3.50-3.45 (qu, 2H), 2.91 (t, 2H).

### Step 2: Synthesis of N-{2-[2-chloro-4-(5-chloropyridin-3-yl)phenyl]ethyl}-2-(trifluoromethyl)benzamide (corresponds to product expl. 1-45)

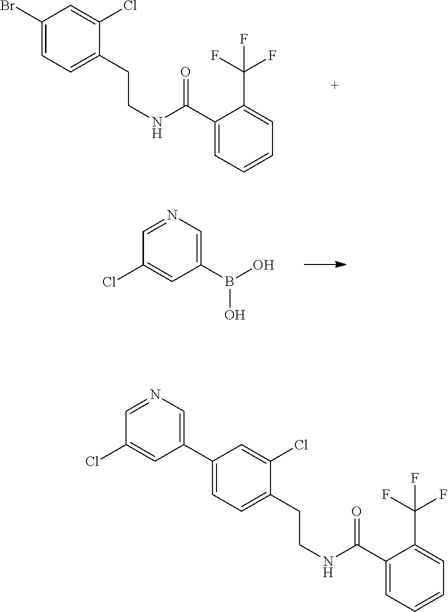 [SMILES: O=C(NCCc1ccc(Br)cc1Cl)c1ccccc1C(F)(F)F ; OB(O)c1cncc(Cl)c1 ; O=C(NCCc1ccc(-c2cncc(Cl)c2)cc1Cl)c1ccccc1C(F)(F)F]

110.9 mg (0.27 mmol) of N-[2-(4-bromo-2-chlorophenyl)ethyl]-2-(trifluoromethyl)benzamide (from step 1) and 47.2 mg (0.30 mmol) of (5-chloropyridin-3-yl)boronic acid were dissolved in 3 mL dioxane. Thereafter, 22 mg (0.03 mmol) of 1,1′-bis-(diphenylphosphino)-ferrocen)-palladium-dichloromethane complex and 286.2 mg (2.7 mmol) of sodium carbonate in 2 mL water were added and treated in a sealed microwave vial in a Biotage microwave oven (Initiator) at 100° C. for 20 minutes. The reaction mixture was filtered over a silica gel sodium sulfate cartridge, the solvents were evaporated and the crude product was purified by preparative HPLC to afford 60.6 mg (46%) of the title compound as off-white solid.

¹H-NMR (400 MHz, d6-DMSO, Method M1); δ 8.91 (s, 1H), 8.68 (t, 1H, NH), 8.64 (s, 1H), 8.33 (s, 1H), 7.94 (s, 1H), 7.83-7.46 (m, 6H), 3.55-3.50 (qu, 2H), 3.00 (t, 2H).

### Preparation Example 2

### Synthesis of N-[2-[2-chloro-4-[4-(trifluoromethyl)pyrazol-1-yl]phenyl]-2,2-difluoro-ethyl]-2-(trifluoromethyl)benzamide (corresponds to product expl. 1-69)

### Step 1: Synthesis of 2-(4-bromo-2-chloro-phenyl)-2,2-difluoro-ethanamine

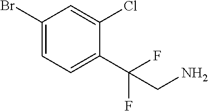 [SMILES: NCC(F)(F)c1ccc(Br)cc1Cl]

Synthesis of 2-(4-bromo-2-chloro-phenyl)-2,2-difluoro-ethanamine was performed in analogy to WO 2013/064460 A1 (referred as intermediates IIa-14 and IIa-15).

¹H-NMR (400 MHz, d6-DMSO, Method M1); δ 7.91 (s, 1H), 7.74 (d, 1H), 7.57 (d, 1H), 4.68 (bs, 2H, NH₂), 3.46 (t, 2H).

### Step 2: Synthesis of N-[2-(4-bromo-2-chloro-phenyl)-2,2-difluoro-ethyl]-2-(trifluoromethyl)benzamide

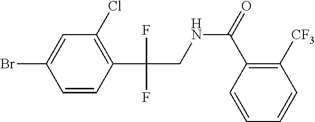 [SMILES: O=C(NCC(F)(F)c1ccc(Br)cc1Cl)c1ccccc1C(F)(F)F]

1.395 g (13.7 mmol) of triethylamine were added to a solution of 1.49 g (5.51 mmol) of 2-(4-bromo-2-chloro-phenyl)-2,2-difluoro-ethanamine (from step 1) in 30 mL dichloromethane at room temperature. 1.15 g (5.51 mmol) of 2-(trifluoromethyl)benzoyl chloride in 10 mL dichloromethane were slowly added to the reaction mixture at room temperature. After completion of reaction, the reaction mixture was diluted with dichloromethane and washed with water. The combined organic layers were evaporated under reduced pressure. The remaining residue was purified by flash silica gel chromatography resulting in 1.08 gas white solid (yield: 43.8%).

¹H-NMR (400 MHz, d6-DMSO, Method M1); δ 8.98 (t, 1H, NH), 7.91 (s, 1H), 7.77-7.56 (m, 5H), 7.36 (d, 1H), 4.17 (dt, 2H).

### Step 3: Synthesis of N-[2-[2-chloro-4-[4-(trifluoromethyl)pyrazol-1-yl]phenyl]-2,2-difluoro-ethyl]-2-(trifluoromethyl)benzamide (corresponds to product expl. 1-69)

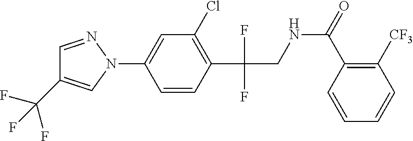 [SMILES: O=C(NCC(F)(F)c1ccc(-n2cc(C(F)(F)F)cn2)cc1Cl)c1ccccc1C(F)(F)F]

149.7 mg (0.33 mmol) of N-[2-(4-bromo-2-chloro-phenyl)-2,2-difluoro-ethyl]-2-(trifluoromethyl) benzamide (from step 3) were dissolved in 4.4 mL acetonitrile. Subsequently, 55.2 mg (0.4 mmol) of 4-trifluoropyrazole, 4.8 mg (0.03 mmol) of copper(I) oxide, 9.3 mg (0.06 mmol) of salicylaldoxime and 165.3 mg (0.5 mmol) of cesium carbonate were added at room temperature. The reaction mixture was kept and stirred in a sealed vial at 100° C. for 18 hours. The reaction mixture was allowed to cool to ambient temperature and was diluted with ethyl acetate followed by filtration. The filtrate was evaporated under reduced pressure and the remaining residue was purified by flash silica gel chromatography resulting in 39 mg as white solid (yield: 21.3%).

¹H-NMR (400 MHz, d6-DMSO, Method M1); δ 9.38 (s, 1H), 9.03 (t, 1H, NH), 8.31 (s, 1H), 8.20 (d, 1H), 8.06 (dd, 1H), 7.83-7.49 (m, 4H), 7.39 (d, 1H), 4.22 (dt, 2H).

### Preparation Example 3

### Synthesis of N-[2-[2-chloro-4-(6-fluoro-3-pyridyl)phenyl]-2,2-difluoro-ethyl]-2-(trifluoromethyl)benzamide (corresponds to product expl. 1-93)

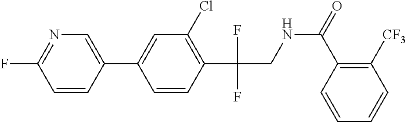 [SMILES: O=C(NCC(F)(F)c1ccc(-c2ccc(F)nc2)cc1Cl)c1ccccc1C(F)(F)F]

138.7 mg (0.31 mmol) N N-[2-(4-bromo-2-chloro-phenyl)-2,2-difluoro-ethyl]-2-(trifluoromethyl) benzamide (from preparation example 2, step 2) and 44.2 mg (0.31 mmol) (6-fluoro-3-pyridyl)boronic acid were dissolved in 5 mL dioxane. Thereafter, 23.1 mg (0.03 mmol) dichloro-bis(tricyclohexylphosphine) palladium(II) and 204.2 mg (0.63 mmol) cesium carbonate in 0.61 mL water were added and treated in a sealed microwave vial in a Biotage microwave oven (Initiator) at 100° C. for 30 minutes. The reaction mixture was filtered over a silica gel sodium sulfate cartridge, the solvents were evaporated and the crude product was purified by preparative HPLC to afford 120 mg (84.3%) of the title compound as off-white solid.

¹H-NMR (400 MHz, d6-DMSO, Method M1); δ 9.03 (t, 1H, NH), 8.67 (s, 1H), 8.42-8.38 (dt, 1H), 8.02 (s, 1H), 7.88 (d, 1H), 7.77-7.62 (m, 4H), 7.39-7.32 (d+d, 2H), 4.26-4.18 (dt, 1H).

### Preparation Example 4

### Synthesis of N-{2-[2-chloro-4-(6-chloropyridin-3-yl)phenyl]-2-fluoroethyl}-2-(trifluoromethyl) benzamide (corresponds to product expl. 1-73)

### Step 1: Synthesis of 2-(4-bromo-2-chlorophenyl)-2-trimethylsilyloxyacetonitrile

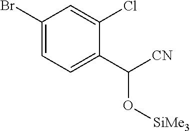 [SMILES: C[Si](C)(C)OC(C#N)c1ccc(Br)cc1Cl]

To vacuum dried potassium cyanide (22.2 g, 342 mmol) in acetonitrile (150 mL), 4-bromo-2-chlorobenzaldehyde (25 g, 114 mmol) and zinc iodide were added. Then trimethylsilylchloride (20 g, 182 mmol) was added dropwise and the mixture was refluxed for 40 hours. The reaction mixture was filtered and concentrated in vacuo to afford the title compound as a brown oil which was used in the next step without additional purification.

### Step 2: Synthesis of 2-(4-bromo-2-chlorophenyl)-2-fluoroacetonitrile

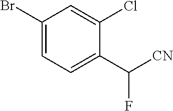 [SMILES: N#CC(F)c1ccc(Br)cc1Cl]

To a solution of 2-(4-bromo-2-chlorophenyl)-2-trimethylsilyloxyacetonitrile (36.3 g, 114 mmol) in dichloromethane (150 mL), DAST (22.2 g, 125 mmol) was added dropwise maintaining the temperature between −3 and 0° C. Then the reaction mixture was allowed to warm to room temperature and stirred over weekend. The reaction mixture was quenched with a saturated aqueous solution of sodium bicarbonate solution. The phases were separated. The organic layer was washed with a saturated sodium bicarbonate solution and some water. The organic layer was dried with sodium sulfate. The solvent was removed in vacuo to afford the title compound as a yellow oil which was used in the next step without additional purification.

### Step 3: Synthesis of 2-(4-bromo-2-chlorophenyl)-2-fluoroethylamine hydrochloride

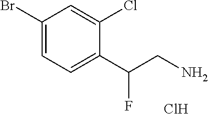 [SMILES: NCC(F)c1ccc(Br)cc1Cl ; Cl]

To a solution of 2-(4-bromo-2-chlorophenyl)-2-fluoroacetonitrile (27.7 g, 111 mmol) in tetrahydrofurane (300 mL), diborane in tetrahydrofurane (444 mmol) was added dropwise maintaining the temperature between −10° C. and −5° C. The reaction mixture was allowed to warm to room temperature and stirred overnight. The reaction mixture was quenched with methanol (100 mL), acidified with a dry solution of HO/ether and evaporated to dryness. Recrystallization of the residue from isopropyl alcohol-ether afforded the title compound as a white solid (19.4 g).

¹H-NMR (400 MHz, d₆-DMSO Method M1); δ 8.60 (sb, 3H), 7.87 (s, 1H), 7.73 (dd, 1H), 7.56 (d, 1H), 6.22 (m, 1H), 3.44-3.21 (m, 2H).

LCMS (M-HCl+H)⁺: 252.0, 254.0

### Step 4: Synthesis of N-[2-(4-bromo-2-chlorophenyl)-2-fluoroethyl]-2-(trifluoromethyl)benzamide

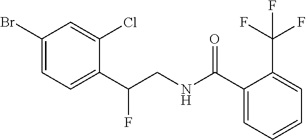 [SMILES: O=C(NCC(F)c1ccc(Br)cc1Cl)c1ccccc1C(F)(F)F]

To a solution of 2-(4-bromo-2-chlorophenyl)-2-fluoroethylamine hydrochloride (5 g, 17.3 mmol) in 75 mL of dichloromethane was added at 0° C. triethylamine (7.235 mL, 51.9 mmol) and dropwise 2-(trifluoromethyl)benzoyl chloride (4.3 g, 20.8 mmol). The reaction mixture was stirred at room temperature overnight. Then 30 mL of dichloromethane were added. The reaction mixture was washed with a solution of 1N HCl and then a saturated solution of NaHCO3. Evaporation led to a residue wish was purified by flash chromatography affording 5.5 g of the title compound.

¹H-NMR (400 MHz, d₆-DMSO Method M1); δ 8.91 (t, 1H), 7.82-7.63 (m, 5H), 7.54-7.45 (m, 2H), 6.00 (dt, 1H), 3.81-3.69 (m, 2H)

### Step 5: Synthesis of N-{2-[2-chloro-4-(6-chloropyridin-3-yl)phenyl]-2-fluoroethyl}-2-(trifluoromethyl) benzamide (corresponds to product expl. 1-73)

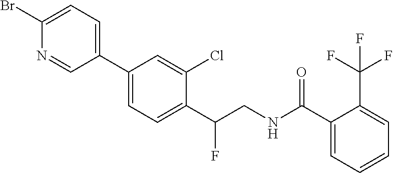 [SMILES: O=C(NCC(F)c1ccc(-c2ccc(Br)nc2)cc1Cl)c1ccccc1C(F)(F)F]

To a solution of 200 mg of N-[2-(4-bromo-2-chlorophenyl)-2-fluoroethyl]-2-(trifluoromethyl)benzamide (0.47 mmol) in degased dioxane (5 mL) was added under argon (6-chloropyridin-3-yl)boronic acid (82 mg, 0.52 mmol), 1,1,-bis-(diphenylphosphino)-(ferrocen)-palladium(II)-dichlormethane (19 mg, 0.024 mmol) and 0.283 mL of an aqueous solution of sodium carbonate (60 mg, 0.57 mmol). The reaction was stirred at 80° C. for 2 hours. The reaction mixture was cooled down and some water and ethyl acetate were added. The two phases were separated and the organic phase was dried and evaporated. The residue obtained was purified by flash chromatography. This afforded 93 mg of the title compound.

¹H-NMR: see NMR peak list in the table, example 1-73

LCMS (M+H)⁺: 257.0, 459.0

### Preparation Example 5

### Synthesis of N-{2-[2-chloro-4-(5-fluoropyridin-3-yl)phenyl]-2-fluoropropyl}-2-(trifluoromethyl) benzamide (corresponds to product Example 1-101)

### Step 1: Synthesis of 4-bromo-2-chloro-N-methoxy-N-methylbenzamide

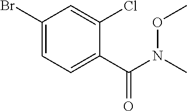

To a solution of 4-bromo-2-chlorobenzoic acid (15 g, 63.69 mmol) in N-,N-dimethylformamide (150 mL) was added diisopropylethylamine (17.3 g, 133.7 mmol) followed by HBTU (26.6 g, 70.06 mmol) followed by MeNHOMe.HCl (6.2 g, 63.69 mmol). The reaction mixture was stirred at 70° C. for 10 hours, and then extracted with ethyl acetate ethyl acetate (2×50 mL). The organic layer was washed with water (50 mL), 1N HCl (50 mL), a saturated aqueous solution of sodium hyrogenocarbonate (50 mL), brine, was dried with sodium sulfate and filtrated. The organic phase was then concentrated in vacuo leading to a residue, which was purified by flash column chromatography (PE:EA=5:1). This afforded the title compound (13.3 g) as oil.

LCMS (M+H): 278.0, 280.0

### Step 2: Synthesis of 1-(4-bromo-2-chlorophenyl)ethanone

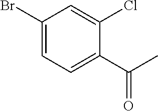

To a solution of 4-bromo-2-chloro-N-methoxy-N-methylbenzamide (13.3 g, 47.76 mmol) in tetrahydrofurane (100 mL) was added methylmagnesiumbromide (71.63 mmol) at −60° C. under nitrogen. The reaction mixture was stirred at −60° C. for 1 hour, then stirred at 25° C. for 3 hours. A saturated aqueous solution of ammonium chloride was added. The reaction mixture was extracted with ethylacetate (100 mL) twice. The organic layer was washed by brine, dried with sodium sulfate and concentrated in vacuo. This afforded the title compound (9.7 g) as brown solid.

LCMS (M+H)⁺: 233.1, 234.9

### Step 3: Synthesis of 1-[2-chloro-4-(5-fluoropyridin-3-yl)phenyl]ethanone

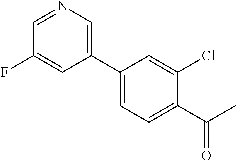 [SMILES: CC(=O)c1ccc(-c2cncc(F)c2)cc1Cl]

To a solution of 1-(4-bromo-2-chlorophenyl)ethanone (9.7 g, 41.54 mmol) in toluene/dioxane/water (60 mL/20 mL/2 mL) was added (5-fluoropyridin-3-yl)boronic acid (7.6 g, 54.00 mmol), cesium carbonate (27 g, 83.08 mmol) and Pd(PPh₃)₄ (4.8 g, 4.154 mmol) under nitrogen. The reaction mixture was heated at 100° C. for 8 hours, filtrated. The reaction mixture was then extracted with EtOAc (50 mL) twice, washed with brine and dried with sodium sulfate and concentrated under vacuo. After purification by flash column chromatography (PE:EA=10:1) this afforded the title compound (2.5 g) as white solid.

¹H-NMR (400 MHz, d₆-DMSO, Method M1); δ 8.90 (s, 1H), 8.65 (d, 1H), 8.22 (d, 1H), 8.02 (d, 1H), 7.82-7.93 (m, 2H), 2.63 (s, 3H)

LCMS (M+H)⁺: 250.0

### Step 4: Synthesis of 2-[2-chloro-4-(5-fluoropyridin-3-yl)phenyl]-2-hydroxypropanenitrile

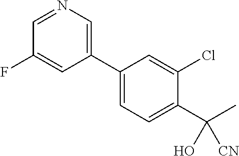 [SMILES: CC(O)(C#N)c1ccc(-c2cncc(F)c2)cc1Cl]

Anhydrous zinc diodide (2.88 mg, 0.009 mmol) was placed in a three-necked round-bottomed flask fitted with an addition funnel under argon atmosphere. A solution of 1-[2-chloro-4-(5-fluoropyridin-3-yl)phenyl]ethanone (1.5 g, 6.01 mmol) in anhydrous dichloromethane (5 mL) was added and the contents were stirred at room temperature. The mixture was cooled to 0° C. and trimethylsilylcyanide (596.04 mL, 6.01 m mol) was added dropwise to the vigorously stirred reaction mixture. After addition, the reaction mixture returned to room temperature and was allowed to stir at that temperature overnight. Then the reaction was cooled down to 0° C. and additional dichloromethane (5 mL), anhydrous zinc diodide (2.88 mg, 0.009 mmol) and trimethylsilylcyanide (596.04 mL, 6.01 mmol) were added. After stirring an additional night at room temperature, another addition of dichloromethane (5 mL), anhydrous zinc diodide (2.88 mg, 0.009 mmol) and trimethylsilylcyanide (596.04 mL, 6.01 mmol) was performed at 0° C. and reaction mixture was stirred a third night at room temperature. 7.4 mL of dichloromethane as well as a solution of Diethylaminosulfur trifluoride (DAST) in dichloromethane (1.07 g, 6.61 mmol in 3.1 ml of dichloromethane) were added at 0° C. After overnight stirring at room temperature, 35 ml of cold water were added. The reaction mixture was extracted with dichloromethane. The phases were separated. The organic phase was washed with an aqueous solution of HCl (0.5N), then water, then a saturated solution of NaHCO3 and again water. After drying with sodium sulfate and evaporation, the residue obtained was purified by flash chromatography. This afforded 883 mg of the title compound.

¹H-NMR (400 MHz, d₆-DMSO, Method M1); δ 8.87 (s, 1H), 8.64 (d, 1H), 8.18 (m, 1H), 8.01 (d, 1H), 8.85 (m, 2H), 7.66 (s, 1H), 1.90 (s, 3H)

LCMS (M+H)⁺: 277.1

### Step 4: Synthesis of 2-[2-chloro-4-(5-fluoropyridin-3-yl)phenyl]-2-fluoropropanenitrile

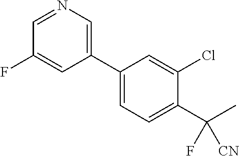 [SMILES: CC(F)(C#N)c1ccc(-c2cncc(F)c2)cc1Cl]

To a solution of 2-[2-chloro-4-(5-fluoropyridin-3-yl)phenyl]-2-hydroxypropanenitrile in dichloromethane was added dropwise at 0° C., DAST in dichloromethane (463.5 mg, 2.88 mmol in 3.1 ml of dichloromethane). The reaction mixture was stirred overnight at room temperature. 35 mL of cold water were then added. The reaction mixture was extracted with dichloromethane. The phases were separated. The organic phase was washed with an aqueous solution of HCl (0.5N), then water, then a saturated solution of NaHCO3 and again water. After drying with sodium sulfate and evaporation, the residue obtained was purified by flash chromatography. This afforded 484 mg of the title compound.

¹H-NMR (400 MHz, CD₃CN, Method M1); δ 8.75 (t, 1H), 8.54 (d, 1H), 7.92 (d, 1H), 7.87 (m, 1H)

LCMS (M+H)⁺: 279.1

### Step 5: Synthesis of 2-[2-chloro-4-(5-fluoropyridin-3-yl)phenyl]-2-fluoropropan-1-amine hydrochloride

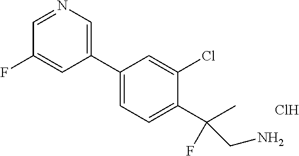 [SMILES: CC(F)(CN)c1ccc(-c2cncc(F)c2)cc1Cl ; Cl]

To a solution of 2-[2-chloro-4-(5-fluoropyridin-3-yl)phenyl]-2-fluoropropanenitrile (437 mg, 1.54 mmol) in dry tetrahydrofurane (20 mL) was added dropwise at 0° C. a solution of BH3.THF (1M, 4.610 mmol). The reaction mixture was brought to room temperature and stirred overnight at room temperature. Some ethanol was added. Some 1M HCL in diethylether was added and the reaction mixture was evaporated. To the residue obtained, was added 10 mL of acetone and MtBE. The solid obtained was separated by decantation leading to the title compound (562 mg).

¹H-NMR (400 MHz, d₆-DMSO, Method M1); δ 8.89 (s, 1H), 8.66 (d, 1H), 8.33 (s, 2H), 8.22 (dd, 1H), 8.04 (m, 1H), 7.93 (dd, 1H), 7.73 (d, 1H), 4.2 (bs, 2H), 1.97 (d, 3H)

LCMS (M+H-HCl)⁺: 283.0

### Step 6: Synthesis of N-{2-[2-chloro-4-(5-fluoropyridin-3-yl)phenyl]-2-fluoropropyl}-2-(trifluoromethyl)benzamide (corresponds to product expl. 1-101)

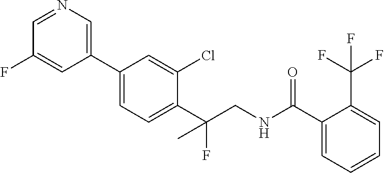 [SMILES: CC(F)(CNC(=O)c1ccccc1C(F)(F)F)c1ccc(-c2cncc(F)c2)cc1Cl]

To a solution 2-[2-chloro-4-(5-fluoropyridin-3-yl)phenyl]-2-fluoropropan-1-amine hydrochloride (90 mg, 0.262 mmol) and triethyalmine (0.110 mL, 0.787 mmol) in dichloromethane (5 mL) was added dropwise 2-(trifluoromethyl)benzoyl chloride (0.046 mL, 0.315 mmol). The reaction was stirred at room temperature. After control by thin layer chromatography some water was added. The reaction mixture was extracted with ethyl acetate. The organic phase was washed with brine and dried with sodium sulfate. Evaporation led to a residue which was purified by flash chromatography. This afforded 68 mg of the title compound.

¹H-NMR: see NMR peak list in the table, example 1-101.

LCMS (M+H)⁺: 455.0

### Preparation Example 6

### Synthesis of N-{2-[2-chloro-4-(6-fluoropyridin-3-yl)phenyl]-2-fluoropropyl}-2-(trifluoromethyl) benzamide (corresponds to product expl. 1-130)

### Step 1: Synthesis of 2-(2,4-dichlorophenyl)-2-fluoropropanenitrile

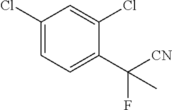

Trimethylsilylcyanide (5.3 mL, 39.7 mmol) was added dropwise to a stirred suspension of ZnI₂ (19.036 mg, 0.06 mmol), 1-(2,4-dichlorophenyl)ethanone (7.516 g, 39.7 mmol) and dichloromethane (3.8 ml) under nitrogen atmosphere at 0° C. The reaction mixture was stirred at room temperature overnight. It was then diluted with dry dichloromethane (49 mL), cooled again to 0° C., and a solution of DAST (5.78 mL, 43.7 mmol) in dichloromethane (20.4 mL) was added dropwise. The reaction mixture was stirred overnight at room temperature. The reaction mixture is poured in 61 mL of iced water and extracted with dichloromethane. The organic layer was washed sequentially with a 0.5N aqueous HCl solution, water, a saturated aqueous solution of NaHCO3, and water again. The organic layer was dried over sodium sulfate, filtered, and concentrated. The residue obtained was further purified by flash chromatography on silicagel (eluent: cyclohexane/ethylacetate). This afforded 8.576 g of the title compound.

¹H-NMR (400 MHz, CD₃CN, Method M1); δ, 7.63 (m, 2H), 7.50 (dd, 1H), 2.20 (d, 3H);

### Step 2: Synthesis of 2-(2,4-dichlorophenyl)-2-fluoropropan-1-amine hydrochloride

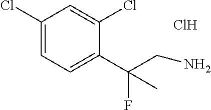

9.79 g of 2-(2,4-dichlorophenyl)-2-fluoropropanenitrile (44.8 mmol) in anhydrous tetrahydrofurane (614 mL) were cooled to 0° C. 1 M borane-THF (134.6 mL, 134 mmol) was added dropwise. The reaction mixture returned to room temperature and was stirred at room temperature for 4 hours. It was then quenched with ethanol and was acidified with 1M HCl in diethyl ether and concentrated in vacuo. The residue was triturated with acetone and filtrated. This led to the 5.176 g of the title compound.

¹H-NMR (400 MHz, d₆-DMSO, Method M1); δ, 8.10 (s, 3H), 7.73 (s, 1H), 7.59 (m, 2H), 3.67-3.50 (m, 2H), 1.87 (d, 3H);

LCMS (M+H-HCl)⁺: 220.0

### Step 3: Synthesis N-[2-(2,4-dichlorophenyl)-2-fluoropropyl]-2-(trifluoromethyl)benzamide

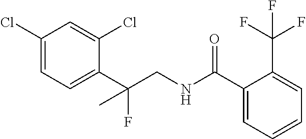 [SMILES: CC(F)(CNC(=O)c1ccccc1C(F)(F)F)c1ccc(Cl)cc1Cl]

155 mg of 2-(2,4-dichlorophenyl)-2-fluoropropan-1-amine hydrochloride (0.56 mmol) were placed in dichloromethane (5 mL). Triethylamine (0.236 mL, 1.69 mmol) and 2-(trifluoromethyl)benzoyl chloride (141 mg, 0.67 mmol) were added. The reaction mixture was left under stirring at room temperature overnight. Some ethyl acetate was added. The organic phase was washed with brine, dried over sodium sulfate and concentrated in vacuo. The residue obtained was purified by flash chromatography on silicagel (eluent: cyclohexane/ethylacetate). This afforded 174 mg (78%) of the title compound.

¹H-NMR (400 MHz, CDCl₃, Method M1); δ, 7.69 (m, 1H), 7.57-7.50 (m, 3H), 7.44 (d, 1H), 7.33 (m, 2H), 5.92 (s, 1H), 4.28-4.16 (m, 2H), 1.91 (d, 3H);

LC-MS: (M+H)⁺: 395.0

### Step 3: Synthesis of N-{2-[2-chloro-4-(6-fluoropyridin-3-yl)phenyl]-2-fluoropropyl}-2-(trifluoromethyl)benzamide (corresponds to product expl. 1-130)

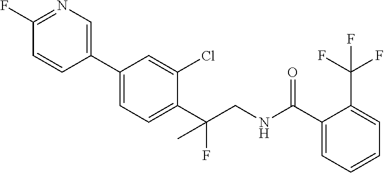 [SMILES: CC(F)(CNC(=O)c1ccccc1C(F)(F)F)c1ccc(-c2ccc(F)nc2)cc1Cl]

To a solution of N-[2-(2,4-dichlorophenyl)-2-fluoropropyl]-2-(trifluoromethyl)benzamide (141 mg, 0.358 mmol) in degassed toluene (2.4 mL) was added under argon (6-fluoropyridin-3-yl)boronic acid (50 mg, 0.358 mmol), palladium acetate (12.0 mg, 0.054 mmol), an aqueous solution of potassium phosphate (276.4 mg, 1.302 mmol in 2.4 mL of water) and dicyclohexyl(2-6-dimethoxy-2-yl)phosphine (39.6 mg, 0.097 mmol). The reaction mixture was heated 30 minutes at reflux. The reaction mixture was cooled down and some methyl tertiary butyl ether was added. After filtration on celite, the reaction mixture was dried with sodium sulfate. After evaporation, the residue obtained was purified by flash chromatography on silicagel (eluent: cyclohexane/ethylacetate). This afforded 10.6 mg of the title compound.

¹H-NMR: see NMR peak list in the table, example 1-130.

LC-MS: (M+H)⁺: 455.1

### Preparation of Intermediates

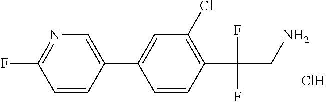 [SMILES: NCC(F)(F)c1ccc(-c2ccc(F)nc2)cc1Cl ; Cl]

A mixture of 2-(4-bromo-2-chlorophenyl)-2,2-difluoro-1-ethanamine (preparation example 2, step 1) (35.2 g, 0.13 mol), 6-fluoropyridin-3-ylboronic acid (23 g, 0.163 mol) and potassium carbonate (19.8 g, 0.143 mol) in toluene (360 ml) and ethanol (125 ml) was de gassed with nitrogen. Tetrakis(triphenylphosphine)palladium(O) (15 g, 13 mmol) and a solution of potassium fluoride (22.6 g, 0.39 mol) in water (57 ml) were added and the mixture was degassed with nitrogen. Then the mixture was stirred at 70° C. for 19 hours and at r.t. for 40 hours.

The reaction mixture was diluted with water (300 ml) and the organic layer was separated. The water layer was extracted with diethyl ether (2*100 ml). The combined organic layers were mixed with 10% hydrochloric acid (500 ml) and the mixture was filtered. The filtercake was washed with ether and toluene. The filtrate was separated.

The water layer was combined with the solid material and the mixture was neutralized with sodium hydrogencarbonate then with sodium carbonate. Crude amine was extracted with ether (3×150 ml). The extract was washed with water (150 ml), brine (150 ml), dried (Na₂SO₄) and treated with an ether-hydrogenchlorid solution.

The crude 2-[2-chloro-4-(6-fluoro-3-pyridyl)phenyl]-2,2-difluoro-1-ethanamine hydrochloride (32 g, 76% yield) was filtered off, washed with ether and dried in vacuo.

¹H-NMR (400.0 MHz, DMSO, Method M1): δ=8.79 (bs, 3H, NH₃), 8.69 (d, 1H), 8.45-8.40 (dt, 1H), 8.08 (s, 1H), 7.93-7.91 (d, 1H), 7.80-7.78 (d, 1H), 7.38-7.35 (dd, 1H), 3.92-3.81 (bt, 2H).

According to the methods described above, the following compounds of general formula (I) and intermediates (INT) have been prepared.

TABLE 1
Compounds of formula (I-1)

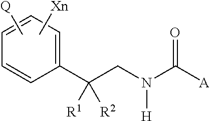

R¹, R², Q, X, n, A are as defined by each individual structure.

| Example no. | Formula |
|---|---|
| 1-1 | 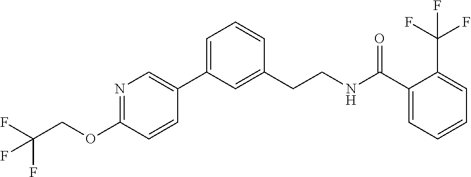 [SMILES: O=C(NCCc1cccc(-c2ccc(OCC(F)(F)F)nc2)c1)c1ccccc1C(F)(F)F] |
| 1-2 | 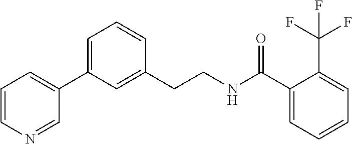 [SMILES: O=C(NCCc1cccc(-c2cccnc2)c1)c1ccccc1C(F)(F)F] |
| 1-3 | 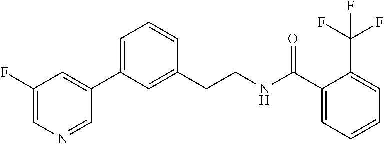 [SMILES: O=C(NCCc1cccc(-c2cncc(F)c2)c1)c1ccccc1C(F)(F)F] |
| 1-4 | 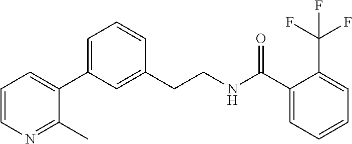 [SMILES: Cc1ncccc1-c1cccc(CCNC(=O)c2ccccc2C(F)(F)F)c1] |
| 1-5 | 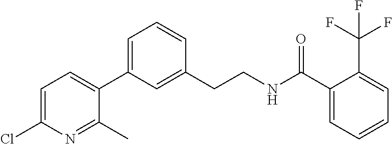 [SMILES: Cc1nc(Cl)ccc1-c1cccc(CCNC(=O)c2ccccc2C(F)(F)F)c1] |
| 1-6 | 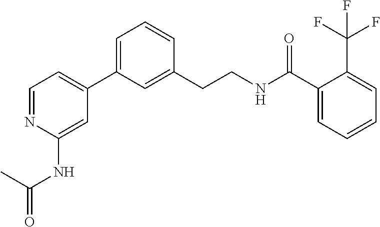 [SMILES: CC(=O)Nc1cc(-c2cccc(CCNC(=O)c3ccccc3C(F)(F)F)c2)ccn1] |
| 1-7 | 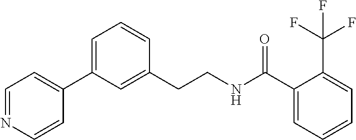 [SMILES: O=C(NCCc1cccc(-c2ccncc2)c1)c1ccccc1C(F)(F)F] |
| 1-8 | 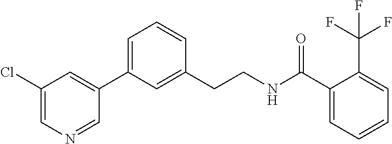 [SMILES: O=C(NCCc1cccc(-c2cncc(Cl)c2)c1)c1ccccc1C(F)(F)F] |
| 1-9 | 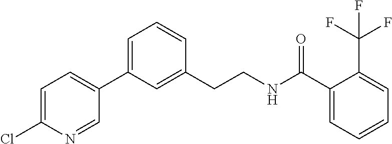 [SMILES: O=C(NCCc1cccc(-c2ccc(Cl)nc2)c1)c1ccccc1C(F)(F)F] |
| 1-10 | 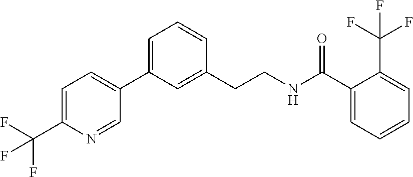 [SMILES: O=C(NCCc1cccc(-c2ccc(C(F)(F)F)nc2)c1)c1ccccc1C(F)(F)F] |
| 1-11 | 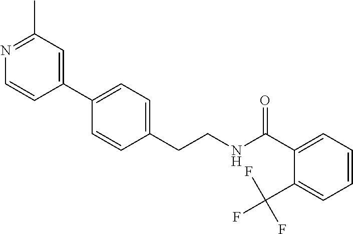 [SMILES: Cc1cc(-c2ccc(CCNC(=O)c3ccccc3C(F)(F)F)cc2)ccn1] |
| 1-12 | 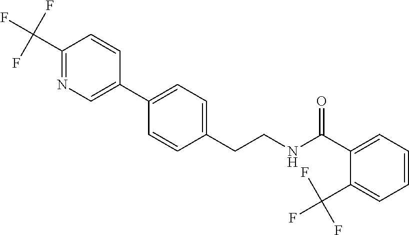 [SMILES: O=C(NCCc1ccc(-c2ccc(C(F)(F)F)nc2)cc1)c1ccccc1C(F)(F)F] |
| 1-13 | 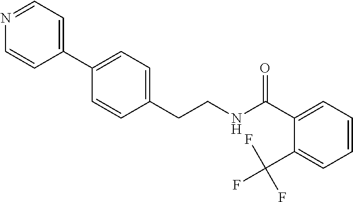 [SMILES: O=C(NCCc1ccc(-c2ccncc2)cc1)c1ccccc1C(F)(F)F] |
| 1-14 | 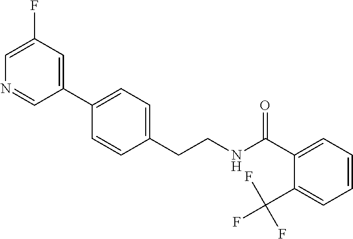 [SMILES: O=C(NCCc1ccc(-c2cncc(F)c2)cc1)c1ccccc1C(F)(F)F] |
| 1-15 | 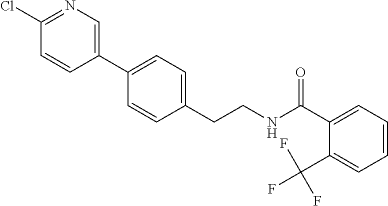 [SMILES: O=C(NCCc1ccc(-c2ccc(Cl)nc2)cc1)c1ccccc1C(F)(F)F] |
| 1-16 | 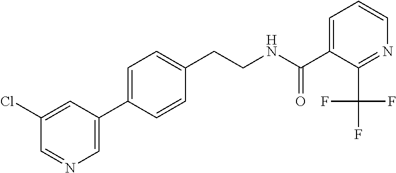 [SMILES: O=C(NCCc1ccc(-c2cncc(Cl)c2)cc1)c1cccnc1C(F)(F)F] |
| 1-17 | 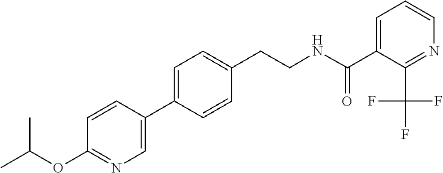 [SMILES: CC(C)Oc1ccc(-c2ccc(CCNC(=O)c3cccnc3C(F)(F)F)cc2)cn1] |
| 1-18 | 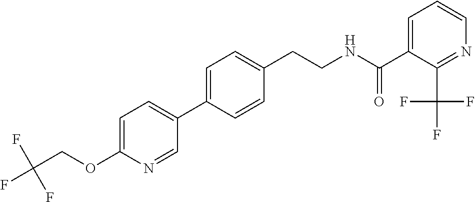 [SMILES: O=C(NCCc1ccc(-c2ccc(OCC(F)(F)F)nc2)cc1)c1cccnc1C(F)(F)F] |
| 1-19 | 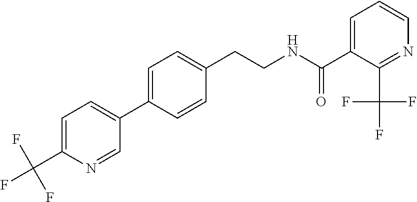 [SMILES: O=C(NCCc1ccc(-c2ccc(C(F)(F)F)nc2)cc1)c1cccnc1C(F)(F)F] |
| 1-20 | 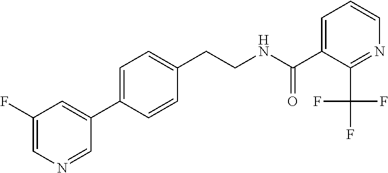 [SMILES: O=C(NCCc1ccc(-c2cncc(F)c2)cc1)c1cccnc1C(F)(F)F] |
| 1-21 | 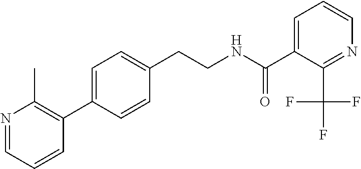 [SMILES: Cc1ncccc1-c1ccc(CCNC(=O)c2cccnc2C(F)(F)F)cc1] |
| 1-22 | 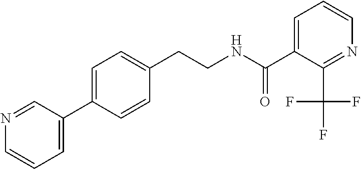 [SMILES: O=C(NCCc1ccc(-c2cccnc2)cc1)c1cccnc1C(F)(F)F] |
| 1-23 | 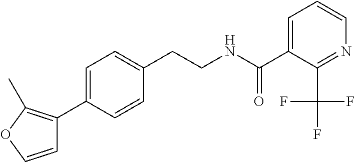 [SMILES: Cc1occc1-c1ccc(CCNC(=O)c2cccnc2C(F)(F)F)cc1] |
| 1-24 | 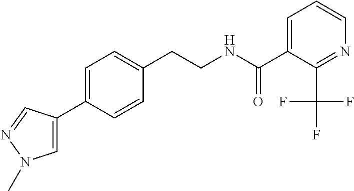 [SMILES: Cn1cc(-c2ccc(CCNC(=O)c3cccnc3C(F)(F)F)cc2)cn1] |
| 1-25 | 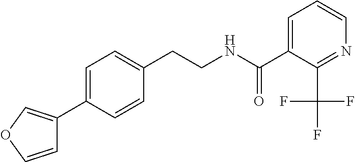 [SMILES: O=C(NCCc1ccc(-c2ccoc2)cc1)c1cccnc1C(F)(F)F] |
| 1-26 | 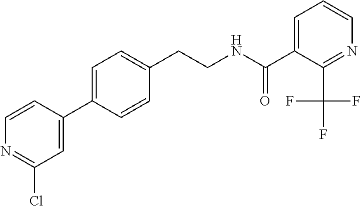 [SMILES: O=C(NCCc1ccc(-c2ccnc(Cl)c2)cc1)c1cccnc1C(F)(F)F] |
| 1-27 | 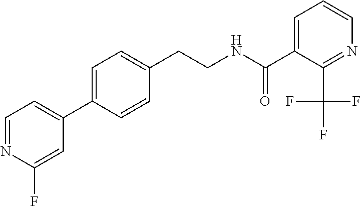 [SMILES: O=C(NCCc1ccc(-c2ccnc(F)c2)cc1)c1cccnc1C(F)(F)F] |
| 1-28 | 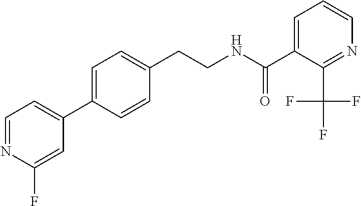 [SMILES: O=C(NCCc1ccc(-c2ccnc(F)c2)cc1)c1cccnc1C(F)(F)F] |
| 1-29 | 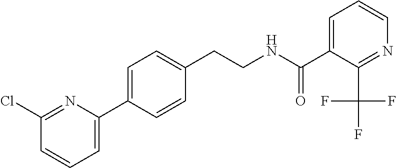 [SMILES: O=C(NCCc1ccc(-c2cccc(Cl)n2)cc1)c1cccnc1C(F)(F)F] |
| 1-30 | 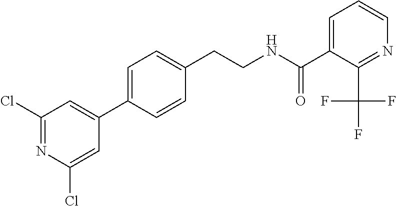 [SMILES: O=C(NCCc1ccc(-c2cc(Cl)nc(Cl)c2)cc1)c1cccnc1C(F)(F)F] |
| 1-31 | 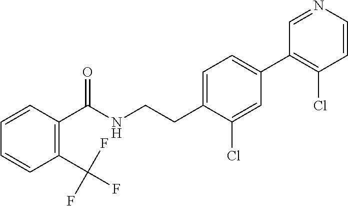 [SMILES: O=C(NCCc1ccc(-c2cnccc2Cl)cc1Cl)c1ccccc1C(F)(F)F] |
| 1-32 | 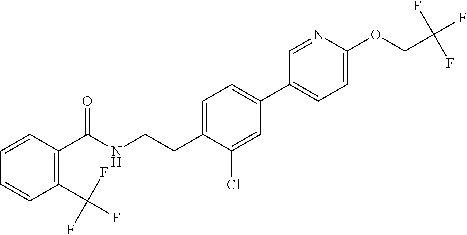 [SMILES: O=C(NCCc1ccc(-c2ccc(OCC(F)(F)F)nc2)cc1Cl)c1ccccc1C(F)(F)F] |
| 1-33 | 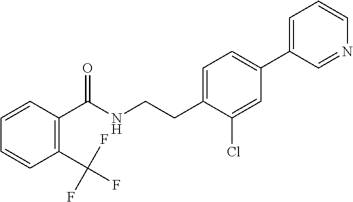 [SMILES: O=C(NCCc1ccc(-c2cccnc2)cc1Cl)c1ccccc1C(F)(F)F] |
| 1-34 | 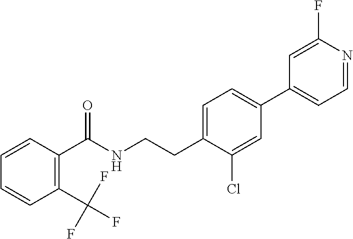 [SMILES: O=C(NCCc1ccc(-c2ccnc(F)c2)cc1Cl)c1ccccc1C(F)(F)F] |
| 1-35 | 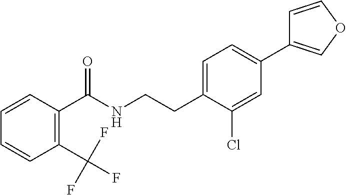 [SMILES: O=C(NCCc1ccc(-c2ccoc2)cc1Cl)c1ccccc1C(F)(F)F] |
| 1-36 | 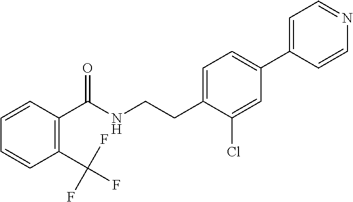 [SMILES: O=C(NCCc1ccc(-c2ccncc2)cc1Cl)c1ccccc1C(F)(F)F] |
| 1-37 | 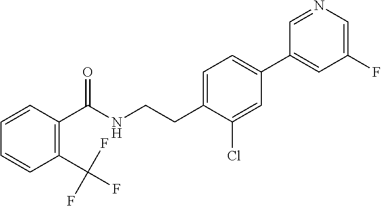 [SMILES: O=C(NCCc1ccc(-c2cncc(F)c2)cc1Cl)c1ccccc1C(F)(F)F] |
| 1-38 | 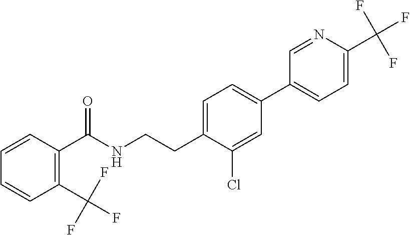 [SMILES: O=C(NCCc1ccc(-c2ccc(C(F)(F)F)nc2)cc1Cl)c1ccccc1C(F)(F)F] |
| 1-39 | 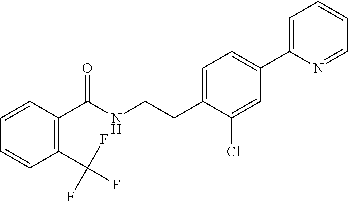 [SMILES: O=C(NCCc1ccc(-c2ccccn2)cc1Cl)c1ccccc1C(F)(F)F] |
| 1-40 | 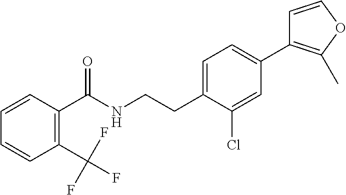 [SMILES: Cc1occc1-c1ccc(CCNC(=O)c2ccccc2C(F)(F)F)c(Cl)c1] |
| 1-41 | 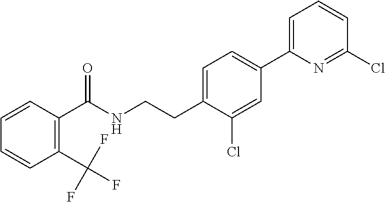 [SMILES: O=C(NCCc1ccc(-c2cccc(Cl)n2)cc1Cl)c1ccccc1C(F)(F)F] |
| 1-42 | 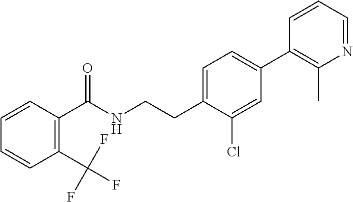 [SMILES: Cc1ncccc1-c1ccc(CCNC(=O)c2ccccc2C(F)(F)F)c(Cl)c1] |
| 1-43 | 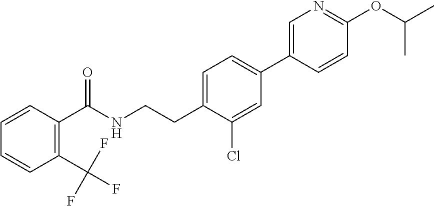 [SMILES: CC(C)Oc1ccc(-c2ccc(CCNC(=O)c3ccccc3C(F)(F)F)c(Cl)c2)cn1] |
| 1-44 | 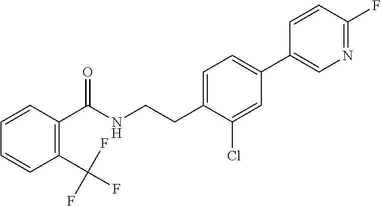 [SMILES: O=C(NCCc1ccc(-c2ccc(F)nc2)cc1Cl)c1ccccc1C(F)(F)F] |
| 1-45 | 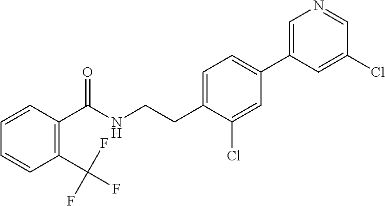 [SMILES: O=C(NCCc1ccc(-c2cncc(Cl)c2)cc1Cl)c1ccccc1C(F)(F)F] |
| 1-46 | 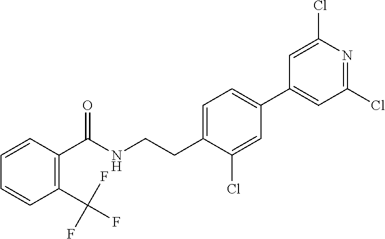 [SMILES: O=C(NCCc1ccc(-c2cc(Cl)nc(Cl)c2)cc1Cl)c1ccccc1C(F)(F)F] |
| 1-47 | 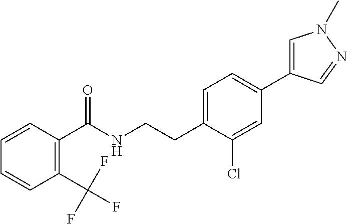 [SMILES: Cn1cc(-c2ccc(CCNC(=O)c3ccccc3C(F)(F)F)c(Cl)c2)cn1] |
| 1-48 | 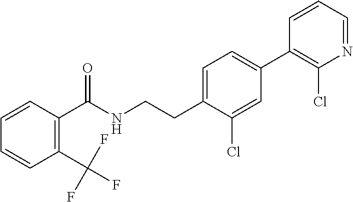 [SMILES: O=C(NCCc1ccc(-c2cccnc2Cl)cc1Cl)c1ccccc1C(F)(F)F] |
| 1-49 | 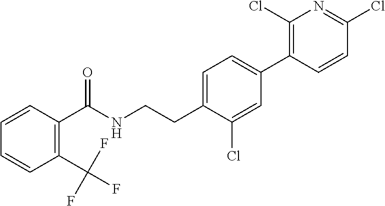 [SMILES: O=C(NCCc1ccc(-c2ccc(Cl)nc2Cl)cc1Cl)c1ccccc1C(F)(F)F] |
| 1-50 | 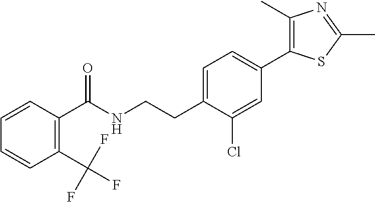 [SMILES: Cc1nc(C)c(-c2ccc(CCNC(=O)c3ccccc3C(F)(F)F)c(Cl)c2)s1] |
| 1-51 | 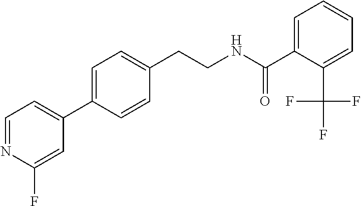 [SMILES: O=C(NCCc1ccc(-c2ccnc(F)c2)cc1)c1ccccc1C(F)(F)F] |
| 1-52 | 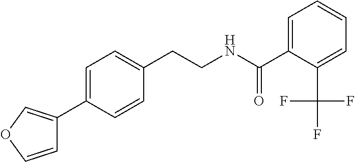 [SMILES: O=C(NCCc1ccc(-c2ccoc2)cc1)c1ccccc1C(F)(F)F] |
| 1-53 | 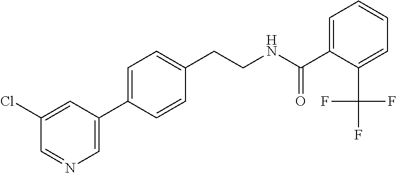 [SMILES: O=C(NCCc1ccc(-c2cncc(Cl)c2)cc1)c1ccccc1C(F)(F)F] |
| 1-54 | 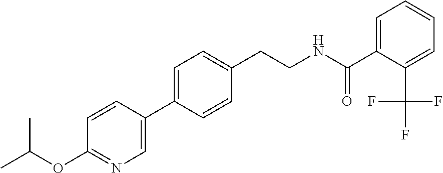 [SMILES: CC(C)Oc1ccc(-c2ccc(CCNC(=O)c3ccccc3C(F)(F)F)cc2)cn1] |
| 1-55 | 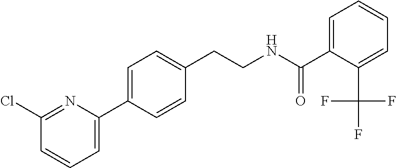 [SMILES: O=C(NCCc1ccc(-c2cccc(Cl)n2)cc1)c1ccccc1C(F)(F)F] |
| 1-56 | 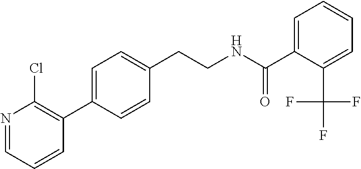 [SMILES: O=C(NCCc1ccc(-c2cccnc2Cl)cc1)c1ccccc1C(F)(F)F] |
| 1-57 | 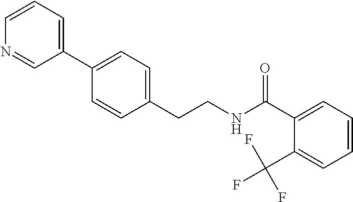 [SMILES: O=C(NCCc1ccc(-c2cccnc2)cc1)c1ccccc1C(F)(F)F] |
| 1-58 | 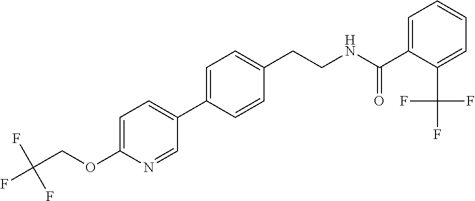 [SMILES: O=C(NCCc1ccc(-c2ccc(OCC(F)(F)F)nc2)cc1)c1ccccc1C(F)(F)F] |
| 1-59 | 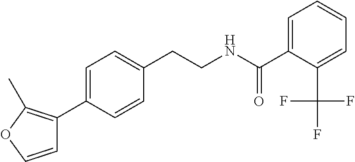 [SMILES: Cc1occc1-c1ccc(CCNC(=O)c2ccccc2C(F)(F)F)cc1] |
| 1-60 | 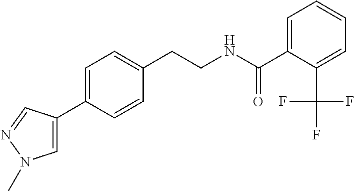 [SMILES: Cn1cc(-c2ccc(CCNC(=O)c3ccccc3C(F)(F)F)cc2)cn1] |
| 1-61 | 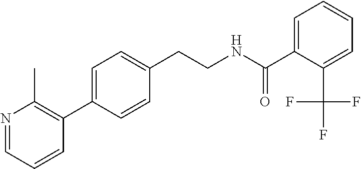 [SMILES: Cc1ncccc1-c1ccc(CCNC(=O)c2ccccc2C(F)(F)F)cc1] |
| 1-62 | 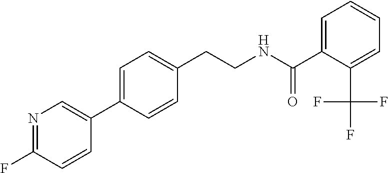 [SMILES: O=C(NCCc1ccc(-c2ccc(F)nc2)cc1)c1ccccc1C(F)(F)F] |
| 1-63 | 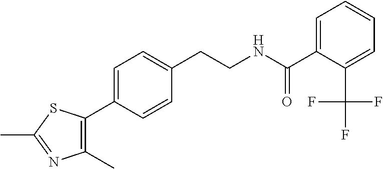 [SMILES: Cc1nc(C)c(-c2ccc(CCNC(=O)c3ccccc3C(F)(F)F)cc2)s1] |
| 1-64 | 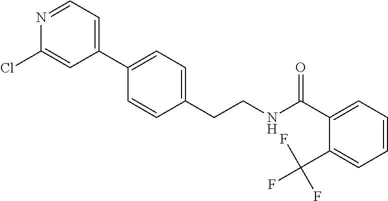 [SMILES: O=C(NCCc1ccc(-c2ccnc(Cl)c2)cc1)c1ccccc1C(F)(F)F] |
| 1-65 | 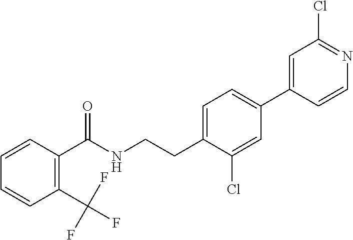 [SMILES: O=C(NCCc1ccc(-c2ccnc(Cl)c2)cc1Cl)c1ccccc1C(F)(F)F] |
| 1-66 | 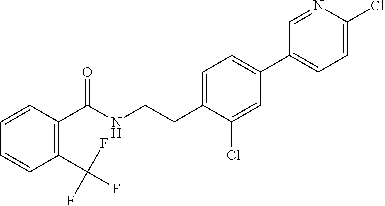 [SMILES: O=C(NCCc1ccc(-c2ccc(Cl)nc2)cc1Cl)c1ccccc1C(F)(F)F] |
| 1-67 | 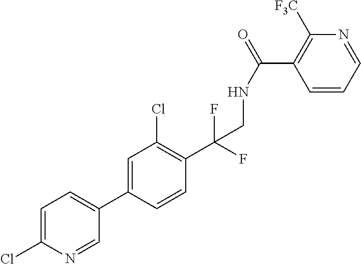 [SMILES: O=C(NCC(F)(F)c1ccc(-c2ccc(Cl)nc2)cc1Cl)c1cccnc1C(F)(F)F] |
| 1-68 | 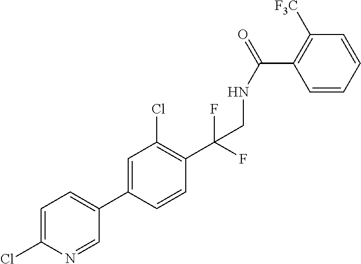 [SMILES: O=C(NCC(F)(F)c1ccc(-c2ccc(Cl)nc2)cc1Cl)c1ccccc1C(F)(F)F] |
| 1-69 | 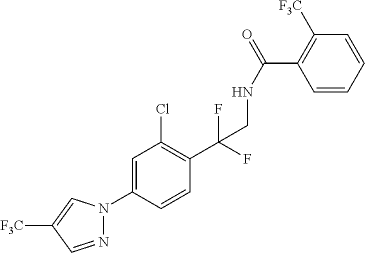 [SMILES: O=C(NCC(F)(F)c1ccc(-n2cc(C(F)(F)F)cn2)cc1Cl)c1ccccc1C(F)(F)F] |
| 1-70 | 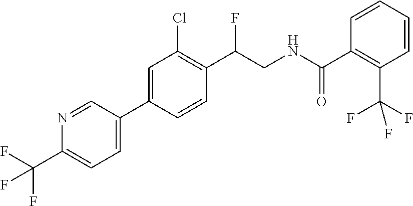 [SMILES: O=C(NCC(F)c1ccc(-c2ccc(C(F)(F)F)nc2)cc1Cl)c1ccccc1C(F)(F)F] |
| 1-71 | 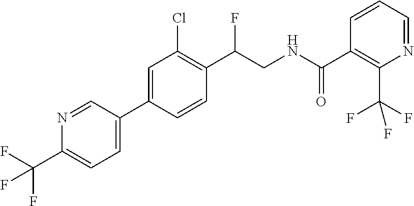 [SMILES: O=C(NCC(F)c1ccc(-c2ccc(C(F)(F)F)nc2)cc1Cl)c1cccnc1C(F)(F)F] |
| 1-72 | 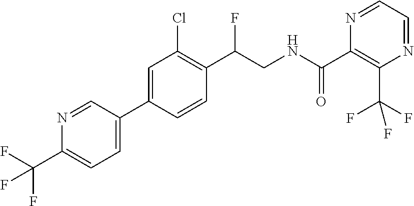 [SMILES: O=C(NCC(F)c1ccc(-c2ccc(C(F)(F)F)nc2)cc1Cl)c1nccnc1C(F)(F)F] |
| 1-73 | 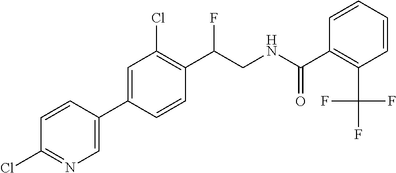 [SMILES: O=C(NCC(F)c1ccc(-c2ccc(Cl)nc2)cc1Cl)c1ccccc1C(F)(F)F] |
| 1-74 | 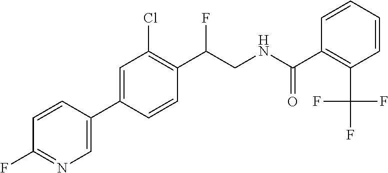 [SMILES: O=C(NCC(F)c1ccc(-c2ccc(F)nc2)cc1Cl)c1ccccc1C(F)(F)F] |
| 1-75 | 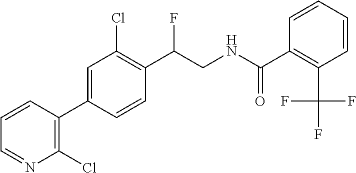 [SMILES: O=C(NCC(F)c1ccc(-c2cccnc2Cl)cc1Cl)c1ccccc1C(F)(F)F] |
| 1-76 | 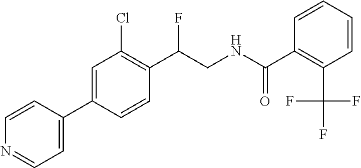 [SMILES: O=C(NCC(F)c1ccc(-c2ccncc2)cc1Cl)c1ccccc1C(F)(F)F] |
| 1-77 | 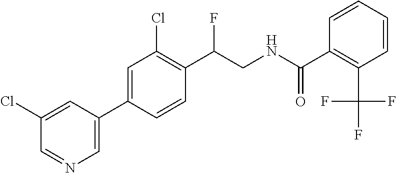 [SMILES: O=C(NCC(F)c1ccc(-c2cncc(Cl)c2)cc1Cl)c1ccccc1C(F)(F)F] |
| 1-78 | 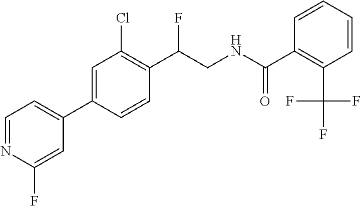 [SMILES: O=C(NCC(F)c1ccc(-c2ccnc(F)c2)cc1Cl)c1ccccc1C(F)(F)F] |
| 1-79 | 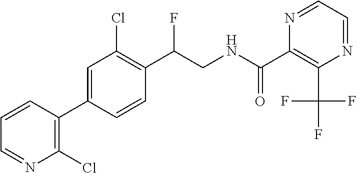 [SMILES: O=C(NCC(F)c1ccc(-c2cccnc2Cl)cc1Cl)c1nccnc1C(F)(F)F] |
| 1-80 | 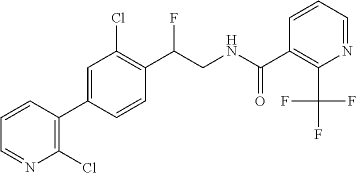 [SMILES: O=C(NCC(F)c1ccc(-c2cccnc2Cl)cc1Cl)c1cccnc1C(F)(F)F] |
| 1-81 | 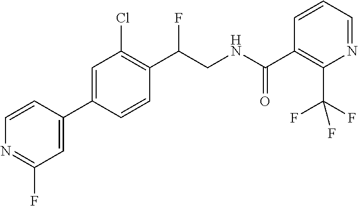 [SMILES: O=C(NCC(F)c1ccc(-c2ccnc(F)c2)cc1Cl)c1cccnc1C(F)(F)F] |
| 1-82 | 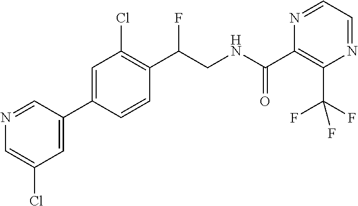 [SMILES: O=C(NCC(F)c1ccc(-c2cncc(Cl)c2)cc1Cl)c1nccnc1C(F)(F)F] |
| 1-83 | 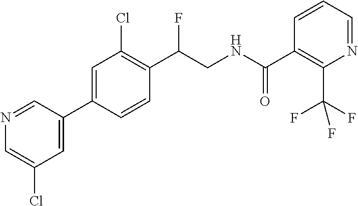 [SMILES: O=C(NCC(F)c1ccc(-c2cncc(Cl)c2)cc1Cl)c1cccnc1C(F)(F)F] |
| 1-84 | 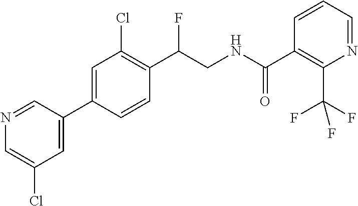 [SMILES: O=C(NCC(F)c1ccc(-c2cncc(Cl)c2)cc1Cl)c1cccnc1C(F)(F)F] |
| 1-85 | 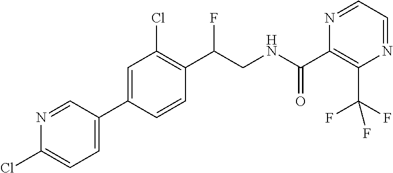 [SMILES: O=C(NCC(F)c1ccc(-c2ccc(Cl)nc2)cc1Cl)c1nccnc1C(F)(F)F] |
| 1-86 | 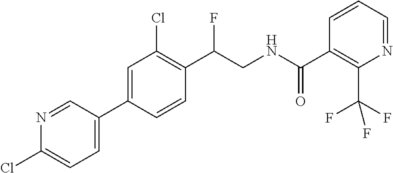 [SMILES: O=C(NCC(F)c1ccc(-c2ccc(Cl)nc2)cc1Cl)c1cccnc1C(F)(F)F] |
| 1-87 | 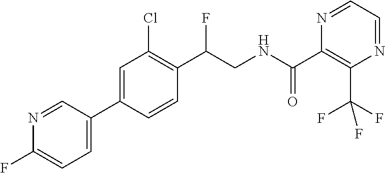 [SMILES: O=C(NCC(F)c1ccc(-c2ccc(F)nc2)cc1Cl)c1nccnc1C(F)(F)F] |
| 1-88 | 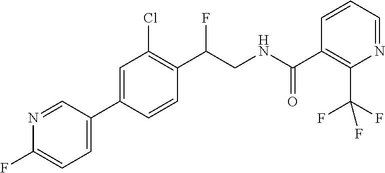 [SMILES: O=C(NCC(F)c1ccc(-c2ccc(F)nc2)cc1Cl)c1cccnc1C(F)(F)F] |
| 1-89 | 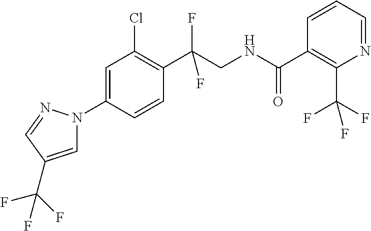 [SMILES: O=C(NCC(F)(F)c1ccc(-n2cc(C(F)(F)F)cn2)cc1Cl)c1cccnc1C(F)(F)F] |
| 1-90 | 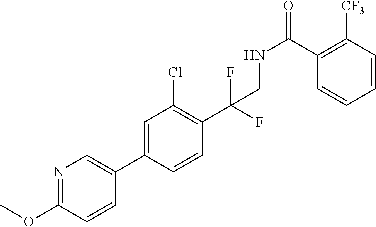 [SMILES: COc1ccc(-c2ccc(C(F)(F)CNC(=O)c3ccccc3C(F)(F)F)c(Cl)c2)cn1] |
| 1-91 | 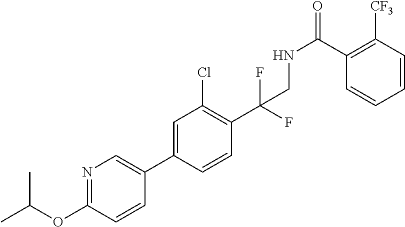 [SMILES: CC(C)Oc1ccc(-c2ccc(C(F)(F)CNC(=O)c3ccccc3C(F)(F)F)c(Cl)c2)cn1] |
| 1-92 | 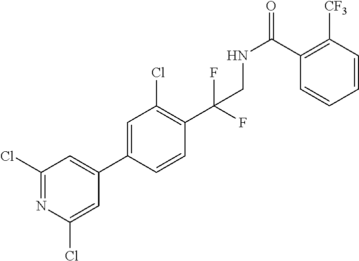 [SMILES: O=C(NCC(F)(F)c1ccc(-c2cc(Cl)nc(Cl)c2)cc1Cl)c1ccccc1C(F)(F)F] |
| 1-93 | 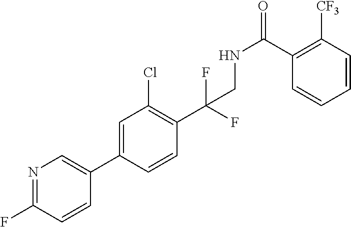 [SMILES: O=C(NCC(F)(F)c1ccc(-c2ccc(F)nc2)cc1Cl)c1ccccc1C(F)(F)F] |
| 1-94 | 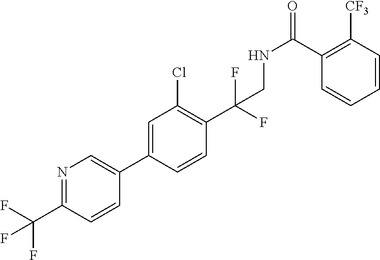 [SMILES: O=C(NCC(F)(F)c1ccc(-c2ccc(C(F)(F)F)nc2)cc1Cl)c1ccccc1C(F)(F)F] |
| 1-95 | 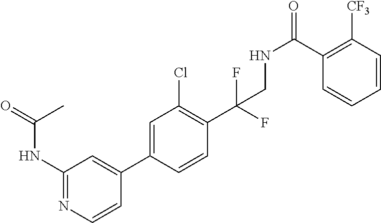 [SMILES: CC(=O)Nc1cc(-c2ccc(C(F)(F)CNC(=O)c3ccccc3C(F)(F)F)c(Cl)c2)ccn1] |
| 1-96 | 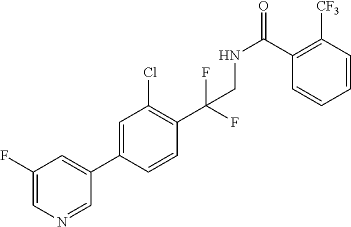 [SMILES: O=C(NCC(F)(F)c1ccc(-c2cncc(F)c2)cc1Cl)c1ccccc1C(F)(F)F] |
| 1-97 | 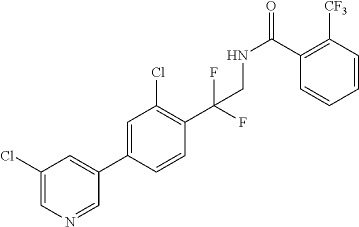 [SMILES: O=C(NCC(F)(F)c1ccc(-c2cncc(Cl)c2)cc1Cl)c1ccccc1C(F)(F)F] |
| 1-98 | 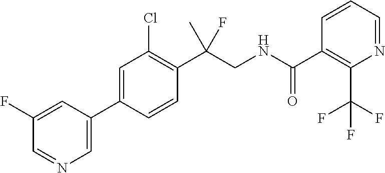 [SMILES: CC(F)(CNC(=O)c1cccnc1C(F)(F)F)c1ccc(-c2cncc(F)c2)cc1Cl] |
| 1-99 | 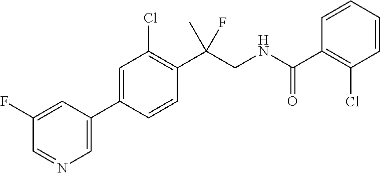 [SMILES: CC(F)(CNC(=O)c1ccccc1Cl)c1ccc(-c2cncc(F)c2)cc1Cl] |
| 1-100 | 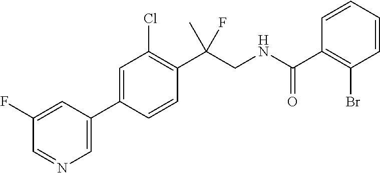 [SMILES: CC(F)(CNC(=O)c1ccccc1Br)c1ccc(-c2cncc(F)c2)cc1Cl] |
| 1-101 | 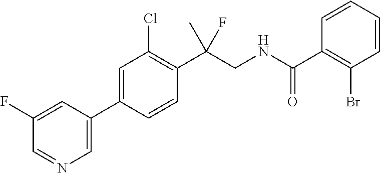 [SMILES: CC(F)(CNC(=O)c1ccccc1Br)c1ccc(-c2cncc(F)c2)cc1Cl] |
| 1-102 | 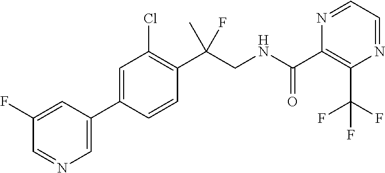 [SMILES: CC(F)(CNC(=O)c1nccnc1C(F)(F)F)c1ccc(-c2cncc(F)c2)cc1Cl] |
| 1-103 | 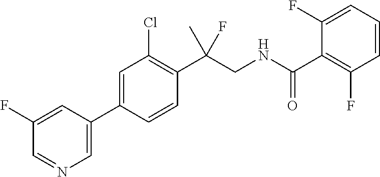 [SMILES: CC(F)(CNC(=O)c1c(F)cccc1F)c1ccc(-c2cncc(F)c2)cc1Cl] |
| 1-104 | 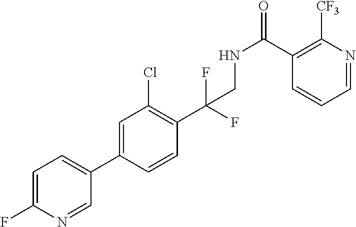 [SMILES: O=C(NCC(F)(F)c1ccc(-c2ccc(F)nc2)cc1Cl)c1cccnc1C(F)(F)F] |
| 1-105 | 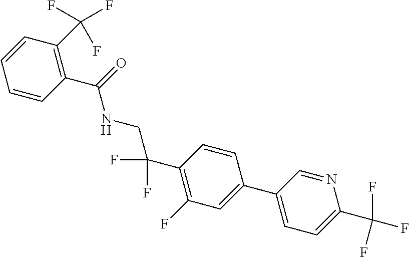 [SMILES: O=C(NCC(F)(F)c1ccc(-c2ccc(C(F)(F)F)nc2)cc1F)c1ccccc1C(F)(F)F] |
| 1-106 | 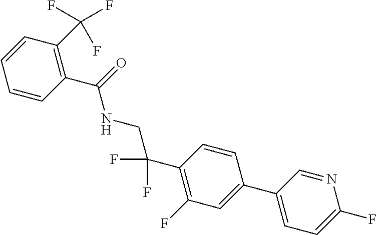 [SMILES: O=C(NCC(F)(F)c1ccc(-c2ccc(F)nc2)cc1F)c1ccccc1C(F)(F)F] |
| 1-107 | 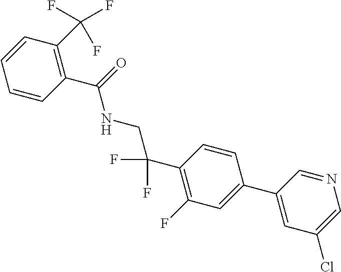 [SMILES: O=C(NCC(F)(F)c1ccc(-c2cncc(Cl)c2)cc1F)c1ccccc1C(F)(F)F] |
| 1-108 | 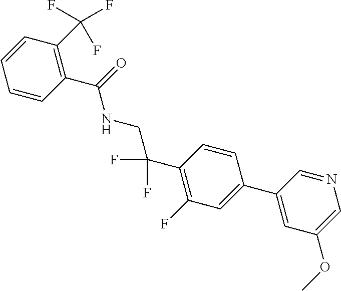 [SMILES: COc1cncc(-c2ccc(C(F)(F)CNC(=O)c3ccccc3C(F)(F)F)c(F)c2)c1] |
| 1-109 | 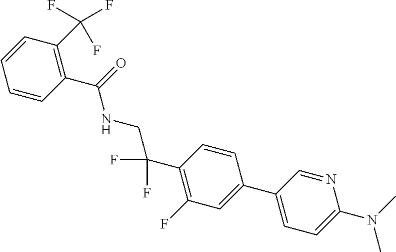 [SMILES: CN(C)c1ccc(-c2ccc(C(F)(F)CNC(=O)c3ccccc3C(F)(F)F)c(F)c2)cn1] |
| 1-110 | 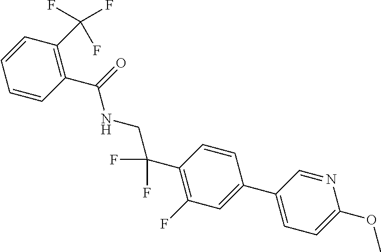 [SMILES: COc1ccc(-c2ccc(C(F)(F)CNC(=O)c3ccccc3C(F)(F)F)c(F)c2)cn1] |
| 1-111 | 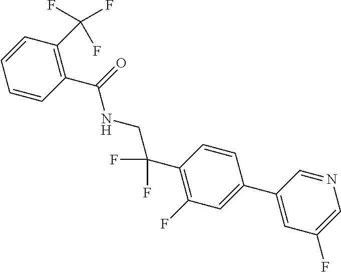 [SMILES: O=C(NCC(F)(F)c1ccc(-c2cncc(F)c2)cc1F)c1ccccc1C(F)(F)F] |
| 1-112 | 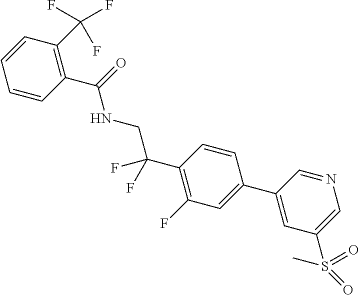 [SMILES: CS(=O)(=O)c1cncc(-c2ccc(C(F)(F)CNC(=O)c3ccccc3C(F)(F)F)c(F)c2)c1] |
| 1-113 | 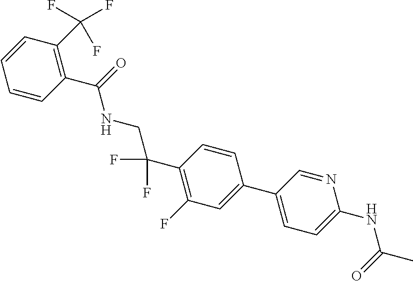 [SMILES: CC(=O)Nc1ccc(-c2ccc(C(F)(F)CNC(=O)c3ccccc3C(F)(F)F)c(F)c2)cn1] |
| 1-114 | 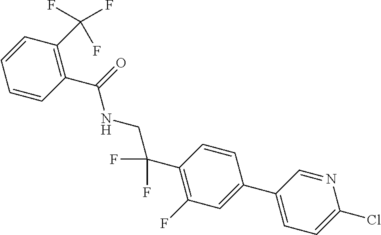 [SMILES: O=C(NCC(F)(F)c1ccc(-c2ccc(Cl)nc2)cc1F)c1ccccc1C(F)(F)F] |
| 1-115 | 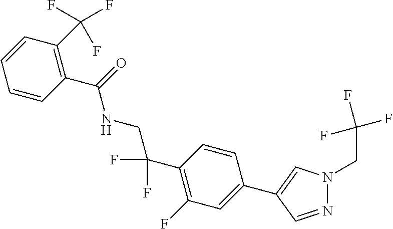 [SMILES: O=C(NCC(F)(F)c1ccc(-c2cnn(CC(F)(F)F)c2)cc1F)c1ccccc1C(F)(F)F] |
| 1-116 | 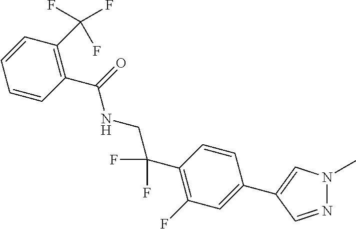 [SMILES: Cn1cc(-c2ccc(C(F)(F)CNC(=O)c3ccccc3C(F)(F)F)c(F)c2)cn1] |
| 1-117 | 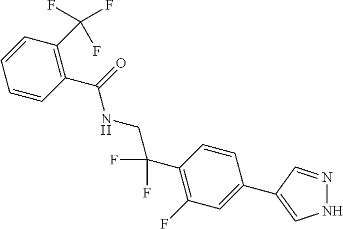 [SMILES: O=C(NCC(F)(F)c1ccc(-c2cn[nH]c2)cc1F)c1ccccc1C(F)(F)F] |
| 1-118 | 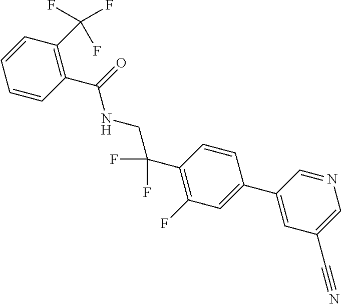 [SMILES: N#Cc1cncc(-c2ccc(C(F)(F)CNC(=O)c3ccccc3C(F)(F)F)c(F)c2)c1] |
| 1-119 | 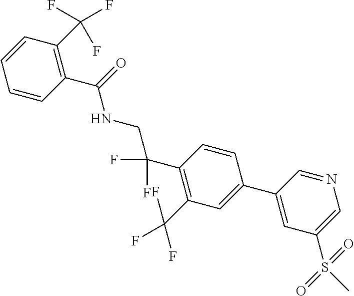 [SMILES: CS(=O)(=O)c1cncc(-c2ccc(C(F)(F)CNC(=O)c3ccccc3C(F)(F)F)c(C(F)(F)F)c2)c1] |
| 1-120 | 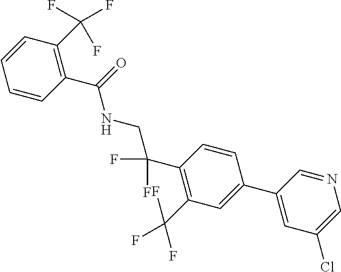 [SMILES: O=C(NCC(F)(F)c1ccc(-c2cncc(Cl)c2)cc1C(F)(F)F)c1ccccc1C(F)(F)F] |
| 1-121 | 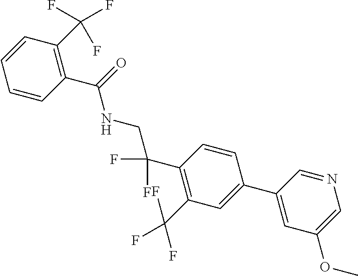 [SMILES: COc1cncc(-c2ccc(C(F)(F)CNC(=O)c3ccccc3C(F)(F)F)c(C(F)(F)F)c2)c1] |
| 1-122 | 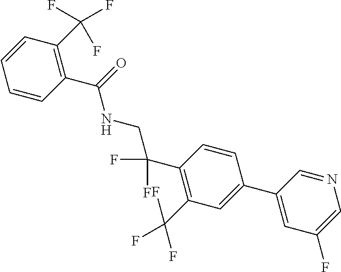 [SMILES: O=C(NCC(F)(F)c1ccc(-c2cncc(F)c2)cc1C(F)(F)F)c1ccccc1C(F)(F)F] |
| 1-123 | 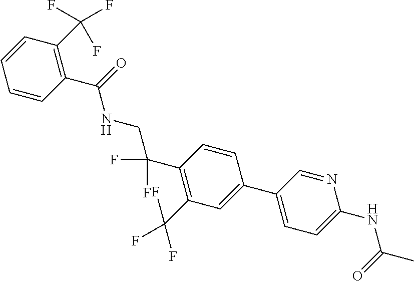 [SMILES: CC(=O)Nc1ccc(-c2ccc(C(F)(F)CNC(=O)c3ccccc3C(F)(F)F)c(C(F)(F)F)c2)cn1] |
| 1-124 | 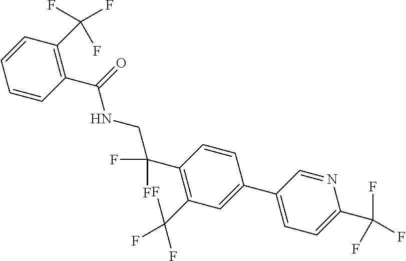 [SMILES: O=C(NCC(F)(F)c1ccc(-c2ccc(C(F)(F)F)nc2)cc1C(F)(F)F)c1ccccc1C(F)(F)F] |
| 1-125 | 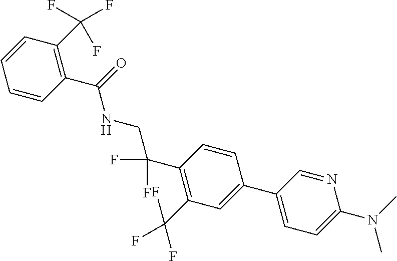 [SMILES: CN(C)c1ccc(-c2ccc(C(F)(F)CNC(=O)c3ccccc3C(F)(F)F)c(C(F)(F)F)c2)cn1] |
| 1-126 | 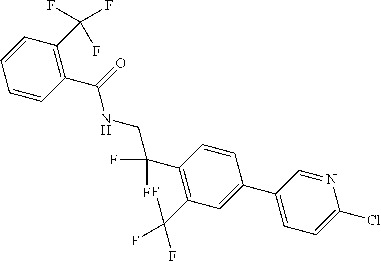 [SMILES: O=C(NCC(F)(F)c1ccc(-c2ccc(Cl)nc2)cc1C(F)(F)F)c1ccccc1C(F)(F)F] |
| 1-127 | 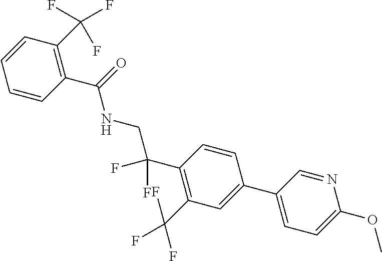 [SMILES: COc1ccc(-c2ccc(C(F)(F)CNC(=O)c3ccccc3C(F)(F)F)c(C(F)(F)F)c2)cn1] |
| 1-128 | 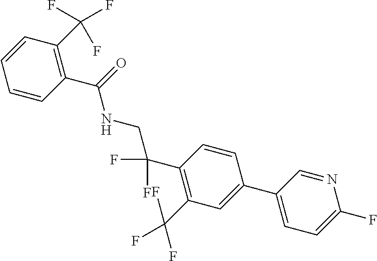 [SMILES: O=C(NCC(F)(F)c1ccc(-c2ccc(F)nc2)cc1C(F)(F)F)c1ccccc1C(F)(F)F] |
| 1-129 | 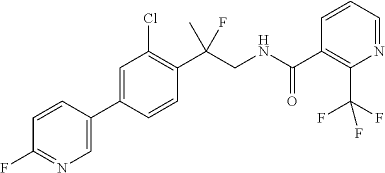 [SMILES: CC(F)(CNC(=O)c1cccnc1C(F)(F)F)c1ccc(-c2ccc(F)nc2)cc1Cl] |
| 130 | 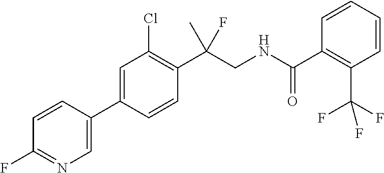 [SMILES: CC(F)(CNC(=O)c1ccccc1C(F)(F)F)c1ccc(-c2ccc(F)nc2)cc1Cl] |
| 1-131 | 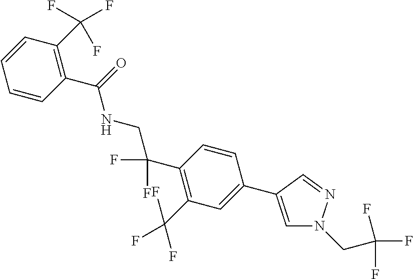 [SMILES: O=C(NCC(F)(F)c1ccc(-c2cnn(CC(F)(F)F)c2)cc1C(F)(F)F)c1ccccc1C(F)(F)F] |
| 1-132 | 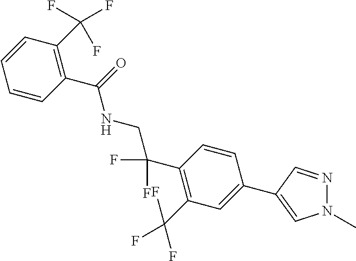 [SMILES: Cn1cc(-c2ccc(C(F)(F)CNC(=O)c3ccccc3C(F)(F)F)c(C(F)(F)F)c2)cn1] |
| 1-133 | 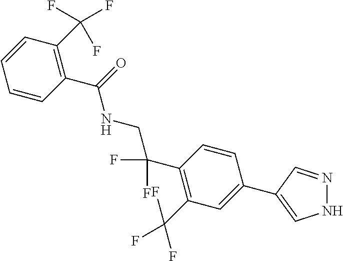 [SMILES: O=C(NCC(F)(F)c1ccc(-c2cn[nH]c2)cc1C(F)(F)F)c1ccccc1C(F)(F)F] |
| 1-134 | 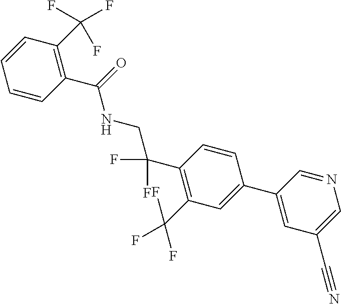 [SMILES: N#Cc1cncc(-c2ccc(C(F)(F)CNC(=O)c3ccccc3C(F)(F)F)c(C(F)(F)F)c2)c1] |
| 1-135 | 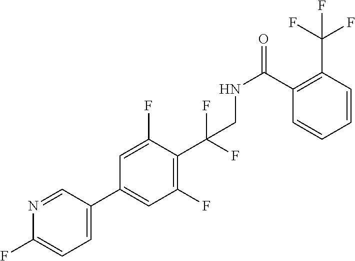 [SMILES: O=C(NCC(F)(F)c1c(F)cc(-c2ccc(F)nc2)cc1F)c1ccccc1C(F)(F)F] |
| 1-136 | 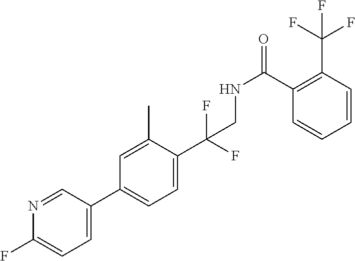 [SMILES: Cc1cc(-c2ccc(F)nc2)ccc1C(F)(F)CNC(=O)c1ccccc1C(F)(F)F] |
| 1-137 | 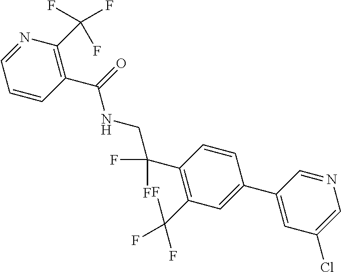 [SMILES: O=C(NCC(F)(F)c1ccc(-c2cncc(Cl)c2)cc1C(F)(F)F)c1cccnc1C(F)(F)F] |
| 1-138 | 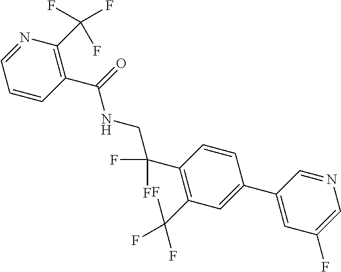 [SMILES: O=C(NCC(F)(F)c1ccc(-c2cncc(F)c2)cc1C(F)(F)F)c1cccnc1C(F)(F)F] |
| 1-139 | 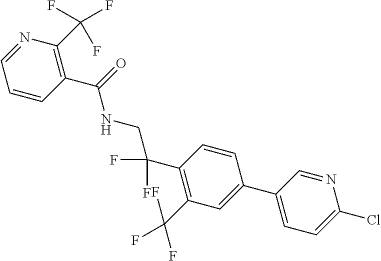 [SMILES: O=C(NCC(F)(F)c1ccc(-c2ccc(Cl)nc2)cc1C(F)(F)F)c1cccnc1C(F)(F)F] |
| 1-140 | 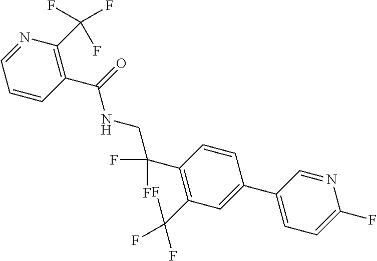 [SMILES: O=C(NCC(F)(F)c1ccc(-c2ccc(F)nc2)cc1C(F)(F)F)c1cccnc1C(F)(F)F] |
| 1-141 | 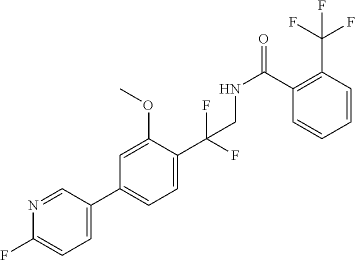 [SMILES: COc1cc(-c2ccc(F)nc2)ccc1C(F)(F)CNC(=O)c1ccccc1C(F)(F)F] |
| 1-142 | 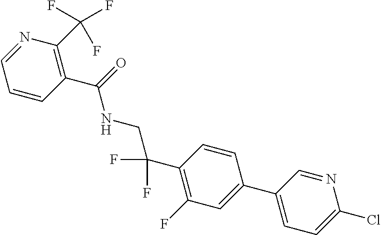 [SMILES: O=C(NCC(F)(F)c1ccc(-c2ccc(Cl)nc2)cc1F)c1cccnc1C(F)(F)F] |
| 1-143 | 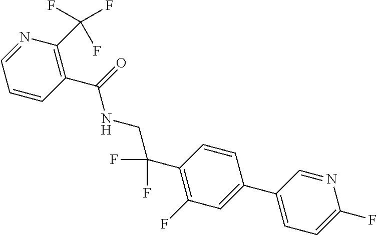 [SMILES: O=C(NCC(F)(F)c1ccc(-c2ccc(F)nc2)cc1F)c1cccnc1C(F)(F)F] |
| 1-144 | 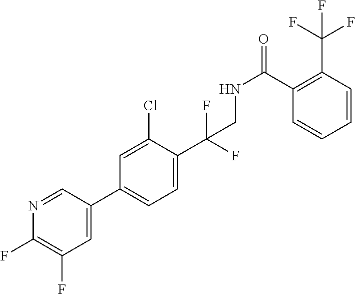 [SMILES: O=C(NCC(F)(F)c1ccc(-c2cnc(F)c(F)c2)cc1Cl)c1ccccc1C(F)(F)F] |
| 1-145 | 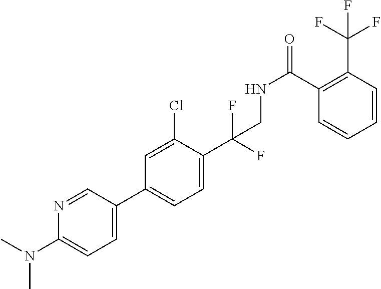 [SMILES: CN(C)c1ccc(-c2ccc(C(F)(F)CNC(=O)c3ccccc3C(F)(F)F)c(Cl)c2)cn1] |
| 1-146 | 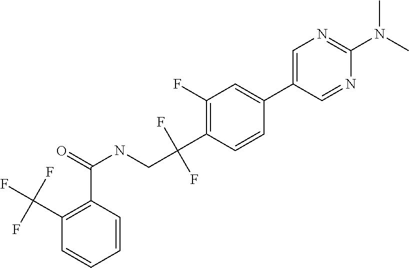 [SMILES: CN(C)c1ncc(-c2ccc(C(F)(F)CNC(=O)c3ccccc3C(F)(F)F)c(F)c2)cn1] |
| 1-147 | 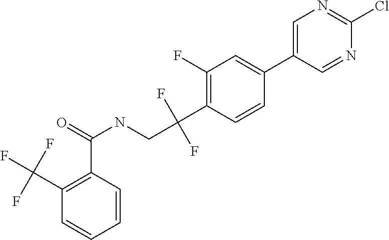 [SMILES: O=C(NCC(F)(F)c1ccc(-c2cnc(Cl)nc2)cc1F)c1ccccc1C(F)(F)F] |
| 1-148 | 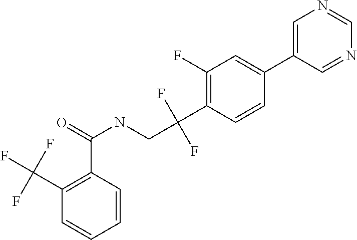 [SMILES: O=C(NCC(F)(F)c1ccc(-c2cncnc2)cc1F)c1ccccc1C(F)(F)F] |
| 1-149 | 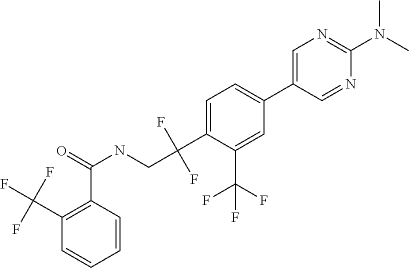 [SMILES: CN(C)c1ncc(-c2ccc(C(F)(F)CNC(=O)c3ccccc3C(F)(F)F)c(C(F)(F)F)c2)cn1] |
| 1-150 | 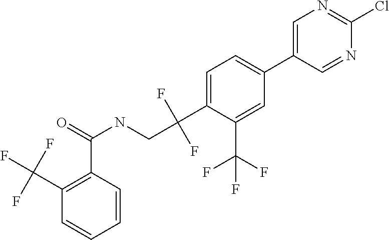 [SMILES: O=C(NCC(F)(F)c1ccc(-c2cnc(Cl)nc2)cc1C(F)(F)F)c1ccccc1C(F)(F)F] |
| 1-151 | 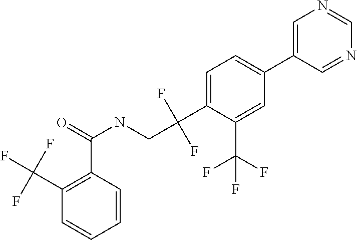 [SMILES: O=C(NCC(F)(F)c1ccc(-c2cncnc2)cc1C(F)(F)F)c1ccccc1C(F)(F)F] |
| 1-152 | 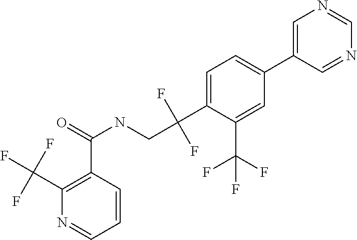 [SMILES: O=C(NCC(F)(F)c1ccc(-c2cncnc2)cc1C(F)(F)F)c1cccnc1C(F)(F)F] |
| 1-153 | 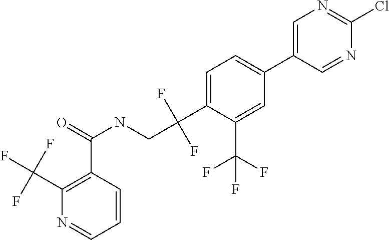 [SMILES: O=C(NCC(F)(F)c1ccc(-c2cnc(Cl)nc2)cc1C(F)(F)F)c1cccnc1C(F)(F)F] |
| 1-154 | 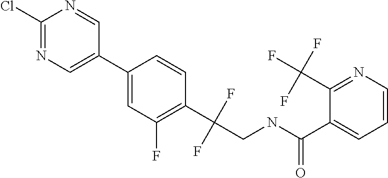 [SMILES: O=C(NCC(F)(F)c1ccc(-c2cnc(Cl)nc2)cc1F)c1cccnc1C(F)(F)F] |
| 1-155 | 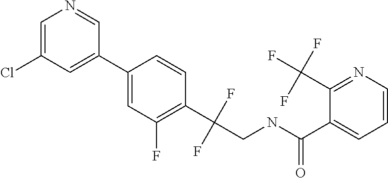 [SMILES: O=C(NCC(F)(F)c1ccc(-c2cncc(Cl)c2)cc1F)c1cccnc1C(F)(F)F] |
| 1-156 | 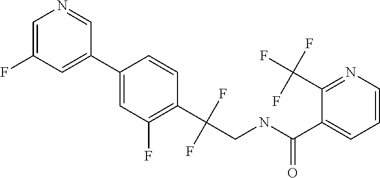 [SMILES: O=C(NCC(F)(F)c1ccc(-c2cncc(F)c2)cc1F)c1cccnc1C(F)(F)F] |
| Known from WO 2014004064 A1 (Cpd 150) | 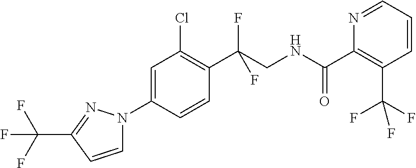 [SMILES: O=C(NCC(F)(F)c1ccc(-n2ccc(C(F)(F)F)n2)cc1Cl)c1ncccc1C(F)(F)F] |

TABLE 2
Compounds of formula (INT)
(INT)

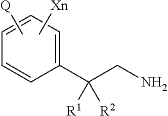

R¹, R², Q, X, n are as defined by each individual structure and might occur as salts like hydrochlorides.

| Example no. | Formula | NMR data |
|---|---|---|
| INT-1 | 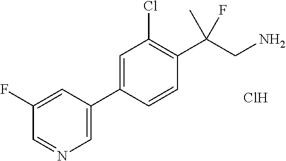 [SMILES: CC(F)(CN)c1ccc(-c2cncc(F)c2)cc1Cl ; Cl] | ¹H-NMR (400 MHz, d₆-DMSO); δ 8.89 (s, 1 H), 8.66 (d, 1 H), 8.33 (s, 2 H), 8.22 (dd, 1 H), 8.04 (m, 1 H), 7.93 (dd, 1H), 7.73 (d, 1H), 4.2 (bs, 2H), 1.97 (d, 3H) |
| INT-2 | 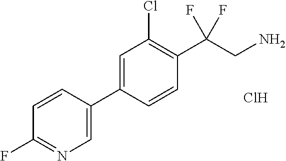 [SMILES: NCC(F)(F)c1ccc(-c2ccc(F)nc2)cc1Cl ; Cl] | ¹H-NMR (400.0 MHz, DMSO): δ = 8.79 (bs, 3H, NH₃⁺), 8.69 (d, 1H), 8.45-8.40 (dt, 1H), 8.08 (s, 1H), 7.93-7.91 (d, 1H), 7.80- 7.78 (d, 1H), 7.38-7.35 (dd, 1H), 3.92-3.81 (bt, 2H). |

### NMR Peak Lists

¹H-NMR data of selected examples are written in form of ¹H-NMR-peak lists. The δ-value in ppm and the signal intensity are listed to each signal peak in round brackets. Between the δ-value-signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

Tetramethylsilane and/or the chemical shift of the used solvent, especially in the case of spectra measured in DMSO, have been used for calibrating. Therefore, tetramethylsilane peak can occur but not necessarily in NMR peak lists.

The ¹H-NMR peak lists are similar to classical ¹H-NMR prints and contain therefore usually all peaks, which are listed at classical NMR-interpretation.

Additionally they can show like classical ¹H-NMR prints signals of solvents, stereoisomers of the target compounds, which are also object of the invention, and/or peaks of impurities.

The usual peaks of solvents, for example peaks of DMSO in DMSO-D6 and the peak of water, are given in the ¹H-NMR peak lists to show compound signals in the delta-range of solvents and/or water. They have usually on average a high intensity.

The peaks of stereoisomers of the target compounds and/or peaks of impurities have usually on average a lower intensity than the peaks of target compounds (for example with a purity>90%).

Such stereoisomers and/or impurities can be typical for the specific preparation process. Therefore, their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists you find in the publication “Citation of NMR Peaklist Data within Patent Applications” of the Research Disclosure Database Number 564025.

TABLE 3
NMR Peaklists (Method M1)

Example 1-1: ¹H-NMR(400.0 MHz, DMSO):
δ = 8.622(2.1); 8.609(4.3); 8.595(2.1); 8.544(0.4); 8.539(0.4); 8.520(8.1); 8.519(8.4); 8.514(8.6); 8.513(8.1);
8.160(0.3); 8.153(0.4); 8.136(6.6); 8.129(6.3); 8.114(6.8); 8.108(6.7); 7.769(4.1); 7.751(5.7); 7.747(5.1);
7.676(1.5); 7.660(4.4); 7.658(4.2); 7.643(5.2); 7.639(6.2); 7.635(4.6); 7.618(4.0); 7.616(4.0); 7.599(1.4);
7.577(8.4); 7.551(3.1); 7.548(3.9); 7.544(2.5); 7.531(4.2); 7.528(5.0); 7.450(0.4); 7.442(4.8); 7.431(0.8);
7.423(9.1); 7.412(0.5); 7.404(4.7); 7.388(5.0); 7.371(4.3); 7.327(0.4); 7.320(0.4); 7.294(5.2); 7.284(0.9);
7.275(4.0); 7.178(0.4); 7.157(0.4); 7.095(8.9); 7.094(8.6); 7.074(8.6); 7.072(8.3); 5.080(4.5); 5.058(14.8);
5.035(15.7); 5.012(5.4); 3.558(2.7); 3.541(5.9); 3.526(5.8); 3.508(2.9); 3.325(81.2); 2.919(5.0); 2.901(8.8);
2.883(4.4); 2.676(0.5); 2.671(0.7); 2.666(0.5); 2.524(2.2); 2.520(3.4); 2.511(40.2); 2.507(81.1); 2.502(107.9);
2.497(77.7); 2.493(36.1); 2.333(0.5); 2.329(0.7); 2.324(0.5); 1.989(0.8); 1.398(16.0); 1.175(0.4); 1.121(0.5);
0.982(0.5); 0.000(3.0)
Example 1-2: ¹H-NMR(400.0 MHz, DMSO):
δ = 8.903(3.6); 8.899(3.4); 8.635(1.0); 8.622(1.9); 8.608(1.1); 8.581(2.5); 8.578(2.6); 8.569(2.7); 8.566(2.5);
8.095(1.6); 8.091(1.9); 8.090(2.0); 8.085(1.5); 8.075(1.8); 8.071(2.1); 8.070(2.2); 8.065(1.5); 7.771(1.9);
7.753(2.5); 7.749(2.3); 7.670(0.7); 7.654(2.1); 7.638(4.3); 7.632(5.0); 7.626(4.7); 7.622(4.0); 7.595(2.4);
7.576(2.5); 7.503(1.8); 7.501(1.9); 7.491(2.0); 7.489(2.0); 7.483(2.0); 7.481(2.1); 7.473(2.7); 7.472(2.7);
7.455(3.9); 7.436(2.0); 7.393(2.1); 7.390(2.3); 7.374(2.0); 7.337(2.6); 7.318(2.0); 3.571(1.3); 3.554(3.1);
3.538(3.2); 3.521(1.5); 3.338(5.8); 2.938(2.5); 2.921(4.6); 2.903(2.3); 2.508(9.4); 2.503(11.8); 2.499(8.8);
2.495(4.6); 2.087(16.0)
Example 1-3: ¹H-NMR(400.0 MHz, DMSO):
δ = 8.938(0.4); 8.817(7.4); 8.813(13.3); 8.809(7.6); 8.682(0.4); 8.676(0.4); 8.628(2.8); 8.614(5.5); 8.601(2.9);
8.580(12.1); 8.573(12.4); 8.092(3.9); 8.086(5.3); 8.081(3.9); 8.066(4.0); 8.061(5.1); 8.059(5.2); 8.055(3.9);
7.770(5.5); 7.752(7.4); 7.749(7.0); 7.693(11.8); 7.676(2.3); 7.659(11.7); 7.639(16.0); 7.619(5.7); 7.601(1.8);
7.482(5.5); 7.463(11.5); 7.444(6.5); 7.392(6.9); 7.375(6.1); 7.363(7.8); 7.344(5.5); 7.260(1.7); 4.056(0.7);
4.038(2.2); 4.020(2.3); 4.002(0.8); 3.572(3.8); 3.555(8.9); 3.539(9.0); 3.522(4.3); 3.324(82.7); 2.935(7.4);
2.917(13.4); 2.899(6.7); 2.675(0.9); 2.671(1.3); 2.666(0.9); 2.524(4.1); 2.506(139.6); 2.502(181.3); 2.497(135.1);
2.333(0.9); 2.329(1.2); 2.324(0.9); 1.989(9.6); 1.397(0.6); 1.279(0.6); 1.262(0.6); 1.192(2.6); 1.175(5.1);
1.157(2.5); 1.138(0.3); 1.120(0.6); 0.982(0.6); 0.879(1.1); 0.870(3.0); 0.858(0.3); 0.000(1.3)
Example 1-4: ¹H-NMR(400.0 MHz, DMSO):
δ = 8.598(0.6); 8.585(1.2); 8.571(0.6); 8.466(1.8); 8.461(1.9); 8.453(1.8); 8.449(1.8); 7.767(1.3); 7.749(1.8);
7.745(1.6); 7.677(0.5); 7.661(1.4); 7.659(1.3); 7.644(1.7); 7.640(1.9); 7.636(1.5); 7.619(1.3); 7.617(1.3);
7.611(1.8); 7.606(1.8); 7.600(0.6); 7.591(2.0); 7.587(1.9); 7.436(1.0); 7.417(2.5); 7.398(1.7); 7.376(1.6);
7.359(1.3); 7.311(2.9); 7.297(2.0); 7.292(2.5); 7.278(2.7); 7.274(2.9); 7.267(2.2); 7.264(1.9); 7.248(1.1);
7.245(1.4); 4.038(0.6); 4.020(0.6); 3.544(0.9); 3.527(2.0); 3.512(2.0); 3.494(1.0); 3.326(14.7); 2.908(1.7);
2.890(3.1); 2.872(1.5); 2.524(0.6); 2.511(10.5); 2.506(20.7); 2.502(26.8); 2.497(19.2); 2.493(9.0); 2.427(16.0);
1.989(2.4); 1.192(0.7); 1.175(1.3); 1.157(0.7); 0.000(4.3)
Example 1-5: ¹H-NMR(400.0 MHz, DMSO):
δ = 8.595(0.7); 8.582(1.3); 8.568(0.7); 7.766(1.4); 7.748(1.8); 7.700(0.3); 7.692(3.5); 7.671(5.1); 7.653(1.4);
7.650(1.2); 7.638(1.4); 7.620(1.4); 7.603(0.5); 7.473(0.4); 7.445(1.0); 7.425(4.2); 7.404(3.0); 7.378(1.7);
7.360(1.4); 7.329(1.7); 7.310(1.3); 7.287(2.8); 7.276(1.7); 7.273(2.0); 7.257(1.1); 7.254(1.3); 4.055(0.4);
4.038(1.1); 4.020(1.1); 4.002(0.4); 3.542(0.9); 3.525(2.0); 3.510(2.0); 3.492(1.0); 3.471(0.4); 3.457(0.4);
3.323(56.8); 2.905(1.6); 2.887(3.0); 2.869(1.5); 2.821(0.5); 2.675(0.6); 2.670(0.7); 2.666(0.6); 2.524(2.2);
2.519(3.5); 2.510(40.4); 2.506(80.9); 2.501(106.2); 2.497(76.5); 2.492(36.2); 2.408(16.0); 2.333(0.5); 2.328(0.7);
2.324(0.5); 2.086(0.4); 1.989(4.8); 1.249(0.4); 1.192(1.3); 1.174(2.7); 1.157(1.3); 0.008(0.5);
0.000(16.4); −0.009(0.5)
Example 1-6: ¹H-NMR(400.0 MHz, DMSO):
δ = 10.565(2.3); 8.625(0.8); 8.612(1.5); 8.598(0.8); 8.386(2.2); 8.365(2.8); 8.352(2.9); 7.765(1.4); 7.747(1.9);
7.744(1.8); 7.671(0.5); 7.654(1.5); 7.637(2.2); 7.633(2.4); 7.613(1.4); 7.591(3.2); 7.572(1.4); 7.553(2.0);
7.498(1.5); 7.479(2.8); 7.460(1.4); 7.408(2.1); 7.404(2.2); 7.395(3.8); 7.391(5.1); 7.372(2.5); 3.545(0.9);
3.528(2.1); 3.512(2.2); 3.495(1.0); 3.323(36.4); 2.936(1.8); 2.918(3.2); 2.900(1.6); 2.675(0.4); 2.671(0.6);
2.666(0.4); 2.510(34.7); 2.506(65.6); 2.501(84.2); 2.497(61.4); 2.493(30.4); 2.333(0.4); 2.328(0.6); 2.324(0.4);
2.121(16.0); 1.069(1.2); 0.000(0.6)
Example 1-7: ¹H-NMR(400.0 MHz, DMSO):
δ = 8.641(13.9); 8.638(9.5); 8.630(11.1); 8.626(16.0); 8.614(4.5); 8.600(2.2); 7.769(4.3); 7.751(5.7); 7.747(5.3);
7.724(15.3); 7.720(9.9); 7.713(10.0); 7.709(15.1); 7.698(9.5); 7.670(6.1); 7.651(9.6); 7.638(8.7); 7.633(8.3);
7.617(4.2); 7.598(1.3); 7.493(4.2); 7.474(9.0); 7.454(5.0); 7.384(11.1); 7.366(8.1); 3.564(3.0); 3.547(7.1);
3.532(7.1); 3.514(3.3); 3.327(24.0); 2.940(5.9); 2.922(10.6); 2.905(5.2); 2.675(0.4); 2.671(0.5); 2.666(0.4);
2.540(0.4); 2.506(59.5); 2.502(75.8); 2.497(55.6); 2.333(0.4); 2.329(0.5); 2.324(0.4); 0.000(0.6)
Example 1-8: ¹H-NMR(400.0 MHz, DMSO):
δ = 9.149(0.6); 8.882(14.8); 8.878(14.8); 8.626(16.0); 8.621(15.5); 8.612(5.8); 8.599(2.8); 8.261(9.5); 8.256(15.9);
8.250(8.9); 7.771(5.5); 7.753(7.5); 7.749(6.8); 7.693(11.7); 7.681(2.3); 7.678(2.3); 7.659(11.1); 7.639(15.0);
7.621(5.3); 7.602(1.7); 7.489(0.4); 7.479(5.8); 7.470(0.7); 7.460(12.1); 7.441(6.7); 7.392(6.8); 7.375(6.0);
7.363(7.7); 7.344(5.3); 4.038(0.4); 4.020(0.4); 3.573(3.8); 3.556(8.7); 3.541(8.7); 3.523(4.1); 3.326(44.9);
2.934(7.2); 2.916(13.1); 2.898(6.4); 2.695(0.6); 2.676(0.6); 2.671(0.8); 2.667(0.6); 2.525(2.9); 2.511(43.4);
2.507(84.7); 2.502(109.9); 2.498(79.0); 2.493(37.6); 2.334(0.5); 2.329(0.7); 2.324(0.5); 1.989(1.6); 1.337(0.5);
1.259(0.3); 1.249(0.6); 1.193(0.5); 1.175(0.9); 1.157(0.4); 0.008(1.3); 0.000(32.9); −0.009(1.1)
Example 1-9: ¹H-NMR(400.0 MHz, DMSO):
δ = 8.745(4.1); 8.740(4.2); 8.628(1.1); 8.614(2.1); 8.600(1.1); 8.185(2.6); 8.179(2.5); 8.164(2.8); 8.158(2.8);
8.142(2.4); 7.954(2.3); 7.770(2.2); 7.752(2.9); 7.683(0.8); 7.666(2.4); 7.642(6.8); 7.621(6.4); 7.600(5.2);
7.587(2.8); 7.476(2.0); 7.457(4.0); 7.438(2.2); 7.392(2.7); 7.374(2.3); 7.351(2.9); 7.332(2.1); 3.756(0.4);
3.744(0.4); 3.732(0.4); 3.564(1.5); 3.547(3.3); 3.531(3.4); 3.514(1.6); 3.329(1.8); 3.088(0.5); 3.079(0.7);
3.062(0.7); 3.025(0.5); 3.011(0.8); 2.997(0.6); 2.930(2.7); 2.912(4.9); 2.891(16.0); 2.732(12.5); 2.547(1.3);
2.507(31.5); 2.503(39.4); 2.498(29.2); 2.409(0.3); 2.087(0.5); 1.646(0.5); 1.633(0.4); 1.255(0.9); 1.239(0.9);
1.188(0.4); 1.170(0.8); 1.152(0.4); 0.000(9.0); −0.008(0.4)
Example 1-10: ¹H-NMR(400.0 MHz, DMSO):
δ = 9.245(0.7); 9.103(12.8); 8.636(3.8); 8.623(7.0); 8.609(3.7); 8.568(0.6); 8.550(0.6); 8.391(6.5); 8.371(7.1);
8.366(6.8); 8.316(1.1); 8.154(0.3); 8.117(0.8); 8.096(0.7); 8.003(12.8); 7.983(11.5); 7.807(0.4); 7.769(7.6);
7.750(10.1); 7.723(14.7); 7.692(7.6); 7.673(9.7); 7.666(9.6); 7.647(8.4); 7.642(8.9); 7.620(7.3); 7.601(2.6);
7.578(0.4); 7.564(0.4); 7.520(6.1); 7.501(12.7); 7.482(7.2); 7.454(0.4); 7.444(0.4); 7.404(11.1); 7.401(11.2);
7.382(9.6); 5.757(1.9); 3.649(0.3); 3.632(0.4); 3.578(4.7); 3.561(11.1); 3.546(11.2); 3.528(5.2); 3.511(0.6);
3.499(0.4); 3.480(0.4); 3.437(0.4); 3.323(347.0); 3.258(0.6); 3.228(0.5); 3.215(0.4); 3.179(0.4); 3.113(0.3);
3.091(0.7); 3.074(0.4); 3.043(0.4); 3.033(0.3); 2.990(0.4); 2.975(0.6); 2.952(9.0); 2.934(16.0); 2.916(8.2);
2.891(0.9); 2.792(0.3); 2.773(0.5); 2.756(0.5); 2.746(0.4); 2.732(0.8); 2.714(0.5); 2.671(5.0); 2.633(1.0);
2.616(1.0); 2.613(1.0); 2.506(593.5); 2.502(697.3); 2.497(502.8); 2.406(1.2); 2.390(0.8); 2.368(0.6); 2.357(0.6);
2.328(4.8); 2.294(0.4); 2.249(0.4); 2.238(0.4); 1.803(1.2); 1.785(1.9); 1.750(1.9); 1.680(0.5); 1.665(1.0);
1.398(0.7); 1.322(0.5); 1.300(1.0); 1.273(1.5); 1.242(2.0); 1.215(2.0); 1.167(0.6); 1.149(0.7); 1.121(0.6);
1.102(0.4); 0.981(0.4); 0.146(0.6); 0.043(0.3); 0.034(0.5); 0.008(7.3); 0.000(116.6); −0.150(0.6)
Example 1-11: ¹H-NMR(601.6 MHz, DMSO):
δ = 8.632(1.9); 8.623(3.7); 8.614(1.9); 8.497(5.5); 8.489(5.7); 7.777(4.2); 7.764(16.0); 7.754(4.2); 7.751(12.6);
7.718(1.8); 7.707(4.4); 7.694(2.9); 7.646(2.7); 7.633(4.1); 7.614(6.3); 7.529(3.4); 7.527(3.3); 7.521(3.4);
7.518(3.2); 7.443(4.5); 7.431(4.2); 7.417(10.9); 7.403(10.2); 3.517(2.6); 3.506(5.0); 3.496(5.0); 3.483(2.9);
3.327(19.1); 2.898(4.6); 2.885(7.9); 2.873(4.4); 2.617(0.4); 2.613(0.6); 2.610(0.4); 2.541(26.0); 2.535(42.3);
2.523(1.3); 2.520(1.3); 2.517(1.1); 2.508(28.8); 2.505(64.7); 2.502(91.5); 2.499(65.6); 2.496(30.9); 2.389(0.4);
2.386(0.6); 2.383(0.4); 1.234(0.4); 0.000(1.8)
Example 1-12: ¹H-NMR(601.6 MHz, DMSO):
δ = 9.099(7.5); 9.096(7.5); 8.644(2.4); 8.635(4.8); 8.625(2.4); 8.372(3.8); 8.369(3.9); 8.359(4.3); 8.355(4.3);
7.984(8.2); 7.970(7.8); 7.799(14.1); 7.785(16.0); 7.780(6.0); 7.766(6.4); 7.722(2.4); 7.710(5.6); 7.697(3.6);
7.648(3.5); 7.636(5.1); 7.623(2.2); 7.456(13.9); 7.453(8.8); 7.443(13.6); 3.528(3.1); 3.516(6.2); 3.507(6.3);
3.494(3.6); 3.331(420.6); 2.997(0.5); 2.915(5.8); 2.902(9.9); 2.890(5.5); 2.615(0.6); 2.542(37.2); 2.524(1.0);
2.521(1.2); 2.518(1.1); 2.509(33.1); 2.506(72.4); 2.503(101.6); 2.500(73.3); 2.497(34.5); 2.387(0.6); 1.234(0.4);
0.000(1.6)
Example 1-13: ¹H-NMR(400.0 MHz, DMSO):
δ = 8.894(4.7); 8.889(4.6); 8.647(1.6); 8.634(3.1); 8.620(1.6); 8.566(3.2); 8.562(3.4); 8.554(3.3); 8.551(3.2);
8.081(2.3); 8.076(3.0); 8.071(2.3); 8.061(2.5); 8.056(3.3); 8.052(2.3); 7.783(3.4); 7.763(4.5); 7.729(1.5);
7.710(4.1); 7.699(9.3); 7.694(5.6); 7.678(10.5); 7.652(2.7); 7.633(3.5); 7.614(1.4); 7.492(3.0); 7.481(3.0);
7.473(3.0); 7.460(5.9); 7.439(3.6); 7.412(9.5); 7.391(8.2); 3.529(2.1); 3.512(4.5); 3.497(4.4); 3.479(2.3);
3.333(9.2); 2.903(4.0); 2.884(6.7); 2.866(3.6); 2.525(0.5); 2.512(8.2); 2.508(15.9); 2.503(20.5); 2.499(14.8);
2.494(7.1); 2.087(16.0); 1.990(0.4); 0.000(1.9)
Example 1-14: ¹H-NMR(400.0 MHz, DMSO):
δ = 8.811(1.3); 8.807(2.4); 8.802(1.3); 8.646(0.5); 8.632(1.0); 8.618(0.5); 8.562(2.2); 8.555(2.3); 8.080(0.7);
8.075(0.9); 8.069(0.7); 8.054(0.7); 8.049(0.9); 8.043(0.7); 7.782(1.1); 7.767(3.6); 7.747(3.5); 7.729(0.5);
7.711(1.2); 7.692(0.9); 7.653(0.9); 7.634(1.1); 7.615(0.4); 7.453(1.3); 7.434(1.2); 7.422(3.1); 7.402(2.7);
3.932(2.5); 3.528(0.7); 3.511(1.4); 3.496(1.4); 3.478(0.7); 3.328(6.7); 2.907(1.3); 2.888(2.2); 2.870(1.1);
2.512(4.4); 2.508(8.9); 2.503(11.9); 2.498(8.6); 2.494(4.2); 1.160(0.5); 1.071(16.0); 0.000(6.6)
Example 1-15: ¹H-NMR(400.0 MHz, DMSO):
δ = 9.099(1.0); 8.737(8.1); 8.731(7.7); 8.637(2.8); 8.624(4.5); 8.610(2.1); 8.375(0.6); 8.356(0.6); 8.316(1.4);
8.169(5.1); 8.163(4.8); 8.148(5.3); 8.142(5.0); 7.986(1.0); 7.966(0.9); 7.802(1.8); 7.781(7.0); 7.761(6.8);
7.726(3.2); 7.713(14.6); 7.692(16.0); 7.651(4.4); 7.632(5.4); 7.606(8.5); 7.585(7.5); 7.450(6.4); 7.432(5.9);
7.414(12.4); 7.394(10.4); 3.519(3.7); 3.502(7.0); 3.487(6.6); 3.469(2.9); 3.322(42.8); 2.919(1.0); 2.897(6.3);
2.879(9.5); 2.861(4.6); 2.675(0.9); 2.671(1.1); 2.667(0.8); 2.506(140.5); 2.502(166.6); 2.498(119.4); 2.333(0.9);
2.329(1.1); 2.324(0.8); 1.989(1.2); 1.398(1.0); 1.193(0.3); 1.175(0.6); 1.157(0.3); 0.146(0.6);
0.000(112.1); −0.150(0.5)
Example 1-16: ¹H-NMR(400.0 MHz, DMSO):
δ = 8.876(10.3); 8.872(10.2); 8.811(2.3); 8.796(8.3); 8.784(7.0); 8.612(9.6); 8.607(9.8); 8.250(6.4); 8.244(10.7);
8.239(5.9); 7.931(4.2); 7.914(5.5); 7.790(4.7); 7.778(5.5); 7.770(16.0); 7.758(4.8); 7.749(14.0); 7.426(12.9);
7.405(11.3); 3.548(2.8); 3.531(6.3); 3.516(6.3); 3.498(3.2); 3.460(0.3); 3.402(0.6); 3.392(0.7); 3.377(1.3);
3.372(1.5); 3.341(835.2); 3.294(0.8); 3.285(0.5); 2.907(5.3); 2.889(9.5); 2.871(4.8); 2.676(1.1); 2.672(1.6);
2.667(1.2); 2.564(0.4); 2.542(71.1); 2.512(99.0); 2.507(195.3); 2.503(251.6); 2.498(179.2); 2.334(1.1);
2.330(1.6); 2.325(1.1); 1.258(0.4); 1.234(1.2); 0.008(0.5); 0.000(14.3)
Example 1-17: ¹H-NMR(400.0 MHz, DMSO):
δ = 8.789(1.9); 8.782(1.6); 8.449(2.0); 8.443(2.0); 7.981(1.4); 7.975(1.3); 7.960(1.4); 7.953(1.4); 7.932(1.0);
7.914(1.3); 7.787(1.1); 7.775(1.1); 7.767(0.9); 7.756(0.9); 7.610(2.9); 7.590(3.5); 7.362(3.1); 7.341(2.7);
6.827(2.0); 6.806(2.0); 5.314(0.4); 5.298(1.1); 5.283(1.5); 5.267(1.1); 5.252(0.4); 3.530(0.7); 3.513(1.5);
3.497(1.5); 3.480(0.8); 3.375(0.8); 3.342(269.1); 2.875(1.3); 2.857(2.2); 2.839(1.1); 2.676(0.4); 2.672(0.5);
2.667(0.4); 2.542(3.1); 2.512(30.9); 2.507(61.9); 2.503(80.6); 2.498(58.2); 2.494(28.1); 2.334(0.4); 2.329(0.5);
2.325(0.3); 1.320(16.0); 1.304(15.9); 0.000(6.5)
Example 1-18: ¹H-NMR(400.0 MHz, DMSO):
δ = 8.805(2.9); 8.792(10.5); 8.780(7.3); 8.511(9.3); 8.505(9.4); 8.123(6.3); 8.116(5.8); 8.101(6.5); 8.095(6.3);
7.933(4.8); 7.916(6.1); 7.788(5.1); 7.776(5.1); 7.768(4.2); 7.756(4.0); 7.653(13.6); 7.633(16.0); 7.386(14.5);
7.366(12.3); 7.089(9.4); 7.068(9.1); 5.075(4.7); 5.053(15.2); 5.030(15.8); 5.007(5.4); 3.537(3.0); 3.520(6.8);
3.504(6.8); 3.486(3.3); 3.468(0.5); 3.457(0.5); 3.438(0.4); 3.425(0.7); 3.403(1.2); 3.386(2.4); 3.350(1189.1);
3.291(1.0); 3.284(0.9); 3.269(0.8); 3.256(0.6); 3.217(0.4); 2.886(5.7); 2.868(10.0); 2.850(5.1); 2.677(1.3);
2.672(1.7); 2.668(1.2); 2.543(35.9); 2.512(110.6); 2.508(215.7); 2.503(275.5); 2.499(194.3); 2.494(90.6);
2.441(0.4); 2.334(1.3); 2.330(1.6); 2.326(1.2); 1.235(1.1); 0.000(4.9)
Example 1-19: ¹H-NMR(400.0 MHz, DMSO):
δ = 9.241(0.4); 9.101(8.0); 9.096(7.9); 8.816(2.4); 8.803(5.2); 8.793(6.9); 8.785(6.4); 8.378(3.9); 8.372(4.0);
8.357(4.3); 8.352(4.4); 8.120(0.5); 8.099(0.4); 7.991(8.6); 7.970(7.8); 7.936(4.6); 7.919(5.8); 7.805(13.6);
7.789(10.2); 7.785(16.0); 7.778(6.9); 7.770(4.2); 7.758(4.0); 7.466(13.9); 7.445(12.2); 3.560(2.9); 3.542(6.6);
3.527(6.7); 3.509(3.2); 3.337(402.3); 3.305(0.7); 2.997(0.7); 2.923(5.6); 2.905(10.1); 2.887(5.1); 2.676(1.0);
2.672(1.3); 2.668(1.0); 2.558(0.4); 2.542(56.5); 2.525(3.8); 2.512(81.2); 2.507(163.7); 2.503(212.9);
2.498(152.6); 2.494(73.1); 2.334(1.0); 2.329(1.3); 2.325(0.9); 1.807(0.4); 1.785(0.6); 1.755(0.5); 1.299(0.4);
1.267(0.5); 1.259(0.5); 1.234(1.3); 1.214(0.6); 1.192(0.4); 0.008(1.3); 0.000(36.3); −0.009(1.4)
Example 1-20: ¹H-NMR(400.0 MHz, DMSO):
δ = 20.011(0.4); 15.549(0.4); 14.283(0.4); 8.804(2.7); 8.791(9.8); 8.779(7.3); 8.739(9.1); 8.732(9.4); 8.171(6.1);
8.164(6.0); 8.150(6.8); 8.143(6.4); 7.931(4.8); 7.911(5.8); 7.787(5.2); 7.775(5.1); 7.768(4.2); 7.756(4.1);
7.716(13.9); 7.695(16.0); 7.610(9.8); 7.589(9.1); 7.420(14.4); 7.400(12.6); 3.722(0.5); 3.700(0.4); 3.672(0.4);
3.639(0.5); 3.603(0.4); 3.542(3.3); 3.525(7.1); 3.509(7.0); 3.491(3.8); 3.446(1.0); 3.433(1.1); 3.419(1.2);
3.406(1.6); 3.392(1.7); 3.337(2062.5); 3.289(1.8); 3.277(1.1); 3.094(0.4); 3.058(0.4); 2.996(0.5); 2.899(5.8);
2.881(10.4); 2.863(5.1); 2.712(0.5); 2.676(4.3); 2.671(5.8); 2.667(4.4); 2.636(0.5); 2.602(0.6); 2.578(0.8);
2.542(49.2); 2.525(14.1); 2.511(354.2); 2.507(724.5); 2.502(947.1); 2.498(672.4); 2.493(317.1); 2.464(1.5);
2.334(4.4); 2.329(5.9); 2.324(4.2); 2.289(0.4); 1.297(0.6); 1.258(0.8); 1.235(3.6); 0.853(0.5); 0.008(1.9);
0.000(68.1); −0.009(2.1); −1.700(0.5); −3.167(0.4)
Example 1-21: ¹H-NMR(400.0 MHz, DMSO):
δ = 8.811(7.8); 8.807(11.5); 8.803(7.4); 8.796(8.7); 8.783(7.1); 8.566(9.1); 8.559(9.2); 8.084(2.8); 8.079(4.0);
8.073(2.6); 8.058(2.9); 8.053(4.0); 8.047(2.6); 7.931(4.2); 7.914(5.5); 7.789(4.6); 7.777(5.6); 7.770(16.0);
7.757(5.1); 7.749(14.0); 7.427(12.8); 7.407(11.3); 3.549(2.8); 3.532(6.4); 3.516(6.4); 3.499(3.1); 3.390(0.5);
3.372(1.2); 3.336(511.2); 2.907(5.4); 2.889(9.6); 2.871(4.8); 2.712(0.4); 2.676(1.2); 2.672(1.5); 2.667(1.2);
2.542(86.1); 2.507(187.1); 2.503(241.3); 2.498(171.9); 2.368(0.3); 2.334(1.0); 2.329(1.5); 2.325(1.0); 1.259(0.3);
1.235(1.1); 0.008(1.5); 0.000(40.4); −0.009(1.4)
Example 1-22: ¹H-NMR(400.0 MHz, DMSO):
δ = 8.809(0.7); 8.793(2.9); 8.780(2.3); 8.473(1.4); 8.469(1.6); 8.461(1.5); 8.457(1.5); 7.927(1.4); 7.910(1.8);
7.786(1.6); 7.775(1.6); 7.767(1.3); 7.755(1.2); 7.625(0.9); 7.605(1.0); 7.388(1.3); 7.367(8.4); 7.359(8.9);
7.338(2.1); 7.325(0.9); 7.319(0.9); 7.305(0.8); 3.568(1.0); 3.551(2.2); 3.535(2.2); 3.518(1.1); 3.498(0.4);
3.491(0.4); 3.466(0.5); 3.445(0.6); 3.430(0.8); 3.410(0.9); 3.341(851.2); 3.283(1.3); 3.260(0.5); 3.243(0.3);
2.909(1.9); 2.890(3.2); 2.872(1.8); 2.712(0.5); 2.676(1.6); 2.672(2.2); 2.667(1.6); 2.542(116.9); 2.525(5.4);
2.520(7.8); 2.512(126.1); 2.507(263.0); 2.503(348.3); 2.498(247.5); 2.494(116.0); 2.468(0.7); 2.464(0.7);
2.444(16.0); 2.367(0.4); 2.338(0.7); 2.334(1.5); 2.329(2.1); 2.325(1.5); 2.290(0.5); 1.258(0.5); 1.235(1.4);
0.008(0.6); 0.000(21.5); −0.009(0.7)
Example 1-23: ¹H-NMR(400.0 MHz, DMSO):
δ = 8.894(8.3); 8.889(8.0); 8.811(2.3); 8.795(8.9); 8.782(7.2); 8.568(6.0); 8.564(6.3); 8.556(6.2); 8.552(6.1);
8.089(3.2); 8.083(4.2); 8.079(3.2); 8.069(3.5); 8.063(4.6); 8.059(3.2); 7.935(4.5); 7.918(5.8); 7.789(5.0);
7.777(4.9); 7.770(4.0); 7.758(3.8); 7.702(13.6); 7.682(16.0); 7.500(4.4); 7.488(4.3); 7.480(4.2); 7.469(3.9);
7.416(14.2); 7.396(12.2); 3.571(0.4); 3.548(3.2); 3.530(6.7); 3.515(6.8); 3.497(3.7); 3.474(0.5); 3.460(0.6);
3.444(1.0); 3.417(1.3); 3.378(4.6); 3.345(1223.4); 3.292(1.8); 3.262(0.8); 3.239(0.8); 3.225(0.5); 3.162(0.4);
3.015(0.6); 2.901(5.8); 2.883(10.1); 2.865(5.2); 2.753(0.5); 2.712(0.9); 2.677(1.9); 2.672(2.7); 2.667(2.0);
2.577(0.3); 2.542(220.5); 2.525(6.4); 2.512(163.1); 2.507(330.0); 2.503(430.1); 2.498(306.5); 2.494(144.3);
2.419(0.4); 2.415(0.4); 2.368(0.9); 2.334(2.0); 2.330(2.7); 2.325(2.0); 2.291(0.6); 1.298(0.4); 1.258(0.7);
1.234(1.9); 0.854(0.3); 0.000(9.9); −0.009(0.3)
Example 1-24: ¹H-NMR(400.0 MHz, DMSO):
δ = 8.787(4.8); 8.776(4.8); 8.748(1.9); 8.734(3.6); 8.720(1.9); 7.910(4.1); 7.891(5.3); 7.785(4.6); 7.773(4.6);
7.765(3.6); 7.753(3.4); 7.509(13.6); 7.488(16.0); 7.245(14.1); 7.224(12.1); 3.494(3.2); 3.476(7.4); 3.461(7.3);
3.444(3.4); 3.406(0.6); 3.398(0.5); 3.391(1.0); 3.338(825.0); 3.307(1.0); 3.293(0.6); 3.281(0.4); 2.811(5.9);
2.793(11.1); 2.775(5.3); 2.675(1.4); 2.672(1.9); 2.667(1.4); 2.542(22.5); 2.525(5.0); 2.511(114.0); 2.507(230.2);
2.502(300.7); 2.498(215.0); 2.494(102.6); 2.333(1.3); 2.329(1.8); 2.325(1.3); 2.320(0.6); 1.258(0.4); 1.236(1.2);
0.000(14.7); −0.009(0.4)
Example 1-25: ¹H-NMR(400.0 MHz, DMSO):
δ = 8.790(2.1); 8.778(2.3); 8.761(0.6); 7.930(1.2); 7.913(1.6); 7.785(1.4); 7.773(1.4); 7.765(1.1); 7.754(1.1);
7.564(3.0); 7.560(3.0); 7.401(3.0); 7.381(4.9); 7.316(4.5); 7.296(2.8); 6.688(2.9); 6.683(2.9); 3.520(0.8);
3.503(1.8); 3.487(1.8); 3.469(1.0); 3.366(0.9); 3.340(197.6); 3.307(0.3); 2.853(1.6); 2.835(2.7); 2.816(1.5);
2.676(0.3); 2.672(0.4); 2.542(9.3); 2.525(1.1); 2.511(26.5); 2.507(53.2); 2.503(68.9); 2.498(48.8); 2.494(23.0);
2.422(16.0); 2.334(0.3); 2.329(0.4); 1.236(0.4); 0.008(0.5); 0.000(14.6); −0.009(0.5)
Example 1-26: ¹H-NMR(400.0 MHz, DMSO):
δ = 8.789(6.3); 8.780(7.2); 8.778(7.0); 8.770(4.8); 8.755(2.3); 8.155(11.1); 7.921(4.6); 7.904(6.0); 7.902(5.9);
7.783(5.1); 7.771(5.1); 7.764(4.2); 7.752(4.0); 7.731(7.8); 7.727(12.9); 7.723(7.5); 7.567(13.6); 7.547(16.0);
7.288(14.4); 7.267(12.4); 6.949(9.2); 6.947(10.0); 6.945(9.9); 6.943(8.7); 3.571(0.4); 3.536(0.5); 3.512(3.5);
3.495(7.5); 3.479(7.3); 3.462(4.2); 3.380(1085.1); 3.312(1.2); 3.300(1.1); 3.289(1.0); 3.270(0.6); 3.259(0.4);
3.248(0.4); 3.232(0.4); 2.840(6.0); 2.822(10.2); 2.804(5.3); 2.679(0.7); 2.674(0.9); 2.670(0.7); 2.544(40.0);
2.527(2.3); 2.514(52.0); 2.510(103.6); 2.505(133.7); 2.501(95.3); 2.496(45.0); 2.336(0.6); 2.332(0.8); 2.327(0.6);
1.234(0.8); 0.008(1.2); 0.000(29.7); −0.009(1.0)
Example 1-27: ¹H-NMR(400.0 MHz, DMSO):
δ = 8.805(2.7); 8.792(10.7); 8.780(8.0); 8.461(9.8); 8.447(10.3); 7.926(4.9); 7.908(6.4); 7.895(0.6); 7.857(11.5);
7.854(12.1); 7.840(14.5); 7.819(16.0); 7.787(5.6); 7.775(6.0); 7.769(10.5); 7.766(9.5); 7.756(11.2); 7.752(7.5);
7.509(0.8); 7.488(0.9); 7.443(14.6); 7.423(13.1); 7.245(0.9); 7.224(0.7); 3.551(3.2); 3.534(7.3); 3.519(7.4);
3.501(3.5); 3.476(0.6); 3.462(0.6); 3.444(0.4); 3.439(0.4); 3.414(0.4); 3.406(0.5); 3.377(1.6); 3.340(941.1);
3.307(1.5); 3.281(0.6); 3.258(0.5); 3.251(0.4); 2.914(6.1); 2.896(11.1); 2.878(5.5); 2.812(0.4); 2.793(0.7);
2.774(0.4); 2.676(1.4); 2.672(1.9); 2.668(1.4); 2.542(46.6); 2.525(5.0); 2.512(117.2); 2.507(236.9); 2.503(309.7);
2.498(223.4); 2.494(108.4); 2.419(0.4); 2.334(1.4); 2.330(2.0); 2.325(1.4); 1.298(0.3); 1.258(0.5); 1.234(1.3);
0.008(1.4); 0.000(35.7); −0.008(1.3)
Example 1-28: ¹H-NMR(400.0 MHz, DMSO):
δ = 8.807(2.5); 8.792(10.1); 8.780(7.8); 8.297(9.3); 8.284(9.8); 7.927(4.9); 7.910(6.3); 7.847(14.3); 7.827(16.0);
7.787(5.3); 7.775(5.2); 7.768(4.3); 7.756(4.1); 7.720(3.6); 7.716(5.6); 7.712(3.9); 7.707(3.8); 7.703(5.4);
7.699(3.4); 7.533(11.6); 7.448(14.6); 7.428(13.2); 3.555(3.2); 3.537(7.4); 3.522(7.4); 3.504(3.5); 3.375(0.9);
3.339(418.0); 3.300(0.3); 2.917(6.2); 2.899(11.2); 2.882(5.6); 2.676(0.8); 2.672(1.1); 2.668(0.8); 2.542(11.0);
2.525(2.6); 2.512(65.9); 2.508(131.5); 2.503(170.5); 2.499(121.4); 2.494(57.5); 2.334(0.7); 2.330(1.0);
2.325(0.8); 1.234(0.8); 0.008(1.2); 0.000(33.1); −0.009(1.1)
Example 1-29: ¹H-NMR(400.0 MHz, DMSO):
δ = 8.805(2.6); 8.791(10.2); 8.778(7.7); 8.033(14.8); 8.012(16.0); 7.998(4.7); 7.996(5.1); 7.978(11.5); 7.976(10.1);
7.955(9.3); 7.936(16.0); 7.917(9.9); 7.788(5.2); 7.776(5.3); 7.769(4.3); 7.757(4.0); 7.465(9.0); 7.463(8.7);
7.446(8.4); 7.444(8.1); 7.424(14.5); 7.403(13.4); 3.628(0.4); 3.614(0.4); 3.596(0.4); 3.551(3.3); 3.534(7.2);
3.518(7.5); 3.501(3.7); 3.462(0.7); 3.438(1.0); 3.414(1.5); 3.401(1.8); 3.396(2.1); 3.386(2.9); 3.343(1848.6);
3.314(3.3); 3.294(1.5); 3.279(0.9); 3.264(0.7); 3.253(0.4); 3.241(0.5); 3.218(0.5); 2.910(6.0); 2.892(10.8);
2.874(5.4); 2.676(2.8); 2.672(3.6); 2.667(2.8); 2.582(0.4); 2.542(24.0); 2.525(10.1); 2.512(227.9); 2.507(455.1);
2.503(586.9); 2.498(414.0); 2.494(191.7); 2.452(0.6); 2.443(0.3); 2.334(2.4); 2.330(3.4); 2.325(2.5); 2.291(0.4);
1.297(0.4); 1.258(0.6); 1.234(1.6); 0.008(1.5); 0.000(37.6); −0.009(1.2)
Example 1-30: ¹H-NMR(400.0 MHz, DMSO):
δ = 8.804(0.9); 8.791(3.2); 8.780(2.3); 7.943(16.0); 7.922(1.4); 7.900(5.9); 7.879(4.4); 7.787(1.7); 7.774(1.6);
7.767(1.3); 7.755(1.2); 7.462(0.4); 7.447(4.2); 7.427(3.9); 7.250(0.3); 3.708(0.4); 3.649(0.3); 3.639(0.4);
3.570(0.4); 3.552(1.2); 3.533(2.4); 3.517(2.4); 3.501(1.2); 3.485(0.8); 3.474(0.6); 3.454(0.7); 3.432(1.3);
3.408(1.3); 3.399(1.4); 3.391(1.9); 3.384(2.5); 3.339(2092.8); 3.309(3.3); 3.303(2.4); 3.293(1.2); 3.283(1.1);
3.275(0.7); 3.269(0.5); 3.258(0.8); 3.233(0.4); 3.211(0.3); 3.199(0.3); 3.160(0.3); 2.918(1.9); 2.900(3.3);
2.882(1.6); 2.676(3.3); 2.672(4.4); 2.667(3.1); 2.598(0.6); 2.542(21.9); 2.511(266.6); 2.507(531.5); 2.502(689.2);
2.498(488.3); 2.494(229.2); 2.465(1.3); 2.456(0.8); 2.334(3.0); 2.329(4.2); 2.325(3.1); 2.290(0.5); 1.298(0.5);
1.258(0.7); 1.254(0.3); 1.233(1.3); 0.146(0.4); 0.008(3.0); 0.000(76.3); −0.009(2.6); −0.149(0.3)
Example 1-31: ¹H-NMR(400.0 MHz, DMSO):
δ = 8.700(1.9); 8.686(3.9); 8.672(1.9); 8.600(16.0); 8.570(9.0); 8.556(9.4); 7.787(4.0); 7.767(5.4); 7.732(1.7);
7.714(4.5); 7.701(9.5); 7.687(8.7); 7.658(3.3); 7.639(4.2); 7.627(8.9); 7.623(10.2); 7.532(4.9); 7.512(9.2);
7.480(5.1); 7.471(6.6); 7.467(6.7); 7.462(4.8); 7.452(3.6); 7.447(3.4); 3.574(2.2); 3.557(5.3); 3.541(5.4);
3.524(2.6); 3.384(0.6); 3.331(847.6); 3.274(0.5); 3.042(4.7); 3.023(8.2); 3.006(4.2); 2.995(2.0); 2.711(0.5);
2.675(1.7); 2.671(2.4); 2.667(1.7); 2.541(102.6); 2.506(274.3); 2.502(356.9); 2.497(258.9); 2.367(0.5);
2.333(1.7); 2.329(2.3); 2.324(1.7); 2.075(0.4); 1.298(0.4); 1.258(0.6); 1.235(2.5); 0.008(0.5); 0.000(11.4)
Example 1-32: ¹H-NMR(400.0 MHz, DMSO):
δ = 8.678(2.6); 8.664(5.4); 8.650(2.6); 8.565(9.3); 8.559(9.5); 8.179(6.1); 8.173(5.8); 8.157(6.3); 8.151(6.2);
8.142(0.4); 7.809(11.6); 7.804(12.0); 7.785(5.3); 7.766(6.9); 7.736(2.3); 7.717(5.8); 7.699(4.2); 7.665(5.6);
7.660(7.4); 7.645(7.1); 7.640(10.0); 7.619(2.1); 7.485(14.1); 7.465(12.7); 7.119(0.4); 7.099(9.9); 7.077(9.4);
5.083(4.8); 5.061(15.3); 5.038(16.0); 5.015(5.4); 3.541(2.8); 3.524(6.6); 3.508(6.7); 3.491(3.2); 3.331(423.8);
3.007(5.9); 2.989(10.1); 2.971(5.1); 2.712(0.5); 2.676(1.0); 2.671(1.4); 2.667(1.0); 2.663(0.5); 2.542(125.6);
2.525(4.0); 2.511(80.9); 2.507(161.8); 2.502(211.6); 2.498(151.9); 2.493(73.2); 2.368(0.5); 2.334(1.0);
2.329(1.4); 2.325(1.0); 1.235(1.4); 0.000(7.6)
Example 1-33: ¹H-NMR(400.0 MHz, DMSO):
δ = 8.931(11.5); 8.925(11.3); 8.684(3.2); 8.670(6.5); 8.656(3.2); 8.598(7.6); 8.594(8.1); 8.586(7.9); 8.582(8.0);
8.139(4.3); 8.134(6.0); 8.129(4.4); 8.119(4.7); 8.114(6.4); 8.109(4.5); 7.852(14.3); 7.848(15.1); 7.785(7.1);
7.766(9.3); 7.737(3.2); 7.718(8.3); 7.712(8.3); 7.707(7.2); 7.700(6.4); 7.692(8.8); 7.688(8.2); 7.657(5.6);
7.638(7.3); 7.619(2.7); 7.516(13.4); 7.510(7.3); 7.497(16.0); 7.489(8.9); 7.484(9.4); 7.480(8.3); 7.465(7.2);
3.553(4.0); 3.536(9.5); 3.520(9.6); 3.503(4.5); 3.407(0.3); 3.333(561.6); 3.022(8.3); 3.004(14.4); 2.986(7.2);
2.711(0.7); 2.676(1.2); 2.671(1.6); 2.667(1.2); 2.542(152.0); 2.507(189.6); 2.502(245.8); 2.498(180.4);
2.368(0.7); 2.333(1.1); 2.329(1.5); 2.325(1.1); 1.258(0.4); 1.234(1.6); 0.000(6.1)
Example 1-34: ¹H-NMR(400.0 MHz, DMSO):
δ = 8.680(3.1); 8.666(6.1); 8.652(3.1); 8.319(11.2); 8.305(11.7); 8.005(13.9); 8.000(14.2); 7.849(6.7); 7.844(6.4);
7.829(7.7); 7.824(7.4); 7.783(6.8); 7.764(13.2); 7.756(7.2); 7.752(4.4); 7.734(2.9); 7.716(7.2); 7.698(5.2);
7.656(5.2); 7.637(6.8); 7.616(16.0); 7.550(12.1); 7.530(10.6); 7.475(7.7); 7.457(6.6); 3.559(3.9); 3.541(9.1);
3.526(9.3); 3.509(4.5); 3.477(0.5); 3.457(0.5); 3.448(0.5); 3.429(0.7); 3.419(0.9); 3.408(1.0); 3.392(1.8);
3.333(1813.8); 3.282(0.7); 3.265(0.5); 3.035(7.6); 3.017(13.7); 2.999(6.7); 2.676(3.4); 2.671(4.6); 2.667(3.4);
2.541(56.3); 2.524(13.2); 2.511(271.9); 2.507(541.2); 2.502(706.5); 2.498(508.5); 2.493(244.9); 2.440(0.4);
2.333(3.3); 2.329(4.4); 2.324(3.3); 2.289(0.5); 2.075(0.5); 1.298(0.7); 1.258(1.1); 1.235(4.9); 0.853(0.5);
0.008(0.6); 0.000(16.8); −0.008(0.6)
Example 1-35: ¹H-NMR(400.0 MHz, DMSO):
δ = 8.652(2.9); 8.638(5.8); 8.624(2.9); 8.271(13.6); 7.782(6.3); 7.763(8.5); 7.752(8.8); 7.748(14.8); 7.744(8.8);
7.732(15.9); 7.728(16.0); 7.712(7.0); 7.694(5.0); 7.653(4.9); 7.634(6.3); 7.616(2.4); 7.572(6.3); 7.568(5.9);
7.552(7.6); 7.548(7.4); 7.470(7.4); 7.451(6.4); 7.388(11.8); 7.368(9.7); 7.025(11.9); 7.022(11.7); 3.518(3.6);
3.501(8.4); 3.485(8.5); 3.468(4.1); 3.393(0.4); 3.333(492.1); 3.287(0.4); 2.996(0.9); 2.963(7.3); 2.945(12.7);
2.927(6.4); 2.711(0.5); 2.676(1.1); 2.671(1.5); 2.667(1.1); 2.541(131.2); 2.524(4.0); 2.511(90.8); 2.507(179.2);
2.502(232.5); 2.498(168.1); 2.494(81.9); 2.368(0.6); 2.333(1.1); 2.329(1.5); 2.324(1.1); 1.258(0.4); 1.235(1.5);
0.000(6.2)
Example 1-36: ¹H-NMR(400.0 MHz, DMSO):
δ = 8.680(1.6); 8.666(3.3); 8.653(10.6); 8.649(6.9); 8.641(6.2); 8.637(9.7); 7.923(7.2); 7.918(7.5); 7.787(5.0);
7.783(6.7); 7.767(16.0); 7.763(14.1); 7.756(6.4); 7.752(9.7); 7.735(1.6); 7.716(3.8); 7.698(2.6); 7.656(2.6);
7.637(3.4); 7.618(1.3); 7.539(6.5); 7.519(5.6); 7.479(4.0); 7.460(3.4); 3.557(2.0); 3.540(4.6); 3.525(4.6);
3.507(2.2); 3.365(0.6); 3.334(254.1); 3.030(4.0); 3.012(7.1); 2.994(3.5); 2.676(0.6); 2.672(0.8); 2.667(0.6);
2.542(21.0); 2.525(2.1); 2.511(47.6); 2.507(96.0); 2.503(125.7); 2.498(90.0); 2.494(42.8); 2.334(0.6); 2.329(0.8);
2.325(0.6); 1.234(1.6); 0.000(4.2)
Example 1-37: ¹H-NMR(400.0 MHz, DMSO):
δ = 8.849(14.5); 8.685(3.8); 8.671(7.4); 8.657(3.7); 8.602(13.1); 8.595(13.2); 8.167(4.7); 8.162(6.6); 8.157(4.3);
8.141(4.8); 8.136(6.7); 8.131(4.2); 7.938(15.9); 7.933(15.9); 7.820(0.4); 7.785(9.2); 7.781(9.9); 7.776(8.5);
7.766(11.9); 7.761(11.1); 7.756(8.9); 7.737(3.6); 7.719(8.8); 7.700(6.3); 7.658(6.3); 7.639(8.1); 7.620(3.2);
7.576(0.4); 7.527(13.6); 7.507(11.9); 7.482(9.5); 7.463(8.1); 7.436(0.5); 3.554(4.5); 3.537(10.7); 3.521(11.0);
3.504(5.1); 3.475(0.5); 3.457(0.4); 3.333(476.2); 3.027(9.1); 3.009(16.0); 2.991(8.1); 2.948(0.4); 2.712(0.9);
2.676(1.7); 2.671(2.2); 2.542(181.3); 2.507(273.2); 2.503(339.2); 2.498(249.8); 2.368(0.9); 2.333(1.6);
2.329(2.1); 2.325(1.5); 2.075(0.4); 2.031(0.4); 1.867(0.4); 1.298(0.4); 1.258(0.6); 1.235(2.1); 0.000(8.7)
Example 1-38: ¹H-NMR(400.0 MHz, DMSO):
δ = 9.140(12.2); 9.136(12.2); 8.690(3.9); 8.676(7.5); 8.663(3.7); 8.438(6.2); 8.433(6.2); 8.417(6.6); 8.413(6.7);
8.316(0.4); 8.006(12.7); 7.986(12.4); 7.977(15.7); 7.972(15.8); 7.831(0.3); 7.810(7.5); 7.806(7.1); 7.786(16.0);
7.767(10.5); 7.737(3.6); 7.718(8.7); 7.700(6.3); 7.659(6.1); 7.639(8.0); 7.620(3.0); 7.565(13.1); 7.545(11.2);
7.484(9.4); 7.466(7.9); 3.687(0.3); 3.631(0.4); 3.596(0.4); 3.563(4.5); 3.546(10.7); 3.530(11.0); 3.513(5.1);
3.459(0.7); 3.437(1.0); 3.398(1.9); 3.330(2223.4); 3.254(0.5); 3.230(0.4); 3.041(8.8); 3.023(15.6); 3.005(7.7);
2.995(4.3); 2.859(0.4); 2.712(0.7); 2.675(6.8); 2.671(8.9); 2.667(6.8); 2.602(0.5); 2.541(114.6); 2.506(1101.2);
2.502(1388.2); 2.498(1042.5); 2.420(0.9); 2.368(0.7); 2.333(6.8); 2.329(8.8); 2.325(6.8); 2.288(0.8); 2.282(0.6);
2.263(0.5); 2.075(0.9); 1.518(0.4); 1.502(0.3); 1.496(0.3); 1.298(1.6); 1.258(2.4); 1.235(9.6); 0.889(0.3);
0.871(0.4); 0.854(1.0); 0.837(0.5); 0.000(42.0)
Example 1-39: ¹H-NMR(400.0 MHz, DMSO):
δ = 8.622(2.2); 8.608(4.2); 8.594(2.1); 7.777(5.2); 7.758(6.8); 7.732(2.3); 7.717(13.8); 7.712(16.0); 7.695(4.0);
7.653(4.0); 7.634(5.2); 7.615(2.0); 7.534(5.4); 7.529(5.2); 7.513(6.6); 7.508(6.4); 7.454(6.0); 7.435(5.2);
7.346(10.6); 7.325(8.6); 3.502(3.3); 3.485(8.2); 3.469(8.3); 3.452(3.8); 3.426(0.5); 3.341(328.7); 3.336(427.1);
2.932(6.5); 2.914(12.4); 2.897(5.7); 2.676(0.9); 2.671(1.3); 2.667(0.9); 2.542(70.7); 2.525(3.2); 2.511(72.6);
2.507(146.7); 2.502(193.3); 2.498(139.9); 2.494(68.0); 2.334(0.9); 2.329(1.2); 2.325(0.9); 1.235(1.2); 0.000(4.4)
Example 1-40: ¹H-NMR(400.0 MHz, DMSO):
δ = 8.661(0.7); 8.647(1.3); 8.633(0.7); 7.783(1.5); 7.763(1.9); 7.731(0.6); 7.713(1.6); 7.695(1.2); 7.655(1.2);
7.635(1.5); 7.616(0.6); 7.588(3.1); 7.583(3.1); 7.499(2.9); 7.496(2.9); 7.476(1.7); 7.457(1.5); 7.425(0.8);
7.405(4.1); 7.398(3.0); 7.394(2.6); 7.378(0.6); 7.374(0.6); 6.762(2.9); 6.757(2.9); 3.526(0.9); 3.509(1.9);
3.493(1.9); 3.476(1.0); 3.331(246.2); 2.995(0.5); 2.977(1.7); 2.959(2.8); 2.941(1.5); 2.675(0.6); 2.671(0.8);
2.666(0.6); 2.541(43.5); 2.524(2.2); 2.511(47.2); 2.506(96.2); 2.502(126.7); 2.497(91.6); 2.493(44.3); 2.468(0.5);
2.436(16.0); 2.333(0.6); 2.329(0.8); 2.324(0.6); 1.235(0.6); 0.000(4.3)
Example 1-41: ¹H-NMR(400.0 MHz, DMSO):
δ = 8.678(2.2); 8.664(4.5); 8.650(2.1); 8.133(9.7); 8.128(10.1); 8.078(6.1); 8.059(8.7); 8.028(5.0); 8.023(4.6);
8.008(5.3); 8.003(4.9); 7.987(6.2); 7.967(10.3); 7.948(4.8); 7.809(0.6); 7.805(0.7); 7.783(4.8); 7.763(6.2);
7.737(2.2); 7.718(5.3); 7.699(3.8); 7.680(0.4); 7.655(4.0); 7.636(4.9); 7.617(1.9); 7.537(8.2); 7.517(16.0);
7.498(7.9); 7.481(5.7); 7.463(4.8); 3.560(2.6); 3.543(6.3); 3.527(6.6); 3.510(3.4); 3.480(0.3); 3.470(0.3);
3.458(0.4); 3.424(0.6); 3.340(726.6); 3.336(727.4); 3.030(5.1); 3.013(9.3); 2.995(6.2); 2.971(0.4); 2.712(0.4);
2.676(1.6); 2.671(2.1); 2.667(1.5); 2.663(0.7); 2.542(85.1); 2.525(5.2); 2.511(127.2); 2.507(255.3); 2.502(333.6);
2.498(238.4); 2.493(113.1); 2.368(0.4); 2.338(0.8); 2.334(1.6); 2.329(2.1); 2.325(1.6); 1.258(0.4); 1.234(2.1);
0.000(9.2)
Example 1-42: ¹H-NMR(400.0 MHz, DMSO):
δ = 8.665(1.7); 8.481(1.9); 8.473(2.0); 7.785(1.8); 7.764(2.5); 7.731(0.9); 7.712(2.1); 7.694(1.6); 7.656(1.7);
7.636(3.6); 7.617(2.8); 7.498(3.8); 7.494(3.9); 7.481(3.3); 7.462(4.1); 7.364(2.3); 7.345(1.8); 7.326(1.6);
7.314(1.8); 7.295(1.4); 3.572(1.1); 3.555(2.6); 3.539(2.8); 3.523(1.3); 3.330(758.3); 3.027(2.3); 3.008(3.9);
2.995(2.5); 2.711(0.4); 2.671(3.3); 2.541(51.4); 2.502(511.1); 2.440(16.0); 2.367(0.4); 2.329(3.1); 2.289(0.4);
1.298(0.5); 1.258(0.8); 1.235(3.1); 0.854(0.3); 0.000(8.7)
Example 1-43: ¹H-NMR(400.0 MHz, DMSO):
δ = 8.674(0.7); 8.660(1.4); 8.646(0.7); 8.500(2.4); 8.493(2.4); 8.035(1.4); 8.029(1.3); 8.014(1.4); 8.007(1.4);
7.785(1.5); 7.765(2.0); 7.755(2.9); 7.750(2.9); 7.735(0.6); 7.717(1.6); 7.698(1.1); 7.656(1.2); 7.637(1.5);
7.622(1.6); 7.618(1.7); 7.602(1.6); 7.598(1.5); 7.480(1.7); 7.461(3.8); 7.441(1.9); 6.835(2.4); 6.814(2.3);
5.322(0.4); 5.307(1.1); 5.291(1.5); 5.276(1.2); 5.261(0.5); 3.535(0.8); 3.518(1.8); 3.502(1.9); 3.485(0.9);
3.333(259.8); 2.997(1.7); 2.979(2.7); 2.961(1.3); 2.671(0.7); 2.542(24.4); 2.507(85.8); 2.502(107.2); 2.498(79.5);
2.333(0.5); 2.329(0.7); 1.322(16.0); 1.306(15.9); 1.235(0.8); 0.000(2.1)
Example 1-44: ¹H-NMR(400.0 MHz, DMSO):
δ = 20.005(0.5); 8.680(3.4); 8.666(7.1); 8.652(3.6); 8.603(10.8); 8.597(11.3); 8.364(3.5); 8.358(3.5); 8.344(6.5);
8.337(6.3); 8.323(4.0); 8.317(4.0); 7.858(15.0); 7.854(15.9); 7.785(7.5); 7.765(10.1); 7.736(3.3); 7.717(8.5);
7.697(12.7); 7.682(8.9); 7.677(8.6); 7.657(6.2); 7.637(8.0); 7.618(3.2); 7.510(13.6); 7.490(11.9); 7.481(9.6);
7.462(8.1); 7.309(6.8); 7.302(6.8); 7.288(6.8); 7.281(6.6); 3.546(4.4); 3.530(10.3); 3.514(10.5); 3.496(4.9);
3.333(1943.2); 3.017(9.0); 2.998(16.0); 2.981(7.9); 2.711(1.6); 2.671(5.6); 2.541(321.8); 2.506(675.4);
2.502(850.6); 2.498(620.0); 2.368(1.5); 2.329(5.4); 2.289(0.4); 2.075(0.6); 1.298(0.8); 1.258(1.1); 1.235(4.3);
0.855(0.5); 0.000(20.4)
Example 1-45: ¹H-NMR(400.0 MHz, DMSO):
δ = 9.016(0.3); 8.916(15.8); 8.911(14.9); 8.711(0.4); 8.684(4.0); 8.671(7.5); 8.657(4.2); 8.644(14.3); 8.639(13.8);
8.332(9.9); 8.327(15.5); 8.322(8.5); 7.946(16.0); 7.941(15.3); 7.830(0.4); 7.786(9.1); 7.780(9.5); 7.775(8.5);
7.767(11.9); 7.760(10.5); 7.756(8.6); 7.738(3.6); 7.719(8.9); 7.700(6.3); 7.658(6.2); 7.639(8.0); 7.620(3.1);
7.523(12.8); 7.503(11.1); 7.481(9.3); 7.463(7.9); 3.551(4.5); 3.534(10.9); 3.519(11.3); 3.502(5.2); 3.472(0.5);
3.449(0.6); 3.430(0.7); 3.406(0.9); 3.332(1259.4); 3.265(1.0); 3.231(0.4); 3.025(9.0); 3.007(15.9); 2.995(6.5);
2.990(8.0); 2.711(0.7); 2.671(4.2); 2.541(157.2); 2.506(549.1); 2.502(657.8); 2.498(486.9); 2.400(0.4);
2.367(0.9); 2.329(4.2); 2.289(0.5); 2.282(0.3); 2.075(0.5); 1.298(0.7); 1.259(1.0); 1.235(4.3); 0.853(0.5);
0.000(15.0)
Example 1-46: ¹H-NMR(400.0 MHz, DMSO):
δ = 8.680(0.9); 8.666(1.7); 8.652(0.9); 8.083(3.5); 8.078(3.7); 8.021(16.0); 7.902(1.7); 7.897(1.6); 7.882(1.9);
7.877(1.8); 7.809(0.4); 7.805(0.4); 7.784(1.9); 7.765(2.5); 7.734(0.8); 7.716(2.1); 7.698(1.5); 7.657(1.6);
7.638(1.9); 7.619(0.7); 7.546(3.0); 7.525(2.7); 7.471(2.1); 7.453(1.8); 3.554(1.0); 3.537(2.3); 3.522(2.5);
3.505(1.2); 3.397(0.4); 3.334(534.8); 3.278(0.4); 3.034(1.9); 3.016(3.5); 2.998(1.8); 2.676(1.0); 2.671(1.4);
2.667(1.0); 2.541(14.4); 2.524(3.1); 2.511(78.2); 2.507(158.8); 2.502(210.2); 2.498(153.5); 2.493(75.5);
2.333(1.0); 2.329(1.3); 2.324(1.0); 1.258(0.4); 1.234(1.5); 0.000(5.3)
Example 1-47: ¹H-NMR(400.0 MHz, DMSO):
δ = 8.648(0.7); 8.634(1.4); 8.620(0.7); 8.202(4.6); 7.912(4.9); 7.781(1.5); 7.762(1.9); 7.729(0.6); 7.710(1.6);
7.692(1.2); 7.659(3.4); 7.654(4.2); 7.633(1.5); 7.615(0.6); 7.508(1.4); 7.504(1.4); 7.488(1.8); 7.484(1.8);
7.470(1.7); 7.451(1.5); 7.348(2.8); 7.328(2.2); 3.852(16.0); 3.505(0.8); 3.488(1.9); 3.472(1.9); 3.455(0.9);
3.332(115.8); 3.311(0.5); 2.995(0.4); 2.941(1.7); 2.923(2.9); 2.906(1.5); 2.671(0.4); 2.667(0.3); 2.541(39.4);
2.524(1.0); 2.511(26.1); 2.506(51.7); 2.502(67.0); 2.497(48.0); 2.493(23.1); 2.329(0.4); 1.235(0.5); 0.000(2.2)
Example 1-48: ¹H-NMR(400.0 MHz, DMSO):
δ = 8.697(2.9); 8.683(6.0); 8.669(2.9); 8.465(7.6); 8.460(8.2); 8.453(8.1); 8.448(7.9); 8.317(0.5); 7.924(7.7);
7.920(7.9); 7.905(8.9); 7.901(8.3); 7.787(6.2); 7.767(8.1); 7.730(2.7); 7.712(6.9); 7.693(5.2); 7.658(5.0);
7.639(6.2); 7.620(2.5); 7.601(13.6); 7.596(13.8); 7.556(8.4); 7.544(8.3); 7.537(7.8); 7.525(7.8); 7.514(7.7);
7.494(14.6); 7.476(7.4); 7.457(16.0); 7.453(12.3); 7.438(5.0); 7.433(4.9); 7.391(0.3); 4.852(0.4); 4.011(0.3);
3.626(0.4); 3.596(0.5); 3.569(3.7); 3.552(8.2); 3.536(8.4); 3.519(4.3); 3.489(0.6); 3.485(0.7); 3.478(0.7);
3.460(0.9); 3.437(0.9); 3.331(2964.6); 3.330(2877.3); 3.298(3.4); 3.264(0.7); 3.232(0.5); 3.035(7.3); 3.016(12.4);
2.999(6.5); 2.995(6.0); 2.711(0.9); 2.675(6.8); 2.671(9.2); 2.666(6.7); 2.662(3.2); 2.607(0.4); 2.541(197.1);
2.524(22.8); 2.511(533.8); 2.506(1080.9); 2.502(1420.4); 2.497(1020.4); 2.493(488.2); 2.420(0.6); 2.403(0.5);
2.380(0.4); 2.367(1.0); 2.333(6.5); 2.328(9.1); 2.324(6.5); 2.289(1.0); 2.282(0.7); 2.266(0.5); 2.180(0.3);
2.075(1.2); 1.298(1.8); 1.258(2.5); 1.235(9.9); 0.889(0.4); 0.870(0.4); 0.854(1.1); 0.836(0.5); 0.008(1.4);
0.000(45.5); −0.008(1.6)
Example 1-49: ¹H-NMR(400.0 MHz, DMSO):
δ = 20.013(1.8); 8.680(5.9); 7.991(8.9); 7.971(10.4); 7.785(5.9); 7.766(7.9); 7.729(2.5); 7.710(6.7); 7.695(13.6);
7.676(9.3); 7.657(5.2); 7.638(6.4); 7.619(14.4); 7.522(5.8); 7.502(11.1); 7.465(10.2); 3.548(8.3); 3.533(8.8);
3.334(5269.3); 3.032(7.5); 3.014(12.7); 2.995(8.3); 2.670(16.0); 2.541(153.4); 2.502(2412.8); 2.329(15.3);
2.074(1.2); 1.298(2.6); 1.258(3.4); 1.235(15.9); 0.854(1.5); 0.000(40.5)
Example 1-50: ¹H-NMR(400.0 MHz, DMSO):
δ = 8.657(0.6); 8.644(1.2); 8.629(0.6); 7.780(1.2); 7.760(1.6); 7.728(0.5); 7.710(1.3); 7.692(1.0); 7.654(1.0);
7.634(1.2); 7.615(0.5); 7.498(2.6); 7.493(2.8); 7.470(1.5); 7.464(2.0); 7.451(1.4); 7.444(2.9); 7.391(1.9);
7.387(1.7); 7.371(1.1); 7.367(1.1); 3.544(0.7); 3.527(1.7); 3.511(1.7); 3.494(0.8); 3.384(0.4); 3.341(182.0);
3.337(196.2); 2.995(1.7); 2.977(2.6); 2.959(1.3); 2.676(0.4); 2.671(0.5); 2.667(0.4); 2.624(14.1); 2.542(24.2);
2.525(1.2); 2.511(29.9); 2.507(61.8); 2.502(82.0); 2.498(59.1); 2.493(28.3); 2.380(16.0); 2.334(0.4); 2.329(0.5);
2.324(0.4); 1.235(0.5); 0.000(2.0)
Example 1-51: ¹H-NMR(400.0 MHz, DMSO):
δ = 8.644(2.2); 8.631(4.2); 8.617(2.2); 8.296(7.2); 8.283(7.6); 7.846(11.2); 7.825(12.4); 7.781(4.7); 7.762(6.1);
7.726(2.5); 7.716(5.4); 7.708(8.1); 7.690(3.9); 7.652(3.7); 7.633(4.7); 7.614(1.8); 7.533(9.9); 7.443(16.0);
7.423(13.1); 3.531(2.7); 3.514(6.3); 3.499(6.3); 3.481(3.0); 3.374(0.3); 3.335(122.0); 2.998(0.9); 2.915(5.2);
2.897(9.3); 2.879(4.7); 2.672(0.5); 2.542(31.7); 2.507(55.2); 2.503(69.6); 2.499(52.5); 2.329(0.4); 0.000(1.1)
Example 1-52: ¹H-NMR(400.0 MHz, DMSO):
δ = 8.617(2.6); 8.604(4.9); 8.590(2.8); 8.179(0.3); 8.152(10.7); 8.137(1.3); 8.117(0.4); 7.783(5.1); 7.764(7.1);
7.753(1.3); 7.750(1.3); 7.747(1.3); 7.730(8.1); 7.726(14.0); 7.722(8.5); 7.708(6.8); 7.689(4.9); 7.653(4.3);
7.635(5.5); 7.616(2.5); 7.566(13.2); 7.546(16.0); 7.471(0.4); 7.442(6.0); 7.424(5.6); 7.285(13.9); 7.265(12.8);
6.976(0.4); 6.948(9.6); 6.945(9.5); 6.932(1.3); 3.876(0.3); 3.833(0.4); 3.818(0.5); 3.806(0.5); 3.792(0.5);
3.771(0.6); 3.763(0.6); 3.747(0.5); 3.737(0.5); 3.724(0.5); 3.707(0.7); 3.699(0.6); 3.662(0.8); 3.637(1.3);
3.627(1.2); 3.605(1.1); 3.589(1.6); 3.584(1.4); 3.561(2.2); 3.489(15.5); 3.411(3110.0); 3.392(392.5); 3.280(3.4);
3.249(2.0); 3.239(1.9); 3.224(2.0); 3.202(1.5); 3.176(1.0); 3.143(0.9); 3.122(0.8); 3.102(0.8); 3.084(0.7);
3.045(0.6); 3.023(0.5); 3.010(0.4); 2.998(3.6); 2.985(0.6); 2.914(0.4); 2.892(0.4); 2.839(6.4); 2.821(10.6);
2.803(6.5); 2.756(0.3); 2.718(0.6); 2.683(1.3); 2.678(1.7); 2.674(1.4); 2.623(0.4); 2.548(105.1); 2.531(19.0);
2.518(104.2); 2.514(187.9); 2.509(239.4); 2.505(178.8); 2.500(99.3); 2.418(0.4); 2.374(0.7); 2.361(0.3);
2.340(1.3); 2.336(1.6); 2.331(1.3); 2.312(0.4); 1.261(0.3); 1.237(0.8); 0.002(0.4)
Example 1-53: ¹H-NMR(400.0 MHz, DMSO):
δ = 8.875(8.9); 8.871(9.5); 8.645(2.2); 8.632(4.5); 8.610(8.4); 8.604(8.8); 8.243(9.1); 7.783(4.7); 7.766(16.0);
7.746(13.6); 7.730(2.6); 7.711(5.8); 7.692(4.2); 7.653(4.1); 7.634(5.3); 7.615(2.2); 7.505(0.5); 7.485(0.5);
7.450(5.8); 7.431(5.5); 7.420(13.0); 7.399(11.7); 7.239(0.6); 7.218(0.6); 3.523(2.9); 3.506(6.8); 3.491(6.9);
3.473(3.5); 3.339(1435.4); 2.995(3.2); 2.903(5.8); 2.885(10.0); 2.867(5.2); 2.789(0.6); 2.712(0.7); 2.671(2.8);
2.542(99.7); 2.502(401.4); 2.368(0.4); 2.329(2.5); 1.235(1.4); 0.000(1.4)
Example 1-54: ¹H-NMR(400.0 MHz, DMSO):
δ = 8.635(0.9); 8.622(1.7); 8.608(0.9); 8.449(2.7); 8.443(2.8); 7.982(1.5); 7.976(1.5); 7.960(1.5); 7.954(1.6);
7.782(1.7); 7.762(2.2); 7.727(0.8); 7.708(1.9); 7.689(1.4); 7.651(1.4); 7.632(1.8); 7.608(4.0); 7.588(4.4);
7.451(2.0); 7.433(1.8); 7.356(4.1); 7.336(3.6); 6.826(2.6); 6.805(2.5); 5.315(0.5); 5.299(1.2); 5.284(1.6);
5.268(1.2); 5.253(0.5); 3.506(1.0); 3.490(2.3); 3.473(2.3); 3.456(1.1); 3.341(233.6); 2.872(1.9); 2.854(3.2);
2.836(1.7); 2.672(0.5); 2.542(0.9); 2.503(66.4); 2.329(0.4); 1.320(16.0); 1.305(15.8)
Example 1-55: ¹H-NMR(400.0 MHz, DMSO):
δ = 8.641(2.4); 8.627(4.9); 8.613(2.5); 8.588(0.4); 8.573(0.6); 8.029(14.1); 8.008(16.0); 7.995(4.2); 7.993(5.5);
7.976(10.5); 7.974(11.1); 7.954(9.8); 7.945(0.6); 7.934(12.5); 7.915(5.3); 7.781(5.4); 7.761(7.0); 7.727(2.5);
7.709(6.3); 7.691(4.5); 7.651(4.6); 7.632(5.9); 7.614(2.2); 7.506(2.0); 7.490(0.7); 7.485(2.4); 7.479(0.4);
7.463(8.5); 7.461(9.7); 7.448(6.7); 7.444(10.1); 7.442(9.5); 7.430(6.0); 7.420(15.7); 7.399(14.3); 7.240(2.1);
7.219(1.9); 3.855(0.3); 3.839(0.3); 3.776(0.4); 3.711(0.5); 3.681(0.6); 3.665(0.7); 3.605(1.0); 3.589(1.1);
3.529(5.2); 3.512(9.3); 3.496(9.5); 3.478(7.5); 3.391(1226.2); 3.388(1172.8); 3.382(1096.2); 3.376(1340.9);
3.138(0.6); 3.098(0.5); 3.092(0.5); 3.049(0.5); 3.032(0.4); 2.996(8.1); 2.959(0.3); 2.907(6.1); 2.889(10.6);
2.871(5.5); 2.809(1.0); 2.790(1.7); 2.772(0.9); 2.714(2.0); 2.679(2.1); 2.674(2.8); 2.670(2.1); 2.665(1.0);
2.596(0.4); 2.592(0.5); 2.582(0.6); 2.579(0.6); 2.573(0.6); 2.564(1.6); 2.561(2.0); 2.544(540.2); 2.5342(2.9);
2.5335(2.8); 2.533(2.8); 2.532(2.8); 2.528(6.7); 2.523(10.7); 2.514(151.1); 2.510(305.9); 2.505(403.7);
2.500(291.2); 2.496(136.5); 2.442(0.5); 2.370(2.0); 2.341(1.0); 2.336(2.0); 2.332(2.7); 2.327(2.0); 2.323(1.0);
2.073(0.3); 1.234(0.9); 0.000(9.0)
Example 1-56: ¹H-NMR(400.0 MHz, DMSO):
δ = 20.012(0.9); 8.639(3.3); 8.435(3.7); 8.430(4.1); 8.423(4.1); 8.418(4.1); 7.880(3.6); 7.876(3.8); 7.861(4.4);
7.857(4.3); 7.782(3.5); 7.763(4.6); 7.721(1.5); 7.702(3.9); 7.684(2.9); 7.652(2.9); 7.633(3.6); 7.615(1.4);
7.540(4.1); 7.528(4.0); 7.521(4.0); 7.509(3.6); 7.455(6.4); 7.435(16.0); 7.420(4.2); 7.397(12.5); 7.377(6.4);
3.541(2.4); 3.524(4.9); 3.509(4.8); 3.491(2.7); 3.338(4723.8); 2.995(1.7); 2.913(4.6); 2.894(7.5); 2.876(4.1);
2.671(9.2); 2.541(23.6); 2.507(1043.0); 2.502(1330.7); 2.498(995.1); 2.329(8.5); 2.290(0.7); 1.298(1.0);
1.236(7.9); 0.854(1.1); 0.000(6.6)
Example 1-57: ¹H-NMR(400.0 MHz, DMSO):
δ = 8.896(9.6); 8.892(9.3); 8.651(2.9); 8.637(5.6); 8.624(3.0); 8.568(6.8); 8.557(7.0); 8.083(5.2); 8.063(5.6);
7.789(6.3); 7.769(8.3); 7.735(2.7); 7.716(7.7); 7.704(15.4); 7.684(16.0); 7.658(5.1); 7.639(6.6); 7.620(2.6);
7.500(4.9); 7.488(5.2); 7.481(5.0); 7.468(5.4); 7.460(7.8); 7.441(6.6); 7.416(15.6); 7.396(13.4); 3.529(3.8);
3.512(8.8); 3.496(8.9); 3.479(4.2); 3.445(0.6); 3.350(782.1); 3.001(1.4); 2.904(7.1); 2.885(12.5); 2.867(6.4);
2.718(0.6); 2.677(1.5); 2.547(84.8); 2.511(202.3); 2.508(213.2); 2.374(0.5); 2.335(1.4); 1.240(0.6); 1.075(0.4)
Example 1-58: ¹H-NMR(400.0 MHz, DMSO):
δ = 8.639(2.2); 8.625(4.4); 8.611(2.2); 8.510(7.3); 8.504(7.5); 8.123(4.1); 8.117(4.0); 8.101(4.3); 8.095(4.3);
7.782(4.4); 7.762(5.8); 7.727(1.9); 7.709(4.9); 7.690(3.5); 7.651(13.5); 7.631(16.0); 7.614(1.9); 7.452(5.2);
7.433(4.6); 7.380(11.1); 7.360(9.8); 7.087(7.1); 7.065(7.0); 5.075(3.3); 5.052(10.3); 5.029(10.6); 5.007(3.7);
3.512(2.5); 3.495(5.7); 3.478(5.8); 3.462(2.8); 3.385(0.5); 3.334(412.2); 2.996(2.3); 2.882(4.8); 2.864(8.2);
2.846(4.4); 2.711(0.6); 2.671(1.1); 2.541(107.5); 2.502(163.0); 2.368(0.6); 2.329(1.1); 1.234(0.5); 0.000(2.3)
Example 1-59: ¹H-NMR(400.0 MHz, DMSO):
δ = 8.621(0.8); 8.608(1.5); 8.594(0.8); 7.780(1.6); 7.760(2.1); 7.723(0.7); 7.705(1.8); 7.687(1.3); 7.650(1.3);
7.631(1.7); 7.612(0.6); 7.562(3.1); 7.557(3.3); 7.446(1.9); 7.427(1.7); 7.399(3.2); 7.378(5.2); 7.310(4.8);
7.290(3.2); 6.687(3.2); 6.683(3.3); 3.496(1.1); 3.479(2.3); 3.461(2.3); 3.445(1.3); 3.429(0.5); 3.356(426.6);
3.352(221.4); 3.270(0.3); 2.995(1.0); 2.849(1.9); 2.830(3.2); 2.812(1.7); 2.672(0.5); 2.668(0.4); 2.542(46.5);
2.507(58.1); 2.503(74.9); 2.499(59.0); 2.422(16.0); 2.400(0.3); 2.330(0.5); 2.326(0.4); 0.000(0.4)
Example 1-60: ¹H-NMR(400.0 MHz, DMSO):
δ = 8.608(0.8); 8.595(1.5); 8.581(0.8); 8.093(4.8); 7.823(4.9); 7.779(1.7); 7.759(2.2); 7.722(0.7); 7.702(1.9);
7.684(1.4); 7.648(1.4); 7.629(1.7); 7.610(0.7); 7.504(3.9); 7.484(4.6); 7.441(2.0); 7.422(1.8); 7.243(4.3);
7.223(3.7); 3.852(16.0); 3.475(1.0); 3.458(2.3); 3.441(2.3); 3.424(1.2); 3.394(0.5); 3.339(362.8); 2.995(1.8);
2.814(1.9); 2.795(3.2); 2.777(1.7); 2.712(0.5); 2.671(0.7); 2.542(103.1); 2.506(80.0); 2.502(100.7); 2.498(78.5);
2.368(0.5); 2.329(0.7); 0.000(0.8)
Example 1-61: ¹H-NMR(400.0 MHz, DMSO):
δ = 20.011(0.4); 8.622(1.5); 8.452(1.8); 8.445(1.8); 8.440(1.8); 7.780(1.5); 7.760(2.1); 7.720(0.7); 7.703(1.8);
7.686(1.2); 7.651(1.3); 7.632(1.7); 7.614(0.7); 7.592(1.7); 7.577(1.8); 7.573(1.9); 7.438(1.9); 7.419(1.6);
7.378(1.1); 7.357(8.3); 7.350(9.4); 7.329(1.4); 7.308(1.4); 7.298(1.5); 7.290(1.5); 7.278(1.3); 3.545(1.1);
3.527(2.3); 3.510(2.3); 3.494(1.2); 3.458(0.7); 3.337(2730.0); 2.995(1.2); 2.904(2.1); 2.886(3.5); 2.867(1.9);
2.671(5.6); 2.667(4.4); 2.541(25.2); 2.537(8.6); 2.506(638.8); 2.502(833.1); 2.498(660.0); 2.437(16.0);
2.329(5.4); 2.324(4.3); 2.290(0.5); 1.297(0.4); 1.235(2.3); 0.000(4.3)
Example 1-62: ¹H-NMR(400.0 MHz, DMSO):
δ = 8.647(2.6); 8.634(5.1); 8.620(2.7); 8.551(7.9); 8.547(8.0); 8.308(2.4); 8.302(2.4); 8.288(4.7); 8.282(4.5);
8.267(2.7); 8.261(2.5); 7.788(5.4); 7.769(7.2); 7.734(2.3); 7.716(6.2); 7.694(16.0); 7.674(14.8); 7.659(4.8);
7.639(5.7); 7.620(2.2); 7.457(6.4); 7.439(5.7); 7.410(14.0); 7.390(12.2); 7.296(4.7); 7.289(4.8); 7.275(4.6);
7.268(4.5); 3.524(3.2); 3.507(7.5); 3.491(7.7); 3.474(3.7); 3.414(1.1); 3.346(1160.7); 3.270(1.0); 3.201(0.3);
3.001(1.9); 2.899(6.2); 2.881(10.9); 2.863(5.6); 2.718(0.5); 2.677(2.3); 2.547(84.3); 2.512(298.0); 2.508(351.2);
2.374(0.5); 2.335(2.3); 1.813(2.3); 1.793(3.9); 1.760(3.5); 1.669(1.6); 1.304(1.6); 1.275(3.0); 1.245(3.8);
1.221(3.7); 1.198(2.1); 0.005(0.6)
Example 1-63: ¹H-NMR(400.0 MHz, DMSO):
δ = 8.618(0.7); 8.605(1.5); 8.591(0.8); 7.776(1.6); 7.757(2.1); 7.718(0.7); 7.700(1.8); 7.682(1.3); 7.648(1.3);
7.629(1.6); 7.611(0.7); 7.434(1.9); 7.415(1.7); 7.396(2.2); 7.375(5.9); 7.351(5.6); 7.330(2.4); 3.512(0.9);
3.495(2.2); 3.479(2.3); 3.462(1.1); 3.335(442.3); 2.995(2.0); 2.870(1.9); 2.852(3.3); 2.834(1.7); 2.671(1.2);
2.612(14.6); 2.541(39.3); 2.502(166.3); 2.371(16.0); 2.329(1.1); 1.235(0.5); 0.000(1.8)
Example 1-64: ¹H-NMR(400.0 MHz, DMSO):
δ = 8.795(0.6); 8.782(0.6); 8.640(2.9); 8.627(5.1); 8.613(2.7); 8.583(0.7); 8.569(1.2); 8.555(1.0); 8.489(0.8);
8.458(8.9); 8.445(9.3); 8.345(0.9); 8.253(0.5); 8.238(0.5); 7.979(0.9); 7.969(0.4); 7.959(1.0); 7.854(12.5);
7.837(13.1); 7.817(14.5); 7.780(6.1); 7.764(10.2); 7.757(10.1); 7.726(2.9); 7.707(7.1); 7.688(5.1); 7.650(5.0);
7.632(6.5); 7.613(2.5); 7.505(2.3); 7.485(2.7); 7.470(1.1); 7.437(16.0); 7.417(13.7); 7.238(2.6); 7.217(2.3);
3.526(3.7); 3.509(8.0); 3.494(7.8); 3.476(3.7); 3.453(1.7); 3.437(1.7); 3.420(0.9); 3.330(716.8); 3.254(0.4);
2.995(0.4); 2.933(0.6); 2.911(6.6); 2.892(11.2); 2.875(5.5); 2.807(1.2); 2.789(2.2); 2.771(1.1); 2.671(2.7);
2.541(26.7); 2.502(405.0); 2.328(2.6); 1.299(0.3); 1.258(0.5); 1.235(1.4); 0.000(6.1)
Example 1-65: ¹H-NMR(400.0 MHz, DMSO):
δ = 8.724(0.3); 8.679(3.5); 8.665(6.9); 8.651(3.4); 8.618(0.4); 8.605(0.4); 8.480(13.0); 8.467(13.6); 8.317(1.0);
8.055(0.4); 8.051(0.5); 8.004(15.5); 7.999(16.0); 7.936(15.2); 7.933(15.6); 7.882(0.3); 7.842(7.5); 7.838(7.1);
7.820(14.6); 7.817(14.6); 7.807(9.4); 7.803(8.8); 7.783(7.5); 7.764(9.7); 7.734(3.2); 7.716(8.2); 7.697(5.8);
7.656(5.8); 7.637(7.5); 7.618(3.0); 7.590(0.4); 7.570(0.4); 7.543(13.1); 7.523(11.6); 7.475(8.5); 7.457(7.4);
6.896(0.4); 5.757(15.7); 3.556(4.1); 3.539(9.9); 3.524(10.2); 3.507(4.6); 3.326(311.2); 3.285(0.4); 3.032(8.3);
3.014(14.9); 2.997(7.2); 2.676(1.7); 2.671(2.3); 2.667(1.7); 2.524(7.0); 2.511(127.9); 2.507(249.7); 2.502(325.9);
2.498(238.1); 2.493(116.4); 2.333(1.5); 2.329(2.1); 2.325(1.5); 1.989(0.4); 1.422(1.3); 1.414(1.6); 1.407(1.9);
1.398(0.6); 1.351(3.4); 1.336(2.0); 1.313(0.4); 1.298(2.0); 1.259(3.2); 1.250(3.6); 1.234(7.7); 1.193(0.6);
1.187(0.7); 1.175(0.7); 1.157(0.5); 1.105(0.4); 0.868(0.8); 0.853(1.4); 0.834(1.0); 0.812(0.7); 0.755(0.3);
0.008(1.9); 0.000(48.0); −0.008(1.7)
Example 1-66: ¹H-NMR(400.0 MHz, DMSO):
δ = 8.785(12.6); 8.779(12.9); 8.681(3.5); 8.667(7.1); 8.653(3.5); 8.313(0.6); 8.226(8.4); 8.219(8.1); 8.205(9.0);
8.198(8.9); 7.882(15.5); 7.877(16.0); 7.784(7.3); 7.765(9.7); 7.735(3.3); 7.723(8.6); 7.718(14.1); 7.703(10.4);
7.698(14.2); 7.658(5.8); 7.638(7.6); 7.624(14.5); 7.603(12.5); 7.521(13.5); 7.501(11.3); 7.480(8.6); 7.462(7.4);
5.754(2.7); 3.549(4.5); 3.532(10.0); 3.516(10.1); 3.499(5.0); 3.468(0.9); 3.451(1.1); 3.357(1210.0); 3.269(0.8);
3.259(0.6); 3.021(8.3); 3.003(14.4); 2.985(7.2); 2.944(0.3); 2.678(1.0); 2.674(1.3); 2.669(0.9); 2.527(4.2);
2.513(72.8); 2.509(140.2); 2.505(181.5); 2.500(132.9); 2.496(65.3); 2.336(0.9); 2.331(1.2); 2.327(0.8);
1.352(0.6); 1.299(0.4); 1.259(0.7); 1.250(0.5); 1.233(1.9); 0.008(0.9); 0.000(21.0); −0.008(0.8)
Example 1-67: 1H-NMR(400.0 MHz, DMSO):
δ = 9.208(2.6); 9.192(5.7); 9.177(2.6); 8.844(9.6); 8.838(9.6); 8.795(5.5); 8.783(5.4); 8.317(3.1); 8.289(6.5);
8.283(6.3); 8.268(6.9); 8.262(7.0); 8.058(9.2); 8.054(10.1); 7.902(4.3); 7.898(4.2); 7.881(5.9); 7.877(6.2);
7.871(4.5); 7.868(4.6); 7.851(6.4); 7.848(6.5); 7.786(6.0); 7.774(16.0); 7.766(4.5); 7.754(11.2); 7.673(10.3);
7.652(9.7); 4.288(2.2); 4.272(2.3); 4.252(5.1); 4.236(5.0); 4.215(2.6); 4.200(2.5); 4.038(0.9); 4.020(0.9);
3.374(0.4); 3.327(896.8); 3.304(2.4); 3.290(0.6); 2.680(1.2); 2.676(2.7); 2.671(3.8); 2.667(2.8); 2.525(7.6);
2.520(11.6); 2.511(197.0); 2.507(417.4); 2.502(564.0); 2.498(413.1); 2.493(201.7); 2.333(2.6); 2.329(3.7);
2.324(2.8); 2.320(1.3); 1.989(3.8); 1.259(0.4); 1.234(0.7); 1.193(1.1); 1.175(2.1); 1.157(1.0); 0.000(2.4)
Example 1-68: 1H-NMR(400.0 MHz, DMSO):
δ = 9.045(2.7); 9.029(6.0); 9.014(2.8); 8.842(10.2); 8.836(10.2); 8.317(0.4); 8.287(6.9); 8.280(6.7); 8.266(7.4);
8.259(7.5); 8.049(10.0); 8.045(10.8); 7.900(4.5); 7.896(4.5); 7.879(6.3); 7.875(6.4); 7.771(16.0); 7.750(15.6);
7.726(2.4); 7.709(6.2); 7.690(4.6); 7.668(10.9); 7.657(5.0); 7.647(10.7); 7.638(5.8); 7.619(2.0); 7.388(6.5);
7.370(5.9); 4.264(2.4); 4.248(2.5); 4.228(5.5); 4.212(5.3); 4.192(2.8); 4.176(2.6); 4.056(0.4); 4.038(1.2);
4.020(1.2); 4.003(0.4); 3.330(170.1); 2.676(0.6); 2.672(0.8); 2.668(0.6); 2.525(1.9); 2.520(2.9); 2.512(43.4);
2.507(89.9); 2.503(119.7); 2.498(87.4); 2.494(42.5); 2.334(0.5); 2.330(0.7); 2.325(0.6); 1.990(5.4); 1.397(3.7);
1.234(1.0); 1.193(1.5); 1.175(2.9); 1.158(1.4); 0.000(0.6)
Example 1-69: 1H-NMR(400.0 MHz, DMSO):
δ = 9.381(13.2); 9.039(3.0); 9.024(6.1); 9.008(2.9); 8.316(1.3); 8.306(16.0); 8.207(9.7); 8.203(10.2); 8.059(4.9);
8.054(4.6); 8.037(5.9); 8.032(5.6); 7.832(10.0); 7.810(8.4); 7.764(5.9); 7.745(8.0); 7.722(2.6); 7.703(6.7);
7.685(4.9); 7.653(5.0); 7.634(5.9); 7.616(2.1); 7.598(0.4); 7.582(0.4); 7.578(0.4); 7.488(0.5); 7.389(6.9);
7.370(6.1); 7.016(0.5); 6.995(0.5); 6.919(0.5); 5.757(1.5); 4.255(2.6); 4.240(2.8); 4.220(5.8); 4.204(5.6);
4.184(3.0); 4.169(2.7); 3.329(217.7); 2.676(1.0); 2.672(1.4); 2.507(152.3); 2.503(195.0); 2.499(147.9);
2.329(1.2); 1.989(0.5); 1.234(0.5); 0.000(15.4)
Example 1-70: 1H-NMR (400.0 MHz, CD3CN):
□ = 9.027 (8.8); 9.022 (8.9); 8.263 (4.8); 8.258 (4.9); 8.243 (5.3); 8.238 (5.5); 7.895 (10.5); 7.875 (9.6); 7.861
(12.4); 7.790 (4.5); 7.786 (3.9); 7.770 (10.2); 7.766 (13.5); 7.744 (8.3); 7.736 (16.0); 7.716 (7.1); 7.692 (2.4);
7.674 (6.7); 7.656 (5.4); 7.630 (5.0); 7.612 (6.0); 7.593 (2.3); 7.487 (7.3); 7.468 (6.0); 7.211 (2.9); 6.146 (2.5);
6.138 (2.7); 6.129 (2.8); 6.121 (2.6); 6.029 (2.5); 6.021 (2.7); 6.012 (2.9); 6.004 (2.6); 5.447 (0.3); 4.031 (1.2);
4.022 (1.3); 4.015 (1.3); 4.007 (1.3); 3.994 (2.0); 3.986 (2.1); 3.979 (2.0); 3.971 (2.0); 3.962 (1.4); 3.954 (1.2);
3.947 (1.4); 3.938 (1.3); 3.925 (2.1); 3.917 (2.1); 3.910 (2.2); 3.902 (2.0); 3.885 (1.9); 3.869 (2.9); 3.851 (2.3);
3.830 (3.5); 3.814 (4.0); 3.796 (2.2); 3.777 (1.8); 3.760 (1.2); 2.473 (0.5); 2.468 (1.0); 2.463 (1.3); 2.458 (1.0);
2.453 (0.5); 2.156 (318.6); 2.120 (1.6); 2.114 (1.9); 2.107 (2.4); 2.101 (1.6); 2.095 (0.9); 1.964 (10.5); 1.958
(26.6); 1.952 (141.2); 1.946 (254.9); 1.940 (341.3); 1.934 (238.2); 1.928 (125.4); 1.781 (0.9); 1.775 (1.5); 1.768
(2.0); 1.762 (1.4); 1.756 (0.8); 1.437 (0.5); 1.372 (0.4); 1.340 (0.6); 1.285 (1.0); 1.270 (3.0); 0.900 (0.4); 0.881
(0.6); 0.858 (0.5); 0.842 (0.4); 0.146 (2.7); 0.008 (22.3); 0.000 (585.7); −0.009 (27.3); −0.150 (2.7)
Example 1-76: 1H-NMR (400.0 MHz, d6-DMSO):
□ = 10.327 (0.5); 9.028 (0.4); 9.011 (0.5); 8.505 (0.4); 8.488 (0.4); 4.056 (1.3); 4.038 (3.7); 4.020 (3.8); 4.002
(1.3); 3.888 (0.3); 3.869 (0.4); 3.851 (0.4); 3.531 (0.3); 3.503 (0.3); 3.449 (0.3); 3.432 (0.3); 3.173 (0.5); 2.515
(5.2); 2.511 (10.7); 2.506 (14.5); 2.502 (11.1); 2.498 (5.7); 1.989 (16.0); 1.910 (7.9); 1.212 (0.6); 1.200 (0.4);
1.193 (4.9); 1.183 (0.7); 1.175 (8.9); 1.165 (0.4); 1.158 (4.3); 1.073 (0.3); 1.056 (0.7); 1.038 (0.3); 0.000 (5.8)
Example 1-71: 1H-NMR (400.0 MHz, CD3CN):
□ = 9.029 (10.1); 9.024 (10.2); 8.759 (7.1); 8.747 (7.2); 8.279 (0.5); 8.265 (5.6); 8.260 (5.7); 8.244 (6.2); 8.240
(6.1); 7.897 (11.5); 7.876 (13.6); 7.868 (12.1); 7.864 (14.5); 7.854 (8.8); 7.793 (5.2); 7.789 (4.7); 7.773 (9.8);
7.769 (9.4); 7.730 (16.0); 7.710 (8.2); 7.673 (6.5); 7.662 (6.6); 7.654 (5.7); 7.642 (5.4); 7.589 (0.7); 7.492 (0.4);
7.480 (0.4); 7.461 (0.3); 7.331 (3.4); 6.149 (2.7); 6.140 (3.1); 6.131 (3.1); 6.123 (3.0); 6.032 (2.8); 6.023 (3.0);
6.014 (3.1); 6.006 (2.9); 5.823 (1.0); 5.706 (1.0); 5.417 (0.4); 4.067 (0.4); 4.049 (1.6); 4.041 (1.4); 4.034 (1.5);
4.025 (1.4); 4.012 (2.2); 4.004 (2.3); 3.997 (2.4); 3.989 (2.2); 3.980 (1.4); 3.972 (1.4); 3.965 (1.5); 3.957 (1.4);
3.943 (2.3); 3.935 (2.4); 3.928 (2.4); 3.920 (2.2); 3.897 (5.2); 3.881 (3.3); 3.865 (2.4); 3.843 (3.4); 3.826 (4.0);
3.809 (2.3); 3.789 (2.0); 3.772 (1.3); 3.361 (0.9); 3.170 (1.9); 2.711 (0.8); 2.693 (1.3); 2.675 (0.9); 2.467 (3.1);
2.463 (4.2); 2.458 (3.1); 2.158 (746.2); 2.120 (3.2); 2.114 (4.4); 2.107 (5.5); 2.101 (4.0); 2.095 (2.3); 2.071 (0.6);
2.049 (0.4); 1.964 (24.7); 1.958 (63.7); 1.952 (335.0); 1.946 (611.8); 1.940 (827.6); 1.934 (583.6); 1.928 (310.5);
1.806 (0.4); 1.781 (2.1); 1.775 (3.5); 1.768 (4.9); 1.762 (3.5); 1.756 (2.0); 1.705 (0.4); 1.687 (0.4); 1.669 (0.5);
1.652 (0.8); 1.634 (0.7); 1.615 (0.4); 1.559 (0.4); 1.539 (0.4); 1.372 (0.6); 1.359 (0.6); 1.339 (0.7); 1.277 (11.9);
1.229 (0.3); 1.222 (0.5); 1.204 (0.8); 1.186 (0.5); 0.899 (1.0); 0.882 (2.8); 0.864 (1.4); 0.841 (0.4); 0.146 (5.8);
0.008 (45.5); 0.000 (1261.0); −0.008 (66.5); −0.083 (0.4); −0.112 (0.3); −0.150 (5.9)
Example 1-72: 1H-NMR (400.0 MHz, CD3CN):
□ = 9.014 (6.8); 9.010 (6.8); 8.840 (16.0); 8.838 (15.9); 8.831 (4.0); 8.252 (3.7); 8.247 (3.7); 8.231 (4.1); 8.227
(4.1); 7.927 (2.3); 7.894 (7.8); 7.873 (6.9); 7.854 (9.3); 7.759 (2.6); 7.755 (2.2); 7.739 (7.7); 7.735 (7.5); 7.719
(11.9); 7.699 (3.7); 6.172 (1.8); 6.164 (2.0); 6.154 (2.1); 6.146 (1.9); 6.055 (1.9); 6.047 (2.0); 6.037 (2.1); 6.028
(1.9); 5.448 (1.4); 4.084 (0.9); 4.075 (0.9); 4.068 (1.0); 4.060 (1.0); 4.047 (1.5); 4.039 (1.5); 4.032 (1.5); 4.023
(1.4); 4.014 (0.9); 4.006 (0.9); 3.999 (0.9); 3.990 (0.9); 3.977 (1.5); 3.969 (1.5); 3.962 (1.6); 3.954 (1.4); 3.938
(1.4); 3.921 (2.0); 3.904 (1.8); 3.886 (2.6); 3.868 (2.8); 3.851 (1.8); 3.832 (1.3); 3.815 (0.8); 2.469 (0.6); 2.464
(0.9); 2.459 (0.7); 2.455 (0.4); 2.159 (498.2); 2.120 (1.2); 2.114 (1.5); 2.108 (1.8); 2.102 (1.2); 2.095 (0.7); 1.964
(8.6); 1.958 (22.7); 1.953 (104.3); 1.947 (185.7); 1.940 (245.6); 1.934 (171.7); 1.928 (90.2); 1.781 (0.6); 1.775
(1.1); 1.769 (1.5); 1.763 (1.1); 1.756 (0.6); 1.673 (0.3); 1.437 (1.4); 1.299 (0.3); 1.271 (1.7); 1.204 (0.3); 0.881
(0.5); 0.146 (1.6); 0.000 (346.7); −0.008 (18.6); −0.150 (1.7)
Example 1-75: 1H-NMR (400.0 MHz, d6-DMSO):
□ = 9.007 (3.5); 8.992 (7.0); 8.978 (3.5); 8.489 (7.2); 8.485 (7.9); 8.478 (7.8); 8.473 (7.8); 8.316 (0.3); 7.955 (7.2);
7.951 (7.6); 7.936 (8.2); 7.932 (8.1); 7.797 (7.1); 7.778 (9.4); 7.748 (3.1); 7.730 (8.2); 7.719 (10.5); 7.712 (6.8);
7.699 (14.2); 7.688 (14.7); 7.675 (6.2); 7.656 (7.2); 7.637 (2.7); 7.610 (8.5); 7.606 (8.1); 7.590 (6.2); 7.586 (6.2);
7.576 (7.8); 7.564 (7.6); 7.557 (7.4); 7.545 (6.9); 7.497 (8.4); 7.478 (7.2); 6.097 (2.2); 6.087 (2.5); 6.079 (2.9);
6.069 (2.3); 5.979 (2.3); 5.963 (3.4); 5.951 (2.3); 5.756 (8.9); 4.056 (1.3); 4.038 (3.8); 4.020 (3.9); 4.003 (1.3);
3.886 (0.4); 3.875 (0.7); 3.862 (0.7); 3.850 (2.2); 3.836 (4.2); 3.827 (2.7); 3.819 (3.4); 3.802 (2.3); 3.782 (4.8);
3.767 (6.3); 3.751 (2.5); 3.730 (0.4); 3.568 (2.5); 3.324 (66.3); 2.675 (0.6); 2.671 (0.8); 2.506 (103.6); 2.502
(135.1); 2.498 (104.9); 2.329 (0.9); 2.325 (0.7); 1.989 (16.0); 1.397 (4.4); 1.299 (0.6); 1.259 (1.0); 1.235 (4.0);
1.193 (4.4); 1.175 (8.5); 1.158 (4.3); 0.868 (0.3); 0.853 (0.6); 0.836 (0.4); 0.000 (33.9)
Example 1-74: 1H-NMR (400.0 MHz, d6-DMSO):
□ = 9.920 (0.5); 8.985 (3.8); 8.971 (7.9); 8.957 (4.0); 8.646 (11.1); 8.640 (11.8); 8.406 (3.8); 8.400 (3.8); 8.386
(6.5); 8.379 (6.4); 8.365 (4.1); 8.359 (4.0); 8.316 (0.5); 7.943 (15.3); 7.848 (6.8); 7.844 (6.5); 7.828 (9.1); 7.824
(9.0); 7.795 (7.9); 7.776 (10.5); 7.753 (3.5); 7.734 (8.8); 7.709 (16.0); 7.689 (11.7); 7.674 (6.6); 7.654 (8.0); 7.635
(3.1); 7.502 (9.3); 7.483 (8.1); 7.336 (7.0); 7.329 (7.2); 7.315 (7.0); 7.308 (6.9); 6.082 (2.5); 6.073 (2.8); 6.064
(3.2); 6.055 (2.7); 5.964 (2.5); 5.953 (2.9); 5.948 (3.6); 5.937 (2.6); 4.038 (0.7); 4.020 (0.8); 3.868 (0.6); 3.858
(0.8); 3.855 (0.8); 3.844 (0.9); 3.832 (2.3); 3.819 (3.1); 3.811 (3.4); 3.796 (3.4); 3.778 (2.7); 3.760 (4.4); 3.752
(4.7); 3.744 (5.8); 3.728 (2.5); 3.708 (0.6); 3.692 (0.5); 3.325 (125.0); 2.676 (0.9); 2.672 (1.2); 2.667 (0.9); 2.507
(147.4); 2.502 (193.9); 2.498 (148.1); 2.334 (0.9); 2.329 (1.3); 2.325 (1.0); 1.989 (3.2); 1.398 (2.3); 1.234 (0.9);
1.193 (0.9); 1.175 (1.7); 1.157 (0.8); 0.008 (1.9); 0.000 (47.2)
Example 1-73: 1H-NMR (400.0 MHz, d6-DMSO):
□ = 10.221 (0.4); 8.985 (1.7); 8.971 (3.5); 8.956 (1.7); 8.824 (6.0); 8.818 (6.1); 8.267 (4.0); 8.261 (3.9); 8.246
(4.3); 8.240 (4.4); 7.968 (6.6); 7.923 (0.4); 7.917 (0.4); 7.869 (3.1); 7.865 (2.9); 7.849 (4.0); 7.845 (3.9); 7.820
(0.3); 7.795 (3.4); 7.775 (4.5); 7.751 (1.5); 7.734 (3.8); 7.717 (8.9); 7.696 (5.3); 7.673 (2.8); 7.654 (9.7); 7.633
(7.1); 7.535 (0.3); 7.514 (0.4); 7.500 (4.0); 7.481 (3.5); 7.287 (0.5); 7.250 (0.4); 7.242 (0.4); 6.083 (1.1); 6.074
(1.2); 6.065 (1.4); 6.056 (1.1); 5.965 (1.1); 5.955 (1.2); 5.949 (1.5); 5.938 (1.1); 4.056 (1.2); 4.038 (3.6); 4.020
(3.6); 4.003 (1.2); 3.858 (0.4); 3.855 (0.4); 3.845 (0.4); 3.832 (1.0); 3.822 (1.3); 3.810 (1.6); 3.795 (1.5); 3.777
(1.3); 3.764 (1.5); 3.758 (2.0); 3.754 (2.0); 3.742 (2.5); 3.727 (1.0); 3.707 (0.4); 3.569 (1.8); 3.326 (42.7); 2.676
(0.4); 2.672 (0.6); 2.667 (0.5); 2.525 (1.5); 2.511 (33.5); 2.507 (69.2); 2.503 (92.7); 2.498 (69.3); 2.494 (35.3);
2.334 (0.4); 2.329 (0.6); 2.325 (0.5); 1.989 (16.0); 1.397 (0.4); 1.234 (0.8); 1.193 (4.3); 1.175 (8.5); 1.158 (4.2);
0.008 (1.1); 0.000 (33.3); −0.008 (1.4)
Example 1-77: 1H-NMR (400.0 MHz, d6-DMSO):
δ = 10.453 (1.6); 8.990 (2.9); 8.975 (5.8); 8.961 (3.0); 8.948 (13.8); 8.943 (14.0); 8.797 (0.7); 8.794 (0.8); 8.678
(12.8); 8.672 (13.3); 8.637 (0.6); 8.631 (0.6); 8.548 (2.0); 8.377 (8.8); 8.371 (15.2); 8.366 (8.5); 8.316 (0.7); 8.141
(0.4); 8.138 (0.4); 8.135 (0.4); 8.131 (0.4); 8.102 (0.9); 8.096 (0.9); 8.072 (0.9); 8.067 (0.9); 8.027 (11.3); 7.922
(5.6); 7.918 (5.2); 7.902 (6.8); 7.897 (6.6); 7.795 (5.8); 7.776 (7.7); 7.753 (2.5); 7.735 (6.5); 7.717 (16.0); 7.697
(9.8); 7.674 (4.6); 7.655 (5.8); 7.636 (2.2); 7.501 (6.9); 7.483 (5.9); 7.333 (1.0); 7.270 (0.7); 7.264 (1.1); 7.258
(0.6); 6.529 (0.5); 6.087 (1.9); 6.078 (2.1); 6.069 (2.4); 6.060 (1.9); 5.969 (1.9); 5.958 (2.1); 5.953 (2.6); 5.942
(1.9); 5.756 (5.9); 3.872 (0.5); 3.862 (0.6); 3.858 (0.6); 3.848 (0.6); 3.836 (1.8); 3.826 (2.2); 3.822 (2.1); 3.814
(2.5); 3.799 (2.4); 3.781 (2.0); 3.768 (2.5); 3.762 (3.2); 3.757 (3.2); 3.746 (4.3); 3.731 (1.8); 3.710 (0.4); 3.324
(116.6); 2.680 (0.4); 2.676 (0.9); 2.671 (1.3); 2.667 (1.0); 2.662 (0.5); 2.525 (3.0); 2.520 (4.8); 2.511 (71.5); 2.507
(148.6); 2.502 (199.1); 2.498 (144.4); 2.493 (69.6); 2.338 (0.4); 2.334 (0.9); 2.329 (1.3); 2.325 (0.9); 1.989 (0.5);
1.259 (0.4); 1.234 (0.8); 0.857 (0.5); 0.839 (0.8); 0.000 (0.7)
Example 1-78: 1H-NMR (400.0 MHz, d6-DMSO):
□ = 8.984 (2.4); 8.969 (4.8); 8.955 (2.4); 8.350 (8.5); 8.336 (9.1); 8.316 (0.5); 8.074 (9.4); 7.982 (4.7); 7.977 (4.4);
7.961 (5.5); 7.957 (5.4); 7.930 (1.8); 7.915 (1.8); 7.803 (3.4); 7.798 (6.2); 7.794 (7.9); 7.791 (7.0); 7.786 (5.8);
7.781 (3.8); 7.773 (6.7); 7.750 (2.4); 7.742 (9.8); 7.733 (5.9); 7.722 (8.7); 7.714 (4.3); 7.672 (4.0); 7.654 (14.8);
7.634 (1.9); 7.495 (5.8); 7.477 (5.0); 6.733 (0.6); 6.728 (1.0); 6.724 (0.7); 6.718 (0.6); 6.714 (1.0); 6.709 (0.6);
6.405 (1.7); 6.400 (1.8); 6.094 (1.6); 6.085 (1.7); 6.077 (2.0); 6.067 (1.6); 5.976 (1.6); 5.966 (1.8); 5.960 (2.2);
5.949 (1.6); 5.756 (16.0); 4.331 (0.8); 4.038 (0.4); 4.020 (0.4); 3.877 (0.4); 3.868 (0.5); 3.864 (0.5); 3.854 (0.5);
3.841 (1.5); 3.831 (2.0); 3.828 (1.9); 3.821 (2.2); 3.805 (2.2); 3.788 (1.7); 3.774 (2.0); 3.768 (2.8); 3.764 (2.7);
3.752 (3.8); 3.737 (1.5); 3.716 (0.4); 3.701 (0.3); 3.325 (66.2); 2.676 (0.7); 2.672 (0.9); 2.667 (0.7); 2.525 (2.0);
2.520 (3.2); 2.511 (51.4); 2.507 (106.9); 2.503 (143.7); 2.498 (105.5); 2.494 (52.0); 2.334 (0.7); 2.329 (0.9); 2.325
(0.7); 1.989 (1.6); 1.398 (0.5); 1.259 (0.5); 1.234 (1.3); 1.193 (0.5); 1.175 (0.9); 1.158 (0.5); 0.000 (0.4)
Example 1-80: 1H-NMR (400.0 MHz, d6-DMSO):
□ = 9.180 (2.7); 9.165 (5.5); 9.151 (2.7); 8.813 (5.9); 8.803 (5.7); 8.801 (5.9); 8.491 (7.1); 8.486 (7.7); 8.479 (7.6);
8.474 (7.7); 8.316 (0.9); 7.973 (5.3); 7.955 (13.1); 7.950 (9.9); 7.936 (8.3); 7.931 (7.9); 7.806 (5.5); 7.795 (5.4);
7.787 (4.7); 7.775 (4.5); 7.721 (8.5); 7.701 (12.6); 7.696 (8.8); 7.693 (11.7); 7.612 (7.3); 7.608 (6.8); 7.592 (5.3);
7.588 (5.3); 7.578 (8.0); 7.566 (7.6); 7.559 (7.4); 7.547 (7.3); 6.097 (1.8); 6.089 (2.0); 6.079 (2.2); 6.070 (1.9);
5.979 (1.9); 5.970 (2.0); 5.962 (2.4); 5.952 (1.9); 5.756 (16.0); 4.056 (0.6); 4.038 (1.7); 4.020 (1.7); 4.002 (0.6);
3.923 (0.5); 3.915 (0.7); 3.910 (0.7); 3.900 (0.7); 3.887 (1.5); 3.878 (1.8); 3.874 (1.7); 3.865 (1.5); 3.855 (1.6);
3.839 (2.5); 3.830 (1.0); 3.817 (2.5); 3.803 (4.1); 3.793 (2.1); 3.786 (3.1); 3.769 (1.6); 3.750 (0.6); 3.733 (0.4);
3.323 (108.6); 2.680 (0.8); 2.676 (1.7); 2.671 (2.4); 2.667 (1.8); 2.662 (0.9); 2.525 (6.4); 2.520 (9.8); 2.511
(133.9); 2.507 (274.4); 2.502 (363.5); 2.498 (266.6); 2.493 (131.8); 2.338 (0.7); 2.333 (1.6); 2.329 (2.3); 2.324
(1.7); 2.086 (0.5); 1.989 (7.6); 1.398 (0.4); 1.298 (1.0); 1.259 (1.6); 1.235 (3.0); 1.193 (2.1); 1.175 (4.1); 1.157
(2.0); 0.854 (0.4); 0.146 (1.6); 0.008 (11.9); 0.000 (369.5); −0.008 (14.2); −0.150 (1.6)
Example 1-79: 1H-NMR (400.0 MHz, d6-DMSO):
□ = 9.353 (3.6); 9.339 (7.3); 9.324 (3.6); 9.044 (11.7); 9.038 (14.4); 8.986 (14.4); 8.980 (12.3); 8.491 (9.4); 8.486
(10.2); 8.479 (10.2); 8.474 (10.2); 8.316 (0.6); 7.952 (9.1); 7.947 (9.7); 7.933 (10.7); 7.928 (10.3); 7.728 (11.6);
7.708 (15.7); 7.695 (15.2); 7.599 (9.5); 7.595 (9.0); 7.578 (16.0); 7.566 (10.2); 7.559 (9.7); 7.547 (9.5); 7.524
(0.5); 6.109 (2.5); 6.101 (2.8); 6.089 (3.0); 6.081 (2.6); 5.990 (2.5); 5.981 (2.7); 5.971 (3.2); 5.962 (2.6); 4.056
(0.6); 4.038 (1.8); 4.020 (1.9); 4.003 (0.6); 3.965 (0.9); 3.956 (1.0); 3.950 (1.0); 3.942 (1.1); 3.928 (2.0); 3.920
(2.3); 3.915 (2.2); 3.906 (2.0); 3.890 (0.9); 3.883 (2.3); 3.876 (1.2); 3.868 (3.0); 3.854 (2.9); 3.847 (4.1); 3.840
(3.4); 3.832 (3.9); 3.819 (3.2); 3.800 (2.4); 3.783 (0.8); 3.780 (0.8); 3.764 (0.7); 3.324 (103.0); 2.681 (0.6); 2.676
(1.2); 2.672 (1.7); 2.667 (1.2); 2.525 (4.6); 2.520 (7.1); 2.512 (93.7); 2.507 (191.6); 2.503 (254.7); 2.498 (188.2);
2.494 (94.5); 2.339 (0.5); 2.334 (1.2); 2.329 (1.6); 2.325 (1.2); 1.989 (8.0); 1.398 (1.4); 1.299 (0.9); 1.259 (1.4);
1.235 (2.4); 1.193 (2.2); 1.175 (4.3); 1.158 (2.2); 0.854 (0.4); 0.146 (1.3); 0.008 (9.8); 0.000 (287.4); −0.008
(12.6); −0.150 (1.3)
Example 1-88: 1H-NMR (400.0 MHz, CD3CN):
□ = 8.758 (4.3); 8.748 (4.3); 8.746 (4.3); 8.499 (5.5); 8.492 (5.8); 8.195 (2.9); 8.188 (2.8); 8.174 (3.9); 8.168 (3.7);
8.167 (3.8); 8.154 (3.0); 8.147 (2.9); 7.873 (3.9); 7.871 (4.1); 7.854 (4.8); 7.852 (4.8); 7.773 (9.0); 7.708 (1.5);
7.704 (1.1); 7.687 (10.2); 7.683 (12.0); 7.680 (16.0); 7.673 (4.9); 7.661 (6.0); 7.653 (3.9); 7.641 (3.7); 7.301 (1.8);
7.151 (4.4); 7.144 (4.4); 7.130 (4.3); 7.123 (4.3); 6.131 (2.1); 6.123 (2.3); 6.113 (2.3); 6.105 (2.2); 6.014 (2.1);
6.006 (2.3); 5.996 (2.4); 5.988 (2.2); 5.448 (3.0); 4.038 (1.1); 4.029 (1.1); 4.022 (1.1); 4.014 (1.1); 4.001 (1.7);
3.993 (1.7); 3.986 (1.6); 3.978 (1.7); 3.969 (1.1); 3.960 (1.0); 3.953 (1.1); 3.945 (1.0); 3.932 (1.7); 3.924 (1.6);
3.917 (1.7); 3.908 (1.6); 3.882 (1.6); 3.866 (2.0); 3.864 (2.0); 3.849 (1.7); 3.845 (1.3); 3.827 (2.8); 3.812 (3.0);
3.794 (1.7); 3.790 (1.3); 3.775 (1.3); 3.773 (1.3); 3.757 (1.0); 2.467 (0.3); 2.462 (0.5); 2.458 (0.3); 2.153 (127.6);
2.120 (0.8); 2.114 (1.0); 2.108 (1.2); 2.102 (0.9); 2.096 (0.5); 1.972 (1.1); 1.965 (4.6); 1.959 (11.9); 1.953 (71.9);
1.947 (131.0); 1.940 (176.9); 1.934 (120.6); 1.928 (62.2); 1.915 (1.3); 1.781 (0.5); 1.775 (0.8); 1.769 (1.1); 1.763
(0.8); 1.756 (0.4); 1.340 (0.5); 1.285 (0.9); 1.269 (1.8); 1.222 (0.3); 1.204 (0.6); 1.186 (0.3); 0.881 (0.4); 0.000
(8.1)
Example 1-87: 1H-NMR (400.0 MHz, CD3CN):
□ = 8.846 (2.4); 8.837 (11.1); 8.830 (2.4); 8.485 (3.9); 8.480 (4.0); 8.183 (1.7); 8.176 (1.6); 8.162 (2.7); 8.156
(2.5); 8.142 (1.8); 8.136 (1.7); 7.926 (1.3); 7.764 (6.1); 7.683 (0.7); 7.663 (16.0); 7.640 (0.5); 7.148 (2.9); 7.141
(2.9); 7.126 (2.8); 7.119 (2.8); 6.155 (1.4); 6.147 (1.4); 6.136 (1.5); 6.128 (1.4); 6.037 (1.4); 6.029 (1.4); 6.019
(1.5); 6.011 (1.4); 5.448 (1.4); 4.071 (0.7); 4.063 (0.6); 4.055 (0.6); 4.048 (0.8); 4.034 (1.0); 4.026 (1.0); 4.019
(0.9); 4.011 (1.0); 4.001 (0.6); 3.993 (0.6); 3.986 (0.6); 3.977 (0.6); 3.964 (1.0); 3.956 (1.0); 3.949 (1.0); 3.941
(0.9); 3.923 (0.9); 3.905 (1.3); 3.888 (1.2); 3.870 (1.6); 3.853 (1.8); 3.836 (1.2); 3.817 (0.8); 3.800 (0.5); 2.469
(0.3); 2.465 (0.5); 2.460 (0.3); 2.167 (252.6); 2.121 (0.6); 2.114 (0.7); 2.108 (0.8); 2.102 (0.6); 2.096 (0.4); 1.972
(2.0); 1.965 (3.1); 1.959 (7.9); 1.953 (45.2); 1.947 (81.9); 1.941 (110.9); 1.935 (76.2); 1.929 (39.5); 1.776 (0.5);
1.769 (0.7); 1.763 (0.5); 1.340 (0.3); 1.285 (0.8); 1.270 (2.7); 1.222 (0.6); 1.204 (1.0); 1.186 (0.5); 0.881 (0.5);
0.858 (0.5); 0.000 (8.0)
Example 1-86: 1H-NMR (400.0 MHz, CD3CN):
□ = 8.757 (5.0); 8.747 (5.0); 8.745 (5.0); 8.678 (8.5); 8.672 (8.7); 8.050 (6.7); 8.043 (6.6); 8.029 (7.2); 8.022 (7.1);
7.870 (4.6); 7.867 (4.7); 7.850 (5.6); 7.848 (5.6); 7.786 (10.6); 7.721 (2.5); 7.717 (1.9); 7.700 (10.1); 7.696 (10.2);
7.688 (16.0); 7.671 (5.5); 7.660 (5.1); 7.652 (4.4); 7.640 (4.1); 7.521 (10.1); 7.500 (9.3); 7.306 (2.2); 6.130 (2.3);
6.122 (2.4); 6.113 (2.5); 6.105 (2.3); 6.013 (2.3); 6.005 (2.4); 5.996 (2.5); 5.988 (2.3); 5.448 (1.3); 4.068 (0.9);
4.050 (0.9); 4.036 (1.2); 4.028 (1.2); 4.021 (1.2); 4.013 (1.2); 4.000 (1.8); 3.992 (1.9); 3.985 (1.8); 3.976 (1.8);
3.968 (1.2); 3.960 (1.1); 3.952 (1.2); 3.944 (1.1); 3.931 (1.9); 3.923 (1.8); 3.916 (1.9); 3.908 (1.8); 3.882 (1.7);
3.866 (2.3); 3.849 (1.9); 3.845 (1.4); 3.827 (2.9); 3.811 (3.2); 3.793 (1.8); 3.790 (1.4); 3.774 (1.5); 3.757 (1.1);
2.467 (0.5); 2.462 (0.7); 2.458 (0.5); 2.157 (163.6); 2.120 (0.7); 2.114 (1.0); 2.108 (1.2); 2.102 (0.9); 2.096 (0.5);
1.972 (4.4); 1.965 (5.7); 1.959 (14.2); 1.953 (80.4); 1.947 (145.3); 1.941 (194.9); 1.934 (132.8); 1.928 (67.6);
1.781 (0.5); 1.775 (0.8); 1.769 (1.1); 1.763 (0.8); 1.757 (0.4); 1.285 (0.5); 1.270 (0.9); 1.222 (1.1); 1.204 (2.1);
1.186 (1.1); 0.000 (8.5)
Example 1-85: 1H-NMR (400.0 MHz, CD3CN):
□ = 8.844 (2.2); 8.836 (12.7); 8.829 (2.1); 8.665 (4.6); 8.659 (4.7); 8.038 (3.9); 8.031 (3.8); 8.017 (4.2); 8.010
(4.1); 7.897 (1.2); 7.779 (5.8); 7.776 (6.1); 7.674 (16.0); 7.671 (15.1); 7.517 (5.4); 7.516 (5.3); 7.497 (5.0); 7.495
(4.9); 6.154 (1.5); 6.146 (1.6); 6.136 (1.7); 6.128 (1.5); 6.037 (1.5); 6.029 (1.6); 6.019 (1.7); 6.011 (1.5); 5.447
(0.5); 4.072 (0.7); 4.064 (0.7); 4.056 (0.7); 4.048 (0.7); 4.035 (1.0); 4.027 (1.1); 4.020 (1.0); 4.012 (1.0); 4.002
(0.7); 3.994 (0.6); 3.987 (0.6); 3.978 (0.6); 3.966 (1.1); 3.957 (1.0); 3.950 (1.1); 3.942 (1.0); 3.922 (1.0); 3.906
(1.3); 3.904 (1.2); 3.888 (1.2); 3.870 (1.8); 3.852 (1.8); 3.836 (1.2); 3.816 (0.8); 3.799 (0.6); 2.591 (0.4); 2.138
(150.4); 2.120 (1.0); 2.114 (1.5); 2.108 (1.9); 2.101 (1.3); 2.095 (0.7); 1.964 (8.8); 1.958 (22.0); 1.952 (123.8);
1.946 (224.6); 1.940 (301.7); 1.934 (206.4); 1.928 (106.4); 1.915 (1.5); 1.781 (0.7); 1.775 (1.3); 1.769 (1.8); 1.762
(1.2); 1.756 (0.6); 1.269 (0.6); 0.008 (0.4); 0.000 (14.0); −0.009 (0.5)
Example 1-84: 1H-NMR (400.0 MHz, CD3CN):
□ = 8.844 (3.6); 8.837 (15.9); 8.835 (16.0); 8.828 (2.9); 8.661 (6.5); 8.647 (6.4); 7.898 (1.8); 7.856 (0.9); 7.838
(7.7); 7.816 (0.7); 7.811 (0.8); 7.799 (0.6); 7.789 (0.6); 7.771 (0.7); 7.767 (0.7); 7.757 (2.7); 7.737 (4.8); 7.733
(4.6); 7.691 (9.7); 7.671 (5.5); 7.629 (8.2); 7.614 (7.9); 7.599 (0.9); 7.584 (1.4); 7.580 (1.3); 7.562 (1.4); 7.552
(1.1); 7.529 (1.3); 7.521 (1.3); 7.511 (1.3); 7.502 (1.4); 7.481 (1.5); 7.461 (1.9); 7.450 (1.9); 7.436 (0.4); 7.417
(0.8); 7.398 (0.8); 7.392 (0.7); 7.380 (0.4); 7.373 (0.3); 6.293 (0.3); 6.288 (0.3); 6.226 (0.4); 6.160 (1.7); 6.152
(1.8); 6.142 (1.8); 6.134 (1.7); 6.043 (1.7); 6.035 (1.7); 6.025 (1.8); 6.017 (1.7); 4.086 (0.7); 4.077 (1.0); 4.068
(2.8); 4.061 (1.0); 4.050 (2.3); 4.040 (1.4); 4.032 (2.2); 4.025 (1.4); 4.016 (1.4); 4.007 (0.9); 3.999 (0.9); 3.992
(0.9); 3.984 (0.9); 3.971 (1.5); 3.963 (1.4); 3.955 (1.4); 3.947 (1.3); 3.927 (1.4); 3.911 (1.8); 3.893 (1.7); 3.874
(2.5); 3.856 (2.6); 3.840 (1.6); 3.821 (1.2); 3.804 (1.0); 3.051 (0.4); 2.138 (349.8); 2.120 (3.8); 2.114 (3.9); 2.108
(4.4); 2.101 (3.0); 2.095 (1.7); 1.972 (10.1); 1.964 (16.6); 1.958 (44.1); 1.952 (247.5); 1.946 (451.2); 1.940
(606.9); 1.934 (417.4); 1.928 (215.6); 1.869 (0.8); 1.844 (0.6); 1.809 (0.6); 1.781 (1.8); 1.775 (3.0); 1.769 (4.0);
1.762 (3.0); 1.756 (1.9); 1.733 (0.4); 1.707 (0.4); 1.655 (0.4); 1.619 (0.6); 1.597 (0.6); 1.564 (0.4); 1.469 (1.1);
1.386 (0.9); 1.372 (0.5); 1.348 (1.0); 1.340 (2.9); 1.285 (4.2); 1.270 (5.6); 1.222 (2.9); 1.204 (5.3); 1.186 (2.7);
0.882 (1.2); 0.857 (1.0); 0.841 (0.7); 0.008 (0.8); 0.000 (25.7); −0.008 (1.0)
Example 1-83: 1H-NMR (400.0 MHz, CD3CN):
□ = 8.801 (12.1); 8.796 (12.4); 8.759 (5.9); 8.747 (5.9); 8.605 (11.6); 8.599 (12.0); 8.103 (7.9); 8.097 (14.2);
8.092 (7.8); 7.868 (5.5); 7.848 (6.6); 7.819 (12.2); 7.749 (4.2); 7.744 (3.6); 7.728 (9.6); 7.724 (9.3); 7.710 (0.6);
7.698 (16.0); 7.678 (7.8); 7.662 (5.9); 7.655 (5.1); 7.643 (4.9); 7.584 (0.5); 7.395 (0.3); 7.277 (2.8); 6.135 (2.5);
6.127 (2.7); 6.118 (2.8); 6.110 (2.6); 6.018 (2.6); 6.010 (2.7); 6.001 (2.8); 5.993 (2.6); 4.086 (0.4); 4.068 (1.2);
4.050 (1.3); 4.041 (1.3); 4.033 (1.6); 4.026 (1.3); 4.018 (1.4); 4.005 (2.1); 3.997 (2.2); 3.989 (2.0); 3.981 (2.0);
3.973 (1.4); 3.965 (1.3); 3.957 (1.4); 3.949 (1.3); 3.936 (2.2); 3.928 (2.0); 3.921 (2.2); 3.913 (2.0); 3.886 (2.0);
3.870 (2.8); 3.853 (2.2); 3.832 (3.3); 3.816 (3.7); 3.798 (2.1); 3.795 (1.7); 3.778 (1.7); 3.761 (1.3); 2.140 (257.9);
2.120 (1.8); 2.114 (2.3); 2.108 (2.7); 2.101 (2.0); 2.095 (1.1); 1.972 (6.5); 1.964 (12.0); 1.958 (30.0); 1.952
(168.1); 1.946 (304.3); 1.940 (408.7); 1.934 (279.9); 1.928 (143.7); 1.781 (0.9); 1.775 (1.7); 1.769 (2.4); 1.762
(1.6); 1.756 (0.8); 1.437 (1.3); 1.340 (0.7); 1.285 (1.2); 1.269 (2.0); 1.222 (1.5); 1.204 (2.9); 1.186 (1.5); 0.881
(0.4); 0.858 (0.3); 0.008 (0.6); 0.000 (17.7); −0.009 (0.6)
Example 1-82: 1H-NMR (400.0 MHz, CD3CN):
□ = 8.846 (3.3); 8.838 (16.0); 8.831 (2.8); 8.787 (8.2); 8.782 (8.0); 8.602 (7.7); 8.596 (7.7); 8.088 (5.4); 8.083
(9.5); 8.077 (5.0); 7.913 (1.3); 7.900 (1.7); 7.809 (8.1); 7.717 (1.5); 7.713 (1.1); 7.696 (8.7); 7.692 (9.7); 7.688
(12.8); 7.668 (1.7); 6.159 (1.8); 6.151 (1.9); 6.141 (2.0); 6.133 (1.8); 6.042 (1.8); 6.034 (1.9); 6.024 (2.0); 6.016
(1.8); 5.447 (1.8); 4.076 (0.9); 4.068 (1.1); 4.061 (0.9); 4.053 (0.9); 4.040 (1.4); 4.032 (1.5); 4.024 (1.4); 4.016
(1.4); 4.007 (0.9); 3.998 (0.8); 3.991 (0.9); 3.983 (0.8); 3.970 (1.5); 3.962 (1.4); 3.954 (1.4); 3.946 (1.3); 3.926
(1.3); 3.908 (1.7); 3.891 (1.6); 3.873 (2.3); 3.855 (2.4); 3.839 (1.6); 3.820 (1.1); 3.803 (0.8); 2.136 (141.1); 2.120
(1.3); 2.114 (1.6); 2.107 (1.9); 2.101 (1.4); 2.095 (0.7); 1.972 (1.8); 1.964 (8.8); 1.958 (22.4); 1.952 (123.3); 1.946
(223.2); 1.940 (299.6); 1.934 (203.7); 1.928 (104.4); 1.781 (0.7); 1.775 (1.3); 1.769 (1.7); 1.762 (1.2); 1.756 (0.6);
1.340 (0.5); 1.285 (0.8); 1.270 (2.2); 1.222 (0.3); 1.204 (0.5); 0.881 (0.4); 0.008 (0.4); 0.000 (12.8); −0.008 (0.4)
Example 1-81: 1H-NMR (400.0 MHz, CD3CN):
□ = 8.759 (5.1); 8.757 (5.4); 8.747 (5.4); 8.745 (5.4); 8.296 (9.7); 8.282 (10.1); 7.872 (7.6); 7.869 (16.0); 7.865
(13.0); 7.848 (6.2); 7.845 (6.1); 7.804 (5.3); 7.799 (4.7); 7.784 (8.2); 7.779 (7.7); 7.714 (13.2); 7.694 (8.4); 7.672
(5.7); 7.660 (5.7); 7.653 (4.8); 7.641 (4.6); 7.593 (4.3); 7.588 (6.9); 7.584 (5.0); 7.580 (4.5); 7.575 (6.7); 7.571
(4.4); 7.340 (12.4); 7.268 (2.5); 6.138 (2.3); 6.130 (2.5); 6.121 (2.6); 6.113 (2.4); 6.021 (2.4); 6.013 (2.5); 6.004
(2.6); 5.996 (2.4); 5.447 (1.2); 4.068 (0.4); 4.050 (0.5); 4.044 (1.3); 4.036 (1.4); 4.029 (1.4); 4.021 (1.4); 4.008
(2.1); 4.000 (2.1); 3.992 (2.1); 3.984 (2.1); 3.976 (1.4); 3.968 (1.3); 3.961 (1.4); 3.953 (1.3); 3.940 (2.2); 3.932
(2.1); 3.924 (2.2); 3.916 (2.0); 3.890 (2.0); 3.874 (2.8); 3.857 (2.2); 3.853 (1.5); 3.834 (2.5); 3.818 (3.2); 3.800
(2.1); 3.797 (1.5); 3.781 (1.8); 3.764 (1.2); 2.141 (385.5); 2.120 (1.7); 2.114 (2.1); 2.108 (2.6); 2.101 (1.8); 2.095
(1.0); 1.972 (2.8); 1.964 (10.7); 1.958 (27.0); 1.953 (154.1); 1.946 (279.6); 1.940 (375.2); 1.934 (256.8); 1.928
(131.5); 1.915 (1.9); 1.781 (0.8); 1.775 (1.6); 1.769 (2.2); 1.763 (1.5); 1.756 (0.8); 1.285 (0.5); 1.269 (0.9); 1.222
(0.5); 1.204 (1.1); 1.186 (0.6); 0.008 (0.4); 0.000 (15.2); −0.009 (0.4)
Example 1-98: 1H-NMR (400.0 MHz, CD3CN):
□ = 8.741 (5.2); 8.707 (3.0); 8.696 (2.9); 8.509 (4.4); 8.502 (4.6); 7.930 (1.2); 7.909 (1.3); 7.855 (1.9); 7.849 (2.5);
7.844 (1.9); 7.830 (1.9); 7.824 (2.8); 7.812 (5.6); 7.776 (3.7); 7.755 (7.2); 7.717 (4.6); 7.712 (4.4); 7.696 (2.6);
7.691 (2.9); 7.668 (3.6); 7.650 (3.7); 7.602 (3.1); 7.591 (3.1); 7.583 (2.1); 7.571 (1.8); 7.553 (0.9); 7.535 (1.4);
7.515 (0.9); 7.451 (0.9); 7.432 (1.3); 7.413 (0.6); 7.061 (1.5); 4.338 (1.0); 4.322 (1.0); 4.302 (1.5); 4.285 (1.4);
4.267 (1.0); 4.250 (1.0); 4.230 (1.5); 4.214 (1.5); 4.146 (1.5); 4.131 (1.6); 4.110 (1.1); 4.093 (2.2); 4.078 (1.6);
4.068 (0.6); 4.056 (1.1); 4.050 (0.7); 4.041 (1.1); 2.769 (0.3); 2.143 (45.2); 2.114 (2.4); 2.108 (2.1); 2.101 (1.5);
1.972 (2.8); 1.964 (5.7); 1.958 (15.1); 1.952 (67.2); 1.946 (119.9); 1.940 (170.7); 1.934 (111.8); 1.928 (58.8);
1.879 (16.0); 1.781 (0.5); 1.775 (0.8); 1.769 (0.9); 1.762 (0.7); 1.756 (0.4); 1.285 (0.4); 1.269 (0.8); 1.222 (0.6);
1.204 (1.1); 1.186 (0.6); 0.000 (25.5)
Example 1-99: 1H-NMR (400.0 MHz, CD3CN):
□ = 8.740 (2.9); 8.736 (5.0); 8.732 (2.8); 8.506 (4.6); 8.499 (4.6); 7.851 (1.7); 7.846 (2.2); 7.845 (2.1); 7.840 (1.8);
7.833 (0.4); 7.826 (1.8); 7.821 (2.2); 7.820 (2.2); 7.815 (2.1); 7.810 (3.7); 7.806 (4.3); 7.781 (4.1); 7.760 (6.8);
7.703 (4.2); 7.698 (3.8); 7.683 (2.4); 7.678 (2.3); 7.504 (0.4); 7.400 (0.4); 7.394 (1.0); 7.381 (5.6); 7.376 (5.3);
7.366 (3.4); 7.361 (3.3); 7.346 (0.8); 7.341 (1.1); 7.324 (1.6); 7.318 (1.2); 7.308 (0.9); 7.305 (3.0); 7.300 (2.6);
7.289 (2.0); 7.284 (1.9); 7.272 (3.2); 7.270 (3.9); 7.267 (2.9); 7.253 (1.6); 7.249 (1.3); 6.963 (1.0); 5.448 (0.9);
4.349 (1.0); 4.332 (1.0); 4.313 (1.4); 4.296 (1.3); 4.278 (1.0); 4.261 (1.0); 4.241 (1.4); 4.224 (1.4); 4.129 (1.6);
4.115 (1.6); 4.093 (1.2); 4.078 (1.6); 4.076 (1.9); 4.061 (1.6); 4.039 (1.1); 4.025 (1.1); 2.468 (0.4); 2.463 (0.5);
2.458 (0.3); 2.152 (116.7); 2.120 (1.3); 2.114 (1.4); 2.108 (1.5); 2.102 (1.1); 2.095 (0.7); 1.965 (5.1); 1.958 (13.6);
1.953 (78.7); 1.946 (134.6); 1.940 (178.2); 1.934 (122.4); 1.928 (63.4); 1.890 (16.0); 1.858 (0.5); 1.781 (0.6);
1.775 (0.8); 1.769 (1.1); 1.763 (0.8); 1.757 (0.4); 1.437 (1.7); 1.270 (1.1); 0.008 (1.1); 0.000 (36.3); −0.009 (1.3)
Example 1-100: 1H-NMR (400.0 MHz, CD3CN):
□ = 8.731 (4.7); 8.503 (4.1); 8.496 (4.1); 7.848 (1.9); 7.843 (2.4); 7.842 (2.5); 7.837 (2.0); 7.823 (2.0); 7.818 (2.5);
7.816 (2.5); 7.812 (2.2); 7.802 (5.3); 7.783 (4.5); 7.763 (6.8); 7.711 (0.6); 7.702 (4.4); 7.697 (4.2); 7.681 (2.7);
7.677 (2.6); 7.568 (3.1); 7.566 (3.3); 7.548 (3.6); 7.546 (3.8); 7.445 (0.4); 7.442 (0.4); 7.426 (0.6); 7.423 (0.5);
7.420 (0.5); 7.407 (0.4); 7.401 (0.4); 7.365 (1.3); 7.362 (1.4); 7.346 (3.6); 7.343 (3.8); 7.328 (2.9); 7.325 (2.8);
7.298 (2.2); 7.293 (2.8); 7.278 (2.7); 7.274 (3.1); 7.259 (1.3); 7.255 (1.3); 7.220 (3.5); 7.216 (3.3); 7.201 (2.6);
7.197 (2.5); 6.924 (1.2); 4.356 (1.1); 4.339 (1.1); 4.320 (1.5); 4.303 (1.4); 4.284 (1.1); 4.267 (1.0); 4.248 (1.5);
4.231 (1.4); 4.114 (1.5); 4.099 (1.5); 4.077 (1.2); 4.067 (0.9); 4.061 (2.1); 4.049 (1.1); 4.046 (1.7); 4.024 (1.2);
4.010 (1.1); 2.251 (0.4); 2.153 (4.6); 2.114 (1.8); 2.108 (1.6); 2.102 (1.2); 2.054 (0.4); 1.972 (3.4); 1.965 (2.0);
1.953 (29.2); 1.947 (41.5); 1.940 (55.2); 1.934 (38.5); 1.928 (20.2); 1.916 (1.0); 1.895 (16.0); 1.858 (0.5); 1.775
(0.4); 1.769 (0.4); 1.436 (5.7); 1.285 (0.3); 1.268 (1.2); 1.221 (0.9); 1.204 (1.7); 1.186 (0.9); 0.000
(8.8); −0.009 (0.4)
Example 1-101: 1H-NMR (400.0 MHz, CD3CN):
□ = 8.742 (3.8); 8.509 (3.3); 8.502 (3.3); 7.855 (1.7); 7.850 (2.0); 7.848 (2.1); 7.843 (1.7); 7.829 (1.8); 7.825 (2.1);
7.823 (2.2); 7.818 (2.0); 7.814 (3.4); 7.810 (4.2); 7.807 (3.8); 7.785 (4.0); 7.764 (7.1); 7.717 (5.3); 7.712 (6.0);
7.696 (4.9); 7.691 (4.7); 7.621 (0.8); 7.604 (2.1); 7.602 (2.2); 7.587 (2.7); 7.582 (3.1); 7.563 (1.9); 7.561 (1.9);
7.544 (0.6); 7.291 (2.4); 7.274 (2.1); 7.003 (1.0); 5.448 (2.1); 4.296 (0.9); 4.279 (0.9); 4.259 (1.5); 4.243 (1.4);
4.226 (0.8); 4.210 (0.8); 4.190 (1.5); 4.173 (1.5); 4.156 (1.7); 4.141 (1.7); 4.119 (1.1); 4.104 (1.2); 4.101 (1.9);
4.086 (1.7); 4.065 (1.0); 4.049 (1.0); 2.469 (0.5); 2.464 (0.8); 2.459 (0.5); 2.164 (179.3); 2.120 (0.5); 2.114 (0.8);
2.108 (1.0); 2.102 (0.7); 2.096 (0.4); 1.965 (4.4); 1.959 (11.2); 1.953 (65.2); 1.947 (118.6); 1.941 (162.5); 1.934
(111.1); 1.928 (56.6); 1.916 (1.1); 1.878 (16.0); 1.781 (0.5); 1.775 (0.9); 1.769 (1.0); 1.763 (0.7); 1.757 (0.4);
1.285 (0.4); 1.269 (1.1); 0.008 (0.7); 0.000 (25.0); −0.009 (0.8)
Example 1-102: 1H-NMR (400.0 MHz, CD3CN):
□ = 8.798 (1.1); 8.792 (16.0); 8.785 (0.9); 8.730 (2.3); 8.725 (4.2); 8.721 (2.3); 8.505 (4.0); 8.498 (4.0); 7.840
(1.5); 7.835 (1.8); 7.834 (1.8); 7.829 (1.4); 7.815 (4.4); 7.811 (5.0); 7.804 (1.6); 7.758 (3.5); 7.737 (5.9); 7.683
(3.6); 7.678 (3.3); 7.662 (2.2); 7.657 (2.1); 7.619 (0.8); 5.448 (1.0); 4.306 (0.4); 4.290 (0.4); 4.269 (2.0); 4.254
(3.1); 4.247 (0.6); 4.239 (1.7); 4.231 (0.5); 4.218 (0.4); 4.211 (1.9); 4.202 (0.4); 4.194 (2.1); 4.191 (1.9); 4.175
(1.6); 4.154 (0.4); 4.138 (0.4); 4.068 (0.3); 4.050 (0.4); 3.078 (0.4); 2.815 (0.4); 2.469 (0.4); 2.465 (0.6); 2.460
(0.5); 2.166 (181.5); 2.121 (0.5); 2.114 (0.7); 2.108 (0.8); 2.102 (0.6); 2.096 (0.3); 1.972 (1.9); 1.965 (3.6); 1.959
(9.1); 1.953 (51.1); 1.947 (92.3); 1.941 (123.9); 1.935 (90.1); 1.928 (44.9); 1.916 (0.7); 1.872 (13.5); 1.775 (0.5);
1.769 (0.8); 1.763 (0.5); 1.437 (1.0); 1.270 (1.1); 1.222 (0.4); 1.204 (0.8); 1.186 (0.4); 0.008 (0.8); 0.000
(24.1); −0.009 (0.8)
Example 1-103: 1H-NMR (400.0 MHz, CD3CN):
□ = 8.745 (2.7); 8.741 (4.8); 8.736 (2.8); 8.508 (4.5); 8.501 (4.6); 7.855 (1.7); 7.850 (2.0); 7.848 (2.1); 7.843 (1.8);
7.830 (1.8); 7.825 (2.1); 7.823 (2.1); 7.818 (1.9); 7.809 (4.1); 7.763 (3.6); 7.742 (6.9); 7.702 (4.4); 7.697 (4.2);
7.681 (2.2); 7.676 (2.2); 7.587 (0.4); 7.459 (0.7); 7.443 (1.5); 7.438 (1.4); 7.426 (0.8); 7.421 (2.9); 7.417 (1.1);
7.405 (1.4); 7.400 (1.6); 7.384 (0.8); 7.167 (1.0); 7.017 (0.7); 7.014 (0.8); 7.007 (4.7); 6.996 (0.7); 6.993 (0.9);
6.987 (6.0); 6.978 (0.7); 6.967 (4.0); 6.959 (0.7); 6.957 (0.5); 5.448 (1.8); 4.264 (0.5); 4.248 (0.5); 4.228 (2.1);
4.212 (2.4); 4.209 (2.3); 4.193 (1.9); 4.169 (2.1); 4.153 (2.1); 4.143 (1.8); 4.128 (1.8); 4.107 (0.5); 4.092 (0.5);
3.169 (1.0); 2.471 (0.4); 2.466 (0.5); 2.461 (0.4); 2.160 (749.4); 2.121 (1.9); 2.114 (2.5); 2.108 (3.0); 2.102 (2.2);
2.096 (1.2); 1.965 (12.4); 1.959 (30.8); 1.953 (180.0); 1.947 (326.7); 1.941 (441.8); 1.934 (304.0); 1.928 (171.5);
1.868 (16.0); 1.781 (1.2); 1.775 (2.0); 1.769 (2.7); 1.763 (1.8); 1.757 (0.9); 1.270 (1.5); 0.008 (1.6); 0.000
(55.9); −0.009(1.8)
Example 1-130: 1H-NMR (400.0 MHz, CDCl3):
□ = 8.425 (4.3); 8.420 (4.4); 7.990 (1.6); 7.984 (1.6); 7.970 (2.6); 7.964 (2.5); 7.950 (1.7); 7.944 (1.7); 7.749 (5.2);
7.729 (6.2); 7.682 (2.5); 7.665 (3.1); 7.660 (3.1); 7.599 (5.9); 7.562 (0.9); 7.544 (3.1); 7.531 (6.2); 7.515 (2.9);
7.503 (4.1); 7.499 (4.3); 7.483 (3.3); 7.478 (3.1); 7.361 (2.9); 7.356 (3.1); 7.340 (2.6); 7.264 (98.3); 7.056 (2.9);
7.049 (2.9); 7.035 (2.7); 7.027 (2.7); 6.999 (0.6); 5.983 (2.0); 5.970 (1.2); 5.302 (9.7); 4.342 (4.7); 4.326 (4.5);
4.279 (4.9); 4.264 (4.7); 3.683 (0.5); 1.973 (15.9); 1.913 (16.0); 1.754 (0.3); 1.588 (127.4); 1.427 (0.7); 1.333
(1.0); 1.284 (1.6); 1.254 (6.9); 1.220 (0.6); 0.895 (0.5); 0.880 (1.0); 0.869 (0.7); 0.853 (0.9); 0.844 (0.8); 0.835
(0.8); 0.146 (0.4); 0.000 (89.5); −0.150 (0.5)
Example 1-129: 1H-NMR (400.0 MHz, CDCl3):
□ = 8.750 (3.4); 8.739 (3.4); 8.427 (4.5); 8.421 (4.7); 7.991 (1.7); 7.985 (1.7); 7.970 (2.8); 7.964 (2.6); 7.951 (1.8);
7.945 (1.7); 7.746 (7.4); 7.726 (9.5); 7.607 (6.0); 7.532 (3.1); 7.521 (4.1); 7.511 (5.7); 7.506 (4.3); 7.490 (3.3);
7.486 (3.2); 7.263 (194.9); 7.060 (2.9); 7.053 (3.0); 7.039 (2.8); 7.032 (2.8); 6.999 (1.1); 6.000 (2.0); 5.302 (7.6);
4.422 (0.7); 4.407 (0.8); 4.386 (1.7); 4.371 (1.7); 4.354 (0.7); 4.336 (2.1); 4.319 (3.0); 4.302 (2.0); 4.281 (2.2);
4.265 (1.9); 4.244 (0.7); 4.228 (0.7); 4.131 (0.4); 4.112 (0.4); 2.582 (0.3); 2.086 (0.8); 2.047 (1.9); 1.978 (15.9);
1.917 (16.0); 1.586 (239.3); 1.332 (0.4); 1.278 (1.0); 1.254 (3.2); 1.243 (1.0); 1.218 (0.4); 0.880 (0.5); 0.852 (0.5);
0.840 (0.5); 0.146 (0.9); 0.000 (178.7); −0.150 (0.9)
Example 1-89: 1H-NMR (400.0 MHz, d6-DMSO):
□ = 9.385 (11.9); 9.204 (2.5); 9.188 (5.5); 9.172 (2.6); 8.795 (5.1); 8.792 (5.6); 8.783 (5.4); 8.781 (5.5); 8.313
(16.0); 8.218 (8.7); 8.212 (9.5); 8.064 (4.5); 8.058 (4.3); 8.042 (5.6); 8.036 (5.4); 7.873 (4.1); 7.871 (4.5); 7.854
(6.5); 7.851 (6.5); 7.836 (10.1); 7.815 (8.5); 7.782 (5.8); 7.771 (5.6); 7.763 (4.0); 7.751 (3.9); 4.283 (2.2); 4.267
(2.3); 4.247 (5.1); 4.231 (4.9); 4.211 (2.6); 4.195 (2.4); 3.330 (217.0); 2.712 (1.3); 2.681 (0.4); 2.677 (0.7); 2.672
(1.0); 2.668 (0.8); 2.663 (0.4); 2.542 (342.1); 2.526 (2.3); 2.521 (3.2); 2.512 (55.7); 2.508 (117.7); 2.503 (158.5);
2.498 (114.8); 2.494 (55.0); 2.368 (1.3); 2.339 (0.3); 2.334 (0.7); 2.330 (1.0); 2.325 (0.7); 0.008 (0.9); 0.000
(31.5); −0.009 (1.0)
Example 1-90: 1H-NMR (400.0 MHz, d6-DMSO):
□ = 9.041 (0.5); 9.026 (1.2); 9.010 (0.6); 8.606 (2.0); 8.600 (2.0); 8.145 (1.3); 8.139 (1.3); 8.124 (1.3); 8.117 (1.3);
7.939 (2.2); 7.823 (0.9); 7.819 (0.8); 7.803 (1.3); 7.799 (1.3); 7.777 (1.2); 7.758 (1.7); 7.729 (2.5); 7.716 (1.5);
7.708 (1.9); 7.697 (1.1); 7.664 (1.0); 7.645 (1.2); 7.626 (0.4); 7.393 (1.3); 7.374 (1.3); 6.963 (2.1); 6.942 (2.0);
4.260 (0.5); 4.244 (0.5); 4.224 (1.1); 4.208 (1.1); 4.188 (0.6); 4.172 (0.6); 3.917 (16.0); 3.904 (0.4); 3.885 (0.6);
3.351 (397.5); 2.683 (0.4); 2.679 (0.5); 2.674 (0.4); 2.549 (57.3); 2.532 (1.2); 2.518 (32.2); 2.514 (66.3); 2.510
(88.4); 2.505 (64.7); 2.501 (32.0); 2.341 (0.4); 2.336 (0.6); 2.332 (0.4)
Example 1-91: 1H-NMR (400.0 MHz, d6-DMSO):
□ = 9.041 (0.5); 9.026 (1.1); 9.010 (0.5); 8.579 (1.9); 8.574 (2.0); 8.110 (1.3); 8.103 (1.3); 8.088 (1.4); 8.081 (1.3);
7.929 (1.9); 7.926 (2.1); 7.813 (0.8); 7.809 (0.8); 7.792 (1.3); 7.788 (1.3); 7.777 (1.1); 7.757 (1.4); 7.733 (0.5);
7.721 (2.4); 7.700 (1.8); 7.663 (0.9); 7.644 (1.1); 7.625 (0.4); 7.391 (1.2); 7.372 (1.1); 6.873 (2.0); 6.852 (2.0);
5.343 (0.4); 5.327 (1.1); 5.312 (1.5); 5.296 (1.1); 5.281 (0.4); 4.258 (0.5); 4.242 (0.5); 4.221 (1.0); 4.206 (1.0);
4.185 (0.5); 4.170 (0.5); 3.350 (277.8); 3.301 (0.3); 2.679 (0.4); 2.549 (44.0); 2.532 (0.9); 2.527 (1.3); 2.519
(23.8); 2.514 (50.4); 2.510 (67.8); 2.505 (49.3); 2.501 (23.8); 2.336 (0.4); 1.333 (16.0); 1.317 (15.9)
Example 1-96: 1H-NMR (400.0 MHz, d6-DMSO):
□ = 9.053 (2.9); 9.037 (6.3); 9.021 (3.0); 8.901 (7.4); 8.897 (13.8); 8.893 (7.5); 8.659 (13.5); 8.652 (13.9); 8.241
(4.2); 8.236 (5.1); 8.234 (5.3); 8.229 (4.1); 8.215 (4.3); 8.210 (5.4); 8.209 (5.1); 8.204 (4.0); 8.099 (10.8); 8.095
(11.8); 7.955 (5.3); 7.951 (5.3); 7.934 (6.6); 7.930 (6.6); 7.776 (14.7); 7.755 (16.0); 7.730 (2.7); 7.712 (6.8); 7.694
(4.9); 7.659 (5.0); 7.640 (6.1); 7.621 (2.2); 7.390 (7.1); 7.371 (6.4); 4.270 (2.6); 4.254 (2.7); 4.234 (6.1); 4.218
(5.9); 4.198 (3.1); 4.182 (2.9); 3.341 (545.8); 3.307 (0.9); 3.288 (0.4); 3.282 (0.4); 2.712 (1.9); 2.681 (0.3); 2.677
(0.7); 2.672 (1.0); 2.668 (0.7); 2.663 (0.4); 2.572 (0.4); 2.543 (467.7); 2.526 (2.6); 2.521 (3.2); 2.512 (54.3); 2.508
(115.3); 2.503 (155.6); 2.499 (112.8); 2.494 (54.5); 2.369 (2.0); 2.339 (0.4); 2.335 (0.8); 2.330 (1.0); 2.325 (0.8);
1.785 (0.6); 1.755 (0.5); 1.752 (0.5); 1.739 (0.4); 1.298 (0.3); 1.268 (0.4); 1.259 (0.5); 1.234 (0.8); 1.214 (0.5);
0.000 (0.6)
Example 1-97: 1H-NMR (400.0 MHz, d6-DMSO):
□ = 9.051 (1.0); 9.035 (2.2); 9.019 (1.1); 8.964 (4.9); 8.959 (5.1); 8.699 (4.7); 8.693 (4.9); 8.403 (3.0); 8.398 (5.3);
8.392 (3.0); 8.110 (3.9); 8.107 (4.3); 7.954 (1.9); 7.950 (1.9); 7.934 (2.4); 7.930 (2.4); 7.770 (6.2); 7.750 (5.8);
7.729 (1.0); 7.711 (2.5); 7.693 (1.8); 7.658 (1.8); 7.639 (2.2); 7.620 (0.8); 7.390 (2.6); 7.371 (2.3); 4.268 (1.0);
4.252 (1.0); 4.232 (2.2); 4.216 (2.1); 4.195 (1.1); 4.179 (1.0); 3.326 (32.6); 2.676 (0.4); 2.671 (0.5); 2.667 (0.4);
2.525 (1.2); 2.511 (28.6); 2.507 (59.2); 2.502 (79.1); 2.498 (58.2); 2.493 (28.7); 2.333 (0.4); 2.329 (0.5); 2.325
(0.4); 1.989 (1.4); 1.398 (16.0); 1.193 (0.4); 1.175 (0.7); 1.157 (0.4); 0.000 (5.0)
Example 1-92: 1H-NMR (400.0 MHz, d6-DMSO):
□ = 9.052 (0.7); 9.036 (1.5); 9.020 (0.8); 8.220 (2.8); 8.075 (16.0); 8.050 (1.4); 8.034 (1.5); 8.030 (1.6); 7.907
(0.3); 7.787 (2.7); 7.766 (3.0); 7.751 (2.3); 7.725 (0.9); 7.708 (1.9); 7.690 (1.4); 7.658 (1.4); 7.639 (1.7); 7.620
(0.6); 7.585 (0.5); 7.565 (0.4); 7.381 (1.7); 7.363 (1.8); 4.265 (0.6); 4.250 (0.6); 4.230 (1.5); 4.214 (1.5); 4.194
(0.8); 4.178 (0.8); 3.334 (1643.1); 2.711 (0.7); 2.676 (2.7); 2.671 (3.8); 2.667 (2.8); 2.541 (180.1); 2.524 (8.6);
2.520 (13.1); 2.511 (209.8); 2.507 (435.9); 2.502 (582.2); 2.497 (418.5); 2.493 (199.4); 2.368 (0.7); 2.333 (2.6);
2.329 (3.7); 2.324 (2.6); 1.298 (0.5); 1.258 (0.7); 1.235 (1.1); 0.000 (3.5)
Example 1-95: 1H-NMR (400.0 MHz, d6-DMSO):
□ = 10.656 (2.1); 9.035 (0.7); 9.019 (1.5); 9.003 (0.7); 8.420 (2.6); 8.407 (3.1); 8.398 (2.1); 7.912 (3.2); 7.811
(7.2); 7.766 (1.4); 7.747 (1.9); 7.725 (0.6); 7.708 (1.6); 7.690 (1.2); 7.654 (1.2); 7.635 (1.4); 7.617 (0.5); 7.486
(1.9); 7.482 (1.8); 7.473 (1.8); 7.468 (1.9); 7.384 (1.7); 7.365 (1.5); 4.266 (0.6); 4.251 (0.6); 4.230 (1.3); 4.215
(1.3); 4.195 (0.7); 4.179 (0.6); 3.337 (126.0); 2.712 (0.6); 2.542 (149.0); 2.526 (0.7); 2.521 (1.1); 2.512 (16.8);
2.507 (35.8); 2.503 (48.4); 2.498 (35.2); 2.494 (17.0); 2.368 (0.6); 2.127 (16.0); 0.000 (0.4)
Example 1-93: 1H-NMR (400.0 MHz, d6-DMSO):
□ = 9.046 (4.0); 9.030 (8.2); 9.015 (4.0); 8.984 (0.9); 8.969 (0.5); 8.669 (12.2); 8.663 (11.8); 8.429 (3.9); 8.422
(3.8); 8.409 (6.8); 8.402 (6.5); 8.388 (4.1); 8.382 (3.7); 8.317 (0.4); 8.026 (15.0); 8.024 (15.0); 7.912 (1.6); 7.881
(6.6); 7.861 (9.2); 7.858 (8.6); 7.818 (0.5); 7.796 (0.6); 7.771 (10.0); 7.763 (16.0); 7.752 (13.0); 7.743 (11.5);
7.728 (4.6); 7.710 (10.4); 7.692 (7.3); 7.658 (7.4); 7.639 (8.9); 7.620 (3.2); 7.587 (1.6); 7.565 (1.2); 7.389 (9.4);
7.370 (9.1); 7.353 (7.8); 7.346 (8.0); 7.331 (7.3); 7.324 (7.1); 4.264 (3.6); 4.248 (3.8); 4.228 (8.1); 4.212 (8.2);
4.192 (4.5); 4.176 (4.7); 4.160 (1.2); 4.141 (0.6); 4.124 (0.5); 3.520 (0.4); 3.504 (0.3); 3.492 (0.3); 3.471 (0.5);
3.458 (0.5); 3.335 (1503.3); 3.277 (1.1); 3.247 (0.4); 2.996 (0.4); 2.712 (3.5); 2.672 (3.3); 2.667 (2.5); 2.608 (0.6);
2.593 (1.0); 2.542 (741.1); 2.525 (17.8); 2.507 (414.3); 2.503 (507.8); 2.499 (372.9); 2.404 (0.4); 2.368 (3.6);
2.329 (3.2); 1.299 (0.4); 1.258 (0.6); 1.235 (1.2); 0.000 (2.3)
Example 1-94: 1H-NMR (400.0 MHz, d6-DMSO):
□ = 9.211 (0.4); 9.183 (12.5); 9.178 (12.6); 9.057 (4.0); 9.042 (8.6); 9.026 (4.1); 9.000 (0.3); 8.983 (0.6); 8.886
(0.6); 8.873 (0.7); 8.495 (6.3); 8.490 (6.2); 8.474 (6.9); 8.469 (7.0); 8.316 (0.9); 8.240 (0.8); 8.162 (0.5); 8.134
(14.2); 8.131 (15.2); 8.072 (0.4); 8.068 (0.5); 8.050 (14.1); 8.030 (12.6); 7.978 (6.7); 7.974 (6.6); 7.957 (8.6);
7.953 (8.6); 7.912 (1.0); 7.908 (1.1); 7.894 (0.3); 7.815 (16.0); 7.794 (12.8); 7.771 (8.5); 7.752 (11.6); 7.728 (3.9);
7.710 (9.4); 7.692 (6.8); 7.659 (7.2); 7.640 (8.8); 7.621 (3.1); 7.586 (1.2); 7.565 (1.0); 7.394 (9.6); 7.376 (8.6);
7.347 (0.7); 4.278 (3.5); 4.263 (3.7); 4.242 (8.0); 4.227 (7.8); 4.206 (4.2); 4.191 (3.9); 4.176 (0.9); 4.159 (0.7);
4.140 (0.4); 4.124 (0.4); 3.483 (0.3); 3.455 (0.5); 3.441 (0.5); 3.428 (0.6); 3.338 (1455.9); 3.299 (1.0); 3.282 (0.4);
3.274 (0.4); 2.996 (0.5); 2.712 (3.7); 2.681 (1.0); 2.676 (2.0); 2.672 (2.9); 2.667 (2.2); 2.663 (1.2); 2.617 (0.4);
2.589 (0.6); 2.573 (1.1); 2.542 (905.4); 2.525 (6.7); 2.520 (9.1); 2.512 (158.0); 2.507 (333.2); 2.503 (449.2); 2.498
(326.9); 2.494 (158.7); 2.445 (0.4); 2.368 (3.7); 2.338 (0.9); 2.334 (2.0); 2.329 (2.8); 2.325 (2.1); 1.298 (0.4);
1.258 (0.6); 1.234 (1.0); 0.000 (2.1)
Example 1-104: 1H-NMR (400.0 MHz, d6-DMSO):
□ = 9.215 (3.0); 9.200 (6.5); 9.184 (3.1); 8.798 (6.5); 8.789 (6.3); 8.786 (6.5); 8.673 (8.8); 8.667 (9.3); 8.432 (3.2);
8.425 (3.2); 8.411 (5.1); 8.405 (5.0); 8.391 (3.4); 8.384 (3.3); 8.035 (10.9); 8.032 (11.7); 7.884 (5.1); 7.880 (5.5);
7.875 (5.7); 7.872 (5.8); 7.864 (7.6); 7.859 (8.8); 7.856 (9.3); 7.790 (6.7); 7.778 (6.9); 7.769 (16.0); 7.758 (5.0);
7.748 (8.9); 7.356 (5.7); 7.349 (5.8); 7.334 (5.6); 7.327 (5.6); 4.293 (2.7); 4.277 (2.8); 4.256 (6.1); 4.240 (5.9);
4.220 (3.1); 4.204 (2.9); 3.334 (39.3); 2.678 (0.4); 2.673 (0.5); 2.669 (0.4); 2.527 (1.8); 2.513 (30.2); 2.509 (60.8);
2.505 (80.1); 2.500 (59.1); 2.496 (29.3); 2.336 (0.3); 2.331 (0.5); 2.327 (0.3); 1.991 (1.1); 1.260 (0.4); 1.232 (0.7);
1.194 (0.4); 1.176 (0.6); 1.158 (0.3); 0.146 (0.6); 0.008 (4.8); 0.000 (122.4); −0.009 (4.8); −0.150 (0.5)
Example 1-136: 1H-NMR (400.0 MHz, d6-DMSO):
□ = 9.087 (1.7); 9.072 (3.5); 9.056 (1.7); 8.610 (4.9); 8.605 (5.2); 8.366 (1.7); 8.359 (1.7); 8.345 (3.0); 8.339 (3.0);
8.325 (1.8); 8.318 (1.9); 7.787 (3.4); 7.767 (4.6); 7.740 (1.6); 7.720 (8.4); 7.704 (3.4); 7.671 (5.3); 7.649 (3.6);
7.630 (1.4); 7.610 (6.3); 7.589 (4.1); 7.389 (4.0); 7.370 (3.6); 7.330 (3.1); 7.323 (3.2); 7.309 (3.1); 7.302 (3.1);
4.109 (1.5); 4.093 (1.6); 4.071 (3.4); 4.056 (3.4); 4.033 (1.8); 4.018 (1.7); 3.334 (154.4); 2.676 (0.5); 2.672 (0.7);
2.668 (0.5); 2.569 (16.0); 2.507 (82.7); 2.503 (107.7); 2.499 (82.1); 2.334 (0.6); 2.330 (0.7); 2.325 (0.6); 1.989
(0.7); 1.397 (7.2); 1.175 (0.4); 0.008 (0.6); 0.000 (12.6)
Example 1-141: 1H-NMR (400.0 MHz, d6-DMSO):
□ = 8.897 (0.9); 8.881 (1.8); 8.866 (0.9); 8.654 (2.7); 8.649 (2.8); 8.406 (0.8); 8.400 (0.8); 8.386 (1.5); 8.379 (1.5);
8.366 (0.9); 8.359 (0.8); 7.757 (1.8); 7.737 (2.4); 7.706 (0.8); 7.688 (2.0); 7.669 (1.5); 7.639 (1.5); 7.620 (1.8);
7.601 (0.7); 7.581 (2.3); 7.561 (2.9); 7.473 (3.6); 7.412 (2.1); 7.392 (1.7); 7.340 (1.6); 7.334 (1.7); 7.318 (3.5);
7.299 (1.9); 4.205 (0.8); 4.190 (0.9); 4.169 (1.8); 4.153 (1.7); 4.132 (0.9); 4.117 (0.9); 3.979 (16.0); 3.335 (76.2);
2.672 (0.4); 2.507 (45.7); 2.503 (56.3); 2.329 (0.4); 1.990 (0.4); 0.000 (7.0)
Example 1-135: 1H-NMR (400.0 MHz, d6-DMSO):
□ = 9.167 (3.5); 9.152 (7.4); 9.136 (3.5); 8.726 (10.8); 8.720 (11.3); 8.471 (3.7); 8.465 (3.7); 8.450 (5.9); 8.445
(5.6); 8.431 (3.9); 8.424 (3.8); 8.317 (0.8); 7.774 (16.0); 7.760 (11.4); 7.746 (15.1); 7.735 (10.6); 7.716 (5.9);
7.671 (6.0); 7.652 (7.5); 7.633 (2.8); 7.581 (0.4); 7.416 (8.8); 7.398 (7.9); 7.369 (6.7); 7.362 (6.9); 7.347 (6.7);
7.340 (6.6); 4.162 (3.1); 4.146 (3.3); 4.127 (7.1); 4.111 (6.9); 4.092 (3.7); 4.076 (3.4); 4.038 (0.4); 4.020 (0.4);
3.331 (351.1); 2.676 (1.5); 2.672 (2.1); 2.667 (1.6); 2.525 (5.6); 2.512 (119.4); 2.507 (241.2); 2.503 (318.2); 2.498
(236.1); 2.494 (119.3); 2.334 (1.5); 2.330 (2.1); 2.325 (1.5); 1.989 (1.5); 1.398 (9.2); 1.193 (0.4); 1.175 (0.8);
1.157 (0.4); 0.008 (1.0); 0.000 (28.8); −0.008 (1.3)

TABLE 4
LC-MS and NMR spectra of selected compounds

| Example no. | LC-MS | NMR (Method M2) |
|---|---|---|
| 1-105 | (Method L2): Rₜ = 3.92 | 1H NMR (300 MHz, DMSO-d6) δ 9.20-9.15 |
| | min; m/z = 493 (M + H)⁺ | (m, 1H), 9.05 (t, J = 6.2 Hz, 1H), 8.50-8.43 (m, |
| | | 1H), 8.04 (d, J = 8.3 Hz, 1H), 7.95 (d, J = 12.5 |
| | | Hz, 1H), 7.87-7.59 (m, 5H), 7.39 (d, J = 7.0 |
| | | Hz, 1H), 4.20-4.07 (m, 2H). |
| 1-106 | (Method L2): Rₜ = 3.27 | 1H NMR (300 MHz, DMSO-d6) δ 9.04 (t, J = |
| | min; m/z = 443 (M + H)⁺ | 6.3 Hz, 1H), 8.67 (d, J = 2.2 Hz, 1H), 8.45-8.31 |
| | | (m, 1H), 7.90-7.54 (m, 6H), 7.42-7.29 (m, |
| | | 2H), 4.20-4.02 (m, 2H). |
| 1-107 | (Method L1): Rₜ = 3.41 | 1H NMR (300 MHz, DMSO-d6) δ 9.09-8.99 |
| | min; m/z = 459 (M + H)⁺ | (m, 1H), 8.96 (s, 1H), 8.69 (s, 1H), 8.38 (s, 1H), |
| | | 7.92 (d, J = 12.4 Hz, 1H), 7.72 (ddt, J = 32.8, |
| | | 14.9, 7.7 Hz, 5H), 7.38 (d, J = 6.9 Hz, 1H), |
| | | 4.22-4.00 (m, 2H). |
| 1-108 | (Method L1): Rₜ = 3.07 | 1H NMR (300 MHz, DMSO-d6) δ 9.04 (t, J = |
| | min; m/z = 455 (M + H)⁺ | 5.4 Hz, 1H), 8.57 (s, 1H), 8.35 (s, 1H), 7.87 (d, |
| | | J = 12.6 Hz, 1H), 7.81-7.57 (m, 6H), 7.38 (d, J = |
| | | 7.4 Hz, 1H), 4.22-4.02 (m, 2H), 3.92 (s, 3H). |
| 1-109 | (Method L1): Rₜ = 2.47 | 1H NMR (300 MHz, DMSO-d6) δ 9.01 (t, J = |
| | min; m/z = 468 (M + H)⁺ | 6.2 Hz, 1H), 8.54 (d, J = 2.6 Hz, 1H), 7.93 (d, |
| | | J = 8.9, 2.7 Hz, 1H), 7.80-7.52 (m, 6H), 7.38 (d, |
| | | J = 7.2 Hz, 1H), 6.73 (d, J = 9.1 Hz, 1H), |
| | | 4.18-3.99 (m, 2H), 3.08 (s, 6H). |
| 1-110 | (Method L1): Rₜ = 3.44 | 1H NMR (300 MHz, DMSO-d6) δ 9.03 (t, J = |
| | min; m/z = 455 (M + H)⁺ | 5.9 Hz, 1H), 8.60 (s, 1H), 8.12 (d, J = 8.7, 2.6 |
| | | Hz, 1H), 7.83-7.56 (m, 6H), 7.38 (d, J = 7.8 |
| | | Hz, 1H), 6.94 (d, J = 8.7 Hz, 1H), 4.20-4.02 |
| | | (m, 2H), 3.91 (s, 3H). |
| 1-111 | (Method L1): Rₜ = 4.51 | 1H NMR (300 MHz, DMSO-d6) δ 9.04 (t, J = |
| | min; m/z = 443 (M + H)⁺ | 6.2 Hz, 1H), 8.90 (d, J = 1.7 Hz, 1H), 8.65 (d, |
| | | J = 2.7 Hz, 1H), 8.20 (d, J = 10.3, 2.3 Hz, 1H), |
| | | 7.92 (d, J = 12.7 Hz, 1H), 7.86-7.57 (m, 5H), |
| | | 7.38 (d, J = 7.4 Hz, 1H), 4.23-4.02 (m, 2H). |
| 1-112 | (Method L1): Rₜ = 2.99 | 1H NMR (300 MHz, DMSO-d6) δ 9.32 (s, 1H), |
| | min; m/z = 503 (M + H)⁺ | 9.13-9.00 (m, 2H), 8.64 (t, J = 2.2 Hz, 1H), |
| | | 8.02 (d, J = 12.4 Hz, 1H), 7.93-7.59 (m, 5H), |
| | | 7.39 (d, J = 7.1 Hz, 1H), 4.28-4.03 (m, 2H), |
| | | 3.42 (s, 3H). |
| 1-113 | (Method L1): Rₜ = 2.98 | 1H NMR (300 MHz, DMSO-d6) δ 10.67 (s, |
| | min; m/z = 482 (M + H)⁺ | 1H), 9.03 (t, J = 6.2 Hz, 1H), 8.74 (s, 1H), 8.18 |
| | | (s, 2H), 7.85-7.57 (m, 6H), 7.38 (d, J = 7.0 Hz, |
| | | 1H), 4.20-4.01 (m, 2H), 2.12 (s, 3H). |
| 1-114 | (Method L1): Rₜ = 3.38 | 1H NMR (300 MHz, DMSO-d6) δ 9.04 (t, J = |
| | min; m/z = 459 (M + H)⁺ | 6.1 Hz, 1H), 8.84 (d, J = 2.6 Hz, 1H), 8.27 (dd, |
| | | J = 8.4, 2.6 Hz, 1H), 7.87 (d, J = 12.5 Hz, 1H), |
| | | 7.79-7.60 (m, 6H), 7.38 (d, J = 7.0 Hz, 1H), |
| | | 4.20-4.04 (m, 2H). |
| 1-115 | (Method L1): Rₜ = 3.23 | 1H NMR (300 MHz, DMSO-d6) δ 9.00 (t, J = |
| | min; m/z = 496 (M + H)⁺ | 6.2 Hz, 1H), 8.45 (s, 1H), 8.18 (s, 1H), |
| | | 7.80-7.50 (m, 6H), 7.37 (d, J = 7.3 Hz, 1H), 5.17 |
| | | (q, J = 9.1 Hz, 2H), 4.17-3.99 (m, 2H). |
| 1-116 | (Method L1): Rₜ = 3.01 | 1H NMR (300 MHz, DMSO-d6) δ 9.00 (t, J = |
| | min; m/z = 428 (M + H)⁺ | 6.0 Hz, 1H), 8.29 (s, 1H), 7.99 (s, 1H), |
| | | 7.79-7.47 (m, 6H), 7.36 (d, J = 7.3 Hz, 1H), |
| | | 4.16-3.96 (m, 2H), 3.87 (s, 3H). |
| 1-117 | (Method L1): Rₜ = 2.87 | 1H NMR (300 MHz, DMSO-d6) δ 13.09 (s, |
| | min; m/z = 414 (M + H)⁺ | 1H), 9.00 (t, J = 6.2 Hz, 1H), 8.36 (s, 1H), 8.06 |
| | | (s, 1H), 7.83-7.49 (m, 6H), 7.37 (d, J = 7.7 Hz, |
| | | 1H), 4.19-3.96 (m, 2H). |
| 1-118 | (Method L1): Rₜ = 3.13 | 1H NMR (300 MHz, DMSO-d6) δ 9.28 (s, 1H), |
| | min; m/z = 450 (M + H)⁺ | 9.10-9.01 (m, 2H), 8.77 (s, 1H), 7.96 (d, J = |
| | | 12.5 Hz, 1H), 7.85 (d, J = 8.2 Hz, 1H), |
| | | 7.78-7.60 (m, 4H), 7.38 (d, J = 7.4 Hz, 1H), |
| | | 4.20-4.05 (m, 2H). |
| 1-119 | (Method L1): Rₜ = 3.49 | 1H NMR (300 MHz, DMSO-d6) δ 9.35 (d, J = |
| | min; m/z = 551 (M − H)⁻ | 2.2 Hz, 1H), 9.18-9.06 (m, 2H), 8.70 (t, J = 2.1 |
| | | Hz, 1H), 8.35 (d, J = 10.5 Hz, 2H), 7.95 (d, J = |
| | | 8.2 Hz, 1H), 7.81-7.62 (m, 3H), 7.46 (d, J = 7.0 |
| | | Hz, 1H), 4.21-4.04 (m, 2H), 3.42 (s, 3H). |
| 1-120 | (Method L1): Rₜ = 3.50 | 1H NMR (300 MHz, DMSO-d6) δ 9.09 (t, J = |
| | min; m/z = 509 (M + H)⁺ | 6.2 Hz, 1H), 8.97 (d, J = 1.9 Hz, 1H), 8.70 (d, |
| | | J = 2.2 Hz, 1H), 8.47-8.41 (m, 1H), 8.26 (d, J = |
| | | 7.4 Hz, 2H), 7.88 (d, J = 8.7 Hz, 1H), 7.79-7.59 |
| | | (m, 3H), 7.43 (d, J = 7.0 Hz, 1H), 4.20-3.96 (m, |
| | | 2H). |
| 1-121 | (Method L1): Rₜ = 3.22 | 1H NMR (300 MHz, DMSO-d6) δ 9.11 (t, J = |
| | min; m/z = 505 (M + H)⁺ | 6.2 Hz, 1H), 8.59 (d, J = 1.8 Hz, 1H), 8.38 (d, |
| | | J = 2.7 Hz, 1H), 8.24 (d, J = 6.7 Hz, 2H), 7.89 (d, |
| | | J = 8.9 Hz, 1H), 7.81-7.61 (m, 4H), 7.45 (d, J = |
| | | 7.4 Hz, 1H), 4.11 (td, J = 14.6, 6.3 Hz, 2H), 3.94 |
| | | (s, 3H). |
| 1-122 | (Method L1): Rₜ = 3.37 | 1H NMR (300 MHz, DMSO-d6) δ 9.11 (t, J = |
| | min; m/z = 493 (M + H)+ | 6.1 Hz, 1H), 8.71 (d, J = 2.5 Hz, 1H), 8.51-8.41 |
| | | (m, 1H), 8.21 (d, J = 4.8 Hz, 2H), 7.89 (d, J = |
| | | 8.8 Hz, 1H), 7.81-7.61 (m, 3H), 7.45 (d, J = 7.0 |
| | | Hz, 1H), 7.36 (d, J = 8.6, 2.9 Hz, 1H), 4.19-4.02 |
| | | (m, 2H). |
| 1-123 | (Method L1): Rₜ = 3.18 | 1H NMR (300 MHz, DMSO-d6) δ 10.70 (s, |
| | min; m/z = 532 (M + H)+ | 1H), 9.10 (t, J = 6.2 Hz, 1H), 8.78 (d, J = 1.5 Hz, |
| | | 1H), 8.29-8.13 (m, 4H), 7.86 (d, J = 9.0 Hz, |
| | | 1H), 7.81-7.60 (m, 3H), 7.45 (d, J = 7.2 Hz, |
| | | 1H), 4.19-3.99 (m, 2H), 2.13 (s, 3H). |
| 1-124 | (Method L1): Rₜ = 3.61 | 1H NMR (300 MHz, DMSO-d6) δ 9.22 (s, 1H), |
| | min; m/z = 543 (M + H)⁺ | 9.12 (t, J = 6.2 Hz, 1H), 8.53 (d, J = 8.2, 2.1 Hz, |
| | | 1H), 8.31 (d, J = 6.7 Hz, 2H), 8.06 (d, J = 8.2 |
| | | Hz, 1H), 7.94 (d, J = 8.9 Hz, 1H), 7.81-7.61 |
| | | (m, 3H), 7.46 (d, J = 7.4 Hz, 1H), 4.22-4.01 (m, |
| | | 2H). |
| 1-125 | (Method L1): Rₜ = 2.68 | 1H NMR (300 MHz, DMSO-d6) δ 9.08 (t, J = |
| | min; m/z = 518 (M + H)⁺ | 6.1 Hz, 1H), 8.57 (d, J = 2.6 Hz, 1H), 8.07 (d, |
| | | J = 10.9 Hz, 2H), 7.98 (dd, J = 8.8, 2.3 Hz, 1H), |
| | | 7.82-7.61 (m, 4H), 7.44 (d, J = 7.7 Hz, 1H), |
| | | 6.76 (d, J = 9.0 Hz, 1H), 4.15-3.99 (m, 2H), |
| | | 3.09 (s, 6H). |
| 1-126 | (Method L1): Rₜ = 3.55 | 1H NMR (300 MHz, DMSO-d6) δ 9.08 (t, J = |
| | min; m/z = 509 (M + H)⁺ | 6.3 Hz, 1H), 8.86 (d, J = 2.2 Hz, 1H), 8.30 (dd, |
| | | J = 8.4, 2.7 Hz, 1H), 8.21 (d, J = 5.0 Hz, 2H), 7.88 |
| | | (d, J = 8.8 Hz, 1H), 7.78-7.59 (m, 4H), 7.43 (d, |
| | | J = 7.6 Hz, 1H), 4.17-3.98 (m, 2H). |
| 1-127 | (Method L1): Rₜ = 3.55 | 1H NMR (300 MHz, DMSO-d6) δ 9.10 (t, J = |
| | min; m/z = 505 (M + H)⁺ | 6.1 Hz, 1H), 8.64 (d, J = 2.2 Hz, 1H), 8.21-8.11 |
| | | (m, 3H), 7.85 (d, J = 8.1 Hz, 1H), 7.81-7.61 (m, |
| | | 3H), 7.45 (d, J = 7.4 Hz, 1H), 6.97 (d, J = 8.6 |
| | | Hz, 1H), 4.17-4.01 (m, 2H), 3.92 (s, 3H). |
| 1-128 | (Method L1): Rₜ = 3.44 | 1H NMR (300 MHz, DMSO-d6) δ 9.11 (t, J = |
| | min; m/z = 493 (M + H)⁺ | 6.1 Hz, 1H), 8.71 (d, J = 2.5 Hz, 1H), 8.51-8.41 |
| | | (m, 1H), 8.21 (d, J = 4.8 Hz, 2H), 7.89 (d, J = |
| | | 8.8 Hz, 1H), 7.81-7.61 (m, 3H), 7.45 (d, J = 7.0 |
| | | Hz, 1H), 7.36 (d, J = 8.6, 2.9 Hz, 1H), |
| | | 4.19-4.02 (m, 2H). |
| 1-131 | (Method L1): Rₜ = 3.30 | 1H NMR (300 MHz, DMSO-d6) δ 9.06 (t, J = |
| | min; m/z = 544 (M − H)⁻ | 6.2 Hz, 1H), 8.58 (s, 1H), 8.27 (s, 1H), |
| | | 8.13-8.03 (m, 2H), 7.85-7.60 (m, 4H), 7.44 (d, |
| | | J = 7.4 Hz, 1H), 5.28-5.10 (m, 2H), |
| | | 4.18-3.96 (m, 2H). |
| 1-132 | (Method L1): Rₜ = 3.09 | 1H NMR (300 MHz, DMSO-d6) δ 9.06 (t, J = |
| | min; m/z = 478 (M + H)⁺ | 6.3 Hz, 1H), 8.41 (s, 1H), 8.07 (d, J = 0.7 Hz, |
| | | 1H), 8.05-7.96 (m, 2H), 7.80-7.61 (m, 4H), |
| | | 7.43 (d, J = 7.3 Hz, 1H), 4.15-3.96 (m, 2H), |
| | | 3.88 (s, 3H). |
| 1-133 | (Method L1): Rₜ = 3.45 | 1H NMR (300 MHz, DMSO-d6) δ 13.17 (s, |
| | min; m/z = 462 (M − H)⁻ | 1H), 9.06 (t, J = 6.3 Hz, 1H), 8.62-8.01 (m, |
| | | 4H), 7.81-7.61 (m, 4H), 7.44 (d, J = 7.2 Hz, |
| | | 1H), 4.14-3.98 (m, 2H). |
| 1-134 | (Method L1): Rₜ = 3.70 | 1H NMR (300 MHz, DMSO-d6) δ 9.32 (d, J = |
| | min; m/z = 498 (M − H)⁻ | 2.3 Hz, 1H), 9.15-9.07 (m, 2H), 8.86 (t, J = 2.1 |
| | | Hz, 1H), 8.37-8.28 (m, 2H), 7.92 (d, J = 8.2 |
| | | Hz, 1H), 7.81-7.61 (m, 3H), 7.45 (d, J = 7.3 |
| | | Hz, 1H), 4.22-7.00 (m, 2H). |
| 1-137 | (Method L1): Rₜ = 3.20 | 1H NMR (300 MHz, DMSO-d6) δ 9.29 (t, J = |
| | min; m/z = 510 (M + H)⁺ | 6.0 Hz, 1H), 9.00 (d, J = 2.0 Hz, 1H), 8.81 (d, |
| | | J = 4.0 Hz, 1H), 8.73 (d, J = 2.2 Hz, 1H), 8.46 |
| | | (t, J = 2.1 Hz, 1H), 8.28 (d, J = 8.5 Hz, 2H), |
| | | 7.91 (d, J = 7.3, 3.6 Hz, 2H), 7.79 (d, J = 7.8, |
| | | 4.7 Hz, 1H), 4.23-4.02 (m, 2H). |
| 1-138 | (Method L1): Rₜ = 3.07 | 1H NMR (300 MHz, DMSO-d6) δ 9.28 (t, J = |
| | min; m/z = 494 (M + H)⁺ | 6.1 Hz, 1H), 8.93 (d, J = 1.6 Hz, 1H), 8.81 (d, |
| | | J = 4.0 Hz, 1H), 8.69 (d, J = 2.6 Hz, 1H), |
| | | 8.34-8.22 (m, 3H), 7.92 (d, J = 7.3 Hz, 2H), |
| | | 7.79 (d, J = 7.8, 4.7 Hz, 1H), 4.24-4.01 (m, 2H). |
| 1-139 | (Method L1): Rₜ = 3.23 | 1H NMR (300 MHz, DMSO-d6) δ 9.29 (t, J = |
| | min; m/z = 510 (M + H)⁺ | 6.2 Hz, 1H), 8.89 (d, J = 2.6 Hz, 1H), 8.82 (d, |
| | | J = 4.0 Hz, 1H), 8.34 (d, J = 8.4, 2.6 Hz, 1H), |
| | | 8.24 (d, J = 6.9 Hz, 2H), 7.98-7.87 (m, 2H), 7.80 |
| | | (d, J = 7.8, 4.7 Hz, 1H), 7.69 (d, J = 8.4 Hz, 1H), |
| | | 4.25-4.03 (m, 2H). |
| 1-140 | (Method L1): Rₜ = 3.14 | 1H NMR (300 MHz, DMSO-d6) δ 9.27 (t, J = |
| | min; m/z = 494 (M + H)⁺ | 6.2 Hz, 1H), 8.79 (d, J = 3.7 Hz, 1H), 8.70 (d, |
| | | J = 2.5 Hz, 1H), 8.45 (t, J = 8.3, 2.7 Hz, 1H), |
| | | 8.20 (d, J = 6.7 Hz, 2H), 7.90 (t, J = 7.0 Hz, 2H), |
| | | 7.78 (d, J = 7.8, 4.7 Hz, 1H), 7.35 (d, J = 8.6, |
| | | 2.8 Hz, 1H), 4.22-4.03 (m, 2H). |
| 1-142 | (Method L1): Rₜ = 3.09 | 1H NMR (300 MHz, DMSO-d6) δ 9.21 (t, J = |
| | min; m/z = 460 (M + H)⁺ | 5.9 Hz, 1H), 8.83 (d, J = 15.1, 3.6 Hz, 2H), 8.28 |
| | | (d, J = 8.4, 2.7 Hz, 1H), 7.93-7.83 (m, 2H), |
| | | 7.82-7.64 (m, 4H), 4.23-4.08 (m, 2H). |
| 1-143 | (Method L1): Rₜ = 2.99 | 1H NMR (300 MHz, DMSO-d6) δ 9.21 (s, 1H), |
| | min; m/z = 444 (M + H)+ | 8.79 (d, J = 4.6 Hz, 1H), 8.66 (s, 1H), 8.40 (td, |
| | | J = 8.2, 2.6 Hz, 1H), 7.89-7.64 (m, 5H), 7.34 (d, |
| | | J = 8.6, 2.8 Hz, 1H), 4.24-4.03 (m, 2H). |
| 1-146 | (Method L1): Rₜ = 3.27 | 1H NMR (300 MHz, DMSO-d6) δ 9.02 (t, J = |
| | min; m/z = 469 (M + H)⁺ | 6.3 Hz, 1H), 8.80 (s, 2H), 7.80-7.56 (m, 6H), |
| | | 7.38 (d, J = 7.3 Hz, 1H), 4.18-4.01 (m, 2H), |
| | | 3.18 (s, 6H). |
| 1-147 | (Method L1): Rₜ = 3.33 | 1H NMR (300 MHz, DMSO-d6) δ 9.22 (s, 2H), |
| | min; m/z = 460 (M + H)⁺ | 9.05 (t, J = 6.3 Hz, 1H), 7.97 (d, J = 12.4 Hz, |
| | | 1H), 7.87-7.60 (m, 5H), 7.38 (d, J = 7.4 Hz, |
| | | 1H), 4.19-4.04 (m, 2H). |
| 1-148 | (Method L1): Rₜ = 2.99 | 1H NMR (300 MHz, DMSO-d6) δ 9.29-9.14 |
| | min; m/z = 426 (M + H)⁺ | (m, 3H), 9.05 (t, J = 6.1 Hz, 1H), 7.96 (d, J = |
| | | 12.4 Hz, 1H), 7.87-7.60 (m, 5H), 7.39 (d, J = |
| | | 7.3 Hz, 1H), 4.21-4.04 (m, 2H). |
| 1-149 | (Method L1): Rₜ = 3.48 | 1H NMR (300 MHz, DMSO-d6) δ 9.08 (t, J = |
| | min; m/z = 519 (M + H)⁺ | 6.1 Hz, 1H), 8.83 (s, 2H), 8.11 (d, J = 6.8 Hz, |
| | | 2H), 7.72 (dq, J = 32.2, 8.3, 7.7 Hz, 4H), 7.44 |
| | | (d, J = 7.5 Hz, 1H), 4.15-4.00 (m, 2H), 3.19 (s, |
| | | 6H). |
| 1-150 | (Method L1): Rₜ = 3.40 | 1H NMR (300 MHz, DMSO-d6) δ 9.26 (s, 2H), |
| | min; m/z = 510 (M + H)⁺ | 9.11 (t, J = 6.3 Hz, 1H), 8.34 (s, 1H), 8.30 (d, |
| | | J = 8.4 Hz, 1H), 7.93 (d, J = 8.2 Hz, 1H), |
| | | 7.81-7.62 (m, 3H), 7.44 (d, J = 7.4 Hz, 1H), |
| | | 4.20-4.00 (m, 2H). |
| 1-151 | (Method L1): Rₜ = 3.10 | 1H NMR (300 MHz, DMSO-d6) δ 9.28 (s, 3H), |
| | min; m/z = 476 (M + H)⁺ | 9.12 (t, J = 6.2 Hz, 1H), 8.37-8.25 (m, 2H), |
| | | 7.93 (d, J = 8.2 Hz, 1H), 7.81-7.61 (m, 3H), |
| | | 7.45 (d, J = 7.3 Hz, 1H), 4.11 (td, J = 14.5, |
| | | 6.1 Hz, 2H). |
| 1-152 | (Method L1): Rₜ = 2.76 | 1H NMR (300 MHz, DMSO-d6) δ 9.34-9.24 |
| | min; m/z = 477 (M + H)⁺ | (m, 4H), 8.81 (d, J = 3.9 Hz, 1H), 8.37-8.26 (m, |
| | | 2H), 7.98-7.87 (m, 2H), 7.79 (d, J = 7.8, 4.7 |
| | | Hz, 1H), 4.24-4.03 (m, 2H). |
| 1-153 | (Method L1): Rₜ = 3.10 | 1H NMR (300 MHz, DMSO-d6) δ 9.32-9.22 |
| | min; m/z = 511 (M + H)⁺ | (m, 3H), 8.84-8.77 (m, 1H), 8.37-8.26 (m, |
| | | 2H), 7.97-7.88 (m, 2H), 7.79 (d, J = 7.8, 4.5 |
| | | Hz, 1H), 4.24-4.03 (m, 2H). |
| 1-154 | (Method L1): Rₜ = 2.94 | 1H NMR (300 MHz, DMSO-d6) δ 9.27-9.15 |
| | min; m/z = 461 (M + H)⁺ | (m, 3H), 8.79 (d, J = 4.7 Hz, 1H), 7.97 (d, J = |
| | | 12.4 Hz, 1H), 7.89-7.70 (m, 4H), 4.15 (td, J = |
| | | 14.9, 6.6 Hz, 2H). |
| 1-155 | (Method L1): Rₜ = 3.06 | 1H NMR (300 MHz, DMSO-d6) δ 9.21 (t, J = |
| | min; m/z = 460 (M + H)⁺ | 6.3 Hz, 1H), 8.97 (d, J = 1.9 Hz, 1H), 8.79 (d, |
| | | J = 4.7 Hz, 1H), 8.70 (d, J = 2.2 Hz, 1H), 8.38 |
| | | (t, J = 2.1 Hz, 1H), 7.98 7.65 (m, 5H), 4.15 (td, |
| | | J = 14.4, 6.0 Hz, 2H). |
| 1-156 | (Method L1): Rₜ = 2.92 | 1H NMR (300 MHz, DMSO-d6) δ 9.22 (t, J = |
| | min; m/z = 444 (M + H)⁺ | 6.1 Hz, 1H), 8.91 (d, J = 1.4 Hz, 1H), 8.80 (d, |
| | | J = 4.7 Hz, 1H), 8.67 (d, J = 2.6 Hz, 1H), |
| | | 8.26-8.17 (m, 1H), 7.98-7.67 (m, 5H), 4.16 (td, |
| | | J = 14.6, 6.4 Hz, 2H). |

LC-MS Methods
Method L1:
MS instrument type: Agilent Technologies 6130 Quadrupole LC-MS; HPLC instrument type: Agilent Technologies 1260 Infinity; column: Waters XSelect (C18, 50 × 2.1 mm, 3.5μ); flow: 0.8 mL/min; column temp: 35° C.; eluent A: 0.1% formic acid in acetonitrile; eluent B: 0.1% formic acid in water; lin. gradient: t = 0 min 5% A, t = 3.5 min 98% A, t = 6 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.
Method L2:
MS instrument type: Agilent Technologies LC/MSD SL; HPLC instrument type: Agilent
Technologies 1100 Series; column: Waters XSelect (C18, 50 × 2.1 mm, 3.5μ; flow: 0.8 mL/min;
column temp: 25° C.; eluent A: 95% acetonitrile + 5% ammoniumbicarbonate in water; eluent B: 10 mmM ammoniumbicarbonate in water pH = 9.0; lin. gradient: t = 0 min 5% A, t = 3.5 min 98% A, t = 6 min 98% A; detection: DAD (220-320 nm); detection: MSD (ESI pos/neg) mass range: 100-800.
NMR spektra (Method M2)
¹H-instrument type: Bruker DMX300 (¹H-NMR: 300 MHz); internal standard: tetramethylsilane; chemical shifts (δ) are displayed in parts per million [ppm]; the following abbreviations are used: s = singlet, d = doublet, t = triplet, q = quartet, m = multiplet, br. = broad; coupling constants are displayed in Hertz [Hz].

### BIOLOGICAL EXAMPLES

### Haemonchus contortus (HAEMCO)

### Assay

Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 larvae of the red stomach worm (Haemonchus contortus) are transferred into a test tube containing compound solution.

After 5 days percentage of larval mortality are recorded. 100% efficacy means all larvae are killed, 0% efficacy means no larvae are killed.

In this test for example, the following compounds from the preparation examples showed good activity of 100% at an application rate of 20 ppm: 1-33, 1-35, 1-37, 1-38, 1-39, 1-40, 1-42, 1-43, 1-44, 1-45, 1-47, 1-48, 1-53, 1-62, 1-66, 1-68, 1-69, 1-72, 1-73, 1-74, 1-75, 1-77, 1-78, 1-79, 1-80, 1-81, 1-82, 1-83, 1-84, 1-85, 1-86, 1-87, 1-88, 1-89, 1-90, 1-92, 1-93, 1-94, 1-96, 1-97, 1-98, 1-99, 1-100, 1-101, 1-102, 1-103, 1-106, 1-107, 1-111, 1-120, 1-122, 1-126, 1-127, 1-128.

In this test for example, the following compounds from the preparation examples showed good activity of 90% at an application rate of 20 ppm: 1-3, 1-5, 1-12, 1-14, 1-32, 1-41, 1-49, 1-67, 1-70, 1-71, 1-104, 1-110.

In this test for example, the following compounds from the preparation examples showed good activity of 80% at an application rate of 20 ppm: 1-1, 1-11, 1-16, 1-20, 1-27, 1-28, 1-56, 1-57, 1-58, 1-60, 1-64, 1-114, 1-121.

In this test for example, the following compounds from the preparation examples showed good activity of 90% at an application rate of 4 ppm: 1-15, 1-124.

### Cooperia curticei (COOPCU)

### Assay

Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 nematode larvae (Cooperia curticei) are transferred into a test tube containing the compound solution.

After 5 days percentage of larval mortality is recorded. 100% efficacy means all larvae are killed; 0% efficacy means no larvae are killed.

In this test for example, the following compounds from the preparation examples showed good activity of 100% at an application rate of 20 ppm: 1-9, 1-12, 1-14, 1-15, 1-16, 1-18, 1-19, 1-20, 1-21, 1-31, 1-32, 1-33, 1-34, 1-35, 1-37, 1-38, 1-39, 1-40, 1-41, 1-42, 1-43, 1-44, 1-45, 1-46, 1-47, 1-48, 1-49, 1-51, 1-52, 1-53, 1-55, 1-57, 1-58, 1-59, 1-60, 1-62, 1-64, 1-66, 1-68, 1-69, 1-70, 1-71, 1-72, 1-73, 1-74, 1-75, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-96, 1-97, 1-98, 1-99, 1-100, 1-101, 1-102, 1-106, 1-146, 1-107, 1-110, 1-111, 1-114, 1-120, 1-122, 1-126, 1-127, 1-128.

In this test for example, the following compounds from the preparation examples showed good activity of 90% at an application rate of 20 ppm: 1-24, 1-26, 1-50, 1-65, 1-105, 1-124.

In this test for example, the following compounds from the preparation examples showed good activity of 80% at an application rate of 20 ppm: 1-1, 1-13, 1-23, 1-27, 1-28, 1-36, 1-56, 1-121, 1-125.

In this test for example, the following compounds from the preparation examples showed good activity of 100% at an application rate of 4 ppm: 1-104.

In this test for example, the following compounds from the preparation examples showed good activity of 90% at an application rate of 4 ppm: 1-67, 1-149

### Nippostrongylus brasiliensis (NIPOBR)

### Assay

Adult Nippostrongylus brasiliensis were washed with saline buffer containing 100 U/ml penicillin, 0.1 mg/ml streptomycin and 2.5 μg/ml amphotericin B. Test compounds were dissolved in DMSO and worms were incubated in medium in a final concentration of 10 μg/ml (10 ppm). An aliquot of the medium was used to determine the acetylcholine esterase activity in comparison to a negative control. The principle of measuring acetylcholine esterase as readout for anthelmintic activity was described in Rapson et al (1986) and Rapson et al (1987).

For the following examples, activity (reduction of AChE compared to negative control) was 60% or higher at 10 ppm:

1-1; 1-9; 1-33; 1-37; 1-44; 1-45; 1-48; 1-49; 1-53; 1-68; 1-69; 1-70; 1-73; 1-74; 1-75; 1-77; 1-78; 1-82; 1-87; 1-88; 1-89; 1-90; 1-92; 1-93; 1-96; 1-97; 1-98; 1-99; 1-100; 1-101; 1-106; 1-107; 1-111

For the following examples, activity (reduction of AChE compared to negative control) was higher than 80% at 10 ppm: 1-1, 1-33, 1-37, 1-45, 1-53, 1-68, 1-69, 1-70, 1-73, 1-74, 1-77, 1-82, 1-93, 1-96, 1-97, 1-99, 1-100, 1-106, 1-107, 1-111.

### Meloidogyne incognita (MELGIN)

### Assay

Solvent: 125.0 parts by weight of acetone

To produce a suitable preparation of active compound, 1 part by weight of active compound is mixed with the stated amount of solvent, and the concentrate is diluted with water to the desired concentration.

Vessels are filled with sand, a solution of the active ingredient, a suspension containing eggs and larvae of the southern root-knot nematode (Meloidogyne incognita) and salad seeds. The salad seeds germinate and the seedlings grow. Galls develop in the roots.

After 14 days the nematicidal activity is determined on the basis of the percentage of gall formation. 100% means no galls were found and 0% means the number of galls found on the roots of the treated plants was equal to that in untreated control plants.

In this test, for example, the following compounds from the preparation examples showed good activity of 100% at an application rate of 20 ppm: 1-39, 1-47, 1-69, 1-70, 1-73, 1-81, 1-82, 1-85, 1-86, 1-87, 1-88, 1-89, 1-93, 1-94, 1-104, 1-120, 1-128, 1-129, 1-130.

In this test, for example, the following compounds from the preparation examples showed good activity of 90% at an application rate of 20 ppm: 1-16, 1-19, 1-20, 1-24, 1-37, 1-42, 1-44, 1-48, 1-66, 1-68, 1-74, 1-80, 1-106, 1-126, 1-136, 1-140.

### In Vivo Efficacy Test

Haemonchus contortus/Trichostrongylus colubriformis/gerbil

Gerbils, experimentally infected with Haemonchus and/or Trichostrongylus, were treated once during late prepatency. Test compounds were formulated as solutions or suspensions and applied intraperitoneally or subcutaneously.

Efficacy was determined per group as reduction of worm count in stomach and small intestine, respectively, after necropsy compared to worm count in an infected and placebo-treated control group.

The following examples were tested and had an activity of 90% or higher after intraperitoneal treatment at the given dosage:

| Treatment | Haemonchus | Trichostrongylus |
|---|---|---|
| 20 mg/kg | 1-69; 1-78; 1-96 | 1-69 |
| intraperitoneally | | |
| 20 mg/kg | 1-93 | 1-93 |
| subcutaneously | | |

### In Vitro Efficacy Test

In vitro assay targeting Dirofilaria immitis microfilariae

≧250 Dirofilaria immitis microfilariae, which were freshly purified from blood, were added to wells of a microtitre plate containing a nutrient medium and the test compound in DMSO. Compounds were tested in a five point concentration-response assay in duplicate. Larvae exposed to DMSO and no test compounds were used as negative controls. Larvae were evaluated after 72 h of incubation with the compound. Efficacy was determined as the reduction of motility in comparison to the negative control. Based on the evaluation of five concentrations, concentration-response curves as well as EC₅₀-values were calculated.

For the following examples, the EC50 was <5 ppm: 1-70; 1-73; 1-74; 1-77; 1-78; 1-89; 1-93; 1-99; 1-100; 1-101

For the following examples, the EC₅₀ was <2.5 ppm: 1-93

### COMPARATIVE EXAMPLES

### Haemonchus contortus-Test (HAEMCO)

Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 larvae of the red stomach worm (Haemonchus contortus) are transferred into a test tube containing compound solution.

After 5 days percentage of larval mortality are recorded. 100% efficacy means all larvae are killed, 0% efficacy means no larvae are killed.

In this test, for example, the following compounds from the preparation examples show a superior level of activity compared to the prior state of the art: see following table 4.

TABLE 4

| | | | | % | |
|---|---|---|---|---|---|
| Substance | Structure | Object | Concentration | Efficacy | dat |
| Ex.-No. 1-49 Known from WO2012/118139 | 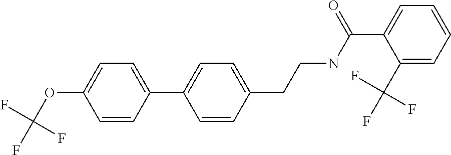 [SMILES: O=C(NCCc1ccc(-c2ccc(OC(F)(F)F)cc2)cc1)c1ccccc1C(F)(F)F] | HAEMCO | 20 ppm 4 ppm 0.8 ppm | 80 60 60 | 5 dat 5 dat 5 dat |
| Ex.-No. 1-37 According to the invention | 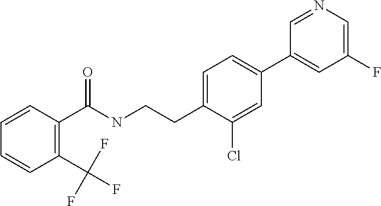 [SMILES: O=C(NCCc1ccc(-c2cncc(F)c2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-38 According to the invention | 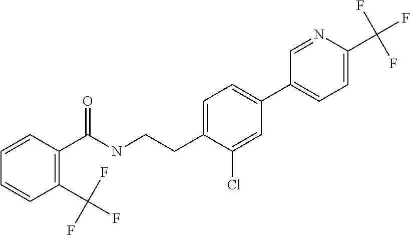 [SMILES: O=C(NCCc1ccc(-c2ccc(C(F)(F)F)nc2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-43 According to the invention | 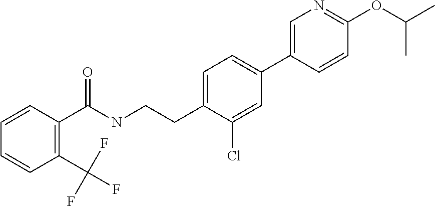 [SMILES: CC(C)Oc1ccc(-c2ccc(CCNC(=O)c3ccccc3C(F)(F)F)c(Cl)c2)cn1] | HAEMCO | 20 ppm 4 ppm 0.8 ppm | 100 100 100 | 5 dat 5 dat 5 dat |
| Ex.-No. 1-44 According to the invention | 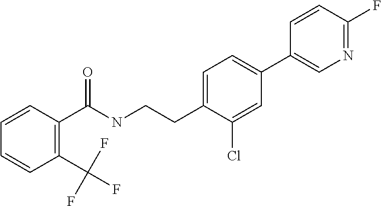 [SMILES: O=C(NCCc1ccc(-c2ccc(F)nc2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-45 According to the invention | 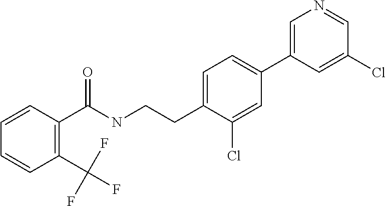 [SMILES: O=C(NCCc1ccc(-c2cncc(Cl)c2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-47 According to the invention | 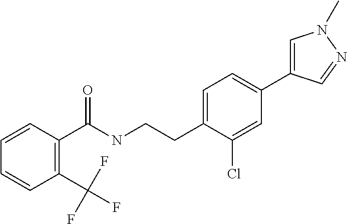 [SMILES: Cn1cc(-c2ccc(CCNC(=O)c3ccccc3C(F)(F)F)c(Cl)c2)cn1] | HAEMCO | 20 ppm 4 ppm 0.8 ppm | 100 100 80 | 5 dat 5 dat 5 dat |
| Ex.-No. 1-48 According to the invention | 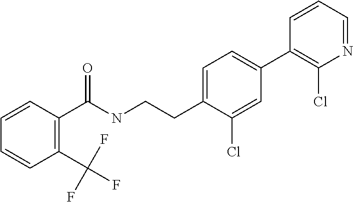 [SMILES: O=C(NCCc1ccc(-c2cccnc2Cl)cc1Cl)c1ccccc1C(F)(F)F] | HAEMCO | 20 ppm 4 ppm 0.8 ppm | 100 100 80 | 5 dat 5 dat 5 dat |
| Ex.-No. 1-35 According to the invention | 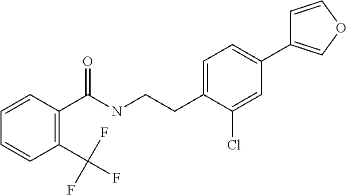 [SMILES: O=C(NCCc1ccc(-c2ccoc2)cc1Cl)c1ccccc1C(F)(F)F] | HAEMCO | 20 ppm 4 ppm 0.8 ppm | 100 90 80 | 5 dat 5 dat 5 dat |
| Ex.-No. 1-62 According to the invention | 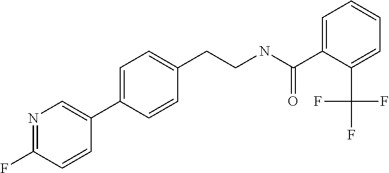 [SMILES: O=C(NCCc1ccc(-c2ccc(F)nc2)cc1)c1ccccc1C(F)(F)F] | HAEMCO | 20 ppm 4 ppm 0.8 ppm | 100 90 80 | 5 dat 5 dat 5 dat |
| Ex.-No. 1-69 According to the invention | 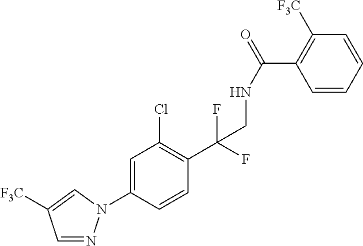 [SMILES: O=C(NCC(F)(F)c1ccc(-n2cc(C(F)(F)F)cn2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-74 According to the invention | 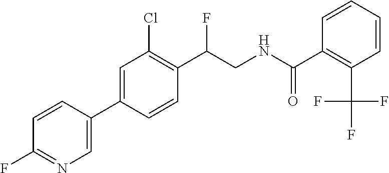 [SMILES: O=C(NCC(F)c1ccc(-c2ccc(F)nc2)cc1Cl)c1ccccc1C(F)(F)F] | HAEMCO | 20 ppm 4 ppm 0.8 ppm | 100 100 100 | 5 dat 5 dat 5 dat |
| Ex.-No. 1-93 According to the invention | 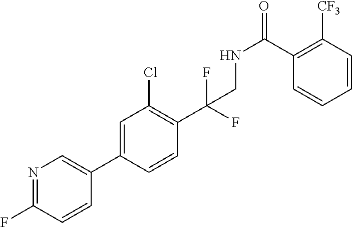 [SMILES: O=C(NCC(F)(F)c1ccc(-c2ccc(F)nc2)cc1Cl)c1ccccc1C(F)(F)F] | HAEMCO | 20 ppm 4 ppm 0.8 ppm | 100 100 100 | 5 dat 5 dat 5 dat |

1. A compound of formula (I) 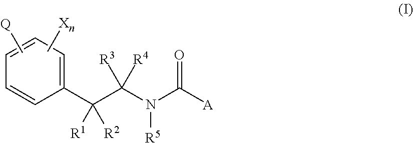
wherein
n is 0, 1, 2, 3 or 4, limited by the number of available positions in the ring to which a substituent X can be connected,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkyl carbonyl amino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic or partially saturated or saturated, 5- or 6-membered heterocyclic ring containing one to four heteroatoms chosen from N, S, and O bearing the substituent Ym with
m is 0, 1, 2, 3 or 4, limited by the number of available positions in Q to which a substituent Y can be connected, and
each Y is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), —NH(C₁-C₈-alkylcarbonyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
R¹, R², R³ and R⁴ are the same or different and are selected from the group consisting of hydrogen, halogen, cyano, hydroxy, amino, —SH, —CHO, —OCHO, —NHCHO, —COOH, —CONH₂, —CONH(OH), —OCONH₂, a (hydroxyimino)-C₁-C₆-alkyl group, C₁-C₆-alkyl, C₂-C₆-alkenyl, C₂-C₆-alkynyl, C₁-C₆-alkylamino, di-(C₁-C₆-alkyl)amino, C₁-C₆-alkoxy, hydroxy-C₁-C₄-alkyl, C₁-C₄-alkoxy-C₁-C₃-alkyl, C₁-C₆-halogenoalkyl having 1 to 5 halogen atoms, C₁-C₆-halogenoalkoxy having 1 to 5 halogen atoms, C₂-C₆-alkenyloxy, C₂-C₆-halogenoalkenyloxy having 1 to 5 halogen atoms, C₃-C₆-alkynyloxy, C₃-C₆-halogenoalkynyloxy having 1 to 5 halogen atoms, C₃-C₆-cycloalkyl, C₃-C₆-halogenocycloalkyl having 1 to 5 halogen atoms, C₃-C₆-cycloalkyl-C₁-C₆-alkyl, C₃-C₆-halogenocycloalkyl-C₁-C₆-alkyl having 1 to 5 halogen atoms, C₁-C₆-alkylcarbonyl, C₁-C₆-halogenoalkylcarbonyl having 1 to 5 halogen atoms, —CONH(C₁-C₆-alkyl), —CON(C₁-C₆-alkyl)₂, —CONH(OC₁-C₆-alkyl), —CON(OC₁-C₆-alkyl)(C₁-C₆-alkyl), C₁-C₆-alkoxycarbonyl, a C₁-C₆-halogenoalkoxycarbonyl having 1 to 5 halogen atoms, —OC(O)—C₁-C₆-alkyl, —OC(O)—C₁-C₆-halogenoalkyl having 1 to 5 halogen atoms, —NHC(O)—C₁-C₆-alkyl, —NHC(O)—C₁-C₆-halogenoalkyl having 1 to 5 halogen atoms, —OCONH(C₁-C₆-alkyl), —OCON(C₁-C₆-alkyl)₂, —OCONH(OC₁-C₆-alkyl), OCO(OC₁-C₆-alkyl), —S—C₁-C₆-alkyl, —S—C₁-C₆-halogenoalkyl having 1 to 5 halogen atoms, —S(O)—C₁-C₆-alkyl, —S(O)—C₁-C₆-halogenoalkyl having 1 to 5 halogen atoms, —S(O)₂—C₁-C₆-alkyl, —S(O)₂—C₁-C₆-halogenoalkyl having 1 to 5 halogen atoms, benzyl, benzyloxy, —S-benzyl, —S(O)-benzyl, —S(O)₂-benzyl, benzyl amino, phenoxy, —S-phenyl, —S(O)-phenyl, —S(O)₂-phenyl, phenylamino, phenylcarbonylamino, 2,6-dichlorophenyl-carbonylamino, 2-chlorophenyl-carbonylamino and phenyl,
with the proviso that R¹ is fluorine and/or R² is fluorine,
R⁵ is selected from the group consisting of hydrogen, cyano, —CHO, —OH, C₁-C₆-alkyl, C₁-C₆-halogenoalkyl having 1 to 5 halogen atoms, C₁-C₆-alkoxy, C₁-C₆-halogenoalkoxy having 1 to 5 halogen atoms, C₃-C₇-cycloalkyl, C₃-C₇-halogenocycloalkyl having 1 to 5 halogen atoms, C₃-C₇-cycloalkyl-C₁-C₆-alkyl, —CONH(C₁-C₆-alkyl), C₂-C₆-alkenyl, C₂-C₆-alkynyl, C₁-C₆-alkoxy-C₁-C₆-alkyl, C₃-C₇-cycloalkyl-C₁-C₆-alkyl, cyano-C₁-C₆-alkyl, amino-C₁-C₆-alkyl, C₁-C₆-alkylamino-C₁-C₆-alkyl, di-(C₁-C₆-alkyl)amino-C₁-C₆-alkyl, C₁-C₆-alkylcarbonyl, C₁-C₆-halogenoalkylcarbonyl having 1 to 5 halogen atoms, C₁-C₆-alkoxycarbonyl, C₁-C₆-benzyloxycarbonyl, C₁-C₆-alkoxy-C₁-C₆-alkylcarbonyl, —S—C₁-C₆-alkyl, —S—C₁-C₆-halogenoalkyl having 1 to 5 halogen atoms, —S(O)₂—C₁-C₆-alkyl, and —S(O)₂—C₁-C₆-halogenoalkyl having 1 to 5 halogen atoms,
A represents a phenyl group of the formula (A1) 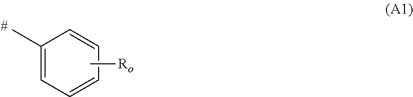
wherein
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1) 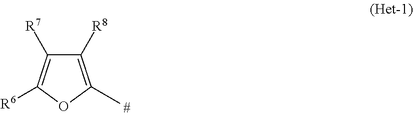
in which
R⁶ and R⁷ may be the same or different and are selected from the group consisting of hydrogen, halogen, amino, nitro, C₁-C₄-alkyl and C₁-C₄-halogenoalkyl having 1 to 5 halogen atoms, and
R⁸ is selected from the group consisting of hydrogen, halogen, nitro, C₁-C₄-alkyl and C₁-C₄-halogenoalkyl having 1 to 5 halogen atoms, or
A represents a heterocycle of the formula (Het-2) 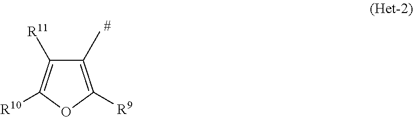
in which
R⁹ is selected from the group consisting of hydrogen, halogen, C₁-C₄-alkyl and C₁-C₄-halogenoalkyl having 1 to 5 halogen atoms, and
R¹⁰ and R¹¹ may be the same or different and are selected from the group consisting of hydrogen, halogen, amino, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-31 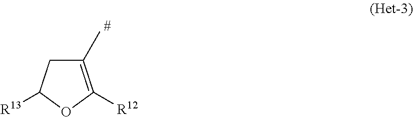
in which
R¹² is selected from the group consisting of halogen, C₁-C₄-alkyl and C₁-C₄-halogenoalkyl having 1 to 5 halogen atoms, and
R¹³ is selected from the group consisting of hydrogen, C₁-C₄-alkyl and C₁-C₄-halogenoalkyl having 1 to 5 halogen atoms, or
A represents a heterocycle of the formula (Het-4) 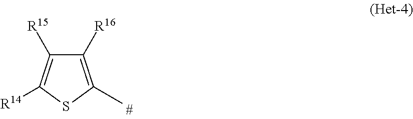
in which
R¹⁴ and R¹⁵ may be the same or different and are selected from the group consisting of hydrogen, halogen, C₁-C₄-alkyl, C₁-C₄-halogenoalkyl having 1 to 5 halogen atoms, —S—C₁-C₄-alkyl, —S(O)₂—C₁-C₄-alkyl, phenyl (optionally substituted by halogen or C₁-C₄-alkyl) and pyridyl (optionally substituted by halogen or C₁-C₄-alkyl), and
R¹⁶ is selected from the group consisting of hydrogen, halogen, cyano, C₁-C₄-alkyl, C₁-C₄-halogenoalkyl having 1 to 5 halogen atoms and C₁-C₄-halogenoalkoxy having 1 to 5 halogen atoms, or
A represents a heterocycle of the formula (Het-5) 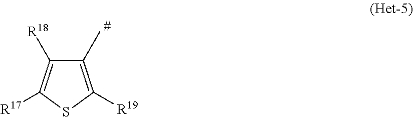
in which
R¹⁷ and R¹⁸ may be the same or different and are selected from the group consisting of hydrogen, halogen, C₁-C₄-alkyl, C₁-C₄-alkyloxy and C₁-C₄-halogenoalkyl having 1 to 5 halogen atoms, and
R¹⁹ is selected from the group consisting of hydrogen, halogen, C₁-C₄-alkyl and C₁-C₄-halogenoalkyl having 1 to 5 atoms, or
A represents a heterocycle of the formula (Het-6) 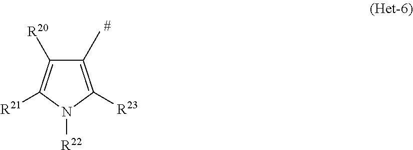
in which
R²⁰ is selected from the group consisting of hydrogen, halogen, cyano, C₁-C₄-alkyl and C₁-C₄-halogenoalkyl having 1 to 5 halogen atoms, and
R²¹ and R²³ may be the same or different and are selected from the group consisting of hydrogen, halogen, C₁-C₄-alkyl and C₁-C₄-halogenoalky having 1 to 5 halogen atoms, and
R²² is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, hydroxyl-C₁-C₄-alkyl, —S(O)₂—C₁-C₄-alkyl, —S(O)₂—N(C₁-C₄-alkyl)₂, C₁-C₆-alkylcarbonyl, —S(O)₂-phenyl (optionally substituted by halogen or C₁-C₄-alkyl) and benzoyl (optionally substituted by halogen or C₁-C₄-alkyl), or
A represents a heterocycle of the formula (Het-7) 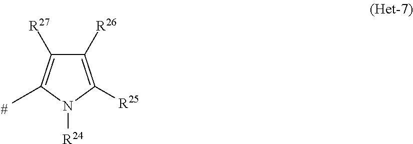
in which
R²⁴ is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, hydroxy-C₁-C₄-alkyl, —S(O)₂—C₁-C₄-alkyl, —S(O)₂—N(C₁-C₄-alkyl)₂, C₁-C₆-alkylcarbonyl, —S(O)₂-phenyl (optionally substituted by halogen or C₁-C₄-alkyl) and benzoyl (optionally substituted by halogen or a C₁-C₄-alkyl), and
R²⁵, R²⁶ and R²⁷ may be the same or different and are selected from the group consisting of hydrogen, halogen, cyano, C₁-C₄-alkyl, C₁-C₄-halogenalkyl having 1 to 5 halogen atoms and C₁-C₄-alkylcarbonyl, or
A represents a heterocycle of the formula (Het-8) 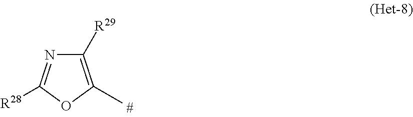
in which
R²⁸ is selected from the group consisting of hydrogen and C₁-C₄-alkyl, and
R²⁹ is selected from the group consisting of halogen, C₁-C₄-alkyl and C₁-C₄-halogenoalkyl having 1 to 5 halogen atoms, or
A represents a heterocycle of the formula (Het-9) 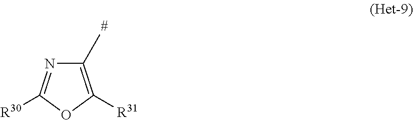
in which
R³⁰ is selected from the group consisting of hydrogen and C₁-C₄-alkyl, and
R³¹ is selected from the group consisting of halogen, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-10) 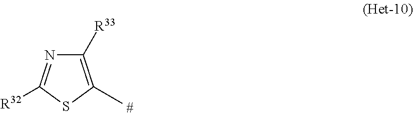
in which
R³² is selected from the group consisting of hydrogen, halogen, amino, cyano, C₁-C₄-alkylamino, di-(C₁-C₄-alkyl)amino, C₁-C₄-alkyl, C₁-C₄-halogenoalkyl having 1 to 5 halogen atoms and phenyl (optionally substituted by halogen or C₁-C₄-alkyl), and
R³³ is selected from the group consisting of halogen, C₁-C₄-alkyl and C₁-C₄-halogenoalkyl having 1 to 5 halogen atoms, C₁-C₅-halogenoalkoxy having 1 to 9 halogen atoms, amino, substituted or unsubstituted C₁-C₅-alkylamino or substituted or unsubstituted di-(C₁-C₅-alkyl)-amino, or
A represents a heterocycle of the formula (Het-11) 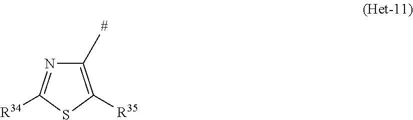
in which
R³⁴ is selected from the group consisting of hydrogen, halogen, amino, cyano, C₁-C₄-alkylamino, di-(C₁-C₄-alkyl)amino, C₁-C₄-alkyl and C₁-C₄-halogenoalkyl having 1 to 5 halogen atoms, and
R³⁵ is selected from the group consisting of halogen, C₁-C₄-alkyl and C₁-C₄-halogenoalkyl having 1 to 5 halogen atoms, or
A represents a heterocycle of the formula (Het-12) 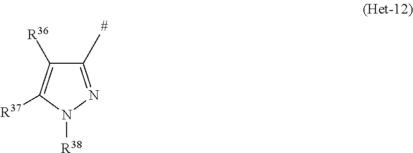
in which
R³⁶ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, —S(O)—C₁-C₄-alkyl, —S(O)₂—C₁-C₄-alkyl, —S—C₁-C₄-halogenoalkyl having 1 to 5 halogen atoms, aminocarbonyl and aminocarbonyl-C₁-C₄-alkyl, and
R³⁷ is selected from the group consisting of hydrogen, halogen, cyano, nitro, C₁-C₄-alkyl, C₁-C₄-alkoxy, —S—C₁-C₄-alkyl, —S(O)—C₁-C₄-alkyl, and —S(O)₂—C₁-C₄-alkyl, and
R³⁸ is selected from the group consisting of hydrogen, phenyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-13) 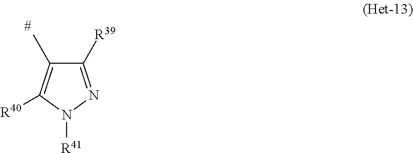
in which
R³⁹ is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, S(O)—C₁-C₄-alkyl, —S(O)₂—C₁-C₄-alkyl, —S—C₁-C₄-halogenoalkyl having 1 to 5 atoms, aminocarbonyl and aminocarbonyl-C₁-C₄-alkyl, and
R⁴⁰ is selected from the group consisting of hydrogen, halogen, cyano, C₁-C₄-alkyl, C₁-C₄-alkoxy, C₁-C₄-halogenoalkoxy having 1 to 5 halogen atoms, —S—C₁-C₄-alkylS(O)—C₁-C₄-alkyl, and —S(O)₂—C₁-C₄-alkyl, and
R⁴¹ is selected from the group consisting of hydrogen, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl, C₁-C₄-halogenoalkoxy-C₁-C₄-alkyl having 1 to 5 halogen atoms and phenyl (optionally substituted by halogen, C₁-C₄-alkyl, C₁-C₄-alkoxy-C₁-C₄-alkyl or nitro), or
A represents a heterocycle of the formula (Het-14) 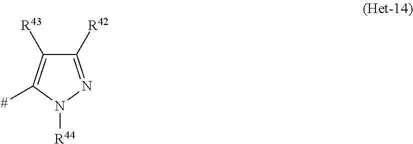
in which
R⁴² is selected from the group consisting of hydrogen, halogen, cyano, nitro, C₁-C₄-alkyl, C₁-C₄-halogenoalkyl having 1 to 5 halogen atoms, C₃-C₆-cycloalkyl, C₁-C₄-alkoxy, C₁-C₄-halogenoalkoxy having 1 to 5 halogen atoms, —S—C₁-C₄-alkyl, S(O)—C₁-C₄-alkyl, —S(O)₂—C₁-C₄-alkyl, —S—C₁-C₄-halogenoalkyl having 1 to 5 halogen atoms, aminocarbonyl and aminocarbonyl-C₁-C₄-alkyl, and
R⁴³ is selected from the group consisting of hydrogen, halogen, cyano, C₁-C₄-alkyl, C₁-C₄-alkoxy, —S—C₁-C₄-alkyl, S(O)—C₁-C₄-alkyl, —S(O)₂—C₁-C₄-alkyl, and C₁-C₄-halogenoalkyl having 1 to 5 halogen atoms, and
R⁴⁴ is selected from the group consisting of hydrogen, phenyl, benzyl, C₁-C₄-alkyl, C₁-C₄-halogenoalkyl having 1 to 5 halogen atoms, hydroxy-C₁-C₄-alkyl, C₂-C₆-alkenyl, C₃-C₆-cycloalkyl, C₁-C₄-alkylthio-C₁-C₄-alkyl, C₁-C₄-alkyl-S(O)—C₁-C₄-alkyl, C₁-C₄-alkyl-S(O)₂—C₁-C₄-alkyl, C₁-C₄-halogenoalkylthio-C₁-C₄-alkyl having 1 to 5 halogen atoms, C₁-C₄-alkoxy-C₁-C₄-alkyl and C₁-C₄-halogenoalkoxy-C₁-C₄-alkyl having 1 to 5 halogen atoms, or
A represents a heterocycle of the formula (Het-15) 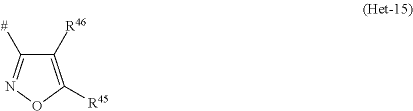
in which
R⁴⁵ and R⁴⁶ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-16) 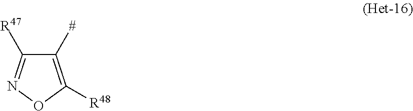
in which
R⁴⁷ and R⁴⁸ may be the same or different and are selected from the group consisting of hydrogen, halogen, C₁-C₄-alkyl, C₁-C₄-halogenoalkyl having 1 to 5 halogen atoms, phenyl (optionally substituted by halogen or a C₁-C₄-alkyl), and heterocyclyl like pyridyl, pyrimidinyl and thiadiazolyl (each optionally substituted by halogen or C₁-C₄-alkyl), or
A represents a heterocycle of the formula (Het-17) 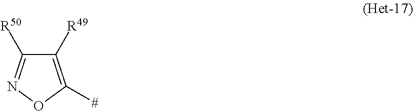
in which
R⁴⁹ and R⁵⁰ may be the same or different and are selected from the group consisting of hydrogen, halogen, C₁-C₄-alkyl and C₁-C₄-halogenoalkyl having 1 to 5 halogen atoms, or
A represents a heterocycle of the formula (Het-18) 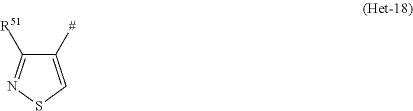
in which
R⁵¹ is selected from the group consisting of halogen, C₁-C₄-alkyl and C₁-C₄-halogenoalkyl having 1 to 5 halogen atoms, or
A represents a heterocycle of the formula (Het-19) 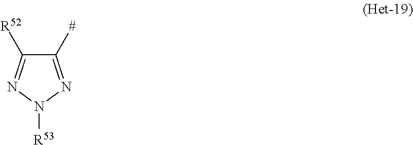
in which
R⁵² is selected from the group consisting of halogen, C₁-C₄-alkyl and C₁-C₄-halogenoalkyl having 1 to 5 halogen atoms, and
R⁵³ is selected from the group consisting of hydrogen, C₁-C₄-alkyl, C₁-C₄-halogenoalkyl having 1 to 5 halogen atoms and phenyl (optionally substituted by halogen or C₁-C₄-alkyl), or
A represents a heterocycle of the formula (Het-20) 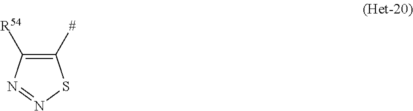
in which
R⁵⁴ is selected from the group consisting of halogen, C₁-C₄-alkyl and C₁-C₄-halogenoalkyl having 1 to 5 halogen atoms, or
A represents a heterocycle of the formula (Het-21) 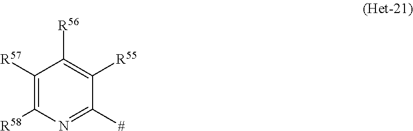
in which
R⁵⁵ is selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, S(O)—C₁-C₄-alkyl, —S(O)₂—C₁-C₄-alkyl, —S—C₁-C₄-halogenoalkyl having 1 to 5 halogen atoms and C₁-C₄-halogenoalkoxy having 1 to 5 halogen atoms, with the proviso that for Het-21, R⁵⁵ is not CF₃, and
R⁵⁶, R⁵⁷ and R⁵⁸, which may be the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-22) 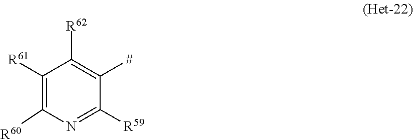
in which
R⁵⁹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and S-phenyl (optionally substituted by halogen or C₁-C₄-alkyl), and
R⁶⁰, R⁶¹ and R⁶², which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, N-morpholine optionally substituted by halogen or C₁-C₄-alkyl, and thienyl (optionally substituted by halogen or a C₁-C₄-alkyl), or
A represents a heterocycle of the formula (Het-23) 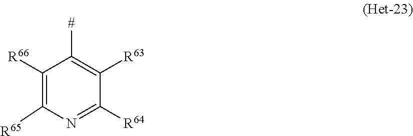
in which
R⁶³, R⁶⁴, R⁶⁵ and R⁶⁶, which may be the same or different, are selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, —S—C₁-C₄-halogenoalkyl having 1 to 5 halogen atoms, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-24) 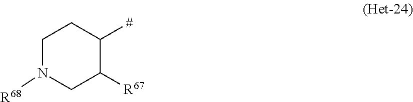
in which
R⁶⁷ is selected from the group consisting of hydrogen, halogen, C₁-C₄-alkyl and C₁-C₄-halogenoalkyl having 1 to 5 halogen atoms, and
R⁶⁸ is selected from the group consisting of hydrogen, C₁-C₄-alkyl, C₁-C₄-halogenoalkyl having 1 to 5 halogen atoms, C₁-C₆-alkoxycarbonyl, benzyl (optionally substituted by 1 to 3 halogen atoms), benzyloxycarbonyl (optionally substituted by 1 to 3 halogen atoms), and heterocyclyl like pyridyl and pyrimidinyl (each optionally substituted by halogen, C₁-C₄-alkyl, C₁-C₄-halogenoalkyl having 1 to 5 halogen atoms), or
A represents a heterocycle of the formula (Het-25) 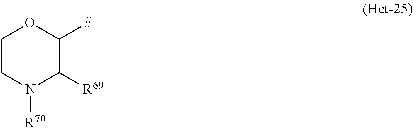
in which
R⁶⁹ is selected from the group consisting of hydrogen, halogen, hydroxy, cyano, C₁-C₄-alkyl, C₁-C₄-halogenoalkyl having 1 to 5 halogen atoms, C₁-C₄-alkoxy, —S—C₁-C₄-alkyl, —S—C₁-C₄-halogenoalkyl having 1 to 5 halogen atoms and C₁-C₄-halogenoalkoxy having 1 to 5 halogen atoms, and
R⁷⁰ is selected from the group consisting of hydrogen, C₁-C₄-alkyl, C₁-C₄-halogenoalkyl having 1 to 5 halogen atoms, and benzyl, or
A represents a heterocycle of the formula (Het-26) 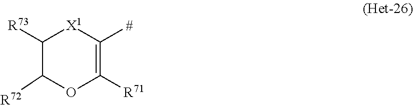
in which
X¹ is selected from the group consisting of sulphur, —SO—, —SO₂— and —CH₂—, and
R⁷¹ is selected from the group consisting of C₁-C₄-alkyl and C₁-C₄-halogenoalkyl having 1 to 5 halogen atoms, and
R⁷² and R⁷³ may be the same or different and are selected from the group consisting of hydrogen and C₁-C₄-alkyl, or
A represents a heterocycle of the formula (Het-27) 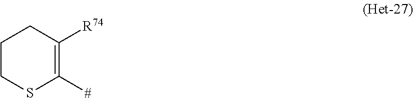
in which
R⁷⁴ is selected from the group consisting of C₁-C₄-alkyl and C₁-C₄-halogenoalkyl having 1 to 5 halogen atoms, or
A represents a heterocycle of the formula (Het-28) 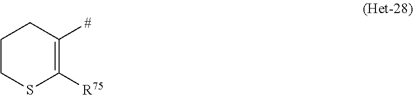
in which
R⁷⁵ is selected from the group consisting of C₁-C₄-alkyl and C₁-C₄-halogenoalkyl having 1 to 5 halogen atoms, or
A represents a heterocycle of the formula (Het-29) 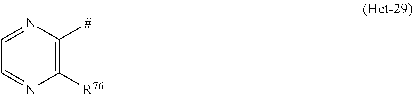
in which
R⁷⁶ is selected from the group consisting of hydrogen, halogen, C₁-C₄-alkyl and C₁-C₄-halogenoalkyl having 1 to 5 halogen atoms,
for controlling nematodes and/or other helminths.

2. The compound of claim 1,
wherein X, n, R¹, R², R³, R⁴, R⁵ and A have the meanings as defined in claim 1;
Q represents an optionally mono- or polysubstituted heteroaromatic ring from the group consisting of Q-1 to Q-64: 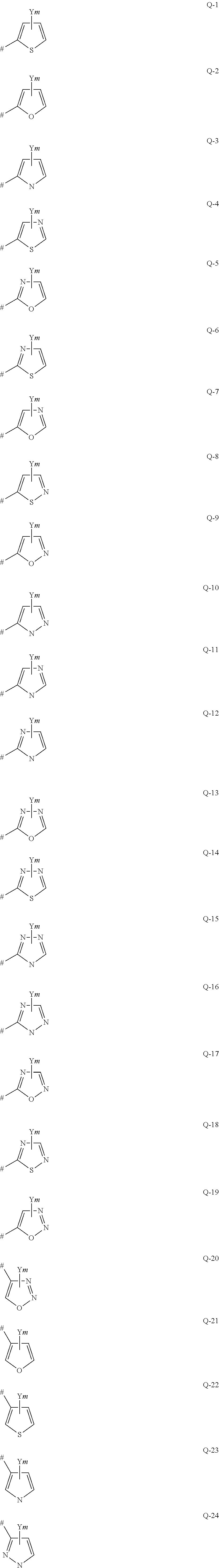 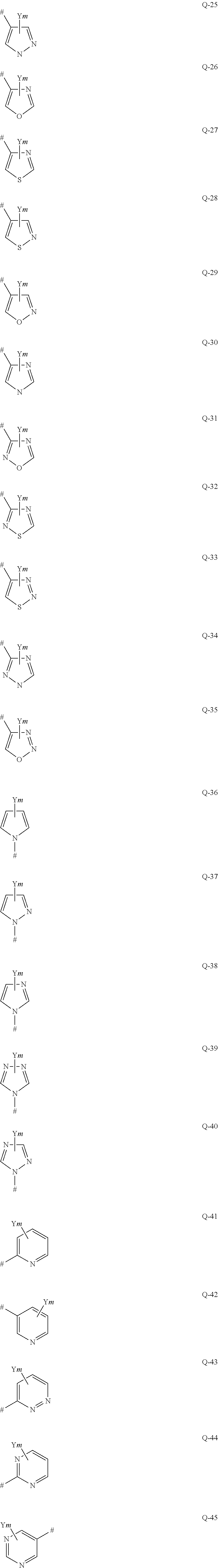 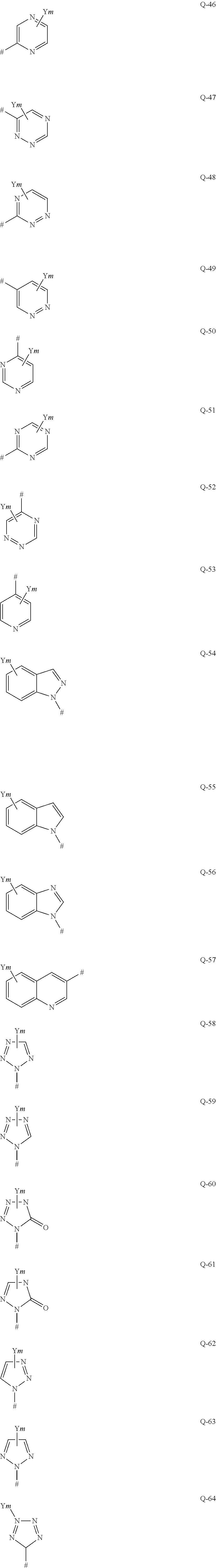
with
m is 0, 1 or 2, limited by the number of available positions in Q to which a substituent Y can be connected, and
each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), —NH(C₁-C₄-alkylcarbonyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyl oxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino.

3. The compound of claim 1, wherein
n is 1 or 2,
each X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents an optionally mono- or polysubstituted heteroaromatic ring from the group consisting of Q-4, Q-11, Q-21, Q-22, Q-25, Q-36, Q-37, Q-38, Q-40, Q-41, Q-42, Q-45, Q-53, Q-58, Q-62, Q-63 and Q-64,
with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine, bromine, iodine, cyano, —OCH₃, —OCH₂CH₃, —OCH(CH₃)₂, —OCH₂CF₃, S(O)₂—CH₃, NHC(O)CH₃, NHCH₃ and N(CH₃)₂,
R¹ and R² are the same or different and are selected from the group consisting of hydrogen, halogen, cyano, hydroxy, C₁-C₄-alkyl, C₂-C₄-alkenyl, C₂-C₄-alkynyl, C₁-C₄-alkoxy, C₃-C₆-cycloalkyl-C₁-C₃-alkyl, C₁-C₄-alkoxycarbonyl, —OC(O)—C₁-C₄-alkyl, —NHC(O)—C₁-C₄-alkyl, and phenyl,
with the proviso that R¹ is fluorine and/or R² is fluorine,
R³ and R⁴ are the same or different and are selected from the group consisting of hydrogen, —COOH, C₁-C₄-alkyl, C₁-C₄-halogenoalkyl C₁-C₄-alkoxy, hydroxy-C₁-C₄-alkyl, C₁-C₄-alkoxy-C₁-C₃-alkyl, —CONH(C₁-C₄-alkyl), C₁-C₄-alkoxycarbonyl, —OC(O)—C₁-C₄-alkyl, and phenyl,
R⁵ is selected from the group consisting of hydrogen, C₁-C₄-alkyl, C₃-C₆-cycloalkyl, C₁-C₄-alkoxy, C₁-C₄-alkoxy-C₁-C₄-alkyl, C₁-C₄-alkylcarbonyl, C₁-C₄-alkoxycarbonyl,
A represents a phenyl group of formula (A1) 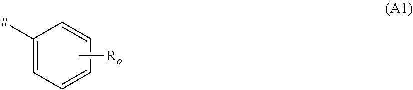
wherein
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1) 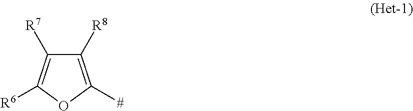
in which
R⁶ and R⁷ may be the same or different and are selected from the group consisting of hydrogen, halogen, nitro, C₁-C₄-alkyl and C₁-C₄-halogenoalkyl having 1 to 5 halogen atoms, and
R⁸ is selected from the group consisting of hydrogen, halogen, C₁-C₄-alkyl and C₁-C₄-halogenoalkyl having 1 to 5 halogen atoms, or
A represents a heterocycle of the formula (Het-2) 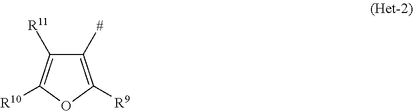
in which
R⁹ is selected from the group consisting of hydrogen, halogen, C₁-C₄-alkyl and C₁-C₄-halogenoalkyl having 1 to 5 halogen atoms, and
R¹⁰ and R¹¹ may be the same or different and are selected from the group consisting of hydrogen, halogen, C₁-C₄-alkyl and C₁-C₄-halogenoalkyl having 1 to 5 halogen atoms, phenyl optionally substituted by halogen or C₁-C₄-alkyl), or
A represents a heterocycle of the formula (Het-4) 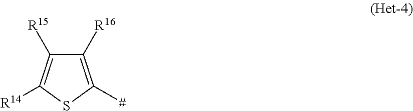
in which
R¹⁴ and R¹⁵ may be the same or different and are selected from the group consisting of hydrogen, halogen, C₁-C₄-alkyl, C₁-C₄-halogenoalkyl having 1 to 5 halogen atoms, —S—C₁-C₄-alkyl, —S(O)₂—C₁-C₄-alkyl, phenyl (optionally substituted by halogen or C₁-C₄-alkyl) and pyridyl (optionally substituted by halogen or C₁-C₄-alkyl), and
R¹⁶ is selected from the group consisting of hydrogen, halogen, cyano, C₁-C₄-alkyl, C₁-C₄-halogenoalkyl having 1 to 5 halogen atoms and C₁-C₄-halogenoalkoxy having 1 to 5 halogen atoms, or
A represents a heterocycle of the formula (Het-5) 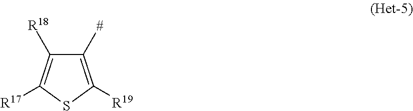
in which
R¹⁷ and R¹⁸ may be the same or different and are selected from the group consisting of hydrogen, halogen, C₁-C₄-alkyl, C₁-C₄-alkyloxy and C₁-C₄-halogenoalkyl having 1 to 5 halogen atoms, and
R¹⁹ is selected from the group consisting of hydrogen, halogen, C₁-C₄-alkyl and C₁-C₄-halogenoalkyl having 1 to 5 atoms, or
A represents a heterocycle of the formula (Het-6) 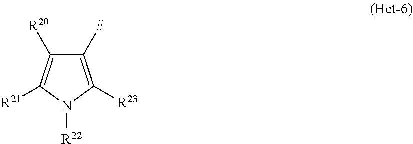
in which
R²⁰ is selected from the group consisting of hydrogen, halogen, cyano, C₁-C₄-alkyl and C₁-C₄-halogenoalkyl having 1 to 5 halogen atoms, and
R²¹ and R²³ may be the same or different and are selected from the group consisting of hydrogen, halogen, C₁-C₄-alkyl and C₁-C₄-halogenoalky having 1 to 5 halogen atoms, and
R²² is selected from the group consisting of hydrogen, cyano, C₁-C₄-alkyl, C₁-C₄-halogenoalkyl having 1 to 5 halogen atoms, C₁-C₄-alkoxy-C₁-C₄-alkyl, or
A represents a heterocycle of the formula (Het-10) 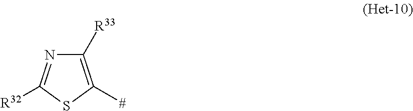
in which
R³² is selected from the group consisting of hydrogen, halogen, amino, cyano, C₁-C₄-alkylamino, di-(C₁-C₄-alkyl)amino, C₁-C₄-alkyl, C₁-C₄-halogenoalkyl having 1 to 5 halogen atoms and phenyl (optionally substituted by halogen or C₁-C₄-alkyl), and
R³³ is selected from the group consisting of halogen, C₁-C₄-alkyl and C₁-C₄-halogenoalkyl having 1 to 5 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, or
A represents a heterocycle of the formula (Het-21) 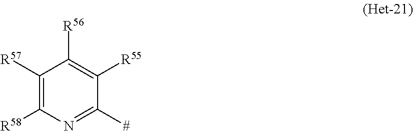
in which
R⁵⁵ is selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, —S(O)—C₁-C₄-alkyl, —S(O)₂—C₁-C₄-alkyl, —S—C₁-C₄-halogenoalkyl having 1 to 5 halogen atoms and C₁-C₄-halogenoalkoxy having 1 to 5 halogen atoms, with the proviso that for Het-21, R⁵⁵ is not CF₃, and
R⁵⁶, R⁵⁷ and R⁵⁸, which may be the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl and S(O)₂—C₁-C₄-alkyl, or
A represents a heterocycle of the formula (Het-22) 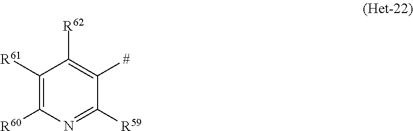
in which
R⁵⁹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and S-phenyl (optionally substituted by halogen or C₁-C₄-alkyl), and
R⁶⁰, R⁶¹ and R⁶², which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, N-morpholine (optionally substituted by halogen or C₁-C₄-alkyl) and thienyl (optionally substituted by halogen or a C₁-C₄-alkyl), or
A represents a heterocycle of the formula (Het-29) 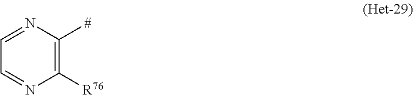
in which
R⁷⁶ is selected from the group consisting of hydrogen, halogen, C₁-C₄-alkyl and C₁-C₄-halogenoalkyl having 1 to 5 halogen atoms.

4. The compound of claim 1, wherein
n is 1 or 2,
each X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q is selected from: 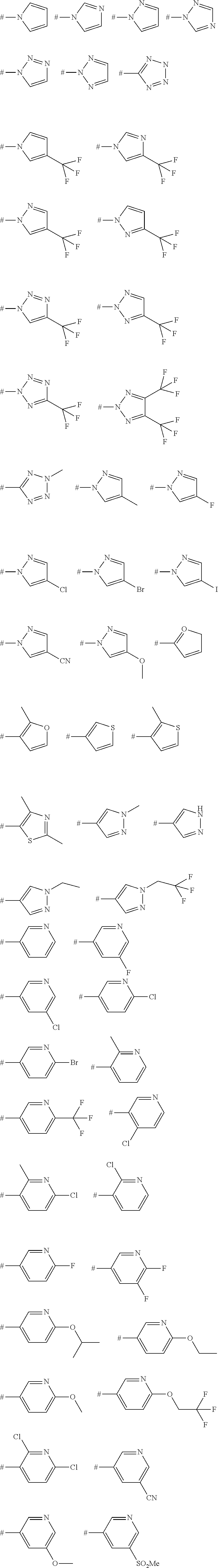 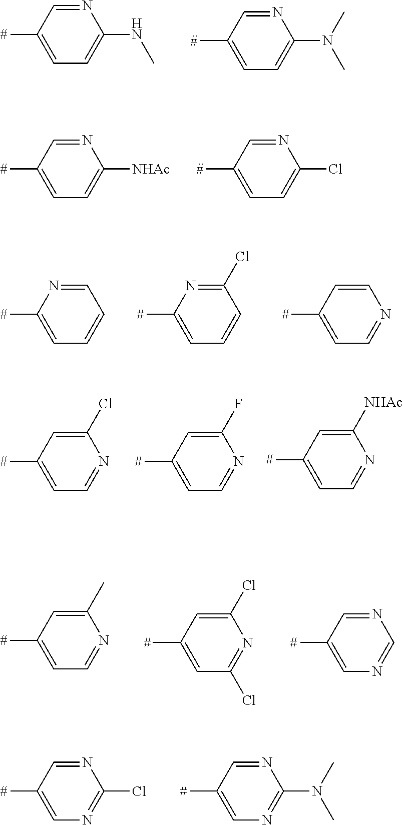
R¹ and R² are the same or different and are selected from the group consisting of hydrogen, methyl, ethyl, methoxy, ethoxy or fluorine,
with the proviso that R¹ is fluorine and/or R² is fluorine,
R³ and R⁴ are the same or different and are selected from the group consisting of hydrogen, methyl or ethyl,
R⁵ is hydrogen, and
A is selected from: 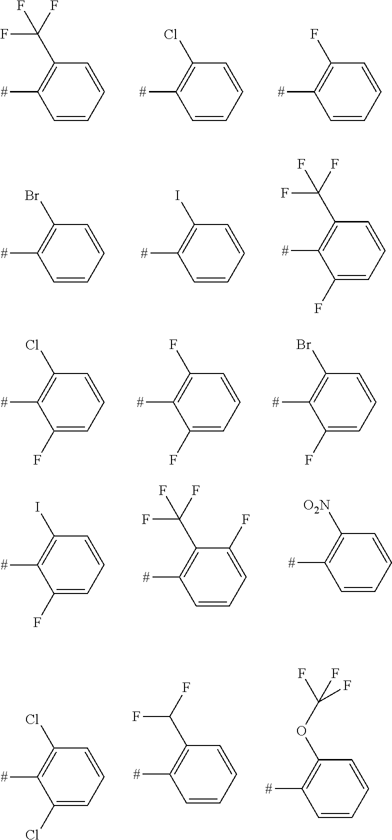
or
A is selected from:

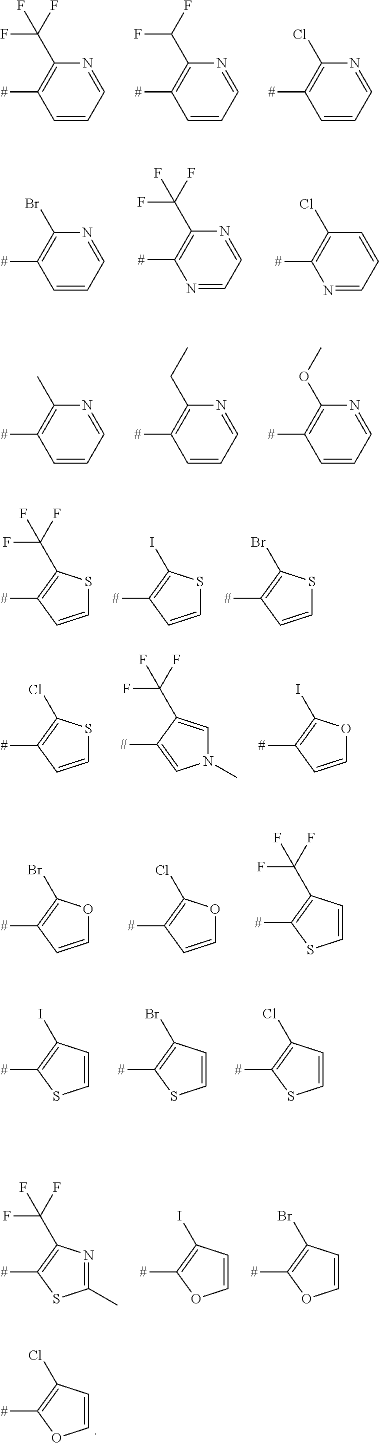

5. A method of protecting crops comprising administering an effective amount of the compound of claim 1.

6. A method of protecting animals comprising administering an effective amount of the compound of claim 1.

7. A compound of formula (Ia) 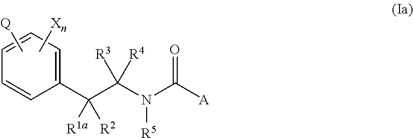
wherein
n, X, Q, R², R³, R⁴, R⁵ and A are as defined in claim 1, and R^{1a} is fluorine.

8. A compound of formula (Ib) 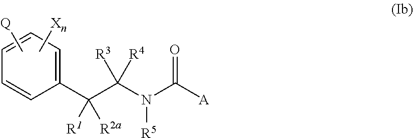
wherein
n, X, Q, R¹, R³, R⁴, R⁵ and A are as defined in claim 1, and R^{2a} is fluorine.

9. A compound of formula (Ic) 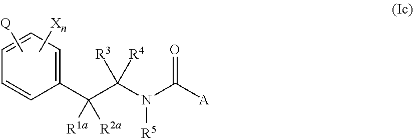
wherein
n, X, Q, R³, R⁴, R⁵ and A are as defined in claim 1, and both R^{1a} and R^{2a} are fluorine.

10. An agrochemical formulation, comprising the compound of claim 1.

11. An agrochemical formulation comprising the compound of claim 7.

12. The agrochemical formulation of claim 10, further comprising at least one extender and/or at least one surfactant.

13. The agrochemical formulation of claim 10, wherein the compound of the formula (I), (Ia), (Ib) or (Ic) is present in a mixture with at least one other active compound.

14. A method for controlling an animal pest, comprising applying an effective amount of the compound of formula (I) as defined in claim 1 to an animal pest and/or a habitat of the animal pest.

15. The method of claim 14, wherein the animal pest comprises a nematode or is a nematode.

16. A method for protecting a seed and/or a germinating plant from attack by a pest comprising the step of contacting the seed with a compound of formula (I) as defined in claim 1.

17. A seed obtained by the method of claim 16.

18. A method for controlling an animal pest comprising administering an effective amount of the compound of claim 7.

19. The method of claim 18, wherein the animal pest comprises a nematode or is a nematode.

20. A method of protecting crops or animals comprising administering an effective amount of the compound of claim 7.

21. A compound of formula (INT) 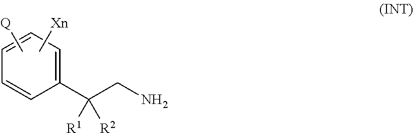
wherein R¹, R², Q, X and n are as defined in claim 1.
